(19) United States
(12) Patent Application Publication
(10) Pub. No.: US 2026/0078122 A1
(43) Pub. Date: Mar. 19, 2026

(54) INDAZOLE COMPOUNDS AS PKMYT1 KINASE INHIBITORS

(71) Applicant: Hoffmann-La Roche Inc., Little Falls, NJ (US)

(72) Inventors: Bo CHEN, Shanghai (CN); Fabian DEY, Zurich (CH); Xiaofeng LIU, Shanghai (CN); Tong MU, Shanghai (CN); Xuefei TAN, Shanghai (CN); Jun WU, Shanghai (CN); Jiasu XU, Shanghai (CN); Dan ZHAO, Shanghai (CN)

(73) Assignee: Hoffmann-La Roche Inc., Little Falls, NJ (US)

(21) Appl. No.: 19/309,956

(22) Filed: Aug. 26, 2025

Related U.S. Application Data
(63) Continuation of application No. PCT/EP2024/054734, filed on Feb. 26, 2024.

(30) Foreign Application Priority Data
Feb. 28, 2023 (WO) PCT/CN2023/078768
Jan. 22, 2024 (WO) PCT/CN2024/073481

(51) Int. Cl. C07D 471/04; A61K 31/4375; A61K 31/444; A61K 31/4545; A61K 31/4709; A61K 31/496; A61K 31/4985; A61K 31/4995; A61K 31/501; A61K 31/519; A61K 31/5377; C07B 59/00; C07D 401/04; C07D 491/107; C07D 519/00

(52) U.S. Cl. CPC C07D 471/04; A61K 31/4375; A61K 31/444; A61K 31/4545; A61K 31/4709; A61K 31/496; A61K 31/4985; A61K 31/4995; A61K 31/501; A61K 31/519; A61K 31/5377; C07B 59/002; C07D 401/04; C07D 491/107; C07D 519/00

(57) ABSTRACT

The present invention provides novel compounds having the general formula:

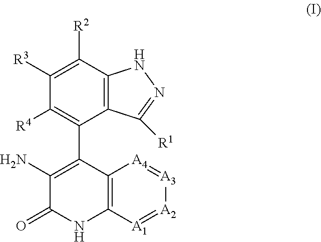

wherein R¹, R², R³, R⁴, A₁, A₂, A₃, and A₄ are as described herein, or a pharmaceutically acceptable salt thereof, compositions including the compounds and methods of using the compounds.

### CROSS-REFERENCE TO RELATED APPLICATIONS

This application is a Continuation of International Application No. PCT/EP2024/054734, filed Feb. 26, 2024, which claims the benefit of priority to International Application No. PCT/CN2023/078768, filed Feb. 28, 2023, and International Application No. PCT/CN2024/073481, filed Jan. 22, 2024, each of which is incorporated by reference herein in its entirety.

### FIELD OF THE INVENTION

The present invention relates to organic compounds, in particular to PKMYT1 (protein kinase, membrane-associated tyrosine and threonine kinase) kinase inhibitors, useful for treatment of cancers in a mammal. In particular, the present invention relates to indazole compounds that have PKMYT1 kinase inhibition activity, as well as their manufacture, pharmaceutical compositions containing them and their potential use as medicaments.

### BACKGROUND OF THE INVENTION

Cells are frequently experiencing both intrinsic and extrinsic genotoxic stresses that cause DNA damage and impair the integrity of our genome. To counter the potential genetic alterations caused by these DNA-damaging factors, cells need to establish various mechanisms to detect DNA lesions and repair DNA damage to maintain genome stability. This sophisticated network, referred to as the DNA damage response (DDR), is orchestrated by multiple highly evolutionarily conserved and coordinated signaling pathways that can correct different types of DNA lesions. One essential component involved in DDR machinery is the activation of cell cycle checkpoints that arrest the cell cycle at a specific stage until damaged DNA has been repaired. At least two types of checkpoints, G1/S and G2/M, participate in DDR signaling. They sense DNA damage and pause the cell cycle before DNA replication and chromosome segregation respectively, thereby ensuring the fidelity of genetic information in the divided cells. In cancer cells, genome instability and replication stress are commonly observed due to the dysregulation of DDR machinery usually induced by endogenous genetic alterations of cancer cells or genotoxic agents. For example, G1/S checkpoints such as p53, are frequently mutated and disrupted in cancer cells. As a result, the survival of cancer cells bearing replication stress and dysfunctional G1/S checkpoints highly depends on G2/M checkpoints for the processing of DNA damage repair. Therefore, disruption the G2/M checkpoint via PKMYT1 kinase activity inhibition has become a promising therapeutic approach especially for genetically targeted cancer therapies.

### SUMMARY OF THE INVENTION

Objects of the present invention are novel compounds of formula (I), their manufacture, medicaments based on a compound in accordance with the invention and their production as well as the use of compounds of formula (I) as PKMYT1 kinase inhibitors for the treatment of a broad spectrum of cancers. The compounds of formula (I) show superior PKMYT1 kinase inhibition activity. In addition, the compounds of formula (I) also show good safety and good PK profiles, e.g., good microsome stability, hepatic clearance and safety margin.

One aspect of the invention pertains to a compound of formula (I),

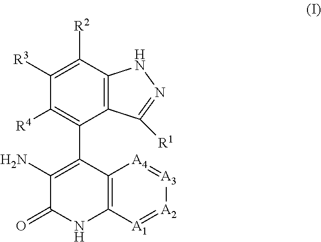

wherein
R¹ is H, halogen or C₁₋₆alkyl;
R² is H, halogen, cyano, C₃₋₇cycloalkyl, C₁₋₆alkyl, C₁₋₆alkoxy, or haloC₁₋₆alkyl;
R³ is H or halogen;
R⁴ is H, halogen, or C₁₋₆alkyl;
A₁ is N or CR⁵;
A₂ is N or CR⁶;
A₃ is N or CR⁷;
A₄ is N or CR⁸;
   each of R⁵, R⁶, R⁷ and R⁸ is independently selected from H, halogen, C₁₋₆alkyl, C₂₋₆alkenyl, C₃₋₇cycloalkyl, C₁₋₆alkoxy, C₁₋₆alkylC₁₋₆alkoxy, C₃₋₇cycloalkylC₁₋₆alkoxy, C₃₋₇cycloalkoxy, C₁₋₆alkylC₃₋₇cycloalkoxy, C₁₋₆alkoxy-oxo-C₁₋₆alkoxy, C₁₋₆alkylamino, C₂₋₆alkynyl, C₁₋₆alkylC₂₋₆alkynyl, aryl, deuterated C₁₋₆alkyl, carbonyl, carboxyl, sulfanyl, sulfinyl, sulfonyl, sulfamoyl, cyano, benzyloxy, 5 to 8 membered heterocyclyl containing one to three heteroatoms selected from N, O, and S, and 5 to 8 membered heteroaryl containing one to three heteroatoms selected from N, O, and S, wherein each of R⁵, R⁶, R⁷ and R⁸ is independently optionally substituted with halogen, C₁₋₆alkyl, C₃₋₇cycloalkyl, cyanoC₃₋₇cycloalkyl, C₁₋₆alkoxy, C₁₋₆alkoxycarbonyl, C₁₋₆alkylcarbamoyl, amino, C₁₋₆alkylamino, haloC₁₋₆alkyl, hydroxyl, cyano, or 5 to 8 membered heterocyclyl containing one to three heteroatoms selected from N, O, and S, wherein each of R⁵, R⁶, R⁷ and R⁸ is independently optionally further substituted with hydroxyl; or
   each of R⁵, R⁶, R⁷ and R⁸ is independently selected from H, halogen, C₁₋₆alkyl, deuterated C₁₋₆alkyl, C₃₋₇cycloalkylC₁₋₆alkyl, (C₁₋₆alkylamino)C₁₋₆alkyl, hydroxyC₁₋₆alkyl, haloC₁₋₆alkyl, C₁₋₆alkoxyC₁₋₆alkyl, C₃₋₇cycloalkyl, haloC₃₋₇cycloalkyl, C₁₋₆alkylC₃₋₇cycloalkyl, C₁₋₆alkoxy, C₃₋₇cycloalkylC₁₋₆alkoxy, (C₁₋₆alkylamino)C₁₋₆alkoxy, (hyroxyl)(C₁₋₆alkyl)C₁₋₆alkoxy, (cyanoC₃₋₇cycloalkyl)C₁₋₆alkoxy, C₁₋₆alkoxy-oxo-C₁₋₆alkoxy, haloC₁₋₆alkoxy, C₃₋₇cycloalkoxy, cyanoC₃₋₇cycloalkoxy, haloC₃₋₇cycloalkoxy, (hyroxyl)(C₁₋₆alkyl)C₃₋₇cycloalkoxy, C₁₋₆alkoxycarbonylC₃₋₇cycloalkoxy, C₂₋₆alkenyl, C₂₋₆alkynyl, hydroxyC₂₋₆alkynyl, C₁₋₆alkoxyC₂₋₆alkynyl, morpholinoC₂₋₆alkynyl, (hyroxyl)(C₁₋₆alkyl)C₂₋₆alkynyl, (C₁₋₆alkoxy)(C₁₋₆alkyl)C₂₋₆alkynyl, (hydroxytetrahydrofuranyl)C₂₋₆alkynyl, (C₁₋₆alkylamino)C₂₋₆alkynyl, (amino)(C₁₋₆alkyl)C₂₋₆alkynyl, C₁₋₆alkoxycarbonyl, C₁₋₆alkylC₂₋₆alkynyl, (hydroxy)(C₁₋₆alkyl)C₂₋₆alkynyl, oxetanylC₂₋₆alkynyl, C₁₋₆alkylamino, acyl, cyano, carboxy, C₁₋₆alkylsulfanyl, C₁₋₆alkylsulfinyl, C₁₋₆alkylsulfonyl, benzyloxy, aryl, 5 to 8 membered heterocyclyl containing one to three heteroatoms selected from N, O, and S, and 5 to 8 membered heteroaryl containing one to three heteroatoms selected from N, O, and S, wherein each of aryl, heterocyclyl, and heteroaryl is independently optionally substituted with halogen, C₁₋₆alkyl, C₃₋₇cycloalkyl, C₁₋₆alkylcarbamoyl, haloC₁₋₆alkyl, or hydroxyl;
or a pharmaceutically acceptable salt thereof.

Another aspect of the invention pertains to a process for the preparation of a compound of formula (I), as well as a compound of formula (I) or a pharmaceutically acceptable salt thereof when manufactured according to the process.

Another aspect of the invention pertains to a pharmaceutical composition comprising a compound of formula (I) or a pharmaceutically acceptable salt thereof, and a pharmaceutically acceptable excipient.

Another aspect of the invention pertains to a compound of formula (I) or a pharmaceutically acceptable salt thereof for use as therapeutically active substance.

Another aspect of the invention pertains to a compound of formula (I) or a pharmaceutically acceptable salt thereof for use in the treatment of cancer.

Another aspect of the invention pertains to the use of a compound of formula (I) or a pharmaceutically acceptable salt thereof for the treatment of cancer.

Another aspect of the invention pertains to the use of a compound of formula (I) or a pharmaceutically acceptable salt thereof for the inhibition of PKMYT1 kinase.

Another aspect of the invention pertains to the use of a compound of formula (I) or a pharmaceutically acceptable salt thereof for the preparation of a medicament for the treatment of cancer.

Another aspect of the invention pertains to the use of a compound of formula (I) or a pharmaceutically acceptable salt thereof for the preparation of a medicament for the inhibition of PKMYT1 kinase.

Another aspect of the invention pertains to a method for the treatment of cancer, which method comprises administering an effective amount of a compound of formula (I) or a pharmaceutically acceptable salt thereof.

### DETAILED DESCRIPTION OF THE INVENTION

### Definitions

Unless otherwise defined, all technical and scientific terms used herein have the same meaning as commonly understood by one of ordinary skill in the art to which this invention belongs. Furthermore, the following definitions are set forth to illustrate and define the meaning and scope of the various terms used to describe the invention.

The nomenclature used in this application is based on IUPAC systematic nomenclature, unless indicated otherwise.

The term “compound(s) of this invention” and “compound(s) of the present invention” refers to compounds of formula (I), and stereoisomers, solvates or salts thereof (e.g., pharmaceutically acceptable salts).

The term “substituent” denotes an atom or a group of atoms replacing a hydrogen atom on the parent molecule.

The term “aryl” denotes a monovalent aromatic carbocyclic mono- or bicyclic ring system comprising 6 to 10 carbon ring atoms. Examples of aryl moieties include phenyl and naphthyl.

As used herein, the term “C₁₋₆alkyl” alone or in combination signifies a saturated, linear- or branched chain alkyl group containing 1 to 6, particularly 2 to 6 or 1 to 4 carbon atoms, for example, methyl, ethyl, propyl, isopropyl, butyl, isobutyl, tert-butyl and the like. Particular “C₁₋₆alkyl” groups are methyl, ethyl, propyl, isopropyl, and isopentyl.

The term “C₁₋₆alkoxy” denotes C₁₋₆alkyl-O—.

The term “C₂₋₆alkenyl” denotes a monovalent linear or branched hydrocarbon group of 2 to 6 carbon atoms with at least one double bond. In particular embodiments, alkenyl has 2 to 4 carbon atoms with at least one double bond. Examples of C₂₋₆alkenyl include ethenyl (or vinyl), propenyl, prop-2-enyl, isopropenyl, n-butenyl, and iso-butenyl.

The term “C₃₋₇cycloalkyl” denotes a monovalent saturated monocyclic or bicyclic hydrocarbon group of 3 to 7 ring carbon atoms. Bicyclic means consisting of two saturated carbocycles having one or more carbon atoms in common. Examples for monocyclic cycloalkyl are cyclopropyl, cyclobutyl, cyclopentyl, cyclohexyl or cycloheptyl. Examples for bicyclic cycloalkyl are bicyclo[1.1.0]butyl, bicyclo[2.2.1]heptanyl, bicyclo[1.1.1]pentanyl, or bicyclo[2.2.2]octanyl.

The term “C₃₋₇cycloalkoxy” denotes C₃₋₇cycloalkyl-O—.

The term “halogen” or “halo” denotes fluoro, chloro, bromo, or iodo.

The term “haloC₁₋₆alkyl” denotes an alkyl group wherein at least one of the hydrogen atoms of the alkyl group is replaced by same or different halogen atoms, particularly fluoro atoms. Examples of haloC₁₋₆alkyl include monochloro-, difluoro- or trifluoro-methyl, -ethyl or -propyl, for example difluoromethyl.

The term “heteroaryl” denotes a monovalent aromatic heterocyclic mono- or bicyclic ring system of 5 to 12 ring atoms, comprising 1, 2, 3 or 4 heteroatoms selected from N, O and S, the remaining ring atoms being carbon. Examples of heteroaryl moieties include, but not limited to, pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl or quinoxalinyl. Heteroaryl can be further substituted by halogen, C₁₋₆alkyl, haloC₁₋₆alkyl, cyano, C₃₋₇cycloalkyl, (C₁₋₆alkyl)₂amino or C₁₋₆alkoxy.

The term “heterocyclyl” denotes a monovalent saturated or partly unsaturated mono- or bicyclic ring system of 3 to 9 ring atoms, comprising 1, 2, or 3 ring heteroatoms selected from N, O and S, the remaining ring atoms being carbon. In particular embodiments, heterocyclyl is a monovalent saturated monocyclic ring system of 4 to 7 ring atoms, comprising 1, 2, or 3 ring heteroatoms selected from N, O and S, the remaining ring atoms being carbon. Examples for monocyclic saturated heterocyclyl are aziridinyl, oxiranyl, azetidinyl, oxetanyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, oxazepanyl, oxopiperidinyl, oxopiperazinyl or oxopyrrolidinyl. Examples for bicyclic saturated heterocyclyl are azaspiro[3.3]heptanyl, 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocyclyl are dihydrofuryl, imidazolinyl, dihydro-oxazolyl, tetrahydro-pyridinyl, or dihydropyranyl.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, trifluoroacetic acid, formic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435. Particular are the sodium salts of the compounds of formula (I).

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

### Inhibitor of PKMYT1 Kinase

The present invention relates to (i) a compound of formula (I),

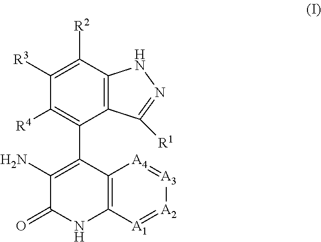

wherein
R¹ is H, halogen or C₁₋₆alkyl;
R² is H, halogen, cyano, C₃₋₇cycloalkyl, C₁₋₆alkyl, C₁₋₆alkoxy, or haloC₁₋₆alkyl;
R³ is H or halogen;
R⁴ is H, halogen, or C₁₋₆alkyl;
A₁ is Nor CR⁵;
A₂ is N or CR⁶;
A₃ is N or CR⁷;
A₄ is N or CR⁸;
   each of R⁵, R⁶, R⁷ and R⁸ is independently selected from H, halogen, C₁₋₆alkyl, C₂₋₆alkenyl, C₃₋₇cycloalkyl, C₁₋₆alkoxy, C₁₋₆alkylC₁₋₆alkoxy, C₃₋₇cycloalkylC₁₋₆alkoxy, C₃₋₇cycloalkoxy, C₁₋₆alkylC₃₋₇cycloalkoxy, C₁₋₆alkoxy-oxo-C₁₋₆alkoxy, C₁₋₆alkylamino, C₂₋₆alkynyl, C₁₋₆alkylC₂₋₆alkynyl, aryl, deuterated C₁₋₆alkyl, carbonyl, carboxyl, sulfanyl, sulfinyl, sulfonyl, sulfamoyl, cyano, benzyloxy, 5 to 8 membered heterocyclyl containing one to three heteroatoms selected from N, O, and S, and 5 to 8 membered heteroaryl containing one to three heteroatoms selected from N, O, and S, wherein each of R⁵, R⁶, R⁷ and R⁸ is independently optionally substituted with halogen, C₁₋₆alkyl, C₃₋₇cycloalkyl, cyanoC₃₋₇cycloalkyl, C₁₋₆alkoxy, C₁₋₆alkoxycarbonyl, C₁₋₆alkylcarbamoyl, amino, C₁₋₆alkylamino, haloC₁₋₆alkyl, hydroxyl, cyano, or 5 to 8 membered heterocyclyl containing one to three heteroatoms selected from N, O, and S, wherein each of R⁵, R⁶, R⁷ and R⁸ is independently optionally further substituted with hydroxyl; or
   each of R⁵, R⁶, R⁷ and R⁸ is independently selected from H, halogen, C₁₋₆alkyl, deuterated C₁₋₆alkyl, C₃₋₇cycloalkylC₁₋₆alkyl, (C₁₋₆alkylamino)C₁₋₆alkyl, hydroxyC₁₋₆alkyl, haloC₁₋₆alkyl, C₁₋₆alkoxyC₁₋₆alkyl, C₃₋₇cycloalkyl, haloC₃₋₇cycloalkyl, C₁₋₆alkylC₃₋₇cycloalkyl, C₁₋₆alkoxy, C₃₋₇cycloalkylC₁₋₆alkoxy, (C₁₋₆alkylamino)C₁₋₆alkoxy, (hyroxyl)(C₁₋₆alkyl)C₁₋₆alkoxy, (cyanoC₃₋₇cycloalkyl)C₁₋₆alkoxy, C₁₋₆alkoxy-oxo-C₁₋₆alkoxy, haloC₁₋₆alkoxy, C₃₋₇cycloalkoxy, cyanoC₃₋₇cycloalkoxy, haloC₃₋₇cycloalkoxy, (hyroxyl)(C₁₋₆alkyl)C₃₋₇cycloalkoxy, C₁₋₆alkoxycarbonylC₃₋₇cycloalkoxy, C₂₋₆alkenyl, C₂₋₆alkynyl, hydroxyC₂₋₆alkynyl, C₁₋₆alkoxyC₂₋₆alkynyl, morpholinoC₂₋₆alkynyl, (hyroxyl)(C₁₋₆alkyl)C₂₋₆alkynyl, (C₁₋₆alkoxy)(C₁₋₆alkyl)C₂₋₆alkynyl, (hydroxytetrahydrofuranyl)C₂₋₆alkynyl, (C₁₋₆alkylamino)C₂₋₆alkynyl, (amino)(C₁₋₆alkyl)C₂₋₆alkynyl, C₁₋₆alkoxycarbonyl, C₁₋₆alkylC₂₋₆alkynyl, (hydroxy)(C₁₋₆alkyl)C₂₋₆alkynyl, oxetanylC₂₋₆alkynyl, C₁₋₆alkylamino, acyl, cyano, carboxy, C₁₋₆alkylsulfanyl, C₁₋₆alkylsulfinyl, C₁₋₆alkylsulfonyl, benzyloxy, aryl, 5 to 8 membered heterocyclyl containing one to three heteroatoms selected from N, O, and S, and 5 to 8 membered heteroaryl containing one to three heteroatoms selected from N, O, and S, wherein each of aryl, heterocyclyl, and heteroaryl is independently optionally substituted with halogen, C₁₋₆alkyl, C₃₋₇cycloalkyl, C₁₋₆alkylcarbamoyl, haloC₁₋₆alkyl, or hydroxyl;
or a pharmaceutically acceptable salt thereof.

A further embodiment of present invention is (ii) the compound according to (i), wherein
R¹ is H or C₁₋₆alkyl;
R² is H, halogen, C₁₋₆alkyl, or haloC₁₋₆alkyl;
R³ is H or halogen;
R⁴ is H, halogen, or C₁₋₆alkyl;
A₁ is Nor CR⁵;
   R⁵ is H, halogen, C₁₋₆alkyl, haloC₁₋₆alkyl, C₁₋₆alkoxy, or C₃₋₇cycloalkyl; A₂ is N or CR⁶;
   R⁶ is H, halogen, C₁₋₆alkyl, hydroxyC₁₋₆alkyl, deuterated C₁₋₆alkyl, haloC₁₋₆alkyl, C₃₋₇cycloalkylC₁₋₆alkyl, C₃₋₇cycloalkyl, haloC₃₋₇cycloalkyl, C₁₋₆alkylC₃₋₇cycloalkyl, C₁₋₆alkoxy, haloC₁₋₆alkoxy, C₃₋₇cycloalkoxy, C₂₋₆alkenyl, C₂₋₆alkynyl, cyano, C₁₋₆alkylsulfanyl, C₁₋₆alkylsulfinyl, C₁₋₆alkylsulfonyl, aryl, 5 to 8 membered heterocyclyl containing one to three heteroatoms selected from N, O, and S, or 5 to 8 membered heteroaryl containing one to three heteroatoms selected from N, O, and S, wherein each of aryl, heterocyclyl, and heteroaryl is independently optionally substituted with halogen, C₁₋₆alkyl, haloC₁₋₆alkyl, or C₁₋₆alkylcarbamoyl;
A₃ is N or CR⁷;
   R⁷ is H, halogen, C₁₋₆alkyl, hydroxyC₁₋₆alkyl, haloC₁₋₆alkyl, (C₁₋₆alkylamino)C₁₋₆alkyl, C₃₋₇cycloalkyl, C₁₋₆alkoxy, haloC₁₋₆alkoxy, C₃₋₇cycloalkylC₁₋₆alkoxy, (C₁₋₆alkylamino)C₁₋₆alkoxy, (hyroxyl)(C₁₋₆alkyl)C₁₋₆alkoxy, (cyanoC₃₋₇cycloalkyl)C₁₋₆alkoxy, C₁₋₆alkoxy-oxo-C₁₋₆alkoxy, C₃₋₇cycloalkoxy, cyanoC₃₋₇cycloalkoxy, haloC₃₋₇cycloalkoxy, (hyroxyl)(C₁₋₆alkyl)C₃₋₇cycloalkoxy, C₁₋₆alkoxycarbonylC₃₋₇cycloalkoxy, C₁₋₆alkylamino, C₂₋₆alkenyl, C₂₋₆alkynyl, hydroxyC₂₋₆alkynyl, C₁₋₆alkoxyC₂₋₆alkynyl, morpholinoC₂₋₆alkynyl, (hyroxyl)(C₁₋₆alkyl)C₂₋₆alkynyl, (C₁₋₆alkoxy)(C₁₋₆alkyl)C₂₋₆alkynyl, (hydroxytetrahydrofuranyl)C₂₋₆alkynyl, (C₁₋₆alkylamino)C₂₋₆alkynyl, (amino)(C₁₋₆alkyl)C₂₋₆alkynyl, C₁₋₆alkylC₂₋₆alkynyl, (hydroxy)(C₁₋₆alkyl)C₂₋₆alkynyl, oxetanylC₂₋₆alkynyl, C₁₋₆alkoxycarbonyl, acyl, carboxy, cyano, or 5 to 8 membered heterocyclyl containing one to three heteroatoms selected from N, O, and S, wherein each of heterocyclyl and heteroaryl is independently optionally substituted with C₁₋₆alkyl, C₃₋₇cycloalkyl, haloC₁₋₆alkyl, or hydroxyl;
A₄ is N or CR^{B};
   R⁸ is H, halogen, C₁₋₆alkyl, C₁₋₆alkoxy, C₁₋₆alkoxyC₁₋₆alkyl, haloC₁₋₆alkoxy, C₃₋₇cycloalkylC₁₋₆alkoxy, C₃₋₇cycloalkoxy, cyano, or benzyloxy;
or a pharmaceutically acceptable salt thereof.

A further embodiment of present invention is (iii) the compound according to (i) or (ii), wherein R¹ is H or methyl.

A further embodiment of present invention is (iv) the compound according to any one of (i)-(iii), wherein R¹ is H.

A further embodiment of present invention is (v) the compound according to any one of (i)-(iv), wherein R² is H, fluoro, chloro, methyl, or trifluoromethyl.

A further embodiment of present invention is (vi) the compound according to any one of (i)-(iv), wherein R² is halogen.

A further embodiment of present invention is (vi) the compound according to (vi), wherein R² is fluoro or chloro.

A further embodiment of present invention is (viii) the compound according to any one of (i)-(vii), wherein R³ is H or fluoro.

A further embodiment of present invention is (ix) the compound according to any one of (i)-(viii), wherein R³ is H.

A further embodiment of present invention is (x) the compound according to any one of (i)-(ix), wherein R⁴ is H, fluoro, chloro, or methyl.

A further embodiment of present invention is (xi) the compound according to any one of (i)-(x), wherein R⁴ is H.

A further embodiment of present invention is (xii) the compound according to any one of (i)-(xi), wherein R⁵ is H, chloro, fluoro, methyl, difluoromethyl, methoxy, or cyclopropyl.

A further embodiment of present invention is (xiii) the compound according to any one of (i)-(xi), wherein R⁵ is H, halogen, or C₁₋₆alkyl; preferably H or C₁₋₆alkyl.

A further embodiment of present invention is (xiv) the compound according to (xiii), wherein R⁵ is H, chloro, or methyl; preferably H or methyl.

A further embodiment of present invention is (xv) the compound according to any one of (i)-(xiv), wherein R⁶ is H, fluoro, chloro, bromo, iodo, methyl, trideuteriomethyl, ethyl, isopropyl, trifluoromethyl, trifluoroethyl, difluoroethyl, hydroxyisopropyl, cyclopropylmethyl, cyclopropyl, cyclopentyl, cyclobutyl, fluorocyclopropyl, methylcyclopropyl, dimethylcyclopropyl, methoxy, ethoxy, isopropoxy, trifluoroethoxy, cyclobutoxy, vinyl, isopropenyl, ethynyl, azetidinyl, oxetanyl, morpholino, cyano, methylsulfanyl, methylsulfinyl, methylsulfonyl, phenyl, or a heteroaryl selected from imidazolyl, pyrazolyl, isothiazolyl, pyridyl, pyridazinyl, and thiazolyl, wherein each of phenyl and heteroaryl is independently optionally substituted with fluoro, methyl, trifluoromethyl, or dimethylcarbamoyl.

A further embodiment of present invention is (xvi) the compound according to any one of (i)-(xv), wherein R⁶ is H, chloro, fluoro, bromo, iodo, methyl, trideuteriomethyl, ethyl, isopropyl, trifluoromethyl, 2,2,2-trifluoroethyl, 1,1-difluoroethyl, 1-hydroxy-1-methyl-ethyl, cyclopropylmethyl, cyclopropyl, cyclopentyl, cyclobutyl, 2-fluorocyclopropyl, 1-methylcyclopropyl, (1R,2R)-2-methylcyclopropyl, methoxy, ethoxy, isopropoxy, 2,2,2-trifluoroethoxy, cyclobutoxy, vinyl, isopropenyl, ethynyl, azetidin-1-yl, oxetan-3-yl, morpholino, cyano, methylsulfanyl, methylsulfinyl, methylsulfonyl, 4-(dimethylcarbamoyl)phenyl, 3-methylimidazol-4-yl, 1H-pyrazol-5-yl, isothiazol-5-yl, 2-(trifluoromethyl)-4-pyridyl, 5-fluoro-2-pyridyl, 2-pyridyl, pyridazin-4-yl, or thiazol-2-yl.

A further embodiment of present invention is (xvii) the compound according to any one of (i)-(xiv), wherein R⁶ is H, halogen, C₁₋₆alkyl, haloC₁₋₆alkyl, deuterated C₁₋₆alkyl, C₃₋₇cycloalkyl, or C₁₋₆alkylsulfanyl; preferably C₁₋₆alkyl, deuterated C₁₋₆alkyl, haloC₁₋₆alkyl, C₃₋₇cycloalkyl, or C₁₋₆alkylsulfanyl.

A further embodiment of present invention is (xviii) the compound according to (xvii), wherein R⁶ is H, chloro, methyl, trideuteriomethyl, ethyl, isopropyl, difluoroethyl, trifluoroethyl, cyclopropyl, or methylsulfanyl; preferably methyl, ethyl, isopropyl, trideuteriomethyl, difluoroethyl, trifluoroethyl, cyclopropyl, or methylsulfanyl.

A further embodiment of present invention is (xix) the compound according to any one of (i)-(xviii), wherein R⁷ is H, chloro, bromo, methyl, ethyl, hydroxyisopentyl, hydroxyisopropyl, trifluoromethyl, cyclopropyl, 3-(dimethylamino)propyl, methoxy, propoxy, isopropoxy, 1-bicyclo[1.1.1]pentanylmethoxy, difluoromethoxy, trifluoroethoxy, difluoroethoxy, difluoropropoxy, (dimethylamino)ethoxy, methylpropoxy, cyclopropylmethoxy, (1-cyanocyclopropyl)methoxy, 2-ethoxy-2-oxo-ethoxy, cyclobutoxy, cyanocyclobutoxy, fluorocyclobutoxy, 3-(1-methyl-ethyl)cyclobutoxy, 3-methoxycarbonylcyclobutoxy, isopropylamino, vinyl, ethynyl, hydroxybutynyl, methoxypropynyl, morpholinopropynyl, (hydroxyl)(methyl)butynyl, methoxymethylbutynyl, 2-(4-hydroxytetrahydrofuran-3-yl)-1-ethynyl, dimethylaminopropynyl, 3-methyl-1-but-1-ynyl, methylbutynyl, 3-hydroxy-3-methyl-butynyl, 3-methoxy-3-methyl-butynyl, 2-(oxetan-3-yl)-ethynyl, methoxycarbonyl, carboxy, cyano, acetyl, thienyl, thiazolyl, oxazolyl, pyridyl, or a heterocyclyl selected from pyrrolidinyl, morpholino, 2-oxa-6-azaspiro[3.3]heptanyl, 2,6-diazaspiro[3.3]heptanyl, 2,5-diazabicyclo[2.2.1]heptanyl, piperazinyl, 2,5-diazabicyclo[2.2.2]octanyl, piperidyl, oxetanyl, azetidinyl, and 2-oxa-6-azaspiro[3.3]heptanyl, wherein heterocyclyl is optionally substituted with methyl, isopropyl, cyclopropyl, trifluoroethyl, or hydroxyl.

A further embodiment of present invention is (xx) the compound according to any one of (i)-(xix), wherein R⁷ is H, chloro, bromo, methyl, ethyl, 3-hydroxy-3-methyl-1-butyl, 1-hydroxy-1-methyl-ethyl, trifluoromethyl, cyclopropyl, 3-(dimethylamino)propyl, methoxy, propoxy, isopropoxy, 1-bicyclo[1.1.1]pentanylmethoxy, difluoromethoxy, trifluoroethoxy, 2,2-difluoroethoxy, 3,3-difluoropropoxy, 2-(dimethylamino)ethoxy, 2-hydroxy-2-methyl-propoxy, cyclopropylmethoxy, (1-cyanocyclopropyl)methoxy, 2-ethoxy-2-oxo-ethoxy, cyclobutoxy, 3-cyanocyclobutoxy, 3-fluorocyclobutoxy, 3-(1-hydroxy-1-methyl-ethyl)cyclobutoxy, 3-methoxycarbonylcyclobutoxy, isopropylamino, vinyl, ethynyl, 3-hydroxybut-1-ynyl, 3-methoxyprop-1-ynyl, 3-morpholinoprop-1-ynyl, 3-hydroxy-3-methyl-1-but-1-ynyl, 3-methoxy-3-methyl-but-1-ynyl, 2-(4-hydroxytetrahydrofuran-3-yl)-1-ethynyl, 3-(dimethylamino)prop-1-ynyl, 3-amino-3-methyl-but-1-ynyl, 3-methylbut-1-ynyl, 3-hydroxy-3-methyl-but-1-ynyl, 3-methoxy-3-methyl-but-1-ynyl, 2-(oxetan-3-yl)-ethynyl, methoxycarbonyl, carboxy, cyano, acetyl, 2-thienyl, thiazol-4-yl, thiazol-2-yl, oxazol-2-yl, 2-pyridyl, 3-hydroxypyrrolidin-1-yl, morpholino, 2-oxa-6-azaspiro[3.3]heptan-6-yl, 2-methyl-2,6-diazaspiro[3.3]heptan-6-yl, 5-methyl-2,5-diazabicyclo[2.2.1]heptan-2-yl, piperazin-1-yl, 4-methylpiperazin-1-yl, 4-isopropylpiperazin-1-yl, 4-cyclopropylpiperazin-1-yl, 5-methyl-2,5-diazabicyclo[2.2.2]octan-2-yl, 4-piperidyl, 1-methyl-4-piperidyl, 1-(2,2,2-trifluoroethyl)-4-piperidyl, oxetan-3-yl, 1-methylazetidin-3-yl, or 2-oxa-6-azaspiro[3.3]heptan-6-yl.

A further embodiment of present invention is (xxi) the compound according to any one of (i)-(xviii), wherein R⁷ is H, halogen, C₁₋₆alkyl, C₃₋₇cycloalkyl, C₃₋₇cycloalkoxy, C₃₋₇cycloalkylC₁₋₆alkoxy, (hyroxyl)(C₁₋₆alkyl)C₂₋₆alkynyl, or hydroxyC₂₋₆alkynyl; preferably H or C₁₋₆alkyl.

A further embodiment of present invention is (xxii) the compound according to (xxi), wherein R⁷ is H, chloro, methyl, cyclopropyl, cyclobutoxy, cyclopropylmethoxy, 3-hydroxy-3-methyl-1-but-1-ynyl, or 3-hydroxybut-1-ynyl; preferably H or methyl.

A further embodiment of present invention is (xxiii) the compound according to any one of (i)-(xxii), wherein R⁸ is H, chloro, fluoro, methyl, ethyl, methoxy, ethoxy, propoxy, isopropoxy, methoxymethyl, difluoromethoxy, trifluoromethoxy, 2,2,2-trifluoroethoxy, 2,2-difluoroethoxy, 3,3-difluoropropoxy, cyclopropylmethoxy, cyclopropoxy, cyclobutoxy, cyano, or benzyloxy.

A further embodiment of present invention is (xxiv) the compound according to any one of (i)-(xxii), wherein R⁸ is C₁₋₆alkoxy, haloC₁₋₆alkoxy, C₃₋₇cycloalkoxy, or cyano; preferably C₁₋₆alkoxy, haloC₁₋₆alkoxy, or C₃₋₇cycloalkoxy.

A further embodiment of present invention is (xxv) the compound according to (xxiv), wherein R⁸ is ethoxy, difluoromethoxy, trifluoromethoxy, 2,2,2-trifluoroethoxy, cyclopropoxy, cyclobutoxy, or cyano; preferably ethoxy, difluoromethoxy, trifluoroethoxy, cyclopropoxy, or cyclobutoxy.

A further embodiment of present invention is (xxvi) a compound according to (i) or (ii), wherein
R¹ is H;
R² is halogen;
R³ is H;
R⁴ is H;
A₁ is CR⁵;
   R⁵ is H, halogen, or C₁₋₆alkyl; preferably H or C₁₋₆alkyl; A₂ is N or CR⁶;
   R⁶ is H, halogen, C₁₋₆alkyl, C₃₋₇cycloalkyl, haloC₁₋₆alkyl, deuterated C₁₋₆alkyl or C₁₋₆alkylsulfanyl; preferably C₁₋₆alkyl, deuterated C₁₋₆alkyl, haloC₁₋₆alkyl, C₃₋₇cycloalkyl, or C₁₋₆alkylsulfanyl;
A₃ is N or CR⁷;
   R⁷ is H, halogen, C₁₋₆alkyl, C₃₋₇cycloalkyl, C₃₋₇cycloalkoxy, C₃₋₇cycloalkylC₁₋₆alkoxy, (hyroxyl)(C₁₋₆alkyl)C₂₋₆alkynyl, or hydroxyC₂₋₆alkynyl; preferably H or C₁₋₆alkyl;
A₄ is N or CR⁸;
R⁸ is C₁₋₆alkoxy, haloC₁₋₆alkoxy, C₃₋₇cycloalkoxy, or cyano; preferably C₁₋₆alkoxy, haloC₁₋₆alkoxy, or C₃₋₇cycloalkoxy.

A further embodiment of present invention is (xxvii) a compound according to (xxvi), wherein
R¹ is H;
R² is fluoro or chloro;
R³ is H;
R⁴ is H;
A₁ is N or CR⁵;
   R⁵ is H, chloro, or methyl; preferably H or methyl;
A₂ is N or CR⁶;
   R⁶ is H, chloro, methyl, trideuteriomethyl, ethyl, isopropyl, difluoroethyl, trifluoroethyl, cyclopropyl, or methylsulfanyl; preferably methyl, ethyl, isopropyl, trideuteriomethyl, difluoroethyl, trifluoroethyl, cyclopropyl, or methylsulfanyl;
A₃ is N or CR⁷;
   R⁷ is H, chloro, methyl, cyclopropyl, cyclobutoxy, cyclopropylmethoxy, 3-hydroxy-3-methyl-1-but-1-ynyl, or 3-hydroxybut-1-ynyl; preferably H or methyl;
A₄ is N or CR⁸;
R⁸ is ethoxy, difluoromethoxy, trifluoromethoxy, 2,2,2-trifluoroethoxy, cyclopropoxy, cyclobutoxy, or cyano; preferably ethoxy, difluoromethoxy, trifluoroethoxy, cyclopropoxy, or cyclobutoxy.

A further embodiment of present invention is (xxviii) a compound selected from:
3-Amino-7-chloro-4-(7-fluoro-1H-indazol-4-yl)-1H-1,5-naphthyridin-2-one,
3-Amino-4-(7-fluoro-1H-indazol-4-yl)-7-methyl-1H-1,5-naphthyridin-2-one,
3-Amino-6-chloro-4-(7-fluoro-1H-indazol-4-yl)-7-methyl-1H-1,5-naphthyridin-2-one,
3-Amino-7-fluoro-4-(7-fluoro-1H-indazol-4-yl)-1H-1,5-naphthyridin-2-one,
3-Amino-4-(7-fluoro-1H-indazol-4-yl)-7-(trifluoromethyl)-1H-1,5-naphthyridin-2-one,
3-Amino-4-(7-fluoro-1H-indazol-4-yl)-7-methoxy-1H-1,5-naphthyridin-2-one, 4-[7-Amino-8-(7-fluoro-1H-indazol-4-yl)-6-oxo-5H-1,5-naphthyridin-3-yl]-N,N-dimethyl-benzamide,
3-Amino-4-(7-fluoro-1H-indazol-4-yl)-7-(3-methylimidazol-4-yl)-1H-1,5-naphthyridin-2-one,
3-Amino-4-(7-fluoro-1H-indazol-4-yl)-7-(1H-pyrazol-5-yl)-1H-1,5-naphthyridin-2-one,
3-Amino-4-(7-fluoro-1H-indazol-4-yl)-7-isothiazol-5-yl-1H-1,5-naphthyridin-2-one,
3-Amino-4-(7-fluoro-1H-indazol-4-yl)-7-[2-(trifluoromethyl)-4-pyridyl]-1H-1,5-naphthyridin-2-one,
3-Amino-4-(7-fluoro-1H-indazol-4-yl)-7-pyridazin-4-yl-1H-1,5-naphthyridin-2-one,
3-Amino-4-(7-fluoro-1H-indazol-4-yl)-7-(2-pyridyl)-1H-1,5-naphthyridin-2-one,
3-Amino-4-(7-fluoro-1H-indazol-4-yl)-7-(5-fluoro-2-pyridyl)-1H-1,5-naphthyridin-2-one,
3-Amino-7-cyclopropyl-4-(7-fluoro-1H-indazol-4-yl)-1H-1,5-naphthyridin-2-one,
3-Amino-4-(7-fluoro-1H-indazol-4-yl)-7-thiazol-2-yl-1H-1,5-naphthyridin-2-one,
3-Amino-4-(7-fluoro-1H-indazol-4-yl)-6,7-dimethyl-1H-1,5-naphthyridin-2-one,
3-Amino-6-cyclopropyl-4-(7-fluoro-1H-indazol-4-yl)-7-methyl-1H-1,5-naphthyridin-2-one,
3-Amino-4-(7-fluoro-1H-indazol-4-yl)-7-morpholino-1H-1,5-naphthyridin-2-one,
3-Amino-4-(7-fluoro-1H-indazol-4-yl)-7-(1-hydroxy-1-methyl-ethyl)-1H-1,5-naphthyridin-2-one,
3-Amino-6-bromo-4-(7-fluoro-1H-indazol-4-yl)-7-methyl-1H-1,5-naphthyridin-2-one,
3-Amino-4-(7-fluoro-1H-indazol-4-yl)-7-methyl-6-(4-methylpiperazin-1-yl)-1H-1,5-naphthyridin-2-one,
3-Amino-4-(7-fluoro-1H-indazol-4-yl)-6-(isopropylamino)-7-methyl-1H-1,5-naphthyridin-2-one,
3-Amino-4-(7-fluoro-1H-indazol-4-yl)-6-(3-hydroxypyrrolidin-1-yl)-7-methyl-1H-1,5-naphthyridin-2-one,
3-Amino-4-(7-fluoro-1H-indazol-4-yl)-7-methyl-6-morpholino-1H-1,5-naphthyridin-2-one,
3-Amino-4-(7-fluoro-1H-indazol-4-yl)-7-methyl-6-(2-oxa-6-azaspiro[3.3]heptan-6-yl)-1H-1,5-naphthyridin-2-one,
3-Amino-4-(7-fluoro-1H-indazol-4-yl)-7-methyl-6-(6-methyl-2,6-diazaspiro[3.3]heptan-2-yl)-1H-1,5-naphthyridin-2-one,
3-Amino-4-(7-fluoro-1H-indazol-4-yl)-6-(4-isopropylpiperazin-1-yl)-7-methyl-1H-1,5-naphthyridin-2-one,
3-Amino-6-(4-cyclopropylpiperazin-1-yl)-4-(7-fluoro-1H-indazol-4-yl)-7-methyl-1H-1,5-naphthyridin-2-one,
3-Amino-4-(7-fluoro-1H-indazol-4-yl)-7-methyl-6-(5-methyl-2,5-diazabicyclo[2.2.1]heptan-2-yl)-1H-1,5-naphthyridin-2-one,
3-Amino-4-(7-fluoro-1H-indazol-4-yl)-7-methyl-6-(5-methyl-2,5-diazabicyclo[2.2.2]octan-2-yl)-1H-1,5-naphthyridin-2-one,
3-Amino-4-(7-fluoro-1H-indazol-4-yl)-6-(3-hydroxy-3-methyl-butyl)-7-methyl-1H-1,5-naphthyridin-2-one,
3-Amino-4-(1H-indazol-4-yl)-7-methyl-1H-1,5-naphthyridin-2-one,
3-Amino-7-cyclopropyl-4-(1H-indazol-4-yl)-1H-1,5-naphthyridin-2-one,
3-Amino-4-(1H-indazol-4-yl)-7-thiazol-2-yl-1H-1,5-naphthyridin-2-one,
3-Amino-7-chloro-4-(6-fluoro-1H-indazol-4-yl)-1H-1,5-naphthyridin-2-one,
3-Amino-7-chloro-4-(7-chloro-1H-indazol-4-yl)-1H-1,5-naphthyridin-2-one,
3-Amino-7-chloro-4-(7-methyl-1H-indazol-4-yl)-1H-1,5-naphthyridin-2-one,
3-Amino-7-chloro-4-(6,7-difluoro-1H-indazol-4-yl)-1H-1,5-naphthyridin-2-one,
3-Amino-7-chloro-4-(5-fluoro-1H-indazol-4-yl)-1H-1,5-naphthyridin-2-one,
3-Amino-7-chloro-4-[7-(trifluoromethyl)-1H-indazol-4-yl]-1H-1,5-naphthyridin-2-one,
3-Amino-7-chloro-4-(7-fluoro-5-methyl-1H-indazol-4-yl)-1H-1,5-naphthyridin-2-one,
3-Amino-7-chloro-4-(5-chloro-7-fluoro-1H-indazol-4-yl)-1H-1,5-naphthyridin-2-one,
3-Amino-4-(7-fluoro-1H-indazol-4-yl)-7-methyl-6-piperazin-1-yl-1H-1,5-naphthyridin-2-one,
3-Amino-7-chloro-4-(3-methyl-1H-indazol-4-yl)-1H-1,5-naphthyridin-2-one,
3-Amino-6-(cyclobutoxy)-4-(7-fluoro-1H-indazol-4-yl)-7-methyl-1H-1,5-naphthyridin-2-one,
3-Amino-6-(cyclopropylmethoxy)-4-(7-fluoro-1H-indazol-4-yl)-7-methyl-1H-1,5-naphthyridin-2-one,
3-Amino-4-(7-fluoro-1H-indazol-4-yl)-6,7-dimethyl-1H-quinolin-2-one,
3-Amino-6-chloro-4-(7-fluoro-1H-indazol-4-yl)-7,8-dimethyl-1H-1,5-naphthyridin-2-one,
3-Amino-8-chloro-4-(7-fluoro-1H-indazol-4-yl)-1H-1,5-naphthyridin-2-one,
3-Amino-4-(7-fluoro-1H-indazol-4-yl)-8-methoxy-7-methyl-1H-1,5-naphthyridin-2-one,
3-Amino-7-chloro-4-(7-fluoro-1H-indazol-4-yl)-6-methyl-1H-1,5-naphthyridin-2-one,
3-Amino-8-chloro-4-(7-fluoro-1H-indazol-4-yl)-7-methyl-1H-1,5-naphthyridin-2-one,
3-Amino-4-(7-fluoro-1H-indazol-4-yl)-7,8-dimethyl-1H-1,5-naphthyridin-2-one,
3-Amino-7-chloro-6-cyclopropyl-4-(7-fluoro-1H-indazol-4-yl)-1H-1,5-naphthyridin-2-one,
3-Amino-7-chloro-4-(7-fluoro-1H-indazol-4-yl)-6,8-dimethyl-1H-1,5-naphthyridin-2-one,
3-Amino-8-chloro-4-(7-fluoro-1H-indazol-4-yl)-6,7-dimethyl-1H-1,5-naphthyridin-2-one,
1-[[7-Amino-8-(7-fluoro-1H-indazol-4-yl)-3-methyl-6-oxo-5H-1,5-naphthyridin-2-yl]oxymethyl]cyclopropanecarbonitrile,
3-Amino-6-(2,2-difluoroethoxy)-4-(7-fluoro-1H-indazol-4-yl)-7-methyl-1H-1,5-naphthyridin-2-one,
3-Amino-4-(7-fluoro-1H-indazol-4-yl)-6-methoxy-7-methyl-1H-1,5-naphthyridin-2-one,
Ethyl 2-[[7-amino-8-(7-fluoro-1H-indazol-4-yl)-3-methyl-6-oxo-5H-1,5-naphthyridin-2-yl]oxy]acetate,
3-Amino-6-(difluoromethoxy)-4-(7-fluoro-1H-indazol-4-yl)-7-methyl-1H-1,5-naphthyridin-2-one,
3-Amino-6-(1-bicyclo[1.1.1]pentanylmethoxy)-4-(7-fluoro-1H-indazol-4-yl)-7-methyl-1H-1,5-naphthyridin-2-one,
3-Amino-4-(7-fluoro-1H-indazol-4-yl)-6-isopropoxy-7-methyl-1H-1,5-naphthyridin-2-one,
3-Amino-4-(7-fluoro-1H-indazol-4-yl)-7-methyl-6-propoxy-1H-1,5-naphthyridin-2-one,
3-Amino-4-(7-fluoro-1H-indazol-4-yl)-7-methyl-6-(2,2,2-trifluoroethoxy)-1H-1,5-naphthyridin-2-one,
Methyl 3-[[7-amino-8-(7-fluoro-1H-indazol-4-yl)-3-methyl-6-oxo-5H-1,5-naphthyridin-2-yl]oxy]cyclobutanecarboxylate,
3-Amino-6-[2-(dimethylamino)ethoxy]-4-(7-fluoro-1H-indazol-4-yl)-7-methyl-1H-1,5-naphthyridin-2-one,
3-Amino-4-(7-fluoro-1H-indazol-4-yl)-7-methyl-6-(4-piperidyl)-1H-1,5-naphthyridin-2-one,
3-Amino-4-(7-fluoro-1H-indazol-4-yl)-6-(3-hydroxy-3-methyl-but-1-ynyl)-7-methyl-1H-1,5-naphthyridin-2-one,
3-Amino-4-(7-fluoro-1H-indazol-4-yl)-6-(3-methoxyprop-1-ynyl)-7-methyl-1H-1,5-naphthyridin-2-one,
3-Amino-4-(7-fluoro-1H-indazol-4-yl)-6-(3-methoxy-3-methyl-but-1-ynyl)-7-methyl-1H-1,5-naphthyridin-2-one,
3-Amino-4-(7-fluoro-1H-indazol-4-yl)-6-[2-(4-hydroxytetrahydrofuran-3-yl)ethynyl]-7-methyl-1H-1,5-naphthyridin-2-one,
3-Amino-4-(7-fluoro-1H-indazol-4-yl)-6-(3-hydroxybut-1-ynyl)-7-methyl-1H-1,5-naphthyridin-2-one,
3-Amino-6-[3-(dimethylamino)prop-1-ynyl]-4-(7-fluoro-1H-indazol-4-yl)-7-methyl-1H-1,5-naphthyridin-2-one,
3-Amino-4-(7-fluoro-1H-indazol-4-yl)-7-methyl-6-(3-morpholinoprop-1-ynyl)-1H-1,5-naphthyridin-2-one,
3-Amino-6-(3-amino-3-methyl-but-1-ynyl)-4-(7-fluoro-1H-indazol-4-yl)-7-methyl-1H-1,5-naphthyridin-2-one,
3-Amino-4-(7-fluoro-1H-indazol-4-yl)-6-(2-hydroxy-2-methyl-propoxy)-7-methyl-1H-1,5-naphthyridin-2-one,
3-[[7-Amino-8-(7-fluoro-1H-indazol-4-yl)-3-methyl-6-oxo-5H-1,5-naphthyridin-2-yl]oxy]cyclobutanecarbonitrile,
trans-3-Amino-6-(3-fluorocyclobutoxy)-4-(7-fluoro-1H-indazol-4-yl)-7-methyl-1H-1,5-naphthyridin-2-one,
3-Amino-6-(3,3-difluoropropoxy)-4-(7-fluoro-1H-indazol-4-yl)-7-methyl-1H-1,5-naphthyridin-2-one,
3-Amino-4-(7-fluoro-1H-indazol-4-yl)-7-methyl-6-(1-methyl-4-piperidyl)-1H-1,5-naphthyridin-2-one,
7-Amino-8-(7-fluoro-1H-indazol-4-yl)-2,3-dimethyl-5H-pyrido[2,3-b]pyrazin-6-one,
7-Amino-8-(7-fluoro-1H-indazol-4-yl)-2,4-dimethyl-5H-pyrido[3,2-d]pyrimidin-6-one,
3-Amino-7-cyclopropyl-4-(7-fluoro-1H-indazol-4-yl)-6-methyl-1H-1,5-naphthyridin-2-one,
3-Amino-6-[3-(dimethylamino)propyl]-4-(7-fluoro-1H-indazol-4-yl)-7-methyl-1H-1,5-naphthyridin-2-one,
Methyl 7-amino-8-(7-fluoro-1H-indazol-4-yl)-3-methyl-6-oxo-5H-1,5-naphthyridine-2-carboxylate,
3-Amino-4-(7-fluoro-1H-indazol-4-yl)-6-(1-hydroxy-1-methyl-ethyl)-7-methyl-1H-1,5-naphthyridin-2-one,
7-Amino-8-(7-fluoro-1H-indazol-4-yl)-3-methyl-6-oxo-5H-1,5-naphthyridine-2-carboxylic acid,
3-Amino-8-chloro-4-(7-fluoro-1H-indazol-4-yl)-6-methyl-1H-1,5-naphthyridin-2-one,
3-Amino-6-chloro-4-(7-fluoro-1H-indazol-4-yl)-8-methyl-1H-1,5-naphthyridin-2-one,
7-Amino-8-(7-fluoro-1H-indazol-4-yl)-3-methyl-6-oxo-5H-1,5-naphthyridine-2-carbonitrile,
3-Amino-4-(7-fluoro-1H-indazol-4-yl)-7-methyl-6-(oxetan-3-yl)-1H-1,5-naphthyridin-2-one,
3-Amino-4-(7-fluoro-1H-indazol-4-yl)-6,8-dimethyl-1H-1,5-naphthyridin-2-one,
3-Amino-6-chloro-7-cyclopropyl-4-(7-fluoro-1H-indazol-4-yl)-1H-1,5-naphthyridin-2-one,
3-Amino-4-(7-fluoro-1H-indazol-4-yl)-6-[3-(1-hydroxy-1-methyl-ethyl)cyclobutoxy]-7-methyl-1H-1,5-naphthyridin-2-one,
3-Amino-4-(7-fluoro-1H-indazol-4-yl)-7-methyl-6-(trifluoromethyl)-1H-1,5-naphthyridin-2-one,
3-Amino-4-(7-fluoro-1H-indazol-4-yl)-7-methyl-6-(1-methylazetidin-3-yl)-1H-1,5-naphthyridin-2-one,
3-Amino-7-chloro-4-(7-fluoro-1H-indazol-4-yl)-8-methyl-1H-1,5-naphthyridin-2-one,
3-Amino-7-cyclopropyl-4-(7-fluoro-1H-indazol-4-yl)-8-methyl-1H-1,5-naphthyridin-2-one, or
3-Amino-4-(7-fluoro-1H-indazol-4-yl)-8-methyl-7-(trideuteriomethyl)-1H-1,5-naphthyridin-2-one,
3-Amino-4-(7-fluoro-1H-indazol-4-yl)-5-methoxy-7-methyl-1H-quinolin-2-one,
3-Amino-5-chloro-4-(7-fluoro-1H-indazol-4-yl)-7-methyl-1H-quinolin-2-one,
3-Amino-6-bromo-4-(7-fluoro-1H-indazol-4-yl)-7-methyl-1H-quinolin-2-one,
3-Amino-4-(7-fluoro-1H-indazol-4-yl)-7-methyl-6-(2-oxa-6-azaspiro[3.3]heptan-6-yl)-1H-quinolin-2-one,
3-Amino-4-(7-fluoro-1H-indazol-4-yl)-7-methyl-6-[1-(2,2,2-trifluoroethyl)-4-piperidyl]-1H-quinolin-2-one,
3-Amino-4-(7-fluoro-1H-indazol-4-yl)-6,7-dimethyl-1H-1,8-naphthyridin-2-one,
3-Amino-6-bromo-8-chloro-4-(7-fluoro-1H-indazol-4-yl)-7-methyl-1H-1,5-naphthyridin-2-one,
3-Amino-8-chloro-6-cyclopropyl-4-(7-fluoro-1H-indazol-4-yl)-7-methyl-1H-1,5-naphthyridin-2-one,
3-Amino-4-(7-fluoro-1H-indazol-4-yl)-6,7,8-trimethyl-1H-1,5-naphthyridin-2-one,
3-Amino-8-chloro-6-ethynyl-4-(7-fluoro-1H-indazol-4-yl)-7-methyl-1H-1,5-naphthyridin-2-one,
6-Acetyl-3-amino-8-chloro-4-(7-fluoro-1H-indazol-4-yl)-7-methyl-1H-1,5-naphthyridin-2-one,
3-Amino-8-chloro-4-(7-fluoro-1H-indazol-4-yl)-7-methyl-6-vinyl-1H-1,5-naphthyridin-2-one,
3-Amino-6,8-dicyclopropyl-4-(7-fluoro-1H-indazol-4-yl)-7-methyl-1H-1,5-naphthyridin-2-one,
3-Amino-7-bromo-4-(7-fluoro-1H-indazol-4-yl)-8-methyl-1H-1,5-naphthyridin-2-one,
3-Amino-4-(7-fluoro-1H-indazol-4-yl)-7-iodo-8-methyl-1H-1,5-naphthyridin-2-one,
3-Amino-7-(azetidin-1-yl)-4-(7-fluoro-1H-indazol-4-yl)-8-methyl-1H-1,5-naphthyridin-2-one,
7-Amino-8-(7-fluoro-1H-indazol-4-yl)-4-methyl-6-oxo-5H-1,5-naphthyridine-3-carbonitrile,
3-Amino-7-cyclobutyl-4-(7-fluoro-1H-indazol-4-yl)-8-methyl-1H-1,5-naphthyridin-2-one,
3-Amino-4-(7-fluoro-1H-indazol-4-yl)-8-methyl-7-pyridazin-4-yl-1H-1,5-naphthyridin-2-one,
3-Amino-4-(7-fluoro-1H-indazol-4-yl)-8-methyl-7-thiazol-2-yl-1H-1,5-naphthyridin-2-one,
3-Amino-4-(7-fluoro-1H-indazol-4-yl)-8-methyl-7-(oxetan-3-yl)-1H-1,5-naphthyridin-2-one,
3-Amino-4-(7-fluoro-1H-indazol-4-yl)-8-methyl-7-vinyl-1H-1,5-naphthyridin-2-one,
3-Amino-7-ethynyl-4-(7-fluoro-1H-indazol-4-yl)-8-methyl-1H-1,5-naphthyridin-2-one,
3-Amino-4-(7-fluoro-1H-indazol-4-yl)-7-isopropenyl-8-methyl-1H-1,5-naphthyridin-2-one,
3-Amino-7-cyclopentyl-4-(7-fluoro-1H-indazol-4-yl)-8-methyl-1H-1,5-naphthyridin-2-one,
3-Amino-4-(7-fluoro-1H-indazol-4-yl)-7-isopropyl-8-methyl-1H-1,5-naphthyridin-2-one,
3-Amino-7-ethyl-4-(7-fluoro-1H-indazol-4-yl)-8-methyl-1H-1,5-naphthyridin-2-one,
3-Amino-4-(7-fluoro-1H-indazol-4-yl)-8-methyl-7-(1-methylcyclopropyl)-1H-1,5-naphthyridin-2-one,
3-Amino-7-(2,2-dimethylcyclopropyl)-4-(7-fluoro-1H-indazol-4-yl)-8-methyl-1H-1,5-naphthyridin-2-one,
3-Amino-7-(cyclopropylmethyl)-4-(7-fluoro-1H-indazol-4-yl)-8-methyl-1H-1,5-naphthyridin-2-one,
3-Amino-7-(2-fluorocyclopropyl)-4-(7-fluoro-1H-indazol-4-yl)-8-methyl-1H-1,5-naphthyridin-2-one,
3-Amino-4-(7-fluoro-1H-indazol-4-yl)-8-methyl-7-(2,2,2-trifluoroethyl)-1H-1,5-naphthyridin-2-one,
3-Amino-7-(1,1-difluoroethyl)-4-(7-fluoro-1H-indazol-4-yl)-8-methyl-1H-1,5-naphthyridin-2-one,
3-Amino-7-fluoro-4-(7-fluoro-1H-indazol-4-yl)-8-methyl-1H-1,5-naphthyridin-2-one,
3-Amino-4-(7-fluoro-1H-indazol-4-yl)-7-methoxy-8-methyl-1H-1,5-naphthyridin-2-one,
3-Amino-4-(7-fluoro-1H-indazol-4-yl)-8-methyl-7-(2,2,2-trifluoroethoxy)-1H-1,5-naphthyridin-2-one,
3-Amino-4-(7-fluoro-1H-indazol-4-yl)-7-isopropoxy-8-methyl-1H-1,5-naphthyridin-2-one,
3-Amino-7-(cyclobutoxy)-4-(7-fluoro-1H-indazol-4-yl)-8-methyl-1H-1,5-naphthyridin-2-one,
3-Amino-7-ethoxy-4-(7-fluoro-1H-indazol-4-yl)-8-methyl-1H-1,5-naphthyridin-2-one,
3-Amino-4-(7-fluoro-1H-indazol-4-yl)-8-methyl-7-methylsulfanyl-1H-1,5-naphthyridin-2-one,
3-Amino-4-(7-fluoro-1H-indazol-4-yl)-8-methyl-7-methylsulfinyl-1H-1,5-naphthyridin-2-one,
3-Amino-4-(7-fluoro-1H-indazol-4-yl)-8-methyl-7-methylsulfonyl-1H-1,5-naphthyridin-2-one,
3-Amino-4-(7-fluoro-1H-indazol-4-yl)-8-methyl-7-[rac-(1R,2R)-2-methylcyclopropyl]-1H-1,5-naphthyridin-2-one,
3-Amino-8-(difluoromethyl)-4-(7-fluoro-1H-indazol-4-yl)-7-methyl-1H-1,5-naphthyridin-2-one,
3-Amino-4-(7-chloro-1H-indazol-4-yl)-7,8-dimethyl-1H-1,5-naphthyridin-2-one,
3-Amino-6-bromo-4-(7-fluoro-1H-indazol-4-yl)-8-methyl-1H-1,5-naphthyridin-2-one,
3-Amino-4-(7-fluoro-1H-indazol-4-yl)-6-(3-hydroxy-3-methyl-but-1-ynyl)-8-methyl-1H-1,5-naphthyridin-2-one,
3-Amino-4-(7-fluoro-1H-indazol-4-yl)-6-(3-methoxy-3-methyl-but-1-ynyl)-8-methyl-1H-1,5-naphthyridin-2-one,
3-Amino-4-(7-fluoro-1H-indazol-4-yl)-8-methyl-6-[2-(oxetan-3-yl)ethynyl]-1H-1,5-naphthyridin-2-one,
3-Amino-4-(7-fluoro-1H-indazol-4-yl)-7-methyl-6-(2-thienyl)-1H-1,5-naphthyridin-2-one,
3-Amino-4-(7-fluoro-1H-indazol-4-yl)-7-methyl-6-thiazol-4-yl-1H-1,5-naphthyridin-2-one,
3-Amino-4-(7-fluoro-1H-indazol-4-yl)-7-methyl-6-thiazol-2-yl-1H-1,5-naphthyridin-2-one,
3-Amino-4-(7-fluoro-1H-indazol-4-yl)-7-methyl-6-oxazol-2-yl-1H-1,5-naphthyridin-2-one,
3-Amino-4-(7-fluoro-1H-indazol-4-yl)-7-methyl-6-(2-pyridyl)-1H-1,5-naphthyridin-2-one,
3-Amino-4-(7-fluoro-1H-indazol-4-yl)-7-methyl-6-(3-methylbut-1-ynyl)-1H-1,5-naphthyridin-2-one,
3-Amino-6-ethyl-4-(7-fluoro-1H-indazol-4-yl)-7-methyl-1H-1,5-naphthyridin-2-one,
3-Amino-7-ethyl-4-(7-fluoro-1H-indazol-4-yl)-1H-1,5-naphthyridin-2-one,
3-Amino-4-(7-chloro-1H-indazol-4-yl)-6,7-dimethyl-1H-1,5-naphthyridin-2-one,
3-Amino-5-chloro-4-(7-fluoro-1H-indazol-4-yl)-7,8-dimethyl-1H-1,6-naphthyridin-2-one,
3-Amino-4-(7-fluoro-1H-indazol-4-yl)-7,8-dimethyl-2-oxo-1H-1,6-naphthyridine-5-carbonitrile,
3-Amino-5-chloro-7-cyclopropyl-4-(7-fluoro-1H-indazol-4-yl)-8-methyl-1H-1,6-naphthyridin-2-one,
3-Amino-5-chloro-7-cyclopropyl-4-(7-fluoro-1H-indazol-4-yl)-1H-1,6-naphthyridin-2-one,
3-Amino-5-chloro-4-(7-fluoro-1H-indazol-4-yl)-8-methyl-1H-1,6-naphthyridin-2-one,
3-amino-7-cyclopropyl-4-(7-fluoro-1H-indazol-4-yl)-8-methyl-1H-1,6-naphthyridin-2-one,
3-Amino-7-cyclopropyl-4-(7-fluoro-1H-indazol-4-yl)-5-methyl-1H-1,6-naphthyridin-2-one,
3-Amino-7-cyclopropyl-4-(7-fluoro-1H-indazol-4-yl)-2-oxo-1H-1,6-naphthyridine-5-carbonitrile,
3-Amino-7-cyclopropyl-5-(difluoromethoxy)-4-(7-fluoro-1H-indazol-4-yl)-1H-1,6-naphthyridin-2-one,
3-Amino-7-cyclopropyl-4-(7-fluoro-1H-indazol-4-yl)-5-methoxy-1H-1,6-naphthyridin-2-one,
3-amino-8-fluoro-4-(7-fluoro-1H-indazol-4-yl)-5-methoxy-1H-1,6-naphthyridin-2-one,
3-Amino-8-chloro-4-(7-fluoro-1H-indazol-4-yl)-5-methoxy-1H-1,6-naphthyridin-2-one,
3-Amino-4-(7-fluoro-1H-indazol-4-yl)-5-methoxy-8-methyl-1H-1,6-naphthyridin-2-one,
3-Amino-4-(7-fluoro-1H-indazol-4-yl)-5-methoxy-8-methyl-1H-1,7-naphthyridin-2-one,
3-Amino-4-(7-fluoro-1H-indazol-4-yl)-5-methoxy-6,8-dimethyl-1H-1,7-naphthyridin-2-one,
3-Amino-6-chloro-4-(7-fluoro-1H-indazol-4-yl)-8-methyl-1H-1,7-naphthyridin-2-one,
3-Amino-5-chloro-4-(7-fluoro-1H-indazol-4-yl)-8-methyl-1H-1,7-naphthyridin-2-one,
3-Amino-4-(7-fluoro-1H-indazol-4-yl)-5,8-dimethyl-1H-1,7-naphthyridin-2-one,
3-Amino-5-ethyl-4-(7-fluoro-1H-indazol-4-yl)-8-methyl-1H-1,7-naphthyridin-2-one,
3-Amino-5-fluoro-4-(7-fluoro-1H-indazol-4-yl)-8-methyl-1H-1,7-naphthyridin-2-one,
3-Amino-5-benzyloxy-8-chloro-4-(7-fluoro-1H-indazol-4-yl)-1H-1,7-naphthyridin-2-one,
3-Amino-5-benzyloxy-4-(7-fluoro-1H-indazol-4-yl)-8-methyl-1H-1,7-naphthyridin-2-one,
3-Amino-4-(7-fluoro-1H-indazol-4-yl)-5-isopropoxy-8-methyl-1H-1,7-naphthyridin-2-one,
3-Amino-4-(7-fluoro-1H-indazol-4-yl)-8-methyl-5-(2,2,2-trifluoroethoxy)-1H-1,7-naphthyridin-2-one,
3-Amino-5-ethoxy-4-(7-fluoro-1H-indazol-4-yl)-8-methyl-1H-1,7-naphthyridin-2-one,
3-Amino-5-(difluoromethoxy)-4-(7-fluoro-1H-indazol-4-yl)-8-methyl-1H-1,7-naphthyridin-2-one,
3-Amino-5-(2,2-difluoroethoxy)-4-(7-fluoro-1H-indazol-4-yl)-8-methyl-1H-1,7-naphthyridin-2-one,
3-Amino-5-(cyclopropoxy)-4-(7-fluoro-1H-indazol-4-yl)-8-methyl-1H-1,7-naphthyridin-2-one,
3-Amino-5-(3,3-difluoropropoxy)-4-(7-fluoro-1H-indazol-4-yl)-8-methyl-1H-1,7-naphthyridin-2-one,
3-Amino-5-(cyclobutoxy)-4-(7-fluoro-1H-indazol-4-yl)-8-methyl-1H-1,7-naphthyridin-2-one,
3-Amino-4-(7-fluoro-1H-indazol-4-yl)-8-methyl-5-propoxy-1H-1,7-naphthyridin-2-one,
3-Amino-5-(cyclopropylmethoxy)-4-(7-fluoro-1H-indazol-4-yl)-8-methyl-1H-1,7-naphthyridin-2-one,
3-Amino-4-(7-fluoro-1H-indazol-4-yl)-8-methyl-5-(trifluoromethoxy)-1H-1,7-naphthyridin-2-one,
3-Amino-4-(7-fluoro-1H-indazol-4-yl)-5-(methoxymethyl)-8-methyl-1H-1,7-naphthyridin-2-one,
3-Amino-7-bromo-4-(7-fluoro-1H-indazol-4-yl)-6-methyl-1H-1,5-naphthyridin-2-one,
3-Amino-7-ethyl-4-(7-fluoro-1H-indazol-4-yl)-6-methyl-1H-1,5-naphthyridin-2-one,
   or a pharmaceutically acceptable salt thereof.

A further embodiment of present invention is (xxix) a process for the preparation of a compound having the structure of formula (I),

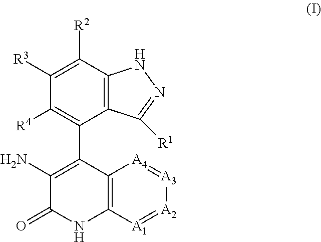

comprising one of the following steps:
(a) decomposition of pyridinium salt of formula (IX)

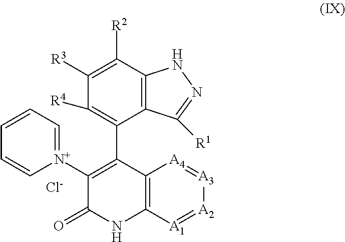

with hydrazine hydrate;
(b) cross coupling of compound of formula (XVII),

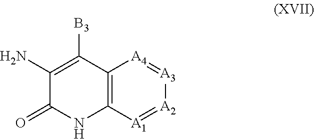

and compound of formula (XVIII),

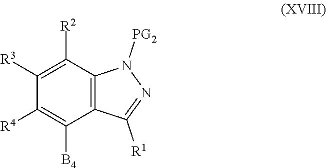

in the presence of a catalyst to provide compound of formula (XIX)

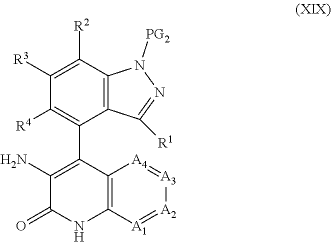

followed by deprotecting of the compound of formula (XIX) in the presence an acid,
(c) decomposition of pyridinium salts of formula (XXIV)

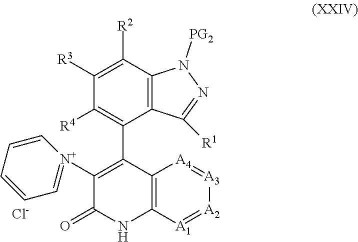

with hydrazine hydrate to afford compound of formula (XIX),

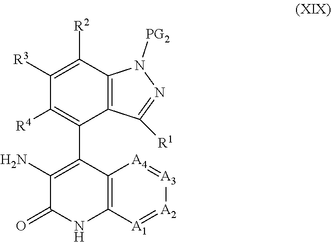

followed by deprotection of compound of formula (XIX) in the presence an acid;
wherein B³ is halogen, preferably bromo or iodide; B⁴ is boronic acid, boronic ester or tributyltin, PG₂ is hydrogen or a protecting group, preferably tetrahydropyranyl group, the acid is preferably trifluoroacetic acid or HCl, the catalyst is preferably Pd(dtbpf)Cl₂, Pd(dppf)Cl₂ or Pd(PPh₃)₄;
wherein B³ is halogen, preferably bromo or chloro; B⁴ is boronic acid, boronic ester or tributyltin, PG₂ is hydrogen or a protecting group, preferably tetrahydropyranyl group, and the acid is preferably trifluoroacetic acid or HCl, the catalyst is preferably Pd(dtbpf)Cl₂, Pd(dppf)Cl₂ or Pd(PPh₃)₄;
wherein R¹, R², R³, R⁴, A₁, A₂, A₃, and A₄ are as defined herein.

A further embodiment of present invention is (xxx) a compound or a pharmaceutically acceptable salt thereof according to any one of (i) to (xxviii), when manufactured according to the process of (xxix).

A further embodiment of present invention is (xxxi) a pharmaceutical composition comprising a compound or a pharmaceutically acceptable salt thereof according to any one of (i) to (xxviii), and a pharmaceutically acceptable excipient.

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit the growth of PKMYT1 dependent cancers (i.e., cancers whose survival depends on PKMYT1 kinase activity). For example, such amount may be below the amount that is toxic to normal cells, or the mammal as a whole.

In one example, the pharmaceutically effective amount of the compound of the invention administered parenterally per dose will be in the range of about 0.1 to 1000 mg/kg, alternatively about 0.1 to 1000 mg/kg of patient body weight per day, with the typical initial range of compound used being 0.3 to 15 mg/kg/day. In another embodiment, oral unit dosage forms, such as tablets and capsules, preferably contain from about 1 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration.

Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 1 to 1000 mg of the compound of the invention compounded with about 1 to 1000 mg anhydrous lactose, about 1 to 1000 mg sodium croscarmellose, about 1 to 1000 mg polyvinylpyrrolidone (PVP) K30, and about 1 to 1000 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a stereoisomer or pharmaceutically acceptable salt thereof. In a further embodiment includes a pharmaceutical composition comprising a compound of formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment of PKMYT1 dependent cancers. Another embodiment includes a pharmaceutical composition comprising a compound of Formula (I) for use in the treatment of PKMYT1 dependent cancers.

The following composition A and B illustrate typical compositions of the present invention, but serve merely as representative thereof.

### Composition A

A compound of the present invention can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Composition B

A compound of the present invention can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Indications and Methods of Treatment

The compounds of the invention bind to the kinase domain of PKMYT1, inhibit its kinase activity and thereby reduce inhibitory CDK1 phosphorylation at Thr14 without grossly affecting CDK1 phosphorylation at Tyr15. Inhibition of PKMYT1, a negative regulator of CDK1, causes unscheduled CDK1 activation that forces cells to enter into mitosis. In cancer cells with high level of replication stress or DNA damage, e.g. cancer cells with amplification of cyclin E1 (CCNE1), inhibition of PKMYT1 further induces CDK1 imbalance and promotes early mitotic entry in cells undergoing DNA synthesis and DNA repair, which eventually results in accumulation of replication stress and leads to catastrophic genome instability. Therefore, the compounds of the invention are useful for treating cancers bearing high level of replication stress and genome instability. Alternatively, the compounds of the invention are useful for combinational therapeutic approaches with agents that induce DNA damage or perturb DNA replication or DNA damage repair process.

Another embodiment includes a method of treating cancer in a mammal in need of such treatment, wherein the method comprises administering to said mammal a therapeutically effective amount of a compound of formula (I), a stereoisomer, tautomer, prodrug or pharmaceutically acceptable salt thereof. Cancer includes but is not limited to sarcomas, adenocarcinomas, blastomas, and carcinomas, of the various organ systems, such as those affecting pancreas, liver, lung, breast, stomach, buliarintestinal (e.g. colon), genitourinary tract (e.g. renal urothelial cells) and ovary.

A further embodiment of present invention is (xxxii) a compound of the invention for use as therapeutically active substance.

A further embodiment of present invention is (xxxiii) a compound of the invention for use in the treatment or cancer.

A further embodiment of present invention is (xxxiv) the use of a compound of the invention for the treatment of cancer.

A further embodiment of present invention is (xxxv) the use of a compound of the invention for the inhibition of PKMYT1.

A further embodiment of present invention is (xxxvi) the use of a compound of the invention for the preparation of a medicament for the treatment of cancer.

A further embodiment of present invention is (xxxvii) the use of a compound of the invention for the preparation of a medicament for the inhibition of PKMYT1.

A further embodiment of present invention is (xxxviii) a method for the treatment of cancer, which method comprises administering an effective amount of a compound of the invention.

A further embodiment of present invention is (xxxix) the use of according to (xxxiv) or (xxxvi), or the method according to (xxxviii), wherein the cancer is a cancer in pancreas, liver, lung, breast, stomach, buliarintestinal, genitourinary tract, or ovary.

### Synthesis

The compounds of the present invention can be prepared by any conventional means. Suitable processes for synthesizing these compounds as well as their starting materials are provided in the schemes below and in the examples. All substituents, in particular, R¹, R², R³, R⁴, A₁, A₂, A₃, and A₄ are defined above. Furthermore, and unless explicitly otherwise stated, all reactions, reaction conditions, abbreviations and symbols have the meanings well known to a person of ordinary skill in organic chemistry.

General synthetic routes for preparing the compound of formula (I) are shown below.

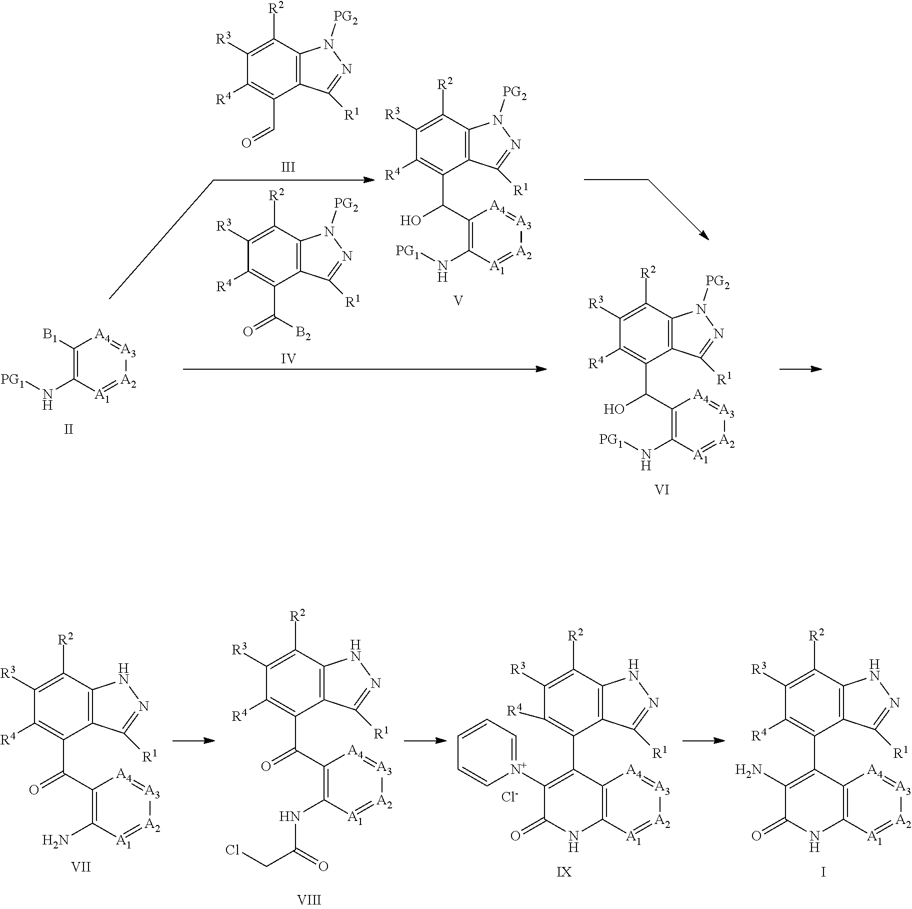

Wherein B¹ is halogen, such as Br or I; B₂ is ester such as —OMe or —OEt, or amide, such as Weinreb Amide

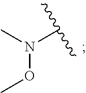 [SMILES: *N(C)OC]

PG¹ is a protecting group, such as tert-butoxycarbonyl and PG² is hydrogen or a protecting group, such as tetrahydropyranyl group.

Compound of formula (V) could be obtained from metal-halogen exchange reaction of compound of formula (II) in the presence of organometallic reagent, such as n-BuLi or i-PrMgBr, followed by nucleophilic addition reacting with compound of formula (III). The oxidation of alcohol of the compound of formula (V) with a suitable oxidant, such as Dess-Martin periodinane or MnO₂, could afford compound of formula (VI).

Alternatively, compound of formula (VI) could be obtained directly from metal-halogen exchange reaction of compound of formula (II) in the presence of organometallic reagent, such as n-BuLi or i-PrMgBr, followed by nucleophilic addition reaction with compound of formula (IV). The deprotection of compound of formula (VI) in the presence of a suitable acid, such as TFA or HCl, could afford compound of formula (VII). Compound of formula (VIII) could be prepared from acylation of compound of formula (VII) with 2-chloroacetyl chloride in the presence of a suitable base such as pyridine, DMAP, TEA or DIPEA. Cyclization of compound of formula (VIII) in the presence of pyridine could afford compound of formula (IX). Compound of formula (I) could be obtained by decomposition of pyridinium salts of formula (IX) with hydrazine hydrate.

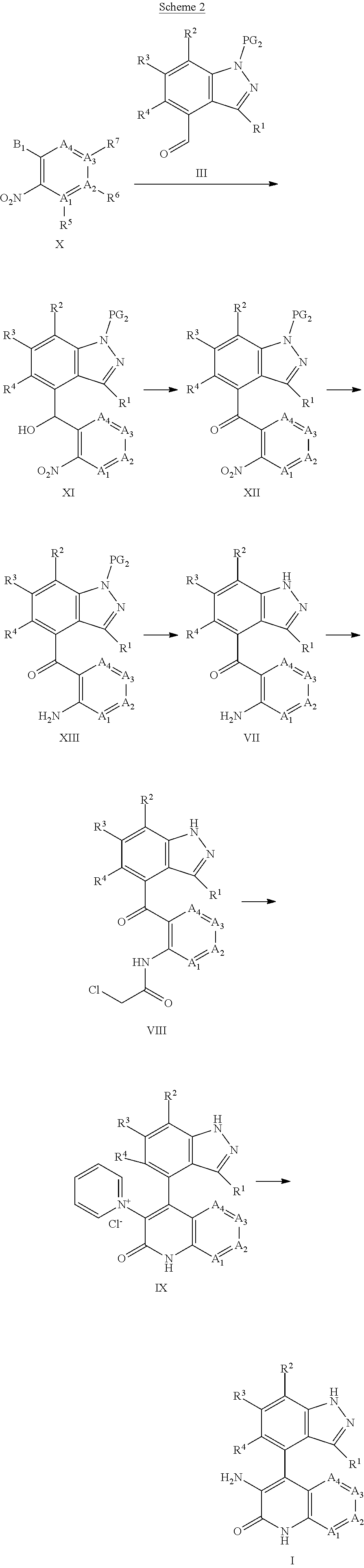

Alternatively, compound of formula (I) could also be prepared in the process illustrated in the scheme 2. Compound of formula (XI) could be obtained from metal-halogen exchange reaction of compound of formula (X) with Grignard reagent, such as PhMgBr or i-PrMgBr, followed by Grignard reaction with compound of formula (III). Oxidation of compound of formula (XI) in the presence of a suitable oxidation reagent, such as Dess-Martin periodinane or MnO₂, could afford compound of formula (XII). Compound of formula (XIII) could be prepared by reduction reaction of compound of formula (XII) in the presence of s suitable reduction reagent, such as iron powder, sodium dithionite or Pd/C with hydrogen. Deprotecting of PG₂ of compound of formula (XIII) could afford compound of formula (VII) in the presence a suitable acid such as TFA or HCl. Compound of formula (VIII) could be prepared from acylation of compound of formula (VII) with 2-chloroacetyl chloride in the presence of a suitable base such as pyridine, DMAP, TEA or DIPEA. Cyclization of compound of formula (VIII) in the presence of pyridine could afford compound of formula (IX). Compound of formula (I) could be obtained by decomposition of pyridinium salts of formula (IX) with hydrazine hydrate.

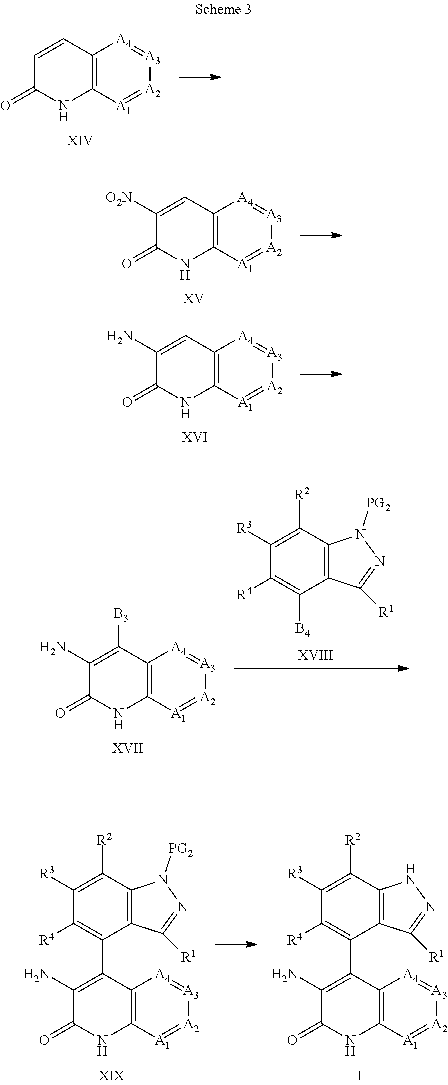

Wherein B³ is halogen, such as bromo or iodide, B⁴ is boronic acid, boronic ester or tributyltin.

Alternatively, the compound of formula (I) could also be prepared according to the scheme 3. The nitration of compound of formula (XIV) in the presence a suitable nitrating reagent, such as nitric acid, could afford compound of formula (XV). The reduction of compound of formula (XV) in the presence a suitable reduction reagent, such as iron powder, sodium dithionite or Pd/C with hydrogen, could afford compound of formula (XVI). Halogenation of compound of formula (XVI) in the presence of suitable reagent, such as NBS or NIS, could afford compound of formula (XVII). Cross coupling reaction of compound of formula (XVII) and compound of formula (XVIII) in the presence of suitable catalyst, such as Pd(dtbpf)Cl₂, Pd(dppf)Cl₂ or Pd(PPh₃)₄, could afford compound of formula (XIX). Compound of formula (I) could be obtained by deprotection of compound of formula (XIX) in the presence a suitable acid such as TFA or HCl.

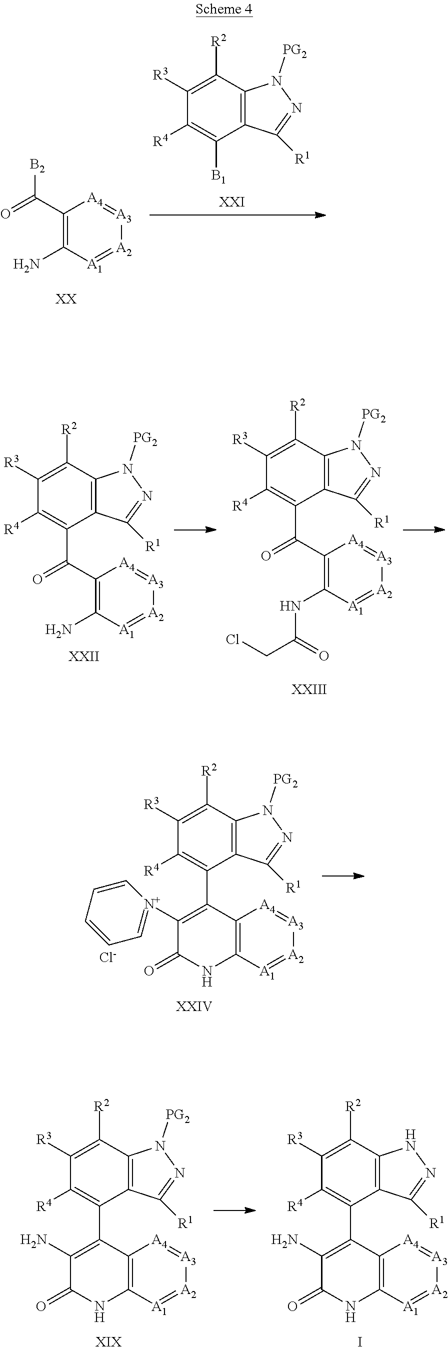

Alternatively, the compound of formula (I) could also be prepared according to scheme 4. Compound of formula (XXII) could be obtained from metal-halogen exchange reaction of compound of formula (XXI) in the presence of organometallic reagent, such as n-BuLi or i-PrMgBr, followed by nucleophilic addition reacting with compound of formula (XX). Compound of formula (XXIII) could be prepared from the acylation of compound of formula (XXII) with 2-chloroacetyl chloride in the presence of a suitable base such as pyridine, DMAP, TEA or DIPEA. Cyclization of compound of formula (XXIII) in the presence of pyridine could afford compound of formula (XXIV). Decomposition of pyridinium salts of formula (XXIV) with hydrazine hydrate could afford compound of formula (XIX)). Compound of formula (I) could be obtained by deprotection of compound of formula (XIX) in the presence a suitable acid such as TFA or HCl.

Compounds of this invention can be obtained as mixtures of diastereomers or enantiomers, which can be separated by methods well known in the art, e.g. (chiral) HPLC or SFC.

This invention also relates to a process for the preparation of a compound of formula (I) comprising one of the following steps:
a) formation of compound of formula (I),

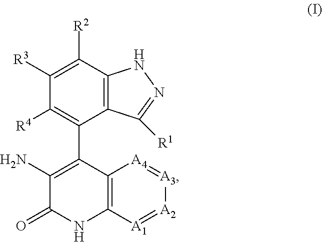

via decomposition reaction of compound of formula (IX),

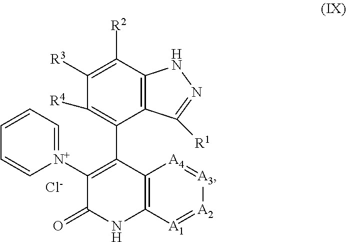

in the presence of hydrazine hydrate;
b) formation of compound of formula (I),

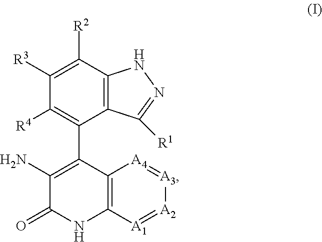

via cross coupling of compound of formula (XVII),

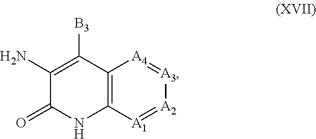

and compound of formula (XVIII),

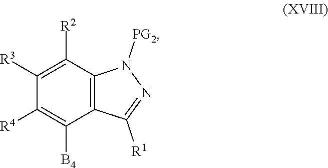

followed by deprotecting of PG₂.
wherein B³ is halogen, such as bromo or iodide; B⁴ is boronic acid, boronic ester or tributyltin and PG2 is hydrogen or a protecting group, such as tetrahydropyranyl group.

### EXAMPLES

The invention will be more fully understood by reference to the following examples. They should not, however, be construed as limiting the scope of the invention.

### Abbreviations

Abbreviations used herein are as follows:
aq.: aqueous
BAST: bis(2-methoxyethyl)aminosulfur trifluoride
BNMO: bis(naphthalen-1-ylmethyl)oxalamide
Boc: tert-butoxycarbonyl group
DCM: dichloromethane
DIPEA: N,N-diisopropylethylamine
DMAP: 4-dimethylaminopyridine
DMP: Dess-Martin periodinane
DMF: N,N-dimethylformamide
EDCI: 1-ethyl-3-(3-dimethylaminopropyl)carbodiimide
EtOAc: ethyl acetate
NCS: N-chlorosuccinimide
NBS: N-bromosuccinimide
NIS: N-iodosuccinimide
NMP: N-methyl-2-pyrrolidone
NaHMDS: sodium bis(trimethylsilyl)amide
NADPH: reduced nicotinamide adenine dinucleotide phosphate
FA: formic acid
THF: tetrahydrofuran
TFA: trifluoroacetic acid
TEA: triethylamine
TsOH: p-toluenesulfonic acid
KOAc: potassium acetate
Pd(dppf)Cl₂: 1,1′-bis(diphenylphosphino)ferrocene-palladium(II)dichloride
Pd(dtbpf)Cl₂: 1,1′-bis(di-tert-butylphosphino)ferrocene palladium dichloride
PhMgBr: phenylmagnesium bromide
i-PrMgBr: isopropylmagnesium bromide
LiHMDS: lithium bis(trimethylsilyl)amide
Na₂CO₃: sodium carbonate
HEPES: 4-(2-hydroxyethyl)-1-piperazineethanesulfonic acid
EGTA: ethylene glycol-bis(O-aminoethyl ether)-N,N,N′,N′-tetraacetic acid
DTT: dithiothreitol
IPA: isopropyl alcohol
rt: room temperature
IC₅₀: the molar concentration of an inhibitor, which produces 50% of the maximum possible response for that inhibitor.
HPLC: high performance liquid chromatography
Prep-TLC: preparative thin layer chromatography
MS (ESI): mass spectroscopy (electron spray ionization)
obsd: observed
PE: petroleum ether
δ: chemical shift
SFC: supercritical fluid chromatography

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Alternatively, intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm); Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water); or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates and final compounds were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃·H₂O in MeOH), back pressure 100 bar, detection UV@254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃·H₂O in H₂O; B: acetonitrile;
Basic condition II: A: 0.025% NH₃·H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (MH)⁺.

NMR Spectra were obtained using Bruker Avance 400/500 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

The following examples are intended to illustrate the meaning of the present invention but should by no means represent a limitation within the meaning of the present invention:

### Intermediate 1: 7-Fluoro-N-methoxy-N-methyl-1-tetrahydropyran-2-yl-indazole-4-carboxamide

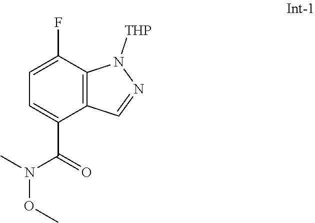

### Step 1: Methyl 7-fluoro-1H-indazole-4-carboxylate

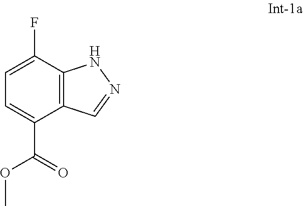

The mixture of 4-bromo-7-fluoro-1H-indazole (20.0 g, 93.0 mmol), TEA (25.86 mL, 186 mmol) and Pd(dppf)Cl₂ (6.81 g, 9.3 mmol) in methanol (200 mL) was stirred at 80° C. for 12 hours under CO atmosphere (50 psi). After completion, the mixture was filtered and the filtrate was concentrated in vacuo to give a residue, which was purified by silica-gel chromatography (elute with EtOAc:PE=0-50%) to give compound Int-1a (18.0 g, 99.6% yield) as a yellow solid. MS (ESI⁺) obsd. [(M+H)⁺]: 195.2.

### Step 2: 7-Fluoro-1H-indazole-4-carboxylic acid

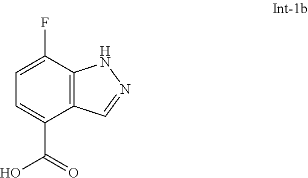 [SMILES: O=C(O)c1ccc(F)c2[nH]ncc12]

The mixture of compound Int-1a (10.0 g, 51.5 mmol) and NaOH (0.8 g, 200 mmol) in methanol (50 mL) and water (50 mL) was stirred at 25° C. for 2 hours. After completion, the pH of the mixture was adjusted to around 3 by HCl aq. (1 M). The suspension was collected by filtration to afford a solid, which was washed with water (30 mL) and concentrated in vacuo to give compound Int-1b (9.0 g, 97.0% yield) as a yellow solid. MS (ESI⁺) obsd. [(M+H)⁺]:181.1

### Step 3: 7-Fluoro-N-methoxy-N-methyl-1H-indazole-4-carboxamide

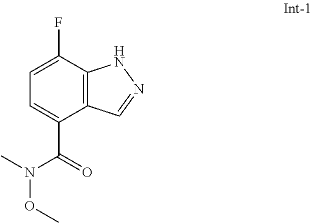 [SMILES: CON(C)C(=O)c1ccc(F)c2[nH]ncc12]

The mixture of Int-1b (8.0 g, 44.4 mmol), O,N-dimethylhydroxylamine HCl salt (5.2 g, 53.3 mmol), TEA (9.26 mL, 66.6 mmol) and EDCI (12.7 g, 66.6 mmol) in DCM (100 mL) was stirred at 25° C. for 2 hours. After completion, the mixture was poured into water (100 mL) and extracted with EtOAc (3×100 mL). The combined organic layer was washed with brine (200 mL), dried over anhydrous sodium sulfate and concentrated in vacuo to give a residue, which was purified by silica-gel chromatography (elute with EtOAc:PE=0-50%) to give compound Int-1 (7.0 g, 70.6% yield) as a white solid. MS (ESI⁺) obsd. [(M+H)⁺]: 224.1.

### Intermediate 2: tert-Butyl N-(2-bromo-5-chloro-3-pyridyl)carbamate

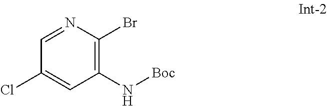 [SMILES: CC(C)(C)OC(=O)Nc1cc(Cl)cnc1Br]

To a solution of 2-bromo-5-chloro-pyridin-3-amine (1.0 g, 4.82 mmol) in THE (20 mL) was added NaHMDS (9.64 mL, 9.64 mmol) dropwise at 0° C. The mixture was stirred at 0° C. for 30 minutes. And then, a solution of di-tert-butyldicarbonate (1.05 g, 4.82 mmol) in THE (5 mL) was added dropwise to the mixture at 0° C. Then the mixture was warmed up to 25° C. and stirred for another 2 hours. After completion, the reaction was quenched with saturated ammonium chloride (10 mL) at 0° C. and extracted with EtOAc (10 mL×3). The combined organic layer was washed with brine (10 mL), dried over anhydrous sodium sulfate and concentrated under in vacuo to give a residue, which was purified by silica-gel chromatography (elute with EtOAc:PE=0-10%) to give compound Int-2 (1.2 g, 80.9% yield) as a white solid. MS (ESI⁺) obsd. [(M+H)⁺]: 307.0.

### Intermediate 3: tert-Butyl (2-bromo-5-methylpyridin-3-yl)carbamate

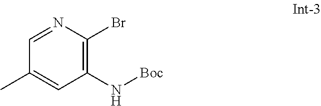 [SMILES: Cc1cnc(Br)c(NC(=O)OC(C)(C)C)c1]

The title compound was prepared in analogy to the preparation of compound Int-2, replacing 2-bromo-5-chloro-pyridin-3-amine with 2-bromo-5-methyl-pyridin-3-amine. Compound Int-3 (9.5 g) was obtained as a white solid. MS (ESI⁺) obsd. [(M+H)⁺]: 287.1.

### Intermediate 4: tert-Butyl N-(2-bromo-5-fluoro-3-pyridyl)carbamate

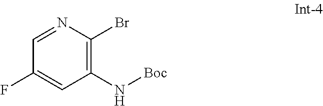 [SMILES: CC(C)(C)OC(=O)Nc1cc(F)cnc1Br]

The title compound was prepared in analogy to the preparation of compound Int-2, replacing 2-bromo-5-chloro-pyridin-3-amine with 2-bromo-5-fluoro-pyridin-3-amine. Compound Int-4 (1.5 g) was obtained as a white solid. MS (ESI⁺) obsd. [(M+H)⁺]: 291.1.

### Intermediate 5: tert-Butyl N-[2-bromo-5-(trifluoromethyl)-3-pyridyl]carbamate

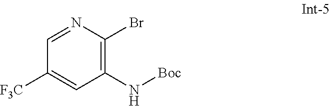 [SMILES: CC(C)(C)OC(=O)Nc1cc(C(F)(F)F)cnc1Br]

The title compound was prepared in analogy to the preparation of compound Int-2, replacing 2-bromo-5-chloro-pyridin-3-amine with 2-bromo-5-(trifluoromethyl)pyridin-3-amine. Compound Int-5 (1.2 g) was obtained as a white solid. MS (ESI⁺) obsd. [(M+H)⁺]: 341.1.

### Intermediate 6: tert-Butyl N-(2-bromo-5-methoxy-3-pyridyl)carbamate

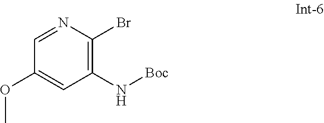 [SMILES: COc1cnc(Br)c(NC(=O)OC(C)(C)C)c1]

The title compound was prepared in analogy to the preparation of compound Int-2, replacing 2-bromo-5-chloro-pyridin-3-amine with 2-bromo-5-methoxy-pyridin-3-amine. Compound Int-6 (1.0 g) was obtained as a white solid. MS (ESI⁺) obsd. [(M+H)⁺]: 303.1.

### Intermediate 7: tert-Butyl N-[2-bromo-6-(cyclobutoxy)-5-methyl-3-pyridyl]carbamate

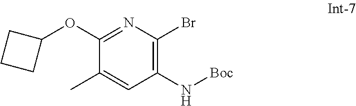 [SMILES: Cc1cc(NC(=O)OC(C)(C)C)c(Br)nc1OC1CCC1]

### Step 1: 2-(Cyclobutoxy)-3-methyl-5-nitro-pyridine

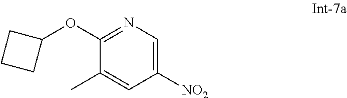

To a mixture of cyclobutanol (1.0 g, 13.9 mmol) in THE (10 mL) was added NaH (0.83 g, 20.8 mmol) at 0° C. The mixture was stirred at 0° C. for 30 minutes. Then a solution of 2-chloro-3-methyl-5-nitropyridine (3.6 g, 20.8 mmol) in THE (25 mL) was added to the mixture and stirred at 25° C. for 12 hours. After completion, the mixture was poured into water (50 mL) and extracted with EtOAc (3×50 mL). The combined organic layer was washed with brine (50 mL), dried over anhydrous sodium sulfate and concentrated in vacuo to give a residue, which was purified by silica-gel chromatography (elute with EtOAc:PE=0-10%) to give compound Int-7a (1.7 g, 58.2% yield) as a white solid. MS (ESI⁺) obsd. [(M+H)⁺]: 209.1.

### Step 2: 6-(Cyclobutoxy)-5-methyl-pyridin-3-amine

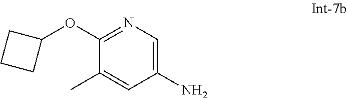

The mixture of compound Int-7a (1.7 g, 8.1 mmol), NH₄Cl (2.16 g, 40.3 mmol) and iron powder (3.8 g, 40.4 mmol) in ethanol (15 mL) and water (15 mL) was stirred at 80° C. for 2 hours. After completion, the mixture was filtered and the filtrate was concentrated under reduced pressure to give a residue, which was purified by silica-gel chromatography (elute with EtOAc:PE=0-30%) to give compound Int-7b (1.4 g, 97.4% yield) as a brown oil. MS (ESI⁺) obsd. [(M+H)⁺]: 179.1.

### Step 3: 2-Bromo-6-(cyclobutoxy)-5-methyl-pyridin-3-amine

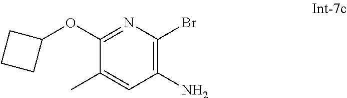

To a solution of compound Int-7b (1.3 g, 7.3 mmol) in MeCN (15 mL) was added the suspension of NBS (1.4 g, 7.7 mmol) in MeCN (5 mL) at 0° C. The mixture was warmed up to 25° C. and stirred for 1 hour. After completion, the reaction mixture was quenched with saturated sodium sulfite (20 mL) and saturated Na₂CO₃ (50 mL). Then the resulting mixture was extracted with EtOAc (1.2 L×3). The combined organic layer was washed with brine (1.2 L), dried over anhydrous sodium sulfate and concentrated in vacuo to give a residue, which was purified by silica-gel chromatography (elute with EtOAc:PE=0-30%) to give compound Int-7c (1.8 g, 96.0% yield) as a brown oil. MS (ESI⁺) obsd. [(M+H)⁺]: 257.0.

### Step 4: tert-Butyl N-[2-bromo-6-(cyclobutoxy)-5-methyl-3-pyridyl]carbamate

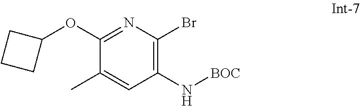 [SMILES: Cc1cc(NC(=O)OC(C)(C)C)c(Br)nc1OC1CCC1]

The title compound was prepared in analogy to the preparation of compound Int-2, replacing 2-bromo-5-chloro-pyridin-3-amine with compound Int-7c. Compound Int-7 (1.0 g) was obtained as a white solid. MS (ESI⁺) obsd. [(M+H)⁺]: 357.2.

### Intermediate 8: tert-Butyl N-[2-bromo-6-(cyclopropylmethoxy)-5-methyl-3-pyridyl]carbamate

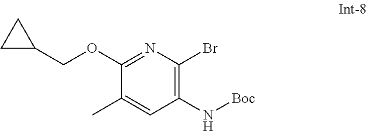 [SMILES: Cc1cc(NC(=O)OC(C)(C)C)c(Br)nc1OCC1CC1]

The title compound was prepared in analogy to the preparation of compound Int-7, replacing cyclobutanol with cyclopropylmethanol. Compound Int-8 (1.0 g) was obtained as a white solid. MS (ESI⁺) obsd. [(M+H)⁺]: 357.2.

### Intermediate 9: tert-Butyl N-[6-chloro-2-(7-fluoro-1-tetrahydropyran-2-yl-indazole-4-carbonyl)-5-methyl-3-pyridyl]carbamate

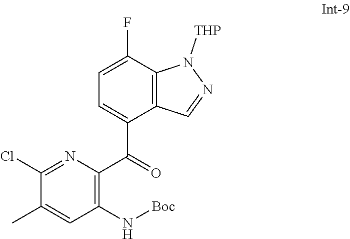

### Step 1: 2-Bromo-4-chloro-5-methyl-aniline

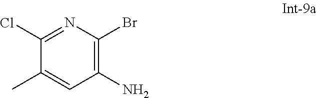

To a mixture of 2-bromo-5-methylaniline (20.0 g, 107.5 mmol) in DMF (300 mL) was added a solution of NCS (15.8 g, 118.3 mmol) in DMF (20 mL) at 0° C. The resulting mixture was stirred at 25° C. for 16 hours. After completion, the reaction mixture was quenched with saturated sodium sulfite (500 mL) and saturated Na₂CO₃ (200 mL), and extracted with EtOAc (600 mL×3). The combined organic layer was dried over anhydrous sodium sulfate and concentrated in vacuo to give a residue, which was purified by silica-gel chromatography (elute with EtOAc:PE=0-30%) to give compound Int-9a (21.0 g, 88.6% yield) as a yellow solid. MS (ESI⁺) obsd. [(M+H)⁺]: 221.0.

### Step 2: tert-Butyl N-(2-bromo-6-chloro-5-methyl-3-pyridyl)carbamate

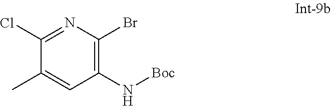 [SMILES: Cc1cc(NC(=O)OC(C)(C)C)c(Br)nc1Cl]

The title compound was prepared in analogy to the preparation of compound Int-2, replacing 2-bromo-5-chloro-pyridin-3-amine with compound Int-9a. Compound Int-9b (6.7 g) was obtained as a white solid. MS (ESI⁺) obsd. [(M+H)⁺]: 322.9.

### Step 3: tert-Butyl N-[6-chloro-2-(7-fluoro-1-tetrahydropyran-2-yl-indazole-4-carbonyl)-5-methyl-3-pyridyl]carbamate

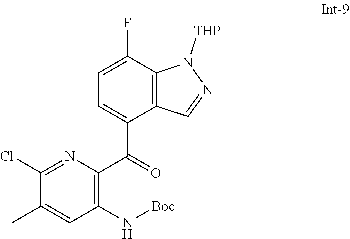

To a solution of compound Int-9b (1.1 g, 3.58 mmol) in tetrahydrofuran (15 mL) was added NaH (156.2 mg, 3.9 mmol) at 0° C. The reaction mixture was stirred at 25° C. for 30 minutes. Then the reaction mixture was cooled to −78° C. and n-BuLi (1.4 mL, 3.6 mmol) was dropwise added at −78° C. and stirred for another 30 minutes. And then, a solution of compound Int-1 (1.0 g, 3.3 mmol) in tetrahydrofuran (2 mL) was added and the mixture was stirred at −78° C. for another 30 minutes. After completion, the reaction mixture was quenched with saturated aqueous ammonium chloride (20 mL), extracted with ethyl acetate (30 mL×3). The combined organic layer was washed with brine (20 mL), dried over anhydrous sodium sulfate and concentrated in vacuo to give the residue, which was purified by silica-gel chromatography (elute with EtOAc:PE=0-10%) to give compound Int-9 (470.0 mg, 29.5% yield) as a yellow oil. MS (ESI⁺) obsd. [(M+H)⁺]: 489.1.

### Intermediate 10: 3-Amino-4-bromo-7-chloro-1H-1,5-naphthyridin-2-one

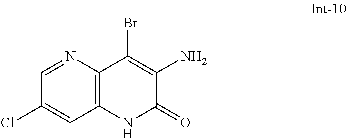 [SMILES: Nc1c(Br)c2ncc(Cl)cc2[nH]c1=O]

### Step 1: 7-Chloro-3-nitro-1H-1,5-naphthyridin-2-one

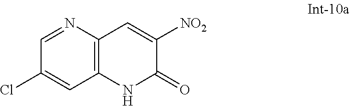 [SMILES: O=c1[nH]c2cc(Cl)cnc2cc1[N+](=O)[O-]]

To a solution of 7-chloro-1H-1,5-naphthyridin-2-one (3.5 g, 19.4 mmol) in con. H₂SO₄ (40 mL) was added KNO₃ (3.9 g, 38.8 mmol). The mixture was stirred at 120° C. and KNO₃ (2.0 g, 19.4 mmol) was added each 6 hours until the starting material consumed completely. After completion, the resulting mixture was poured into ice water (200 mL) and the suspension was filtered to afford the solid, which was washed with water and dried in vacuo to afford crude compound Int-10a (2.8 g, 63.8% yield) as a yellow solid and used for next step without further purification. MS (ESI⁺) obsd. [(M+H)⁺]: 226.0.

### Step 2: 3-Amino-7-chloro-1H-1,5-naphthyridin-2-one

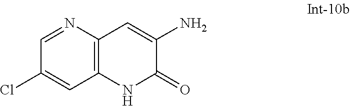 [SMILES: Nc1cc2ncc(Cl)cc2[nH]c1=O]

The mixture of compound Int-10a (2.70 g, 12.0 mmol), ammonium chloride (6.4 g, 119.7 mmol) and iron powder (5.4 g, 95.8 mmol) in MeOH (100 mL) and water (20 mL) was stirred at 75° C. for 2 hours. After completion, the resulting mixture was filtered and the filtrate was concentrated in vacuo to give a residue, which was washed with water and dried in vacuo to afford crude compound Int-10b (1.5 g, 63.9% yield) as a yellow solid, which was used for next step without further purification. MS (ESI⁺) obsd. [(M+H)⁺]: 196.0.

### Step 3: 3-Amino-4-bromo-7-chloro-1H-1,5-naphthyridin-2-one

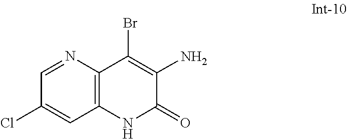 [SMILES: Nc1c(Br)c2ncc(Cl)cc2[nH]c1=O]

To a suspension of compound Int-10b (420.0 mg, 2.2 mmol) in acetonitrile (30 mL) was added NBS (401.0 mg, 2.3 mmol) at 0° C. The mixture was stirred at 0° C. for 1 hour. After completion, the mixture was concentrated in vacuo to afford residue, which was washed with water and MeOH. The solid was dried in vacuo to afford crude compound Int-10 (280.0 mg, 46.5% yield) as a brown solid, which was used for the next step without further purification. MS (ESI⁺) obsd. [(M+H)⁺]: 273.9.

### Intermediate 11: 4-Bromo-7-fluoro-5-methyl-1-tetrahydropyran-2-yl-indazole

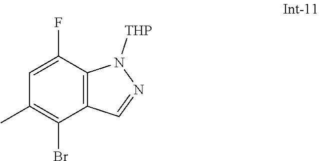

### Step 1: 2-Bromo-5,6-difluoro-3-methyl-benzaldehyde

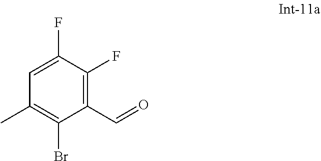

To a solution of 2,2,6,6-tetramethylpiperidine (3.6 g, 25.4 mmol) in anhydrous THE (50 mL) was added n-BuLi (2.0 M in hexane, 12.7 mL) dropwise under N₂ atmosphere at −70° C. and the mixture was stirred for 10 minutes. And then, a solution of 1-bromo-4,5-difluoro-2-methyl-benzene (5.0 g, 24.2 mmol) in THE (15 mL) was added to the mixture dropwise and the mixture was stirred at −70° C. for another 2 hours, followed by adding DMF (2.0 g, 26.6 mmol) dropwise. After completion, the mixture was warmed up to −20° C. and quenched with HCl (1 M aqueous, 20 mL) dropwise. The mixture was extracted with EtOAc (100 mL×3). The combined organic layers were washed with HCl aq. (1M) and brine, dried over anhydrous sodium sulfate and concentrated in vacuo to afford compound Int-11a (5.2 g, 87.1% yield) as a yellow solid, which was used in next step without further purification.

### Step 2: 4-Bromo-7-fluoro-5-methyl-1H-indazole

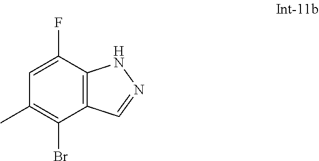

A mixture of compound Int-11a (5.2 g, 22.0 mmol) in hydrazine monohydrate (3.3 g, 3.2 mL) and 1,4-dioxane (50 mL) was stirred at 110° C. for 12 hours. After completion, the resulting mixture was concentrated in vacuo to give a crude residue, which was redissolved in EtOAc (100 mL) and washed with water (100 mL×3). The organic layer was dried over anhydrous sodium sulfate and concentrated in vacuo to afford compound Int-11b (4.9 g, 97.3% yield) as a yellow solid, which was used in next step without further purification. MS (ESI⁺) obsd. [(M+H)⁺]: 228.9.

### Step 3: 4-Bromo-7-fluoro-5-methyl-1-tetrahydropyran-2-yl-indazole

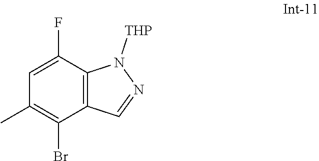

The mixture of Int-11b (4.5 g, 19.7 mmol), 3,4-dihydro-2H-pyran (5.0 g, 59.0 mmol) and p-toluenesulfonic acid monohydrate (374.0 mg, 2.0 mmol) in anhydrous DCM (90 mL) was stirred at rt for 2 hours. After completion, the reaction was quenched with the saturated solution of NaHCO₃ and extracted with DCM (100 mL×3). The combined organic layers were concentrated in vacuo to give a residue, which was purified by silica-gel chromatography (elute with EtOAc:PE=0-10%) to afford compound Int-11 (3.8 g, 61.6% yield) as a white solid. MS (ESI⁺) obsd. [(M+H)⁺]: 313.1.

### Intermediate 12: 4-Bromo-5-chloro-7-fluoro-1-tetrahydropyran-2-yl-indazole

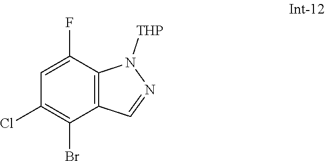

### Step 1: N-benzyl-7-fluoro-1-tetrahydropyran-2-yl-indazol-4-amine

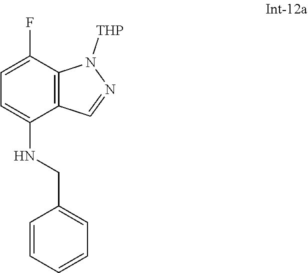

A mixture of compound 4-bromo-7-fluoro-1-tetrahydropyran-2-yl-indazole (4.1 g, 13.7 mmol), benzylamine (2.2 g, 20.6 mmol), Cs₂CO₃ (11.2 g, 34.2 mmol), tris(dibenzylideneacetone)dipalladium (0) chloroform adduct (1.4 g, 1.4 mmol) and RuPhos (959.3 mg, 2.1 mmol) in toluene (100 mL) was stirred at 100° C. under N₂ atmosphere for 12 hours. After completion, the resulting mixture was filtered and the filtrate was concentrated in vacuo to afford a crude residue, which was purified by silica-gel chromatography (elute with EtOAc:PE=0-25%) to afford compound Int-12a (4.1 g, 91.8% yield) as a yellow oil. MS (ESI⁺) obsd. [(M+H)⁺]:326.1.

### Step 2: 7-Fluoro-1-tetrahydropyran-2-yl-indazol-4-amine

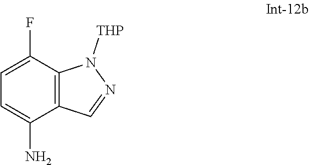

A mixture of compound Int-12a (4.4 g, 13.6 mmol), Pd/C (440 mg, 10% wt) in THE (100 mL) was stirred at rt under H₂ atmosphere for 48 hours. After completion, the resulting mixture was filtered and the filtrate was concentrated in vacuo to give a crude residue, which was purified by silica-gel chromatography (elute with EtOAc:PE=5-50%) to afford compound Int-12b (2.5 g, 78.4% yield) as a yellow solid. MS (ESI⁺) obsd. [(M+H)⁺]:236.1.

### Step 3: 5-Chloro-7-fluoro-1-tetrahydropyran-2-yl-indazol-4-amine

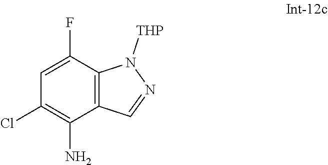

To a cooled solution of compound Int-12b (500.0 mg, 2.1 mmol) in anhydrous DCM (15 mL) was added NCS (284.0 mg, 2.13 mmol). The mixture was stirred at rt for 2 hours. After completion, the mixture was quenched with the saturated NaHCO₃ solution and extracted with DCM (15 mL×3). The combined organic layers were concentrated in vacuo to give crude residue, which was purified by silica-gel chromatography (elute with EtOAc:PE=0-20%) to afford compound Int-12c (510.0 mg, 88.7% yield) as a light yellow solid. MS (ESI⁺) obsd. [(M+H)⁺]:270.0.

### Step 4: 4-Bromo-5-chloro-7-fluoro-1-tetrahydropyran-2-yl-indazole

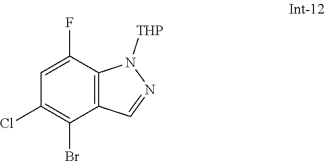

To a suspension of CuBr (745.0 mg, 5.2 mmol) in anhydrous acetonitrile (15 mL) was added nitrous acid tert-butyl ester (1.34 g, 12.98 mmol) dropwise at 50° C. The mixture was stirred at 50° C. for 30 minutes. And then, compound Int-12c (700 mg, 2.6 mmol) in anhydrous acetonitrile (5 mL) was added to the mixture dropwise and the mixture was stirred at 80° C. for 6 hours. After completion, the mixture was concentrated in vacuo to give crude residue, which was purified by silica-gel chromatography (elute with EA:PE=0-10%) to afford compound Int-12 (400.0 mg, 46.1% yield) as a white solid. MS (ESI⁺) obsd. [(M-THP+H)⁺]:248.9.

### Intermediate 13: tert-Butyl (2-(7-fluoro-1-(tetrahydro-2H-pyran-2-yl)-1H-indazole-4-carbonyl)-6-hydroxy-5-methylpyridin-3-yl)carbamate

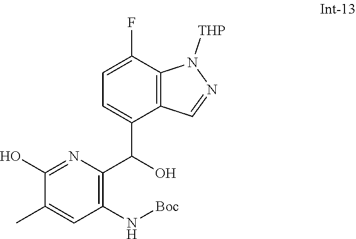

### Step 1: 2-Benzyloxy-3-methyl-5-nitro-pyridine

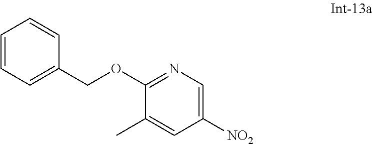 [SMILES: Cc1cc([N+](=O)[O-])cnc1OCc1ccccc1]

The title compound was prepared in analogy to the preparation of compound Int-7a, replacing 2-cyclobutanol with benzyl alcohol. Compound Int-13a (8.0 g) was obtained as a yellow solid. MS (ESI⁺) obsd. [(M+H)⁺]: 245.1.

### Step 2: 6-(Benzyloxy)-5-methylpyridin-3-amine

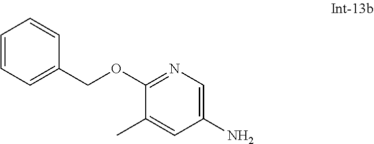 [SMILES: Cc1cc(N)cnc1OCc1ccccc1]

The title compound was prepared in analogy to the preparation of Int-7b, replacing compound Int-7a with compound Int-13a. Compound Int-13b (4.7 g) was obtained as a yellow solid. MS (ESI⁺) obsd. [(M+H)⁺]: 215.3.

### Step 3: 6-(Benzyloxy)-2-bromo-5-methylpyridin-3-amine

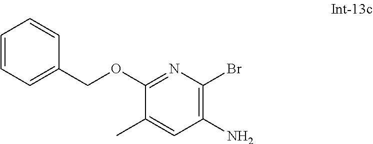 [SMILES: Cc1cc(N)c(Br)nc1OCc1ccccc1]

The title compound was prepared in analogy to the preparation of Int-7c, replacing compound Int-7b with compound Int-13b. Compound Int-13c (3.6 g) was obtained as a yellow solid. MS (ESI⁺) obsd. [(M+H)⁺]: 215.3.

### Step 4: tert-Butyl (6-(benzyloxy)-2-bromo-5-methylpyridin-3-yl)carbamate

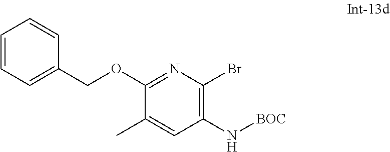 [SMILES: Cc1cc(NC(=O)OC(C)(C)C)c(Br)nc1OCc1ccccc1]

The title compound was prepared in analogy to the preparation of Int-2, replacing 2-bromo-5-chloro-pyridin-3-amine with compound Int-13c. Compound Int-13d (4.3 g) was obtained as a light yellow solid. MS (ESI⁺) obsd. [(M+H)⁺]: 393.1.

### Step 5: tert-Butyl (6-(benzyloxy)-2-((7-fluoro-1-(tetrahydro-2H-pyran-2-yl)-1H-indazol-4-yl)(hydroxy)methyl)-5-methylpyridin-3-yl)carbamate

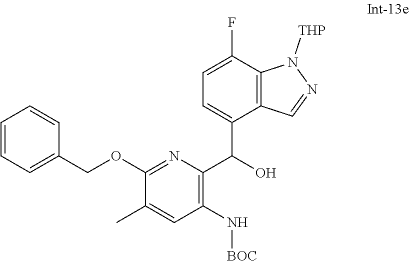

To a solution of compound Int-13d (4.3 g, 10.9 mmol) in THE (40 mL) was added n-BuLi (9.1 mL, 22.8 mmol) at −78° C. and the mixture was stirred at −78° C. for 1 hour. And then, 7-fluoro-1-tetrahydropyran-2-yl-indazole-4-carbaldehyde (2.3 g, 9.1 mmol) (vendor: PharmaBlock (Nanjing) R&D Co. Ltd, catalog PCS1710) in THE (30 mL) was added to the mixture and the mixture was stirred at −78° C. for another 30 minutes. After completion, the reaction mixture was quenched with saturated ammonium chloride (40 mL) and extracted with EtOAc (50 mL×3). The combined organic layer was washed with brine (40 mL), dried over anhydrous sodium sulfate and concentrated under reduced pressure to give a residue, which was purified by silica-gel chromatography (elute with EtOAc:PE=0-20%) to give compound Int-13e (4.1 g, 66.9% yield) as a light yellow oil. MS (ESI⁺) obsd. [(M+H)⁺]: 563.3.

### Step 6: tert-Butyl (6-(benzyloxy)-2-(7-fluoro-1-(tetrahydro-2H-pyran-2-yl)-1H-indazole-4-carbonyl)-5-methylpyridin-3-yl)carbamate

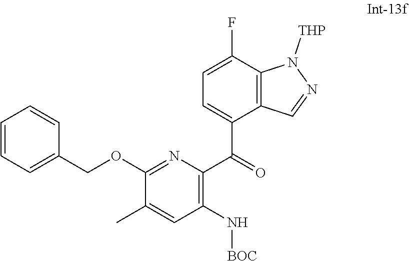

The mixture of compound Int-13e (4.1 g, 7.3 mmol) and MnO₂ (12.7 g, 145.7 mmol) in chloroform (50 mL) was stirred at 50° C. for 3 hours. After completion, the mixture was filtered and the filtrate was concentrated in vacuo to give a residue, which was purified by silica-gel chromatography (elute with EtOAc:PE=0-10%) to give compound Int-13f (3.4 g, 83.0% yield) as a yellow solid. MS (ESI⁺) obsd. [(M+H)⁺]: 561.4.

### Step 7: tert-Butyl (2-(7-fluoro-1-(tetrahydro-2H-pyran-2-yl)-1H-indazole-4-carbonyl)-6-hydroxy-5-methylpyridin-3-yl)carbamate

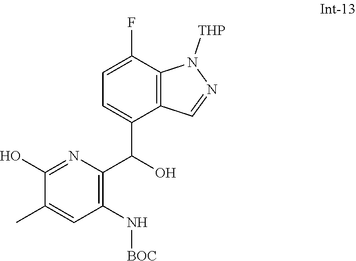

The mixture of compound Int-13f (3.4 g, 6.1 mmol) and Pd/C (0.81 g, 5% on wet carbon) in EtOAc (60.0 mL) under H₂ (15 psi) atmosphere was stirred at rt for 1 hour. After completion, the mixture was filtered and the filtrate was concentrated in vacuo to give a residue, which was purified by silica-gel chromatography (elute with EtOAc:PE=0-20%) to give compound Int-13 (2.4 g, 84.2% yield) as a yellow solid. MS (ESI⁺) obsd. [(M+H)⁺]: 471.4.

### Intermediate 14: tert-Butyl N-(2-bromo-6-chloro-4,5-dimethyl-3-pyridyl)carbamate

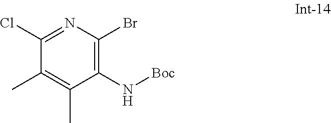 [SMILES: Cc1c(Cl)nc(Br)c(NC(=O)OC(C)(C)C)c1C]

### Step 1: 5-Bromo-6-chloro-4-methyl-pyridin-3-amine

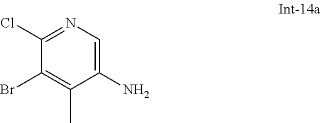

To a solution of compound 3-bromo-2-chloro-4-methyl-5-nitro-pyridine (2.0 g, 7.9 mmol) in MeOH (20 mL) and water (6 mL) were added Fe powder (4.4 g, 79.5 mmol) and NH₄Cl (4.3 g, 79.5 mmol) at 25° C. The mixture was stirred at 75° C. for 2 hours. After completion, the mixture was filtered and the filtrate was concentrated in vacuo to give compound Int-14a (1.7 g, 94.2% yield) as yellow oil. MS (ESI⁺) obsd. [(M+H)⁺]: 220.9.

### Step 2: 6-Chloro-4,5-dimethyl-pyridin-3-amine

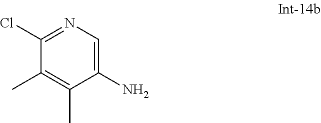

To a solution of compound Int-14a (2.0 g, 8.9 mmol) in 1,4-dioxane (25 mL) and water (2.5 mL) were added 2,4,6-trimethyl-1,3,5,2,4,6-trioxatriborinane (1.7 g, 13.3 mmol), potassium carbonate (3.7 g, 26.5 mmol) and 1,1′-bis(diphenylphosphino)ferrocene palladium dichloride (647.0 mg, 885.0 μmol). The mixture was stirred at 80° C. for 3 hours. After completion, the mixture was filtered and the filtrate was concentrated under vacuum to give compound Int-14b (1.4 g, 100% yield) as yellow oil. MS (ESI⁺) obsd. [(M+H)⁺]: 157.0.

### Step 3: 2-Bromo-6-chloro-4,5-dimethyl-pyridin-3-amine

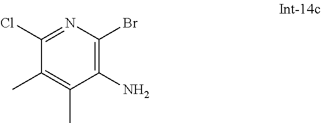

To a solution of compound Int-14b (400.0 mg, 2.6 mmol) in acetonitrile (12 mL) were added NBS (500.0 mg, 2.8 mmol). The mixture was stirred at 25° C. for 1 hour. After completion, the mixture was poured into water (100 mL) and extracted with EtOAc (50 mL×3). The combined organic layer was washed by brine, dried over anhydrous sodium sulfate and concentrated under vacuum to give a residue, which was purified by silica-gel chromatography (elute with EtOAc:PE=0-50%) to give compound Int-14c (400.0 mg, 66.5% yield) as yellow oil. MS (ESI⁺) obsd. [(M+H)⁺]: 235.0.

### Step 4: tert-Butyl-N-(2-bromo-6-chloro-4,5-dimethyl-3-pyridyl)carbamate

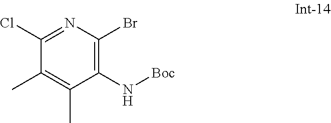 [SMILES: Cc1c(Cl)nc(Br)c(NC(=O)OC(C)(C)C)c1C]

To a solution of compound Int-14c (800 mg, 3.40 mmol) in THE (20 mL) were added (Boc)₂O (2.97 g, 13.6 mmol) and 4-dimethylaminopyridine (830 mg, 6.79 mmol) at rt. The mixture was stirred at 70° C. for 2 hours. After completion, the mixture was poured into water (100 mL) and extracted with EtOAc (50 mL×3). The combined organic layer was washed by brine, dried over Na₂SO₄ and concentrated under vacuum to give a residue. The residue was re-dissolved in THE (7 mL), methanol (21 mL) and water (7 mL), lithium hydroxide hydrate (2.48 g, 59.1 mmol) was added and the mixture was stirred at 25° C. for 2 hours. After completion, the mixture was poured into water (100 mL) and extracted with EtOAc (50 mL×3). The combined organic layer was washed by brine, dried over Na₂SO₄ and concentrated in vacuo to give a residue, which was purified by silica-gel chromatography (elute with EtOAc:PE=0-50%) to give compound Int-14 (1.1 g, 90.8% yield) as yellow oil. MS (ESI⁺) obsd. [(M+H)⁺]: 335.0.

### Intermediate 15: tert-Butyl N-(2-bromo-4-methoxy-5-methyl-3-pyridyl)carbamate

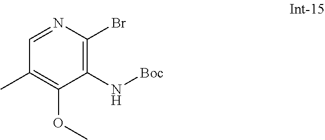 [SMILES: COc1c(C)cnc(Br)c1NC(=O)OC(C)(C)C]

### Step 1: 3-Bromo-4-methoxy-5-nitropyridine

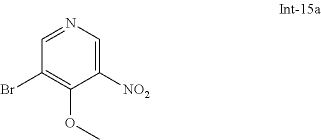

A mixture of 3-bromo-4-chloro-5-nitropyridine (7.7 g, 32.4 mmol) and MeONa (7.2 mL, 38.9 mmol) in methanol (100 mL) was stirred at 25° C. for 2 hours. After completion, the mixture was poured into water (120 mL) and extracted with EtOAc (100 mL×3). The combined organic layer was washed with brine (100 mL), dried over anhydrous sodium sulfate and concentrated under reduced pressure to give a residue, which was purified by silica-gel chromatography (elute with EtOAc:PE=0-20%) to give compound Int-15a (7.0 g, 92.7% yield) as a yellow gum.

### Step 2: 4-Methoxy-3-methyl-5-nitropyridine

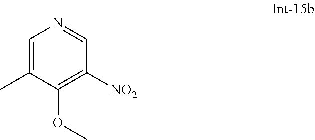

A mixture of compound Int-15a (1.0 g, 4.29 mmol), trimethylboroxine (2.45 mL, 8.58 mmol), Pd(dppf)Cl₂ (313.71 mg, 0.43 mmol) and K₂CO₃ (1186.25 mg, 8.58 mmol) in 1,4-dioxane (2 mL) was stirred at 100° C. for 12 hours under N₂ protection. After completion, the mixture was poured into water (50 mL) and extracted with EtOAc (50 mL×3). The combined organic layer was washed with brine (50 mL), dried over anhydrous sodium sulfate and concentrated under reduced pressure to give a residue, which was purified by silica-gel chromatography (elute with EtOAc:PE=0-20%) to give compound Int-15b (600.0 mg, 83.15% yield) as a yellow gum. MS (ESI⁺) obsd. [(M+H)⁺]: 169.1.

### Step 3: 4-Methoxy-5-methylpyridin-3-amine

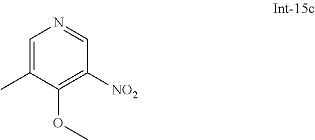

A mixture of compound Int-15b (2.0 g, 13.1 mmol), Fe (4.4 g, 78.9 mmol) and NH₄Cl (4.2 g, 78.9 mmol) in ethanol (30 mL) and water (10 mL) was stirred at 80° C. for 3 hours. After completion, the mixture was filtered and the filtrate was concentrated under reduced pressure to give a residue, which was purified by silica-gel chromatography (elute with EtOAc:PE=0-50%) to give compound Int-15c (0.5 g, 27.4% yield) as a yellow gum. MS (ESI⁺) obsd. [(M+H)⁺]: 139.2.

### Step 4: 2-Bromo-4-methoxy-5-methylpyridin-3-amine

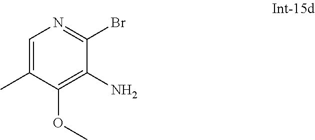

To a solution of compound Int-15c (1.2 g, 8.7 mmol) in MeCN (15 mL) was dropwise added NBS (1.5 g, 8.7 mmol) at 0° C. Then the mixture was warmed up to 25° C. and stirred for 1 hour. After completion, the reaction was quenched with saturated sodium sulfite (20 mL) and extracted with EtOAc (30 mL×3). The combined organic layer was washed with brine (20 mL), dried over anhydrous sodium sulfate and concentrated under reduced pressure to give a residue, which was purified by silica gel-chromatography (elute with EtOAc:PE=0-50%) to give compound Int-15d (310.0 mg, 16.4% yield) as a yellow gum. MS (ESI⁺) obsd. [(M+H)⁺]: 217.0 Step 5: tert-butyl (2-bromo-4-methoxy-5-methylpyridin-3-yl)carbamate

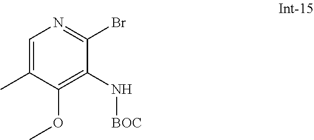 [SMILES: COc1c(C)cnc(Br)c1NC(=O)OC(C)(C)C]

The title compound was prepared in analogy to the preparation of compound Int-2, replacing 2-bromo-5-chloro-pyridin-3-amine with compound Int-15d. Compound Int-15 (310.0 mg) was obtained as a yellow gum. MS (ESI⁺) obsd. [(M+H)⁺]: 317.0.

### Intermediate 16: tert-Butyl N-(2-bromo-5-chloro-6-methyl-3-pyridyl)carbamate

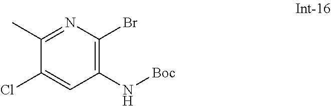 [SMILES: Cc1nc(Br)c(NC(=O)OC(C)(C)C)cc1Cl]

The title compound was prepared in analogy to the preparation of compound Int-15, replacing compound Int-15b with 3-chloro-2-methyl-5-nitropyridine in step 3. Compound Int-16 (1.5 g) was obtained as a yellow gum. MS (ESI⁺) obsd. [(M+H)⁺]: 321.0.

### Intermediate 17: tert-Butyl N-(2-bromo-4,5-dimethyl-3-pyridyl)carbamate

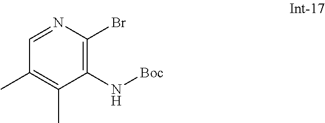 [SMILES: Cc1cnc(Br)c(NC(=O)OC(C)(C)C)c1C]

The title compound was prepared in analogy to the preparation of compound Int-15, replacing compound Int-15b with 3,4-dimethyl-5-nitropyridine in step 3. Compound Int-17 (1.6 g) was obtained as a yellow gum. MS (ESI⁺) obsd. [(M+H)⁺]: 301.2.

### Intermediate 18: tert-Butyl N-(2-bromo-5-chloro-6-cyclopropyl-3-pyridyl)carbamate

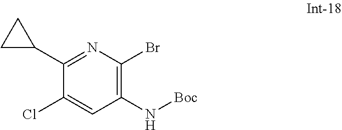 [SMILES: CC(C)(C)OC(=O)Nc1cc(Cl)c(C2CC2)nc1Br]

### Step 1: 5-Chloro-6-cyclopropylpyridin-3-amine

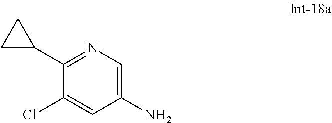 [SMILES: Nc1cnc(C2CC2)c(Cl)c1]

A mixture of 6-bromo-5-chloro-pyridin-3-amine (10.0 g, 48.2 mmol), cyclopropylboronic acid (4.1 g, 48.2 mmol), Pd(dppf)Cl₂ (3.5 g, 4.8 mmol) and Cs₂CO₃ (3.1 g, 96.4 mmol) in 1,4-dioxane (100 mL) was stirred at 100° C. for 1 hour under N₂ protection. After completion, the mixture was filtered and the filtrate was concentrated in vacuo to give a residue, which was purified by-silica gel chromatography (elute with EtOAc:PE=20-40%) to give compound Int-18a (9.5 g, 71.8% yield) as a white solid. MS (ESI⁺) obsd. [(M+H)⁺]: 169.1.

### Step 2: 2-Bromo-5-chloro-6-cyclopropylpyridin-3-amine

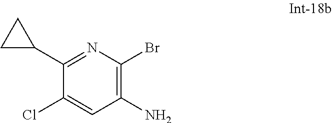 [SMILES: Nc1cc(Cl)c(C2CC2)nc1Br]

To a mixture of compound Int-18a (700.0 mg, 4.1 mmol) in DMF (7 mL) was added NBS (665.0 mg, 3.7 mmol) portionwise. The reaction mixture was stirred at 0° C. for 0.5 hour. After completion, the reaction mixture was quenched with saturated sodium sulfite (10 mL) and extracted with EtOAc (10 mL×3). The combined organic layer was washed with brine (30 mL), dried over anhydrous sodium sulfate and concentrated under reduced pressure to give a residue, which was purified by silica-gel chromatography (elute with EtOAc:PE=20-40%) to give compound Int-18b (790.0 mg, 76.9% yield) as a yellow solid. MS (ESI⁺) obsd. [(M+H)⁺]: 246.9.

### Step 3: tert-Butyl (2-bromo-5-chloro-6-cyclopropylpyridin-3-yl)carbamate

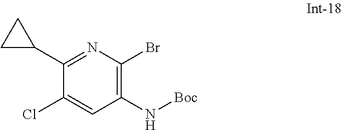 [SMILES: CC(C)(C)OC(=O)Nc1cc(Cl)c(C2CC2)nc1Br]

The title compound was prepared in analogy to the preparation of compound Int-2, replacing 2-bromo-5-chloro-pyridin-3-amine with compound Int-18b. Compound Int-18 (940.0 mg) was obtained as a yellow gum. MS (ESI⁺) obsd. [(M+H)⁺]: 347.1.

### Intermediate 19: tert-Butyl N-(2-bromo-5-chloro-4,6-dimethyl-3-pyridyl)carbamate

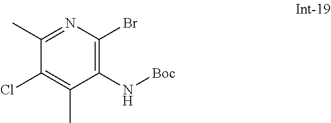 [SMILES: Cc1nc(Br)c(NC(=O)OC(C)(C)C)c(C)c1Cl]

The title compound was prepared in analogy to the preparation of compound Int-15, replacing compound Int-15a with 2-bromo-3-chloro-4-methyl-5-nitro-pyridine in step 2. Compound Int-19 (1.5 g) was obtained as a yellow gum. MS (ESI⁺) obsd. [(M+H)⁺]: 335.0.

### Intermediate 20: tert-Butyl N-(2-bromo-4-chloro-5,6-dimethyl-3-pyridyl)carbamate

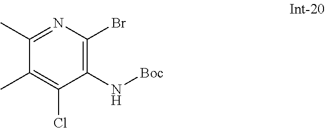 [SMILES: Cc1nc(Br)c(NC(=O)OC(C)(C)C)c(Cl)c1C]

### Step 1: 2-Bromo-5,6-dimethylpyridin-3-amine

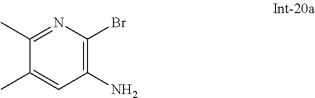

To a solution of 5,6-dimethylpyridin-3-amine (4.0 g, 32.7 mmol) in DCM (40 mL) was added a solution of NBS (6.4 g, 36.0 mmol) in DCM (60 mL) dropwise at 0° C. And the reaction was stirred at 0° C. for 1.5 hour. After completion, the reaction mixture was quenched with sodium sulfite solution (40 mL) and extracted with EtOAc (20 mL×3). The combined organic layer was washed with brine (40 mL), dried over anhydrous sodium sulfate and concentrated under reduced pressure to give a residue, which was purified by prep-TLC (PE/EA=5/1) to give compound Int-20a (4.3 g, 65.3% yield) as a yellow solid. MS (ESI⁺) obsd. [(M+H)⁺]: 201.0.

### Step 2: 2-Bromo-4-chloro-5,6-dimethylpyridin-3-amine

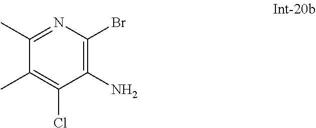

To a solution of compound Int-20a (4.2 g, 20.9 mmol) in NMP (50 mL) was dropwise added a solution of NCS (3.1 g, 23.0 mmol) in NMP (30 mL) at 25° C. Then the mixture was heated to 80° C. and stirred for 12 hours. After completion, the reaction mixture was quenched with saturated sodium sulfite (40 mL) and extracted with ethyl acetate (60 mL×3). The combined organic layer was washed with brine (50 mL), dried over anhydrous sodium sulfate and concentrated under reduced pressure to give a residue, which was purified by silica-gel chromatography (elute with EtOAc:PE=0-20%) to give compound Int-20b (500.0 mg, 10.2% yield) as a white solid. MS (ESI⁺) obsd. [(M+H)⁺]: 235.0

### Step 3: tert-Butyl (2-bromo-4-chloro-5,6-dimethylpyridin-3-yl)carbamate

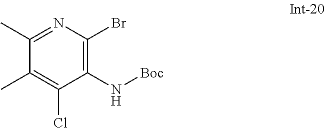 [SMILES: Cc1nc(Br)c(NC(=O)OC(C)(C)C)c(Cl)c1C]

The title compound was prepared in analogy to the preparation of compound Int-2, replacing 2-bromo-5-chloro-pyridin-3-amine with compound Int-20b. Compound Int-20 (518.0 mg) was obtained as a white solid. MS (ESI⁺) obsd. [(M+H)⁺]: 335.0.

### Intermediate 21: tert-Butyl N-[2-(7-fluoro-1-tetrahydropyran-2-yl-indazole-4-carbonyl)-4-methyl-3-pyridyl]carbamate

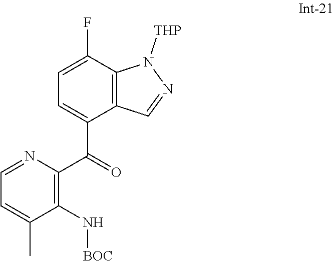

The title compound was prepared in analogy to the preparation of compound Int-13f, replacing compound Int-13c with 2-bromo-4-methyl-pyridin-3-amine in step 4. Compound Int-21 (1.5 g) was obtained as a yellow gum. MS (ESI⁺) obsd. [(M+H)⁺]: 455.2.

### Intermediate 22: (5-Cyclopropyl-4-methyl-3-nitro-2-pyridyl)-(7-fluoro-1-tetrahydropyran-2-yl-indazol-4-yl)methanone

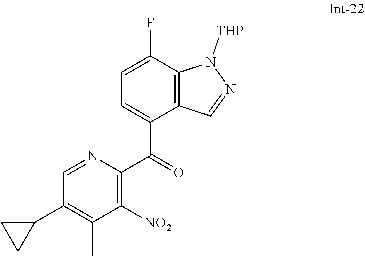

### Step 1: (5-Bromo-4-methyl-3-nitropyridin-2-yl)(7-fluoro-1-(tetrahydro-2H-pyran-2-yl)-1H-indazol-4-yl)methanol

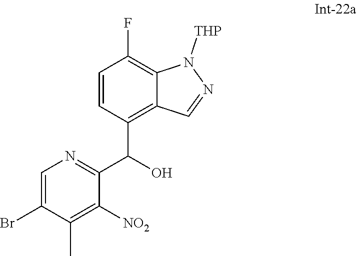

To a solution of 5-bromo-2-iodo-4-methyl-3-nitro-pyridine (1.1 g, 3.2 mmol) in THE (10 mL) was added PhMgBr (1.2 mL, 3.5 mmol) at −40° C. under nitrogen atmosphere. And then, a solution of 7-fluoro-1-tetrahydropyran-2-yl-indazole-4-carbaldehyde (1.1 g, 4.5 mmol) in THE (10 mL) was added to the mixture at −40° C. The reaction mixture was slowly warmed to 25° C. and stirred for another 30 min. After completion, the reaction mixture was quenched with saturated ammonium chloride (30 mL) and extracted with EtOAc (50 mL×3). The combined organic layer was washed with brine (30 mL), dried over anhydrous sodium sulfate and concentrated in vacuo to give a residue, which was purified by silica-gel chromatography (elute with EtOAc:PE=0-30%) to give compound Int-22a (680.0 mg, 83.5% yield) as an orange solid. MS (ESI⁺) obsd. [(M+H)⁺]: 465.0.

### Step 2: (5-Bromo-4-methyl-3-nitropyridin-2-yl)(7-fluoro-1-(tetrahydro-2H-pyran-2-yl)-1H-indazol-4-yl)methanone

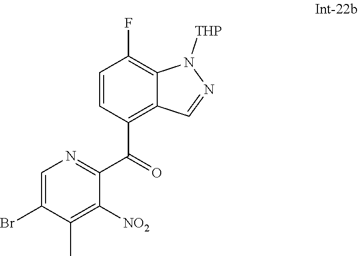

A mixture of compound Int-22a (680.0 mg, 1.5 mmol) in chloroform (10 mL) was added MnO₂ (1.9 g, 21.9 mmol) was stirred at 50° C. for 12 hours, the mixture was filtered and the filtrate was concentrated in vacuo to give compound Int-22b (650.0 mg, crude) as a yellow solid. MS (ESI⁺) obsd. [(M+H)⁺]: 463.0.

### Step 3: (5-Cyclopropyl-4-methyl-3-nitropyridin-2-yl)(7-fluoro-1-(tetrahydro-2H-pyran-2-yl)-1H-indazol-4-yl)methanone

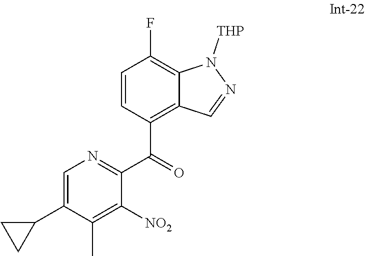

A mixture of compound Int-22b (300 mg, 0.65 mmol), cyclopropylboronic acid (61.2 mg, 0.71 mmol), Pd(dppf)Cl₂ (47.3 mg, 0.06 mmol) and K₂CO₃ (179 mg, 1.3 mmol) in 1,4-dioxane (5 mL) was stirred at 80° C. for 12 hours under N₂ protection. After completion, the mixture was poured into water (20 mL) and the mixture was EtOAc (20 mL×3). The combined organic layer was washed with brine (30 mL), dried over anhydrous sodium sulfate and concentrated under reduced pressure to give a residue, which was purified by silica-gel chromatography (elute with EtOAc:PE=0-30%) to give compound Int-22 (170 mg, 61.8% yield) as a yellow solid. MS (ESI⁺) obsd. [(M+H)⁺]: 425.1.

### Intermediate 23: [3-Amino-4-methyl-5-(trideuteriomethyl)-2-pyridyl]-(7-fluoro-1-tetrahydropyran-2-yl-indazol-4-yl)methanone

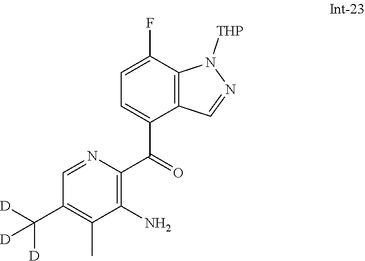

### Step 1: (3-Amino-5-bromo-4-methylpyridin-2-yl)(7-fluoro-1-(tetrahydro-2H-pyran-2-yl)-1H-indazol-4-yl)methanone

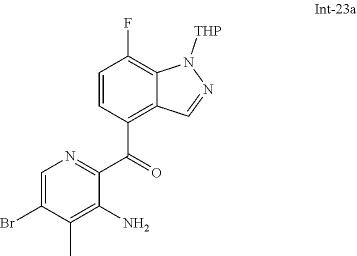

The title compound was prepared in analogy to the preparation of compound Int-15c, replacing compound Int-15b with compound Int-22b. Compound Int-23a (0.5 g) was obtained as a yellow gum. MS (ESI⁺) obsd. [(M+H)⁺]: 433.1.

### Step 2: (3-Amino-4-methyl-5-(4,4,5,5-tetramethyl-1,3,2-dioxaborolan-2-yl)pyridin-2-yl)(7-fluoro-1-(tetrahydro-2H-pyran-2-yl)-1H-indazol-4-yl)methanone

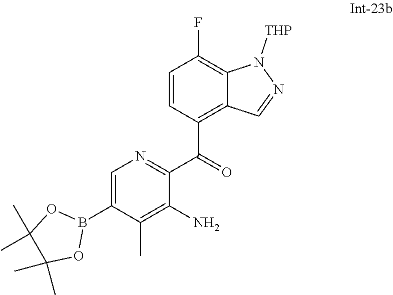

A mixture of compound Int-23a (260.0 mg, 0.6 mmol), bis(pinacolato)diboron (304.8 mg, 1.2 mmol), Pd(dppf)Cl₂ (43.9 mg, 0.06 mmol) and KOAc (206.1 mg, 2.1 mmol) in 1,4-dioxane (5 mL) was stirred at 110° C. for 12 hours under N₂ protection. After completion, the suspension was filtered and the filtrate was concentrated in vacuo to give compound Int-23b (280.0 mg, crude) as a black oil. MS (ESI⁺) obsd. [(M+H)⁺]: 481.4.

### Step 3: [3-Amino-4-methyl-5-(trideuteriomethyl)-2-pyridyl]-(7-fluoro-1-tetrahydropyran-2-yl-indazol-4-yl)methanone

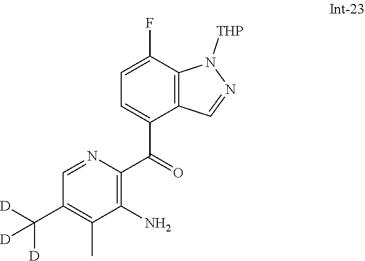

A mixture of compound Int-23b (280.0 mg, 0.58 mmol), iodomethane-d₃ (0.11 mL, 1.8 mmol), Pd(dppf)Cl₂ (42.7 mg, 0.06 mmol) and K₃PO₄ (371.2 mg, 1.8 mmol) in N,N-dimethylformamide (3 mL) and water (0.3 mL) was stirred at 80° C. for 2 hours under N₂ protection. After completion, the reaction mixture was filtered and the filtrate was concentrated in vacuo to give a residue, which was purified by silica-gel chromatography (elute with EtOAc:PE=0-30%) to give compound Int-23 (65.0 mg, 30.0% yield) as a yellow solid. MS (ESI⁺) obsd. [(M+H)⁺]: 372.3.

### Intermediate 24: tert-Butyl N-(2-chloro-3-iodo-5,6-dimethyl-4-pyridyl)carbamate

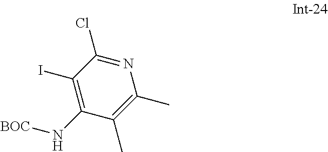 [SMILES: Cc1nc(Cl)c(I)c(NC(=O)OC(C)(C)C)c1C]

### Step 1: 6-Chloro-3-iodo-2-methylpyridin-4-amine

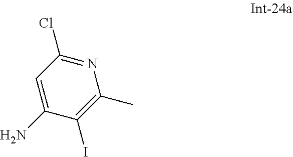

To a solution of 2-chloro-6-methyl-pyridin-4-amine (4.0 g, 28.05 mmol) and toluene-4-sulfonic acid (241.54 mg, 1.4 mmol) in MeCN (40 mL) was added NIS (7.57 g, 33.66 mmol), the mixture was stirred at 70° C. for 12 h. After completion, the mixture was quenched with saturated sodium sulfite (50 mL) and extracted with EtOAc (50 mL×3). The combined organic layer was washed with brine (60 mL), dried over anhydrous Na₂SO₄ and concentrated under reduced pressure to give a residue, which was purified by silica-gel chromatography (elute with EtOAc:PE=0-30%) to give Int-24a (2.6 g, 34.5% yield) as a yellow solid.

### Step 2: 6-Chloro-2,3-dimethylpyridin-4-amine

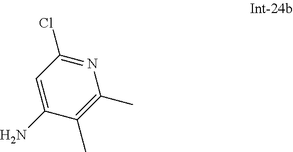

A mixture of Int-24a (2.4 g, 8.94 mmol), trimethylboroxine (3.83 mL, 13.41 mmol), Pd(dppf)Cl₂ (654.1 mg, 0.89 mmol) and Cs₂CO₃ (5.83 g, 17.88 mmol) in 1,4-dioxane (30 mL) was stirred at 80° C. for 2 h under N₂ protection. After completion, the mixture was filtered and the filtrate was concentrated under vacuum to give a residue, which was purified by silica-gel chromatography (elute with EtOAc:PE=0-50%) to give Int-24b (1.05 g, 75.0% yield) as a yellow solid. MS (ESI⁺) obsd. [(M+H)⁺]: 157.1.

### Step 3: 2-Chloro-3-iodo-5,6-dimethylpyridin-4-amine

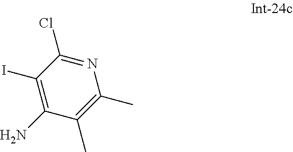

To a solution of Int-24b (1.0 g, 6.39 mmol) and toluene-4-sulfonic acid (54.98 mg, 0.32 mmol) in MeCN (10 mL) was added NIS (1.72 g, 7.66 mmol), the mixture was stirred at 70° C. for 2 h. After completion, the reaction mixture was quenched with saturated sodium sulfite (20 mL), extracted with EtOAc (30 mL×3). The combined organic layer was dried over anhydrous sodium sulfate and concentrated under reduced pressure to give a residue, which was purified by silica-gel chromatography (elute with EtOAc:PE=0-30%) to give Int-24c (1.8 g, 99.8% yield) as a yellow solid. MS (ESI⁺) obsd. [(M+H)⁺]: 282.9.

### Step 4: tert-Butyl (2-chloro-3-iodo-5,6-dimethylpyridin-4-yl)carbamate

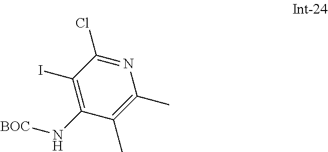 [SMILES: Cc1nc(Cl)c(I)c(NC(=O)OC(C)(C)C)c1C]

To a solution of Int-24c (1.7 g, 6.02 mmol) in THE (20 mL) was added dropwise NaHMDS (12.03 mL, 12.03 mmol) at 0° C. After stirred at 0° C. for 30 min, a solution of di-t-butyldicarbonate (1.44 g, 6.62 mmol) in THE (20 mL) was added dropwise to the above mixture at 0° C., then the mixture was stirred for another 1 h at 0° C. After completion, the reaction mixture was quenched with saturated aqueous ammonium chloride (50 mL) and extracted with ethyl acetate (40 mL×3). The combined organic layer was dried over anhydrous sodium sulfate and concentrated under reduced pressure to give a residue, which was purified by silica-gel chromatography (elute with EtOAc:PE=0 to 30%) to give Int-24 (1.8 g, 78.2% yield) as a yellow solid. MS (ESI⁺) obsd. [(M+H)⁺]: 383.1.

### Intermediate 25: tert-Butyl (2-chloro-6-cyclopropyl-3-iodopyridin-4-yl)carbamate

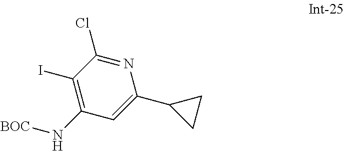 [SMILES: CC(C)(C)OC(=O)Nc1cc(C2CC2)nc(Cl)c1I]

### Step 1: 2-chloro-6-cyclopropyl-3-iodopyridin-4-amine

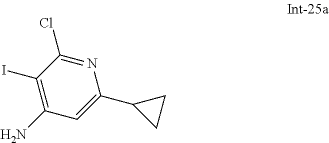 [SMILES: Nc1cc(C2CC2)nc(Cl)c1I]

To a solution of 2-chloro-6-cyclopropyl-pyridin-4-amine (1.86 g, 11.0 mmol) and toluene-4-sulfonic acid (95.2 mg, 0.55 mmol) in MeCN (3 mL) was added NIS (2.5 g, 11.0 mmol), the mixture was stirred at 70° C. for 2 h. After completion, the mixture was concentrated under reduced pressure to give a residue, which was purified by silica-gel chromatography (elute with EtOAc:PE=0 to 30%) to give Int-25a (2.1 g, 64.43% yield) as a yellow solid. MS (ESI⁺) obsd. [(M+H)⁺]: 295.1.

### Step 2: tert-Butyl (2-chloro-6-cyclopropyl-3-iodopyridin-4-yl)carbamate

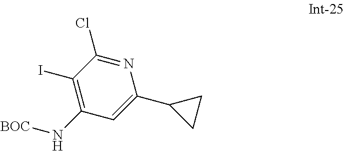 [SMILES: CC(C)(C)OC(=O)Nc1cc(C2CC2)nc(Cl)c1I]

The title compound was prepared in analogy to the preparation of Int-24, replacing compound Int-24c with Int-25a in step 4. Int-25 (2.4 g) was obtained as a yellow solid. MS (ESI⁺) obsd. [(M+H)⁺]: 395.1.

### Intermediate 26: tert-Butyl N-[4-(7-fluoro-1-tetrahydropyran-2-yl-indazole-4-carbonyl)-5-hydroxy-2-methyl-3-pyridyl]carbamate

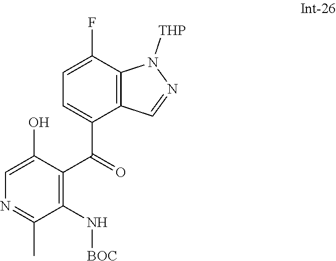

### Step 1: 5-(Benzyloxy)-3-bromo-2-chloropyridine

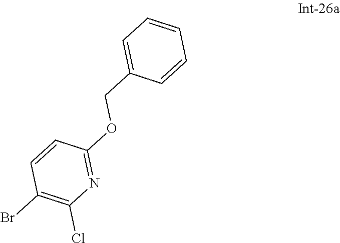 [SMILES: Clc1nc(OCc2ccccc2)ccc1Br]

A mixture of 5-bromo-6-chloro-pyridin-3-ol (10.0 g, 47.98 mmol), bromomethylbenzene (8.55 mL, 71.96 mmol) and potassium carbonate (13.26 g, 95.95 mmol) in MeCN (100 mL) was stirred at 25° C. for 12 h. After completion, the reaction mixture was filtered and the filtrate was concentrated under reduced pressure to give a residue, which was purified by silica-gel chromatography (elute with EtOAc:PE=0 to 10%) to give compound Int-26a (13.5 g, 94.2% yield) as a yellow solid. MS (ESI⁺) obsd. [(M+H)⁺]: 299.9.

### Step 2: tert-Butyl (5-(benzyloxy)-2-chloropyridin-3-yl)carbamate

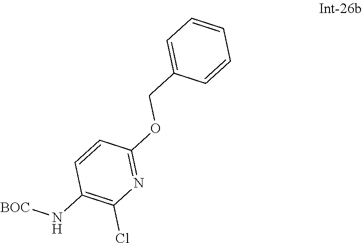 [SMILES: CC(C)(C)OC(=O)Nc1ccc(OCc2ccccc2)nc1Cl]

A mixture of compound Int-26a (16.0 g, 53.6 mmol), Cs₂CO₃ (52.4 g, 160.8 mmol), tert-butyl carbamate (6.3 g, 53.6 mmol), Pd₂(dba)₃ (4.9 g, 5.4 mmol) and Xantphos (3.1 g, 5.36 mmol) in 1,4-dioxane (180 mL) was stirred at 100° C. for 12 h under N₂ protection. After completion, the mixture was filtered and the filtrate was concentrated under reduced pressure to give a residue, which was purified by silica-gel chromatography (elute with EtOAc:PE=0 to 50%) to give Int-26b (13.0 g, 72.5% yield) as a light yellow solid. MS (ESI⁺) obsd. [(M+H)⁺]: 335.1.

### Step 3: tert-Butyl (5-(benzyloxy)-2-chloro-4-((7-fluoro-1-(tetrahydro-2H-pyran-2-yl)-1H-indazol-4-yl)(hydroxy)methyl)pyridin-3-yl)carbamate

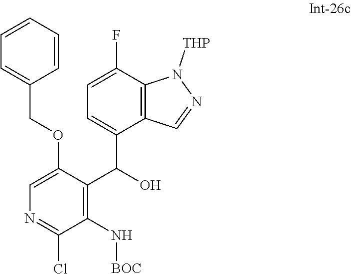

To a solution of Int-26b (5.4 g, 16.13 mmol) in tetrahydrofuran (50 mL) was added n-BuLi (14.19 mL, 35.48 mmol) dropwise at −78° C. Then the mixture was stirred at −78° C. for 1 h. A solution of 7-fluoro-1-tetrahydropyran-2-yl-indazole-4-carbaldehyde (4.8 g, 19.35 mmol) in tetrahydrofuran (15 mL) was added dropwise to the above mixture at −78° C. Then the mixture was stirred at −78° C. for another 0.5 h. After completion, the reaction mixture was quenched with saturated ammonium chloride (40 mL) and extracted with ethyl acetate (30 mL×3). The combined organic layer was dried over anhydrous sodium sulfate and concentrated under reduced pressure to give a residue, which was purified by silica-gel chromatography (elute with EtOAc:PE=0 to 50%) to give compound Int-26c (5.0 g, 53.2% yield) as a yellow solid. MS (ESI⁺) obsd. [(M+H)⁺]: 583.2.

### Step 4: tert-Butyl (5-(benzyloxy)-2-chloro-4-(7-fluoro-1-(tetrahydro-2H-pyran-2-yl)-1H-indazole-4-carbonyl)pyridin-3-yl)carbamate

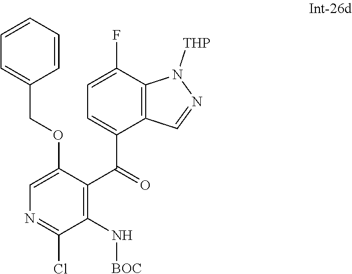

To a solution of compound Int-26c (5.0 g, 8.58 mmol) in DCM (50 mL) was added DMP (4.36 g, 10.29 mmol) at 0° C. The mixture was stirred at 25° C. for 0.5 h. After completion, the reaction mixture was quenched with saturated sodium sulfite (20 mL) and extracted with ethyl acetate (30 mL×3). The combined organic layer was washed with brine (30 mL), dried over anhydrous Na₂SO₄ and concentrated under reduced pressure to give a residue, which was purified by silica-gel chromatography (elute with EtOAc:PE=0 to 50%) to give compound Int-26d (2.0 g, 40.1% yield) as a yellow solid. MS (ESI⁺) obsd. [(M+H)⁺]: 581.3.

### Step 5: tert-Butyl (5-(benzyloxy)-4-(7-fluoro-1-(tetrahydro-2H-pyran-2-yl)-1H-indazole-4-carbonyl)-2-methylpyridin-3-yl)carbamate

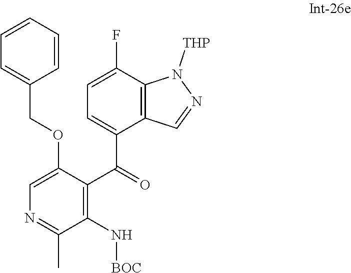

A mixture of compound Int-26d (1.6 g, 2.75 mmol), Cs₂CO₃ (1.79 g, 5.51 mmol), trimethylboroxine (1.57 mL, 5.51 mmol) and Pd(dppf)Cl₂ (201.49 mg, 0.28 mmol) in 1,4-dioxane (30 mL) was stirred 100° C. for 2 h. After completion, the mixture was filtered and the filtrate was concentrated under vacuum to give a residue, which was purified by silica-gel chromatography (elute with EtOAc:PE=0 to 50%) to give compound Int-26e (1.1 g, 68.1% yield) as a yellow solid. MS (ESI⁺) obsd. [(M+H)⁺]: 561.3.

### Step 6: tert-Butyl (4-(7-fluoro-1-(tetrahydro-2H-pyran-2-yl)-1H-indazole-4-carbonyl)-5-hydroxy-2-methylpyridin-3-yl)carbamate

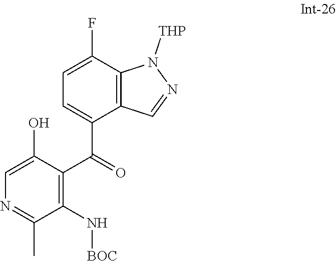

A mixture of compound Int-26e (1.05 g, 1.87 mmol) and Pd/C (210 mg, 10% purity) in EtOAc (20 mL) was stirred at 25° C. for 2 h under H₂ balloon. After completion, the mixture was filtered and the filtrate was concentrated under reduced pressure to give a residue, which was purified by silica-gel chromatography (elute with EtOAc:PE=0 to 60%) to give compound Int-26 (700.0 mg, 83.4% yield) as a yellow solid. MS (ESI⁺) obsd. [(M+H)⁺]: 471.2.

### Intermediate 27: tert-Butyl (2-chloro-5-(methoxymethyl)pyridin-3-yl)carbamate

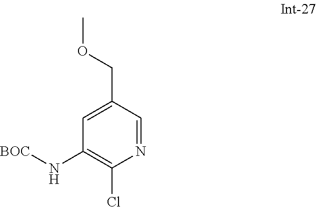 [SMILES: COCc1cnc(Cl)c(NC(=O)OC(C)(C)C)c1]

### Step 1: (5-Bromo-6-chloropyridin-3-yl)methanol

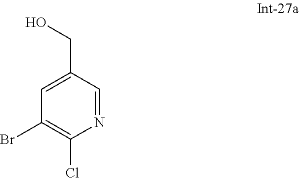

To a solution of methyl 5-bromo-6-chloronicotinate (2.0 g, 7.98 mmol) in tetrahydrofuran (20 mL) was added diisobutylaluminium hydride (19.96 mL, 19.96 mmol) at −78° C. The reaction was stirred at 0° C. for 1 h. After completion, the mixture was quenched with hydrochloric acid (3 M, 20 mL) at 0° C., then extracted with ethyl acetate (30 mL×3). The combined organic layer was dried over anhydrous sodium sulfate and concentrated under reduced pressure to give a residue, which was purified by silica-gel chromatography (elute with EtOAc:PE=0 to 50%) to give Int-27a (1.5 g, 84.4% yield) as a white solid. MS (ESI⁺) obsd. [(M+H)⁺]: 223.9.

### Step 2: 3-Bromo-2-chloro-5-(methoxymethyl)pyridine

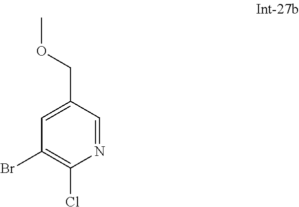

To a solution of Int-27a (1.5 g, 6.74 mmol) in THE (20 mL) was added NaH (350.61 mg, 8.77 mmol) at 0° C. in portions and the mixture was stirred at 0° C. for 30 min. Then iodomethane (0.63 mL, 10.11 mmol) was added to the above mixture at 0° C. and the mixture was stirred at 25° C. for another 1 h. After completion, the mixture was quenched with saturated ammonium chloride (20 mL) and extracted with ethyl acetate (20 mL×3). The combined organic layer was dried over anhydrous sodium sulfate and concentrated under reduced pressure to give Int-27b (1.5 g, crude) as a yellow oil. MS (ESI⁺) obsd. [(M+H)⁺]: 237.9.

### Step 3: tert-Butyl (2-chloro-5-(methoxymethyl)pyridin-3-yl)carbamate

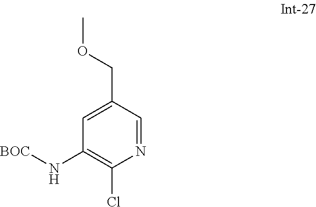 [SMILES: COCc1cnc(Cl)c(NC(=O)OC(C)(C)C)c1]

A mixture of compound Int-27b (1.55 g, 6.55 mmol), Cs₂CO₃ (6.41 g, 19.66 mmol), tert-butyl carbamate (767.82 mg, 6.55 mmol), Pd₂(dba)₃ (0.6 g, 0.66 mmol) and Xantphos (0.38 g, 0.66 mmol) in 1,4-dioxane (1 mL) was stirred at 100° C. for 12 h. After completion, the suspension was filtered and the filtrate was concentrated under reduced pressure to give a residue, which was purified by prep-HPLC (NH₄HCO₃ as additive) to afford Int-27 (470.0 mg, 26.3% yield) as a yellow oil. MS (ESI⁺) obsd. [(M+H)⁺]: 273.1.

### Example 001: 3-Amino-7-chloro-4-(7-fluoro-1H-indazol-4-yl)-1H-1,5-naphthyridin-2-one

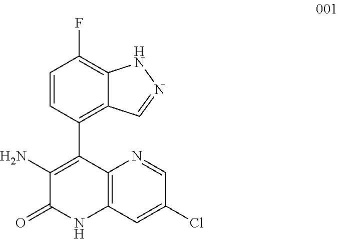 [SMILES: Nc1c(-c2ccc(F)c3[nH]ncc23)c2ncc(Cl)cc2[nH]c1=O]

### Step 1: tert-Butyl N-[5-chloro-2-(7-fluoro-1-tetrahydropyran-2-yl-indazole-4-carbonyl)-3-pyridyl]carbamate

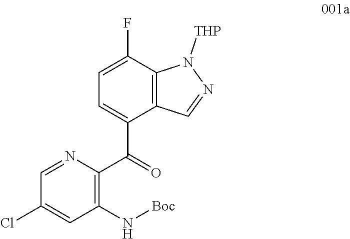

To a solution of compound Int-2 (4.9 g, 15.9 mmol) in THE (50 mL) was added NaH (60%, 794.0 mg, 19.8 mmol) at 0° C. The mixture was stirred at 0° C. for 30 minutes. n-BuLi (2.0 M in cyclohexane, 9.3 mL) was added to the mixture dropwise at −78° C. and the mixture was stirred at −78° C. for another 30 minutes. And then, a solution of compound Int-1 (4.1 g, 13.2 mmol) in THE (50 mL) was added to the mixture at −78° C. The mixture was slowly warmed to 25° C. and stirred for another 1 hour. After completion, the mixture was poured into water (100 mL) and extracted with EtOAc (50 mL×3). The combined organic layer was washed with brine, dried over anhydrous sodium sulfate and concentrated in vacuo to give a residue, which was purified by silica-gel chromatography (elute with EtOAc:PE=0-10%) to give compound 001a (2.5 g, 40.0% yield) as yellow oil. MS (ESI⁺) obsd. [(M+H)⁺]: 475.1.

### Step 2: (3-Amino-5-chloro-2-pyridyl)-(7-fluoro-1H-indazol-4-yl)methanone

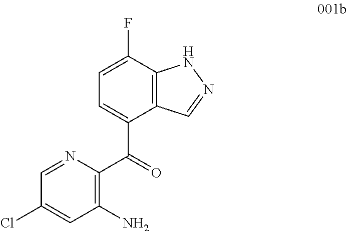 [SMILES: Nc1cc(Cl)cnc1C(=O)c1ccc(F)c2[nH]ncc12]

The mixture of compound 001a (1.6 g, 3.4 mmol) in DCM (15 mL) and TFA (2.6 mL, 33.7 mmol) was stirred at 25° C. for 2 hours. After completion, the mixture was concentrated in vacuo to afford crude compound 001b (0.98 g, 100% yield) as yellow solid, which was used for next step without further purification. MS (ESI⁺) obsd. [(M+H)⁺]: 290.9.

### Step 3: 2-Chloro-N-[5-chloro-2-(7-fluoro-1H-indazole-4-carbonyl)-3-pyridyl]acetamide

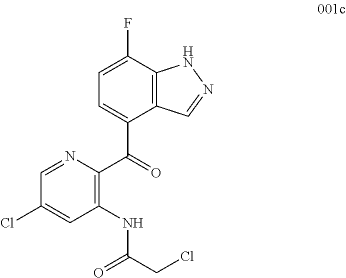 [SMILES: O=C(CCl)Nc1cc(Cl)cnc1C(=O)c1ccc(F)c2[nH]ncc12]

To a solution of compound 001b (980.0 mg, 3.4 mmol) in DCM (10 mL) and DMF (10 mL) were added TEA (0.94 mL, 6.7 mmol) and chloroacetyl chloride (0.30 mL, 3.7 mmol) dropwise. The mixture was stirred at 25° C. for 1 hour. After completion, the mixture was concentrated in vacuo to afford crude compound 001c (1.3 g, 100% yield) as yellow oil, which was used in next step without further purification. MS (ESI⁺) obsd. [(M+H)⁺]: 367.0.

### Step 4: 7-Chloro-4-(7-fluoro-1H-indazol-4-yl)-3-pyridin-1-ium-1-yl-1H-1,5-naphthyridin-2-one; chloride

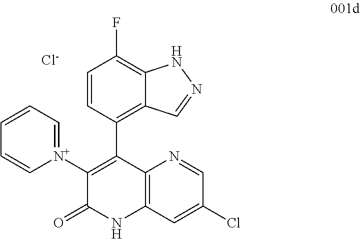 [SMILES: O=c1[nH]c2cc(Cl)cnc2c(-c2ccc(F)c3[nH]ncc23)c1-[n+]1ccccc1 ; [Cl-]]

The solution of crude compound 001c (1.3 g, 3.4 mmol) in pyridine (10 mL) was stirred at 120° C. for 5 hours. After completion, the mixture was concentrated in vacuo to afford crude compound 001d (1.3 g, 100% yield) as brown oil, which was used in the next step without further purification. MS (ESI⁺) obsd. [(M-Cl)⁺]: 392.0.

### Step 5: 3-Amino-7-chloro-4-(7-fluoro-1H-indazol-4-yl)-1H-1,5-naphthyridin-2-one

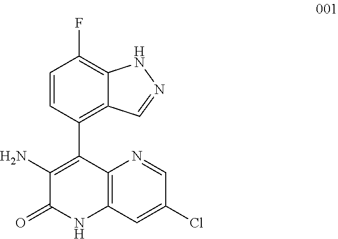 [SMILES: Nc1c(-c2ccc(F)c3[nH]ncc23)c2ncc(Cl)cc2[nH]c1=O]

The mixture of compound 001d (1.31 g, 3.37 mmol) and hydrazine monohydrate (1.6 mL, 33.7 mmol) in ethanol (10 mL) was stirred at 70° C. for 2 hours. After completion, the mixture was poured into water (20 mL) and extracted with EtOAc (20 mL×3). The combined organic layer was washed with brine, dried over anhydrous sodium sulfate and concentrated in vacuo to give the crude product, which was purified by prep-HPLC (NH₃ as additive) to afford Example 001 (453 mg, 40.8% yield) as yellow solid. MS (ESI⁺) obsd. [(M+H)⁺]: 330.0.

Example 001: ¹H NMR (400 MHz, DMSO-d₆) δ ppm 13.62 (br s, 1H), 12.25 (br s, 1H), 8.13 (d, J=2.25 Hz, 1H), 7.63 (d, J=2.25 Hz, 2H), 7.28 (dd, J=11.07, 7.94 Hz, 1H), 7.00 (dd, J=7.75, 4.13 Hz, 1H), 5.44 (br s, 2H). ¹⁹F NMR (376 MHz, DMSO-d₆) δ ppm −132.64 (s, 1F).

The following Examples 002 to 006 were prepared in analogy to the procedure described for the preparation of Example 001, replacing compound Int-2 with HALOCPD indicated in Table 1 in step1.

TABLE 1
Compound synthesis and characterization

| | Compound Names and | | |
|---|---|---|---|
| Ex. | Structures | HALOCPD | NMR and (ESI⁺) |
| 002 | 3-Amino-4-(7-fluoro- | Compound Int-3 | ¹H NMR (400 MHz, DMSO- |
| | 1H-indazol-4-y1)-7- | | d₆) δ ppm 13.08 (s, 1H), 7.99 |
| | methyl-1H-1,5- | | (s, 1H), 7.96 (s, 1H), 7.88 (d, |
| | naphthyridin-2-one | | J = 3.2 Hz, 1H), 7.40 (dd, |
| | | | J = 7.6, 11.2 Hz, 1H), 7.12 |
| | 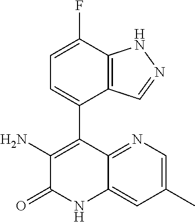 [SMILES: Cc1cnc2c(-c3ccc(F)c4[nH]ncc34)c(N)c(=O)[nH]c2c1] | | (dd, J = 4.0, 7.6 Hz, 1H), 6.78 (s, 1H), 2.42 (s, 3H). ¹⁹F NMR (376 MHz, DMSO-d₆) δ ppm −130.43 (s, 1F). MS (ESI⁺) obsd. [(M + H)⁺]: 310.0. |
| 003 | 3-Amino-6-chloro-4-(7- | Compound Int-9b | ¹H NMR (400 MHz, DMSO- |
| | fluoro-1H-indazol-4- | | d₆) δ ppm 13.64 (s, 1H), |
| | yl)-7-methyl-1H-1,5- | | 12.25 (s, 1H), 7.66 (d, J = |
| | naphthyridin-2-one | | 3.6 Hz, 1H), 7.54 (s, 1H), |
| | | | 7.30 (dd, J = 8.0, 11.2 Hz, |
| | 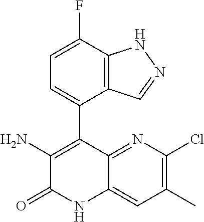 [SMILES: Cc1cc2[nH]c(=O)c(N)c(-c3ccc(F)c4[nH]ncc34)c2nc1Cl] | | 1H), 7.01 (dd, J = 4.4, 8.0 Hz, 1H), 5.38 (s, 2H), 2.31 (s, 3H). ¹⁹F NMR (376 MHz, DMSO-d₆) δ ppm −132.39 (s, 1F). MS (ESI⁺) obsd. [(M + H)⁺]: 344.1. |
| 004 | 3-Amino-7-fluoro-4-(7- | Compound Int-4 | ¹H NMR (400 MHz, DMSO- |
| | fluoro-1H-indazol-4- | | d₆) δ ppm 13.62 (br s, 1H), |
| | y1)-1H-1,5- | | 12.25 (br s, 1H), 8.16 (d, J = |
| | naphthyridin-2-one | | 2.63 Hz, 1 H), 7.62 (br s, 1 |
| | | | H), 7.44 (dd, J = 9.63, 2.63 |
| | 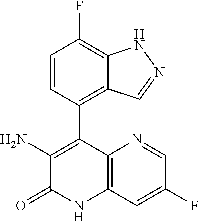 [SMILES: Nc1c(-c2ccc(F)c3[nH]ncc23)c2ncc(F)cc2[nH]c1=O] | | Hz, 1 H), 7.28 (dd, J = 11.01, 7.88 Hz, 1 H), 7.00 (dd, J = 7.82, 4.19 Hz, 1 H), 5.26 (s, 2H). ¹⁹F NMR (376 MHz, DMSO-d₆) δ ppm −73.41 (s, 1F), −132.73 (s, 1F). MS (ESI⁺) obsd. [(M + H)⁺]: 314.0. |
| 005 | 3-Amino-4-(7-fluoro- | Compound Int-5 | ¹H NMR (400 MHz, DMSO- |
| | 1H-indazol-4-y1)-7- | | d₆) δ ppm 13.62 (br s, 1H), |
| | (trifluoromethyl)-1H- | | 12.37 (br s, 1H), 8.44 (d, J = |
| | 1,5-naphthyridin-2-one | | 1.25 Hz, 1 H), 7.83 (d, J = |
| | | | 2.00 Hz, 1 H), 7.66 (br s, 1 |
| | 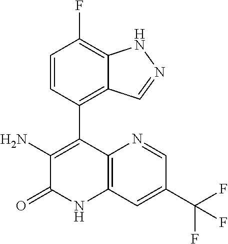 [SMILES: Nc1c(-c2ccc(F)c3[nH]ncc23)c2ncc(C(F)(F)F)cc2[nH]c1=O] | | H), 7.29 (dd, J = 11.07, 7.82 Hz, 1 H), 7.01 (dd, J = 7.75, 4.13 Hz, 1 H), 5.80 (s, 2H). ¹⁹F NMR (376 MHz, DMSO-d₆) δ ppm −59.78 (s, 3F), −132.62 (s, 3F). MS (ESI⁺) obsd. [(M + H)⁺]: 364.1. |
| 006 | 3-Amino-4-(7-fluoro- | Compound Int-6 | ¹H NMR (400 MHz, DMSO- |
| | 1H-indazol-4-yl)-7- | | d₆) δ ppm 13.61 (br s, 1H), |
| | methoxy-1H-1,5- | | 12.00 (br s, 1H), 7.93 (d, J = |
| | naphthyridin-2-one | | 2.75 Hz, 1 H), 7.60 (br s, 1 |
| | | | H), 7.27 (dd, J = 11.26, 7.75 |
| | 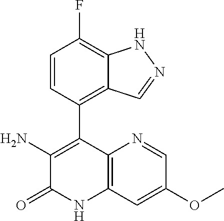 [SMILES: COc1cnc2c(-c3ccc(F)c4[nH]ncc34)c(N)c(=O)[nH]c2c1] | | Hz, 1 H), 7.18 (d, J = 2.63 Hz, 1 H), 6.99 (dd, J = 7.82, 4.06 Hz, 1 H), 4.96 (s, 2H), 3.81 (s, 3 H). ¹⁹F NMR (376 MHz, DMSO-d₆) δ ppm −132.87 (s, 1F). MS (ESI⁺) obsd. [(M + H)⁺]: 326.1. |

### Example 007: 4-[7-Amino-8-(7-fluoro-1H-indazol-4-yl)-6-oxo-5H-1,5-naphthyridin-3-yl]-N,N-dimethyl-benzamide

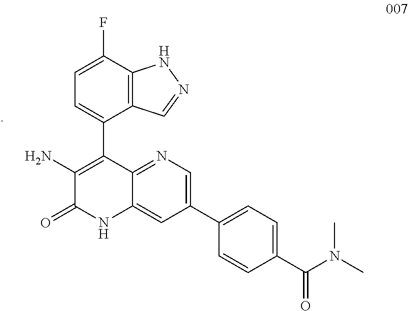 [SMILES: CN(C)C(=O)c1ccc(-c2cnc3c(-c4ccc(F)c5[nH]ncc45)c(N)c(=O)[nH]c3c2)cc1]

To a solution of Example 001 (30.0 mg, 91.0 μmol) in 1,4-dioxane (2.0 mL) and H₂O (0.2 mL) were added 4-(N,N-dimethylaminocarbonyl)phenylboronic acid (21.1 mg, 109.2 μmol), potassium phosphate tribasic (48.3 mg, 227.5 μmol) and 1,1′-bis(di-t-butylphosphino)ferrocene palladium dichloride (5.9 mg, 9.1 μmol). The mixture was stirred at 100° C. for 2 hours. After completion, the mixture was filtered and concentrated in vacuo to give the crude product, which was purified by prep-HPLC (FA as additive) to afford compound Example 007 (10.0 mg, 24.8% yield) as yellow powder. MS (ESI⁺) obsd. [(M+H)⁺]: 443.1.

Example 007: ¹H NMR (400 MHz, DMSO-d₆) δ ppm 13.63 (br s, 1H), 12.22 (br s, 1H), 8.51 (d, J=1.88 Hz, 1H), 7.85 (d, J=1.88 Hz, 1H), 7.71 (d, J=8.25 Hz, 2H), 7.52 (d, J=8.13 Hz, 2H), 7.30 (dd, J=10.94, 7.94 Hz, 1H), 7.04 (dd, J=7.69, 4.06 Hz, 1H), 5.41 (br s, 2H), 2.99 (s, 3H), 2.96 (s, 3H). ¹⁹F NMR (376 MHz, DMSO-d₆) δ ppm −132.81 (s, 1F).

The following Examples 008 to 014 were prepared in analogy to the procedure described for the preparation of Example 007, replacing compound 4-(N,N-dimethylaminocarbonyl)phenyl-boronic acid with BORONICCPD indicated in Table 2.

TABLE 2
Compound synthesis and characterization

| | Compound Names and | | |
|---|---|---|---|
| Ex. | Structures | BORONICCPD | NMR and (ESI⁺) |
| 008 | 3-Amino-4-(7-fluoro- | 1-Methyl-5-(4,4,5,5- | ¹H NMR (400 MHz, DMSO-d₆) δ |
| | 1H-indazol-4-yl)-7-(3- | tetramethyl-1,3,2- | ppm 13.63 (br s, 1H), 12.17 (br s, |
| | methylimidazol-4-yl)- | dioxaborolan-2- | 1H), 8.30 (d, J = 2.0 Hz, 1H), 7.75 |
| | 1H-1,5-naphthyridin-2- | yl)imidazole | (s, 1H), 7.69 (d, J = 2.0 Hz, 1H), |
| | one | | 7.67 (br s, 1H), 7.29 (dd, J = 7.9, |
| | 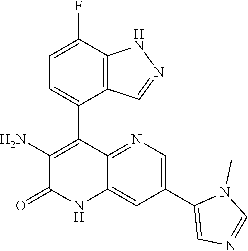 [SMILES: Cn1cncc1-c1cnc2c(-c3ccc(F)c4[nH]ncc34)c(N)c(=O)[nH]c2c1] | | 10.9 Hz, 1H), 7.11 (s, 1H), 7.03 (dd, J = 4.1, 7.8 Hz, 1H), 5.40 (br s, 2H), 3.68 (s, 3H). ¹⁹F NMR (376 MHz, DMSO-d₆) δ ppm −132.77 (s, 1F). MS (ESI⁺) obsd. [(M + H)⁺]: 376.1. |
| 009 | 3-Amino-4-(7-fluoro- | 3-(4,4,5,5- | ¹H NMR (400 MHz, DMSO-d₆) δ |
| | 1H-indazol-4-yl)-7- | Tetramethyl-1,3,2- | ppm 13.62 (br s, 1H), 12.99 (br s, |
| | (1H-pyrazol-5-yl)-1H- | dioxaborolan-2-yl)- | 1H), 12.17 (br s, 1H), 8.58 (br s, |
| | 1,5-naphthyridin-2-one | 1H-pyrazole | 1H), 8.02 (br s, 1H), 7.81 (br s, |
| | 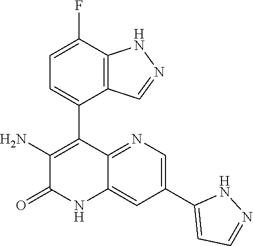 [SMILES: Nc1c(-c2ccc(F)c3[nH]ncc23)c2ncc(-c3ccn[nH]3)cc2[nH]c1=O] | | 1H), 7.65 (br s, 1H), 7.29 (dd, J = 7.7, 11.1 Hz, 1H), 7.03 (dd, J = 3.9, 7.6 Hz, 1H), 6.69 (br s, 1H), 5.30 (br s, 2H). ¹⁹F NMR (376 MHz, DMSO-d₆) δ ppm −102.64 (br d, J = 13.62 Hz, 2F). ¹⁹F NMR (376 MHz, DMSO-d₆) δ ppm −132.88 (s, 1F). MS (ESI⁺) obsd. [(M + H)⁺]: 362.1. |
| 010 | 3-Amino-4-(7-fluoro- | 5-(4,4,5,5- | ¹H NMR (400 MHz, DMSO-d₆) δ |
| | 1H-indazol-4-yl)-7- | Tetramethyl-1,3,2- | ppm 13.63 (br s, 1H), 12.25 (br s, |
| | isothiazol-5-yl-1H-1,5- | dioxaborolan-2- | 1H), 8.62 (d, J = 1.8 Hz, 1H), 8.55 |
| | naphthyridin-2-one | yl)isothiazole | (d, J = 2.1 Hz, 1H), 7.83 (d, J = 2.1 |
| | 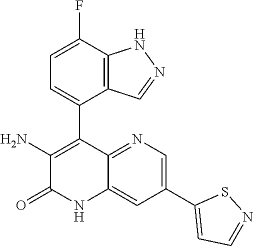 [SMILES: Nc1c(-c2ccc(F)c3[nH]ncc23)c2ncc(-c3ccns3)cc2[nH]c1=O] | | Hz, 1H), 7.77 (d, J = 1.8 Hz, 1H), 7.67 (br s, 1H), 7.29 (dd, J = 7.9, 11.3 Hz, 1H), 7.03 (dd, J = 4.3, 7.8 Hz, 1H), 5.60 (br s, 2H). ¹⁹F NMR (376 MHz, DMSO-d₆) δ ppm −132.70 (s, 1F). MS (ESI⁺) obsd. [(M + H)⁺]: 379.1. |
| 011 | 3-Amino-4-(7-fluoro- | 4-(4,4,5-Trimethyl- | ¹H NMR (400 MHz, DMSO-d₆) δ |
| | 1H-indazol-4-yl)-7-[2- | 1,3,2-dioxaborolan-2- | ppm 13.64 (br s, 1H), 12.26 (br s, |
| | (trifluoromethyl)-4- | yl)-2-trifluoromethyl | 1H), 8.83 (d, J = 5.1 Hz, 1H), 8.67 |
| | pyridyl]-1H-1,5- | pyridine | (d, J = 2.1 Hz, 1H), 8.15 (d, J = 0.8 |
| | naphthyridin-2-one | | Hz, 1H), 8.03-8.00 (m, 1H), 7.98 |
| | 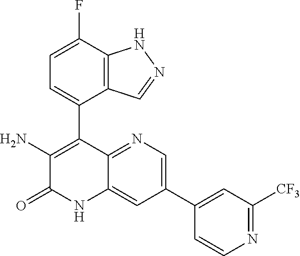 [SMILES: Nc1c(-c2ccc(F)c3[nH]ncc23)c2ncc(-c3ccnc(C(F)(F)F)c3)cc2[nH]c1=O] | | (d, J = 2.1 Hz, 1H), 7.67 (br s, 1H), 7.30 (dd, J = 7.9, 11.1 Hz, 1H), 7.03 (dd, J = 4.2, 7.7 Hz, 1H), 5.62 (s, 2H). ¹⁹F NMR (376 MHz, DMSO-d₆) δ ppm −66.67 (s, 3F), −128.74 (s, 1F). MS (ESI⁺) obsd. [(M + H)⁺]: 441.1. |
| 012 | 3-Amino-4-(7-fluoro- | 4-(4,4,5,5- | ¹H NMR (400 MHz, DMSO-d₆) δ |
| | 1H-indazol-4-yl)-7- | Tetramethyl-1,3,2- | ppm 13.64 (br s, 1H), 12.35 (br s, |
| | pyridazin-4-yl-1H-1,5- | dioxaborolan-2- | 1H), 12.43-12.26 (m, 1H), 9.63- |
| | naphthyridin-2-one | yl)pyridazine | 9.55 (m, 1H), 9.32-9.24 (m, 1H), |
| | 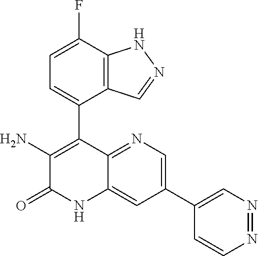 [SMILES: Nc1c(-c2ccc(F)c3[nH]ncc23)c2ncc(-c3ccnnc3)cc2[nH]c1=O] | | 8.67 (d, J = 2.1 Hz, 1H), 7.99-7.95 (m, 2H), 7.67 (br s, 1H), 7.30 (dd, J = 7.8, 11.1 Hz, 1H), 7.03 (dd, J = 4.1, 7.8 Hz, 1H), 5.63 (br s, 2H). ¹⁹F NMR (376 MHz, DMSO-d₆) δ ppm −132.70 (s, 1F). MS (ESI⁺) obsd. [(M + H)⁺]: 374.1. |
| 013 | 3-Amino-4-(7-fluoro- | 2-Pyridylboronic acid | ¹H NMR (400 MHz, DMSO-d₆) δ |
| | 1H-indazol-4-yl)-7-(2- | | ppm 13.63 (br s, 1H), 12.29 (br s, |
| | pyridyl)-1H-1,5- | | 1H), 8.82 (d, J = 2.0 Hz, 1H), 8.69 |
| | naphthyridin-2-one | | (d, J = 4.8 Hz, 1H), 8.33 (d, J = 2.0 |
| | 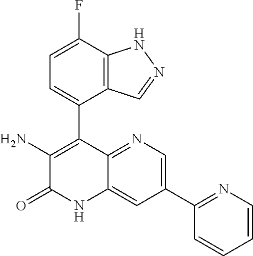 [SMILES: Nc1c(-c2ccc(F)c3[nH]ncc23)c2ncc(-c3ccccn3)cc2[nH]c1=O] | | Hz, 1H), 7.96 (d, J = 8.0 Hz, 1H), 7.92-7.85 (m, 1H), 7.67 (br s, 1H), 7.40-7.34 (m, 1H), 7.34-7.27 (m, 1H), 7.04 (dd, J = 4.1, 7.8 Hz, 1H), 5.47 (br s, 2H). ¹⁹F NMR (376 MHz, DMSO-d₆) δ ppm −132.82 (s, 1F). MS (ESI⁺) obsd. [(M + H)⁺]: 373.1. |
| 014 | 3-Amino-4-(7-fluoro- | 5-Fluoropyridine-2- | ¹H NMR (400 MHz, DMSO-d₆) δ |
| | 1H-indazol-4-yl)-7-(5- | boronic acid | ppm 13.63 (br s, 1H), 12.30 (br s, |
| | fluoro-2-pyridyl)-1H- | | 1H), 8.79 (d, J = 2.0 Hz, 1H), 8.69 |
| | 1,5-naphthyridin-2-one | | (d, J = 3.0 Hz, 1H), 8.27 (d, J = 2.1 |
| | 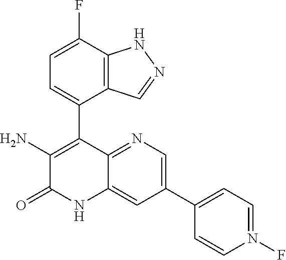 | | Hz, 1H), 8.04 (dd, J = 4.4, 8.9 Hz, 1H), 7.83 (dt, J = 3.0, 8.8 Hz, 1H), 7.67 (br s, 1H), 7.30 (dd, J = 7.8, 11.2 Hz, 1H), 7.04 (dd, J = 4.1, 7.8 Hz, 1H), 5.48 (br s, 2H). ¹⁹F NMR (376 MHz, DMSO-d₆) δ ppm −129.08 (dd, J = 4.2, 8.3 Hz, 1F), −132.82 (s, 1F). MS (ESI⁺) obsd. [(M + H)⁺]: 391.1. |

### Example 015: 3-Amino-7-cyclopropyl-4-(7-fluoro-1H-indazol-4-yl)-1H-1,5-naphthyridin-2-one

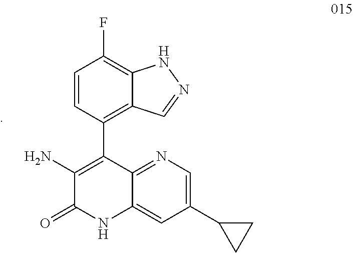 [SMILES: Nc1c(-c2ccc(F)c3[nH]ncc23)c2ncc(C3CC3)cc2[nH]c1=O]

### Step 1: tert-Butyl N-[5-cyclopropyl-2-(7-fluoro-1-tetrahydropyran-2-yl-indazole-4-carbonyl)-3-pyridyl]carbamate

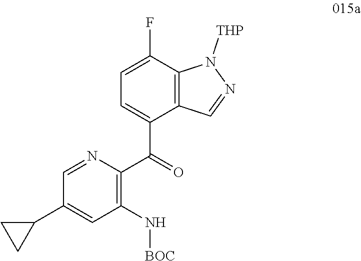

The mixture of compound 001a (300.0 mg, 0.63 mmol), cyclopropylboronic acid (108.53 mg, 1.26 mmol, 2.0 eq), Pd(dppf)Cl₂ (46.22 mg, 0.06 mmol) and K₂CO₃ (174.6 mg, 1.26 mmol) in 1,4-dioxane (6 mL) was stirred at 100° C. for 12 hours under N₂ protection. After completion, the mixture was diluted with water (10 mL) and extracted with EtOAc (7 mL×3). The combined organic layer was washed with brine (20 mL), dried over anhydrous sodium sulfate and concentrated in vacuo to give a residue, which was purified by silica-gel chromatography (elute with EtOAc:PE=0-20%) to give compound 015a (300.0 mg, 98.8% yield) as a white solid. MS (ESI⁺) obsd. [(M+H-Me)⁺]: 481.3.

### Step 2: (3-Amino-5-cyclopropylpyridin-2-yl)(7-fluoro-1H-indazol-4-yl)methanone

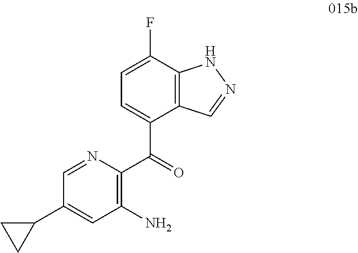 [SMILES: Nc1cc(C2CC2)cnc1C(=O)c1ccc(F)c2[nH]ncc12]

The title compound was prepared in analogy to the preparation of compound 001b, replacing compound 001a with compound 015a. Compound 015b (120.0 mg) was obtained as a yellow solid. MS (ESI⁺) obsd. [(M+H)⁺]: 297.1.

### Step 3: 2-Chloro-N-[5-cyclopropyl-2-(7-fluoro-1H-indazole-4-carbonyl)-3-pyridyl]acetamide

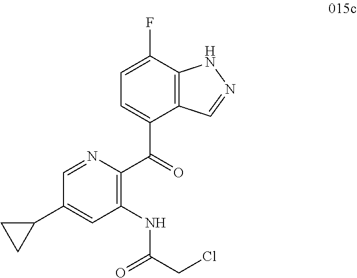 [SMILES: O=C(CCl)Nc1cc(C2CC2)cnc1C(=O)c1ccc(F)c2[nH]ncc12]

The title compound was prepared in analogy to the preparation of compound 001c, replacing compound 001b with compound 015b. Compound 015c (110.0 mg, crude) was obtained as a grey solid. MS (ESI⁺) obsd. [(M+H)⁺]: 373.1.

### Step 4: 7-Chloro-4-(7-fluoro-1H-indazol-4-yl)-3-pyridin-1-ium-1-yl-1H-1,5-naphthyridin-2-one;chloride

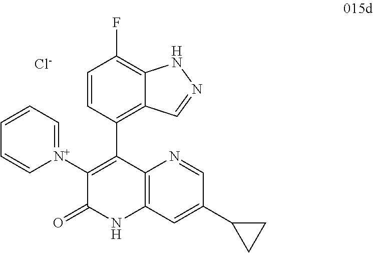 [SMILES: O=c1[nH]c2cc(C3CC3)cnc2c(-c2ccc(F)c3[nH]ncc23)c1-[n+]1ccccc1 ; [Cl-]]

The title compound was prepared in analogy to the preparation of 001d, replacing compound 001c with compound 015c. Compound 015d (80.0 mg, crude) was obtained as a black solid. MS (ESI⁺) obsd. [(M-Cl)⁺]: 398.1.

### Step 5: 3-Amino-7-cyclopropyl-4-(7-fluoro-1H-indazol-4-yl)-1H-1,5-naphthyridin-2-one

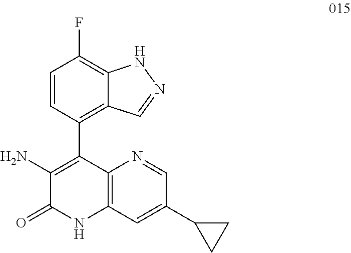 [SMILES: Nc1c(-c2ccc(F)c3[nH]ncc23)c2ncc(C3CC3)cc2[nH]c1=O]

The title compound was prepared in analogy to the preparation of Example 001, replacing compound 001d with compound 015d in step 5. Example 015 (80.0 mg, crude) was obtained as a light red solid. MS (ESI⁺) obsd. [(M+H)⁺]: 336.2.

Example 015: ¹H NMR (400 MHz, DMSO-d₆) δ ppm 13.60 (br s, 1H), 12.00 (br d, J=5.6 Hz, 1H), 7.98 (d, J=2.0 Hz, 1H), 7.59 (s, 1H), 7.27 (dd, J=7.6, 10.8 Hz, 1H), 7.22 (d, J=2.0 Hz, 1H), 6.98 (dd, J=4.4, 7.6 Hz, 1H), 5.14 (s, 2H), 2.00-1.91 (m, 1H), 1.02-0.96 (m, 2H), 0.67-0.65-0.96 (in, 2H). ¹⁹F NMR (376 MHz, DMSO-d₆) δ ppm −132.931

The following Examples 016 to 018 were prepared in analogy to the procedure described for the preparation of Example 015, replacing compound 001a with INTERMEDIATE and cyclopropylboronic acid with TAIL in step1, both INTERMEDIATE and TAIL are indicated in Table 3.

TABLE 3
Compound synthesis and characterization

| | Compound Names and | INTER- | | |
|---|---|---|---|---|
| Ex. | Structures | MEDIATE | TAIL | NMR and (ESI⁺) |
| 016 | 3-Amino-4-(7-fluoro- | Compound 001a | 2- | ¹H NMR (400 MHz, DMSO- |
| | 1H-indazol-4-yl)-7- | | (Tributyl- | d₆) δ ppm 13.66 (br, s, 1H), |
| | thiazol-2-yl-1H-1,5- | | stannyl) | 12.25 (br, s, 1H), 8.68 (d, J = |
| | naphthyridin-2-one | | thiazole | 2.0 Hz, 1H), 8.14 (d, J = 2.0 |
| | 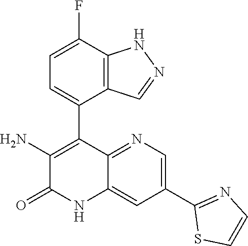 [SMILES: Nc1c(-c2ccc(F)c3[nH]ncc23)c2ncc(-c3nccs3)cc2[nH]c1=O] | | | Hz, 1H), 7.95 (d, J = 3.4 Hz, 1H), 7.80 (d, J = 3.2 Hz, 1H), 7.68 (d, J = 3.2 Hz, 1H), 7.30 (dd, J = 7.6, 11.2 Hz, 1H), 7.03 (dd, J = 4.4, 8.0 Hz, 1H), 5.60 (s, 2H). ¹⁹F NMR (376 MHz, DMSO-d₆) δ ppm −132.660. MS (ESI⁺) obsd. [(M + H)⁺]: 379.2. |
| 017 | 3-Amino-4-(7-fluoro- | Compound Int-9 | Trimethyl- | ¹H NMR (400 MHz, DMSO- |
| | 1H-indazol-4-yl)-6,7- | | boroxine | d₆) δ ppm 13.60 (s, 1H), |
| | dimethyl-1H-1,5- | | | 12.00 (s, 1H), 7.60 (s, 1H), |
| | naphthyridin-2-one | | | 7.34 (s, 1H), 7.28 (dd, J = |
| | 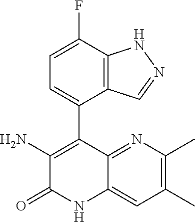 [SMILES: Cc1cc2[nH]c(=O)c(N)c(-c3ccc(F)c4[nH]ncc34)c2nc1C] | | | 8.0, 11.2 Hz, 1H), 7.01 (dd, J = 4.0, 7.6 Hz, 1H), 5.04 (s, 2H), 2.24 (s, 3H), 2.16 (s, 3H). ¹⁹F NMR (376 MHz, DMSO-d₆) δ ppm −132.98 (s, 1F). MS (ESI⁺) obsd. [(M + H)⁺]: 324.0. |
| 018 | 3-Amino-6- | Compound Int-9 | Cyclo- | ¹H NMR (400 MHz, DMSO- |
| | cyclopropyl-4-(7- | | propyl- | d₆) δ ppm 12.01 (s, 1H), 7.54 |
| | fluoro-1H-indazol-4- | | boronic | (d, J = 2.4 Hz, 1H), 7.33 (s, |
| | yl)-7-methyl-1H-1,5- | | acid | 1H), 7.27 (dd, J = 8.0, 11.3 |
| | naphthyridin-2-one | | | Hz, 1H), 7.02 (dd, J = 4.0, |
| | 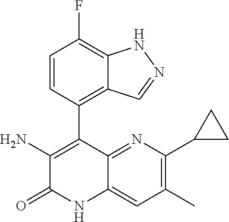 [SMILES: Cc1cc2[nH]c(=O)c(N)c(-c3ccc(F)c4[nH]ncc34)c2nc1C1CC1] | | | 8.0 Hz, 1H), 2.38 (s, 3H), 1.97 (t, J = 7.6 Hz, 1H), 0.62- 0.53 (m, 2H), 0.37 (d, J = 4.8 Hz, 1H), 0.27 (d, J = 2.8 Hz, 1H). ¹⁹F NMR (376 MHz, DMSO-d₆) δ ppm −74.60(s, 1F). MS (ESI⁺) obsd. [(M + H)⁺]: 350.2. |

### Example 019: 3-Amino-4-(7-fluoro-1H-indazol-4-yl)-7-morpholino-1H-1,5-naphthyridin-2-one

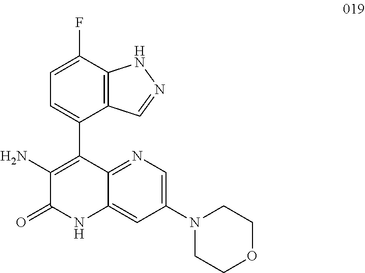 [SMILES: Nc1c(-c2ccc(F)c3[nH]ncc23)c2ncc(N3CCOCC3)cc2[nH]c1=O]

### Step 1: tert-Butyl N-[2-(7-fluoro-1-tetrahydropyran-2-yl-indazole-4-carbonyl)-5-morpholino-3-pyridyl]carbamate

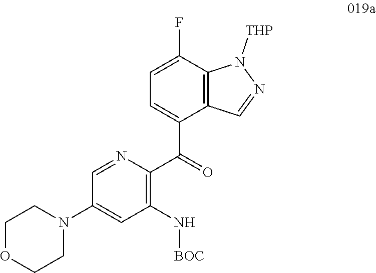

The mixture of compound 001a (200.0 mg, 0.42 mmol), RuPhos-Pd-G4 (35.81 mg, 0.04 mmol), morpholine (36.69 mg, 0.42 mmol) and t-BuONa (0.42 mL, 0.84 mmol) in 1,4-Dioxane (2 mL) was stirred at 90° C. for 6 hours under N₂ protection. After completion, the mixture was filtered and the filtrate was concentrated in vacuo to give a residue, which was purified by silica-gel chromatography (elute with EtOAc:PE=0-40%) to give compound 019a (120.0 mg, 54.2% yield) as a yellow solid. MS (ESI⁺) obsd. [(M+H)⁺]: 526.5.

### Step 2: (3-Amino-5-morpholino-2-pyridyl)-(7-fluoro-1H-indazol-4-yl)methanone

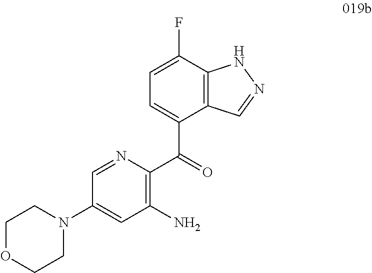 [SMILES: Nc1cc(N2CCOCC2)cnc1C(=O)c1ccc(F)c2[nH]ncc12]

The title compound was prepared in analogy to the preparation of compound 001b, replacing compound 001a with compound 019a. Compound 019b (120.0 mg) was obtained as a yellow solid. MS (ESI⁺) obsd. [(M+H)⁺]: 342.1.

### Step 3: 2-Chloro-N-[2-(7-fluoro-1H-indazole-4-carbonyl)-5-morpholino-3-pyridyl]acetamide

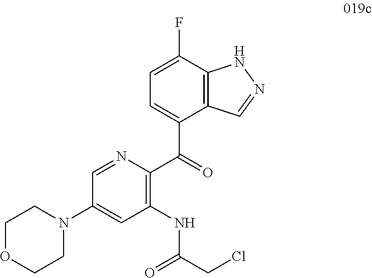 [SMILES: O=C(CCl)Nc1cc(N2CCOCC2)cnc1C(=O)c1ccc(F)c2[nH]ncc12]

The title compound was prepared in analogy to the preparation of compound 001c, replacing compound 001b with compound 019b. Compound 019c (80.0 mg, crude) was obtained as a yellow solid. MS (ESI⁺) obsd. [(M+H)⁺]: 418.1.

### Step 4: 4-(7-Fluoro-1H-indazol-4-yl)-7-morpholino-3-pyridin-1-ium-1-yl-1H-1,5-naphthyridin-2-one;chloride

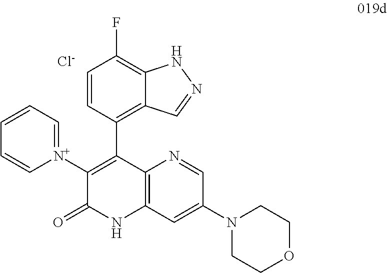 [SMILES: O=c1[nH]c2cc(N3CCOCC3)cnc2c(-c2ccc(F)c3[nH]ncc23)c1-[n+]1ccccc1 ; [Cl-]]

The title compound was prepared in analogy to the preparation of 001d, replacing compound 001c with compound 019c. Compound 019d (70.0 mg, crude) was obtained as a black solid. MS (ESI⁺) obsd. [(M-Cl)⁺]: 443.3.

### Step 5: 3-Amino-4-(7-fluoro-1H-indazol-4-yl)-7-morpholino-1H-1,5-naphthyridin-2-one

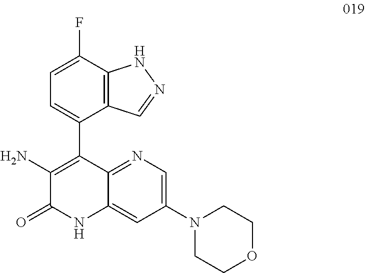 [SMILES: Nc1c(-c2ccc(F)c3[nH]ncc23)c2ncc(N3CCOCC3)cc2[nH]c1=O]

The title compound was prepared in analogy to the preparation of Example 001, replacing compound 001d with compound 019d in step 5. Example 019 (29.7 mg) was obtained as a light red solid. MS (ESI⁺) obsd. [(M+H)⁺]: 381.1.

Example 019: ¹H NMR (400 MHz, DMSO-d₆) δ ppm 8.04 (d, J=2.4 Hz, 1H), 7.60 (d, J=3.2 Hz, 1H), 7.27 (dd, J=8.0, 11.6 Hz, 1H), 7.05 (d, J=2.8 Hz, 1H), 6.99 (dd, J=4.4, 8.0 Hz, 1H), 4.89 (s, 2H), 3.78-3.72 (m, 4H), 3.14-3.08 (m, 4H). ¹⁹F NMR (376 MHz, DMSO-d₆) δ ppm −132.876.

### Example 020: 3-Amino-4-(7-fluoro-1H-indazol-4-yl)-7-(1-hydroxy-1-methyl-ethyl)-1H-1,5-naphthyridin-2-one

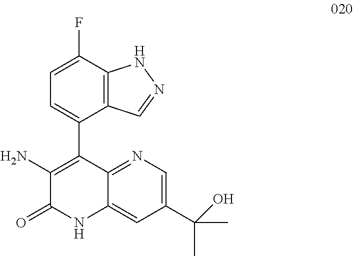 [SMILES: CC(C)(O)c1cnc2c(-c3ccc(F)c4[nH]ncc34)c(N)c(=O)[nH]c2c1]

### Step 1: Ethyl 5-(tert-butoxycarbonylamino)-6-(7-fluoro-1-tetrahydropyran-2-yl-indazole-4-carbonyl)pyridine-3-carboxylate

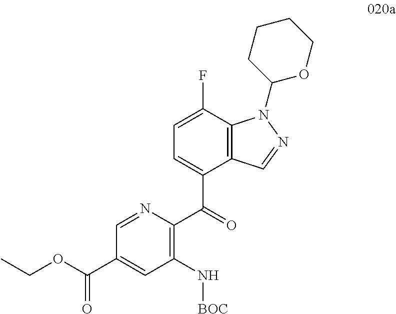 [SMILES: CCOC(=O)c1cnc(C(=O)c2ccc(F)c3c2cnn3C2CCCCO2)c(NC(=O)OC(C)(C)C)c1]

A mixture of compound 001a (300.0 mg, 0.63 mmol), Pd(dppf)Cl₂ (46.22 mg, 0.06 mmol, 0.1 eq) and TEA (0.18 mL, 1.26 mmol) in ethyl alcohol (29.1 mg, 0.63 mmol) was stirred at 80° C. for 12 hours under CO atmosphere (50 psi). After completion, the mixture was filtered and the filtrate was concentrated in vacuo to give a residue, which was purified by silica-gel chromatography (elute with EtOAc:PE=0-20%) to give compound 020a (320.0 mg, 98.8% yield) as a yellow solid. MS (ESI⁺) obsd. [(M+H)⁺]: 513.3.

### Step 2: Ethyl 5-amino-6-(7-fluoro-1H-indazole-4-carbonyl)pyridine-3-carboxylate

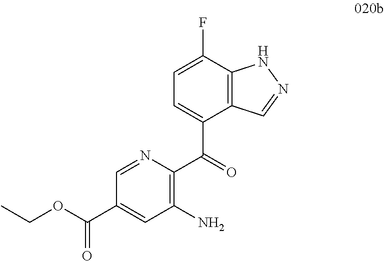 [SMILES: CCOC(=O)c1cnc(C(=O)c2ccc(F)c3[nH]ncc23)c(N)c1]

The title compound was prepared in analogy to the preparation of compound 001b, replacing compound 001a with compound 020a. Compound 020b (200.0 mg) was obtained as a yellow solid. MS (ESI⁺) obsd. [(M+H)⁺]: 329.0.

### Step 3: Ethyl 5-[(2-chloroacetyl)amino]-6-(7-fluoro-1H-indazole-4-carbonyl)pyridine-3-carboxylate

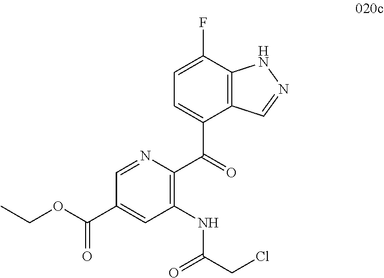 [SMILES: CCOC(=O)c1cnc(C(=O)c2ccc(F)c3[nH]ncc23)c(NC(=O)CCl)c1]

The title compound was prepared in analogy to the preparation of compound 001c, replacing compound 001b with compound 020b. Compound 020c (210.0 mg, crude) was obtained as a brown solid. MS (ESI⁺) obsd. [(M+H)⁺]: 405.0.

### Step 4: Ethyl 8-(7-fluoro-1H-indazol-4-yl)-6-oxo-7-pyridin-1-ium-1-yl-5H-1,5-naphthyridine-3-carboxylate;chloride

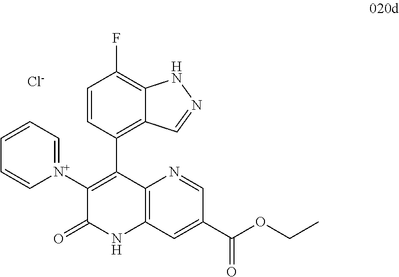 [SMILES: CCOC(=O)c1cnc2c(-c3ccc(F)c4[nH]ncc34)c(-[n+]3ccccc3)c(=O)[nH]c2c1 ; [Cl-]]

The title compound was prepared in analogy to the preparation of 001d, replacing compound 001c with compound 020c. Compound 020d (70.0 mg, crude) was obtained as a black solid. MS (ESI⁺) obsd. [(M-Cl)⁺]: 430.0.

### Step 5: Ethyl 7-amino-8-(7-fluoro-1H-indazol-4-yl)-6-oxo-5H-1,5-naphthyridine-3-carboxylate

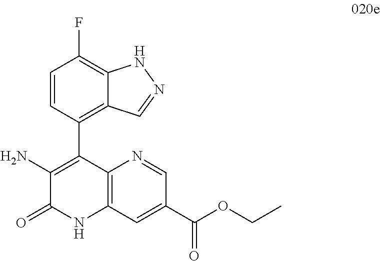 [SMILES: CCOC(=O)c1cnc2c(-c3ccc(F)c4[nH]ncc34)c(N)c(=O)[nH]c2c1]

The title compound was prepared in analogy to the preparation of Example 001, replacing compound 001d with compound 020d in step 5. Compound 020e (97.0 mg) was obtained as a white solid. MS (ESI⁺) obsd. [(M+H)⁺]: 368.1.

### Step 6: 3-Amino-4-(7-fluoro-1H-indazol-4-yl)-7-(1-hydroxy-1-methyl-ethyl)-1H-1,5-naphthyridin-2-one

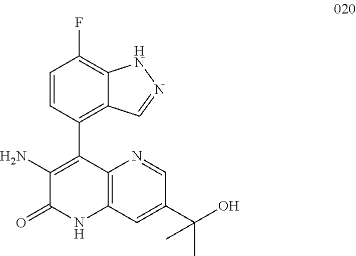 [SMILES: CC(C)(O)c1cnc2c(-c3ccc(F)c4[nH]ncc34)c(N)c(=O)[nH]c2c1]

To a solution of compound 020e (40.0 mg, 0.11 mmol) in THE (3 mL) was added methyl magnesium bromide (0.36 mL, 1.09 mmol) at 0° C. under nitrogen atmosphere, then the mixture was stirred at 0° C. for 2 hours. After completion, the reaction mixture was quenched with saturated ammonium chloride (5 mL) and the resulting mixture was extracted with EtOAc(5 mL×3). The combined organic layer was washed with brine (15 mL), dried over anhydrous sodium sulfate and concentrated in vacuo to give a residue, which was purified by prep-HPLC (NH₃ as additive) and lyophilized to give Example 020 (8.2 mg, 0.02 mmol, 20.76% yield) as a light grey solid. MS (ESI⁺) obsd. [(M+H)⁺]: 354.3

Example 020: ¹H NMR (400 MHz, MeOD-d₄) δ ppm 8.31 (d, J=2.0 Hz, 1H), 7.84 (d, J=2.0 Hz, 1H), 7.64 (d, J=2.8 Hz, 1H), 7.29 (dd, J=7.6, 10.8 Hz, 1H), 7.12 (dd, J=4.0, 8.0 Hz, 1H), 1.57 (s, 6H). ¹⁹F NMR (376 MHz, MeOD-d₄) δ ppm −134.560.

### Example 021: 3-Amino-6-bromo-4-(7-fluoro-1H-indazol-4-yl)-7-methyl-1H-1,5-naphthyridin-2-one

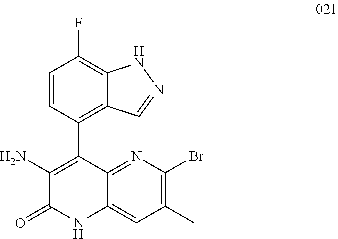 [SMILES: Cc1cc2[nH]c(=O)c(N)c(-c3ccc(F)c4[nH]ncc34)c2nc1Br]

### Step 1: tert-Butyl N-[2-(7-fluoro-1-tetrahydropyran-2-yl-indazole-4-carbonyl)-5-methyl-3-pyridyl]carbamate

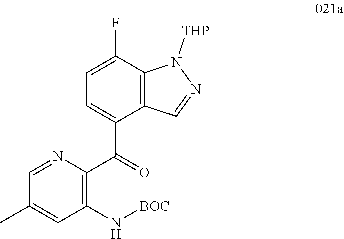

The title compound was prepared in analogy to the preparation of compound 001a, replacing compound Int-2 with compound Int-3. Compound 021a (400 mg) was obtained as a white solid. MS (ESI⁺) obsd. [(M+H)⁺]: 455.2.

### Step 2: (3-Amino-5-methyl-2-pyridyl)-(7-fluoro-1H-indazol-4-yl)methanone

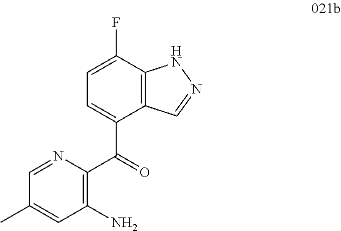 [SMILES: Cc1cnc(C(=O)c2ccc(F)c3[nH]ncc23)c(N)c1]

The title compound was prepared in analogy to the preparation of compound 001b, replacing compound 001a with compound 021a. Compound 021b (400 mg) was obtained as a white solid. MS (ESI⁺) obsd. [(M+H)⁺]: 271.1.

### Step 3: (3-Amino-6-bromo-5-methyl-2-pyridyl)-(7-fluoro-1H-indazol-4-yl)methanone

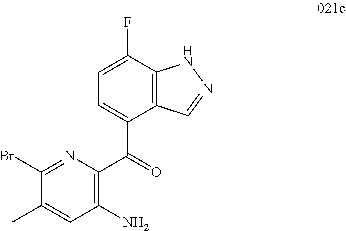 [SMILES: Cc1cc(N)c(C(=O)c2ccc(F)c3[nH]ncc23)nc1Br]

To a mixture of compound 021b (400.0 mg, 1.5 mmol) in DMF (5 mL) was added NBS (263.0 mg, 1.5 mmol). The reaction mixture was stirred at 25° C. for 12 hours. After completion, the reaction mixture was quenched with saturated sodium sulfite (20 mL) and extracted with EtOAc (20 mL×3). The combined organic layer was washed with brine (40 mL), dried over anhydrous sodium sulfate and concentrated in vacuo to give a residue, which was purified by prep-HPLC (TFA as additive) and lyophilized to give compound 021c (340.0 mg, 65.8% yield) as a yellow solid. MS (ESI⁺) obsd. [(M+H)⁺]: 349.0.

### Step 4: N-[6-bromo-2-(7-fluoro-1H-indazole-4-carbonyl)-5-methyl-3-pyridyl]-2-chloro-acetamide

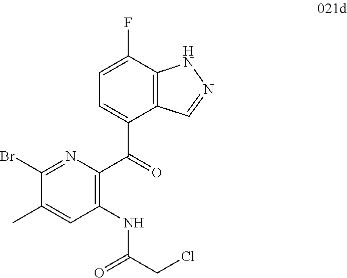 [SMILES: Cc1cc(NC(=O)CCl)c(C(=O)c2ccc(F)c3[nH]ncc23)nc1Br]

The title compound was prepared in analogy to the preparation of compound 001c, replacing compound 001b with compound 021c. Compound 021d (420.0 mg, crude) was obtained as a grey solid. MS (ESI⁺) obsd. [(M+H)⁺]: 425.1.

### Step 5: 6-Bromo-4-(7-fluoro-1H-indazol-4-yl)-7-methyl-3-pyridin-1-ium-1-yl-1H-1,5-naphthyridin-2-one;chloride

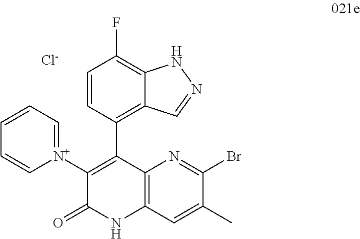 [SMILES: Cc1cc2[nH]c(=O)c(-[n+]3ccccc3)c(-c3ccc(F)c4[nH]ncc34)c2nc1Br ; [Cl-]]

The title compound was prepared in analogy to the preparation of compound 001d, replacing compound 001c with compound 021d. Compound 021e (400.0 mg, crude) was obtained as a brown solid. MS (ESI⁺) obsd. [(M-Cl)⁺]: 451.9.

### Step 6: 3-Amino-6-bromo-4-(7-fluoro-1H-indazol-4-yl)-7-methyl-1H-1,5-naphthyridin-2-one

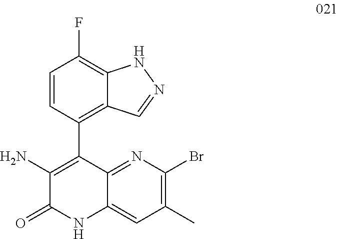 [SMILES: Cc1cc2[nH]c(=O)c(N)c(-c3ccc(F)c4[nH]ncc34)c2nc1Br]

The title compound was prepared in analogy to the preparation of Example 001, replacing compound 001d with compound 021e in step 5. Example 021 (190.0 mg) was obtained as a white solid. MS (ESI⁺) obsd. [(M+H)⁺]: 388.0.

Example 021: ¹H NMR (400 MHz, DMSO-d₆) δ ppm 13.65 (s, 1H), 12.25 (s, 1H), 7.66 (s, 1H), 7.51 (s, 1H), 7.30 (dd, J=8.0, 11.2 Hz, 1H), 7.02 (dd, J=4.0, 7.6 Hz, 1H), 5.40 (s, 2H), 2.31 (s, 3H). ¹⁹F NMR (376 MHz, DMSO-d₆) δ ppm −132.42 (s, 1F).

### Example 022: 3-Amino-4-(7-fluoro-1H-indazol-4-yl)-7-methyl-6-(4-methylpiperazin-1-yl)-1H-1,5-naphthyridin-2-one

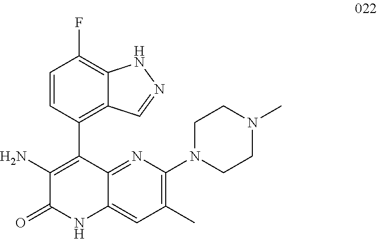 [SMILES: Cc1cc2[nH]c(=O)c(N)c(-c3ccc(F)c4[nH]ncc34)c2nc1N1CCN(C)CC1]

The mixture of Example 021 (50.0 mg, 0.13 mmol), 1-methylpiperazine (19.4 mg, 0.19 mmol), t-BuONa (0.64 mL, 1.3 mmol) and CPhos-Pd-G3 (10.4 mg, 0.01 mmol) in 1,4-dioxane (1 mL) was stirred at 100° C. for 2 hours under N₂ protection. After completion, the reaction mixture was poured into saturated ammonium chloride (15 mL) and the resulting mixture was extracted with EtOAc(15 mL×3). The combined organic layer was washed with brine (30 mL), dried over anhydrous sodium sulfate and concentrated in vacuo to give a residue, which was purified by prep-HPLC (NH₃ as additive) and lyophilized to give Example 022 (8.1 mg, 15.3% yield) as a yellow solid. MS (ESI⁺) obsd. [(M+H)⁺]: 408.2.

Example 022: ¹H NMR (400 MHz, DMSO-d₆) δ ppm 13.49 (s, 1H), 11.94 (s, 1H), 7.59 (d, J=3.2 Hz, 1H), 7.35 (s, 1H), 7.27 (dd, J=8.0, 11.2 Hz, 1H), 7.08 (dd, J=4.0, 7.6 Hz, 1H), 5.14 (s, 2H), 2.64-2.59 (m, 4H), 2.23 (s, 7H), 2.09 (s, 3H). ¹⁹F NMR (376 MHz, DMSO-d₆) δ ppm −132.96 (s, 1F).

The following Examples 023 to 031 were prepared in analogy to the procedure described for the preparation of Example 022, replacing 1-methylpiperazine with AMINE indicated in Table 4.

TABLE 4
Compound synthesis and characterization

| | Compound Names and | | |
|---|---|---|---|
| Ex. | Structures | AMINE | NMR and (ESI⁺) |
| 023 | 3-Amino-4-(7-fluoro- | Isopropyl amine | ¹H NMR (400 MHz, DMSO-d₆) |
| | 1H-indazol-4-yl)-6- | | δ ppm 7.59 (s, 1H), 7.26 (dd, |
| | (isopropylamino)-7- | | J = 8.0, 10.8 Hz, 1H), 7.20 (s, |
| | methyl-1H-1,5- | | 1H), 7.02 (dd, J = 4.0, 7.6 Hz, |
| | naphthyridin-2-one | | 1H), 3.36-3.30 (m, 1H), 2.04 (s, |
| | 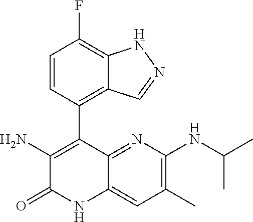 [SMILES: Cc1cc2[nH]c(=O)c(N)c(-c3ccc(F)c4[nH]ncc34)c2nc1NC(C)C] | | 3H), 0.84 (d, J = 6.4 Hz, 3H), 0.72 (d, J = 6.8 Hz, 3H). ¹⁹F NMR (376 MHz, DMSO- d₆) δ ppm −133.18 (s, 1F). MS (ESI⁺) obsd. [(M + H)⁺]: 367.1. |
| 024 | 3-Amino-4-(7-fluoro- | Pyrrolidin-3-ol | ¹H NMR (400 MHz, DMSO-d₆) |
| | 1H-indazol-4-yl)-6-(3- | | δ ppm 13.53 (s, 1H), 11.80 (s, |
| | hydroxypyrrolidin-1- | | 1H), 7.59 (d, J = 15.2 Hz, 1H), |
| | yl)-7-methyl-1H-1,5- | | 7.28-7.24 (m, 2H), 7.09-7.04 |
| | naphthyridin-2-one | | (m, 1H), 5.03 (d, J = 5.6 Hz, |
| | 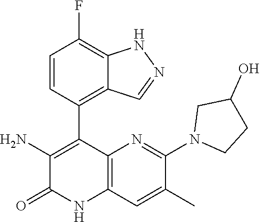 [SMILES: Cc1cc2[nH]c(=O)c(N)c(-c3ccc(F)c4[nH]ncc34)c2nc1N1CCC(O)C1] | | 2H), 4.67 (dd, J = 4.0, 5.2 Hz, 1H), 4.11-4.08 (m, 1H), 3.22- 3.09 (m, 2H), 2.94-2.80 (m, 2H), 2.27 (s, 3H), 1.75-1.65 (m, 1H), 1.57-1.52 (m, 1H). ¹⁹F NMR (376 MHz, DMSO- d₆) δ ppm −133.14 (s, 1F). MS (ESI⁺) obsd. [(M + H)⁺]: 395.2. |
| 025 | 3-Amino-4-(7-fluoro- | Morpholine | ¹H NMR (400 MHz, DMSO- |
| | 1H-indazol-4-yl)-7- | | d₆) δ ppm 13.55 (s, 1H), 11.96 |
| | methyl-6-morpholino- | | (s, 1H), 7.59 (d, J = 2.4 Hz, |
| | 1H-1,5-naphthyridin-2- | | 1H), 7.37 (s, 1H), 7.28 (dd, J = |
| | one | | 8.0, 11.2 Hz, 1H), 7.09 (dd, J = |
| | 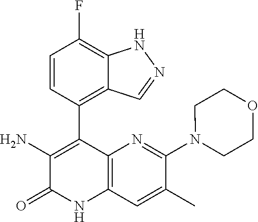 [SMILES: Cc1cc2[nH]c(=O)c(N)c(-c3ccc(F)c4[nH]ncc34)c2nc1N1CCOCC1] | | 4.4, 7.6 Hz, 1H), 5.17 (s, 2H), 3.50 (t, J = 4.4 Hz, 4H), 2.67- 2.62 (m, 4H), 2.25 (s, 3H) ¹⁹F NMR (376 MHz, DMSO- d₆) δ ppm −132.94 (s, 1F). MS (ESI⁺) obsd. [(M + H)⁺]: 395.2. |
| 026 | 3-Amino-4-(7-fluoro- | 2-Oxa-6- | ¹H NMR (400 MHz, DMSO-d₆) |
| | 1H-indazol-4-yl)-7- | azaspiro[3.3] | δ ppm 13.56 (s, 1H), 11.85 (s, |
| | methyl-6-(2-oxa-6- | heptane | 1H), 7.60 (s, 1H), 7.29 (dd, J = |
| | azaspiro[3.3]heptan-6- | | 8.0, 11.2 Hz, 1H), 7.23 (s, 1H), |
| | yl)-1H-1,5- | | 7.08 (dd, J = 4.4, 8.0 Hz, 1H), |
| | naphthyridin-2-one | | 5.09 (s, 2H), 4.57-4.51 (m, 4H), |
| | 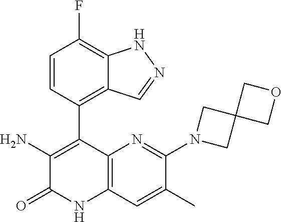 [SMILES: Cc1cc2[nH]c(=O)c(N)c(-c3ccc(F)c4[nH]ncc34)c2nc1N1CC2(COC2)C1] | | 3.8-3.72 (m, 4H), 2.13 (s, 3H) ¹⁹F NMR (376 MHz, DMSO- d₆) δ ppm −133.04 (s, 1F). MS (ESI⁺) obsd. [(M + H)⁺]: 407.0. |
| 027 | 3-Amino-4-(7-fluoro- | 2-Methyl-2,6- | ¹H NMR (400 MHz, MeOD- |
| | 1H-indazol-4-yl)-7- | diazaspiro[3.3] | d₄) δ ppm 7.71 (s, 1H), 7.38 (s, |
| | methyl-6-(6-methyl- | heptane | 1H), 7.33-7.27 (m, 1H), 7.20- |
| | 2,6- | | 7.14 (m, 1H), 4.10-3.94 (m, |
| | diazaspiro[3.3]heptan- | | 1H), 3.61-3.43 (m, 3H), 3.14- |
| | 2-yl)-1H-1,5- | | 2.84 (m, 4H), 2.68-2.43 (m, |
| | naphthyridin-2-one | | 3H), 2.31-2.23 (m, 3H) |
| | 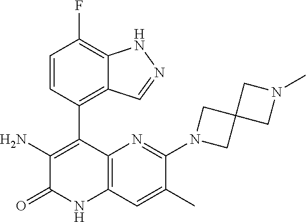 [SMILES: Cc1cc2[nH]c(=O)c(N)c(-c3ccc(F)c4[nH]ncc34)c2nc1N1CC2(CN(C)C2)C1] | | ¹⁹F NMR (376 MHz, MeOD- d₄) δ ppm −77.12 (s, 3F), −134.25 (s, 1F) MS (ESI⁺) obsd. [(M + H)⁺]: 420.2. |
| 028 | 3-Amino-4-(7-fluoro- | 1- | ¹H NMR (400 MHz, DMSO-d₆) |
| | 1H-indazol-4-yl)-6-(4- | Isopropyl- | δ = 12.03 (s, 1H), 9.71 (br s, |
| | isopropylpiperazin-1- | piperazine | 1H), 7.65 (d, J = 3.3 Hz, 1H), |
| | yl)-7-methyl-1H-1,5- | | 7.41 (s, 1H), 7.28 (dd, J = 3.6, |
| | naphthyridin-2-one | | 11.6 Hz, 1H), 7.07 (dd, J = 4.0, |
| | 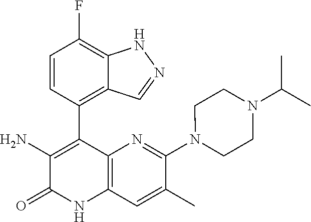 [SMILES: Cc1cc2[nH]c(=O)c(N)c(-c3ccc(F)c4[nH]ncc34)c2nc1N1CCN(C(C)C)CC1] | | 8.0 Hz, 1H), 3.25 (d, J = 2.4 Hz, 2H), 3.18 (s, 1H), 3.16-3.00 (m, 4H), 2.98-2.80 (m, 4H), 2.25 (s, 3H), 1.10 (t, J = 6.0 Hz, 6H). ¹⁹F NMR (376 MHz, DMSO- d₆) δ ppm −132.799 (s, 1F). MS (ESI⁺) obsd. [(M + H)⁺]: 436.3. |
| 029 | 3-Amino-6-(4- | 1- | ¹H NMR (400 MHz, DMSO-d₆) |
| | cyclopropylpiperazin-1- | Cyclopropyl- | δ = 7.59 (d, J = 3.2 Hz, 1H), |
| | yl)-4-(7-fluoro-1H- | piperazine | 7.35 (s, 1H), 7.27 (d, J = 8.0, |
| | indazol-4-yl)-7-methyl- | | 1H), 7.07 (d, J = 12.0 Hz, 1H), |
| | 1H-1,5-naphthyridin-2- | | 5.12 (s, 2H), 2.64-2.55 (m, 4H), |
| | one | | 2.46 (d, J = 4.4 Hz, 4H), 2.24 |
| | 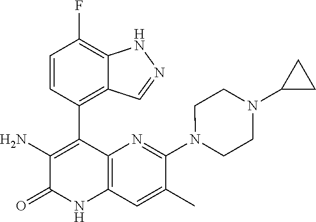 [SMILES: Cc1cc2[nH]c(=O)c(N)c(-c3ccc(F)c4[nH]ncc34)c2nc1N1CCN(C2CC2)CC1] | | (s, 3H), 1.53 (s, 1H), 0.39-0.31 (m, 2H), 0.22 (d, J = 13.4 Hz, 2H). ¹⁹F NMR (376 MHz, DMSO- d₆) δ ppm −132.932 (s, 1F). MS (ESI⁺) obsd. [(M + H)⁺]: 434.4. |
| 030 | 3-Amino-4-(7-fluoro- | 2-Methyl-2,5- | ¹H NMR (400 MHz, MeOD-d₄) |
| | 1H-indazol-4-yl)-7- | diazabicyclo | δ ppm 7.67 (dd, J = 3.2, 16.4 |
| | methyl-6-(5-methyl- | [2.2.1]heptane | Hz, 1H), 7.38 (s, 1H), 7.30-7.23 |
| | 2,5- | | (m, 1H), 7.18-7.10 (m, 1H), |
| | diazabicyclo[2.2.1] | | 3.98-3.78 (m, 1H), 3.66 (s, 1H), |
| | heptan-2-yl)-1H-1,5- | | 3.54-3.48 (m, 1H), 3.33 (s, 2H), |
| | naphthyridin-2-one | | 3.19-2.81 (m, 1H), 2.45-2.40 |
| | 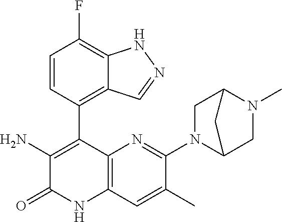 [SMILES: Cc1cc2[nH]c(=O)c(N)c(-c3ccc(F)c4[nH]ncc34)c2nc1N1CC2CC1CN2C] | | (m, 3H), 2.30 (s, 3H), 1.79 (d, J = 20.0 Hz, 2H). ¹⁹F NMR (376 MHz, DMSO- d₆) δ ppm −76.94 (s, 1F). MS (ESI⁺) obsd. [(M + H)⁺]: 420.4. |
| 031 | 3-Amino-4-(7-fluoro- | 2-Methyl-2,5- | ¹H NMR (400 MHz, MeOH-d₄) |
| | 1H-indazol-4-yl)-7- | diazabicyclo | δ ppm 7.74-7.63 (m, 1H), 7.48 |
| | methyl-6-(5-methyl- | [2.2.2]octane | (d, J = 7.6 Hz, 1H), 7.32-7.24 |
| | 2,5- | | (m, 1H), 7.20-7.11 (m, 1H), |
| | diazabicyclo[2.2.2] | | 3.57-3.33 (m, 6H), 2.79-2.55 |
| | octan-2-yl)-1H-1,5- | | (m, 3H), 2.34 (s, 3H), 2.11-1.96 |
| | naphthyridin-2-one | | (m, 2H), 1.83-1.50 (m, 2H). |
| | 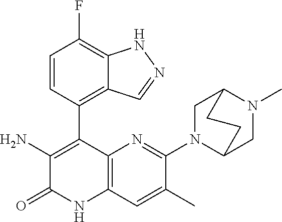 [SMILES: Cc1cc2[nH]c(=O)c(N)c(-c3ccc(F)c4[nH]ncc34)c2nc1N1CC2CCC1CN2C] | | ¹⁹F NMR (376 MHz, MeOH- d₄) δ ppm −134.617 (s, 1F). MS (ESI⁺) obsd. [(M + H)⁺]: 434.4. |

### Example 032: 3-Amino-4-(7-fluoro-1H-indazol-4-yl)-6-(3-hydroxy-3-methyl-butyl)-7-methyl-1H-1,5-naphthyridin-2-one

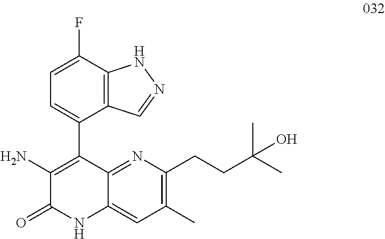 [SMILES: Cc1cc2[nH]c(=O)c(N)c(-c3ccc(F)c4[nH]ncc34)c2nc1CCC(C)(C)O]

### Step 1: [3-Amino-6-(3-hydroxy-3-methyl-but-1-ynyl)-5-methyl-2-pyridyl]-(7-fluoro-1H-indazol-4-yl)methanone

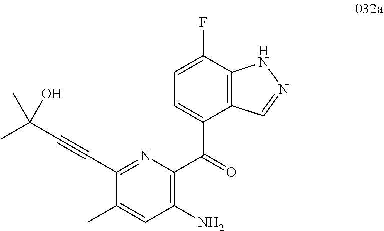 [SMILES: Cc1cc(N)c(C(=O)c2ccc(F)c3[nH]ncc23)nc1C#CC(C)(C)O]

The mixture of compound 021c (150.0 mg, 0.43 mmol), 2-methyl-3-butyn-2-ol (144.6 mg, 1.7 mmol), TEA (2.0 mL, 14.4 mmol), Pd(PPh₃)₂Cl₂ (15.1 mg, 0.02 mmol) and CuI (8.2 mg, 0.04 mmol) in THE (2 mL) was stirred at 60° C. for 12 hours under N₂ protection. After completion, the reaction mixture was quenched with saturated ammonium chloride (15 mL) and extracted with EtOAc(15 mL×3). The combined organic layer was washed with brine (30 mL), dried over anhydrous sodium sulfate and concentrated in vacuo to give a residue, which was purified by silica-gel chromatography (elute with EtOAc:PE=0-40%) to give compound 032a (120.0 mg, 79.3% yield) as a yellow solid. MS (ESI⁺) obsd. [(M+H)⁺]: 353.2.

### Step 2: 2-Chloro-N-[2-(7-fluoro-1H-indazole-4-carbonyl)-6-(3-hydroxy-3-methyl-but-1-ynyl)-5-methyl-3-pyridyl]acetamide

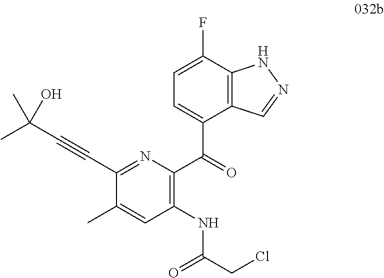 [SMILES: Cc1cc(NC(=O)CCl)c(C(=O)c2ccc(F)c3[nH]ncc23)nc1C#CC(C)(C)O]

The title compound was prepared in analogy to the preparation of compound 001c, replacing compound 001b with compound 032a. Compound 032b (140.0 mg, crude) was obtained as a brown solid. MS (ESI⁺) obsd. [(M+H)⁺]: 429.1.

### Step 3: 1-(4-(7-Fluoro-1H-indazol-4-yl)-6-(3-hydroxy-3-methylbut-1-yn-1-yl)-7-methyl-2-oxo-1,2-dihydro-1,5-naphthyridin-3-yl)pyridin-1-ium chloride

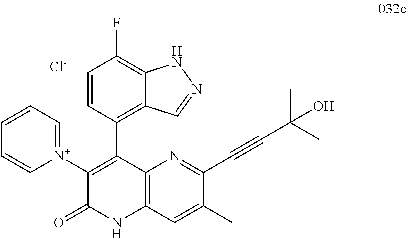 [SMILES: Cc1cc2[nH]c(=O)c(-[n+]3ccccc3)c(-c3ccc(F)c4[nH]ncc34)c2nc1C#CC(C)(C)O ; [Cl-]]

The title compound was prepared in analogy to the preparation of compound 001d, replacing compound 001c with compound 032b. Compound 032c (150.0 mg, crude) was obtained as a brown solid. MS (ESI⁺) obsd. [(M-Cl)⁺]: 454.0.

### Step 4: 3-Amino-4-(7-fluoro-1H-indazol-4-yl)-6-(3-hydroxy-3-methyl-butyl)-7-methyl-1H-1,5-naphthyridin-2-one

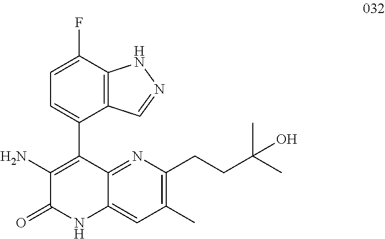 [SMILES: Cc1cc2[nH]c(=O)c(N)c(-c3ccc(F)c4[nH]ncc34)c2nc1CCC(C)(C)O]

The mixture of compound 032c (300 mg, 0.05 mmol) in ethanol (5 mL) and hydrazine monohydrate (1 mL) was stirred at 70° C. for 2 hours. After completion, the mixture was poured into water (10 mL) and extracted with EtOAc (10 mL×3). The combined organic layer was concentrated in vacuo to give a residue, which was purified by prep-HPLC (NH₄HCO₃ as additive) and lyophilized to give Example 032 (6.9 mg, 3.5% yield) as a yellow solid. MS (ESI⁺) obsd. [(M+H)⁺]: 396.1.

Example 032: ¹H NMR (400 MHz, DMSO-d₆) δ ppm 13.56 (s, 1H), 11.99 (s, 1H), 7.56 (s, 1H), 7.33 (s, 1H), 7.27 (dd, J=8.0, 11.2 Hz, 1H), 7.04 (dd, J=4.4, 8.0 Hz, 1H), 5.11 (s, 2H), 4.01 (s, 1H), 2.53 (t, J=8.4 Hz, 2H), 2.28 (s, 3H), 1.39 (t, J=8.4 Hz, 2H), 0.94 (s, 3H), 0.91 (s, 3H). ¹⁹F NMR (376 MHz, DMSO-d₆) δ ppm −133.04 (s, 1F).

### Example 033: 3-Amino-4-(1H-indazol-4-yl)-7-methyl-1H-1,5-naphthyridin-2-one

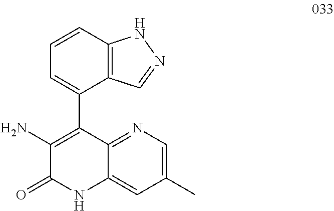 [SMILES: Cc1cnc2c(-c3cccc4[nH]ncc34)c(N)c(=O)[nH]c2c1]

### Step 1: tert-Butyl N-[2-(7-fluoro-1H-indazole-4-carbonyl)-5-methyl-3-pyridyl]carbamate

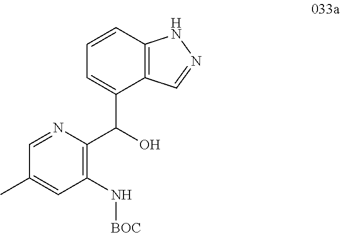 [SMILES: Cc1cnc(C(O)c2cccc3[nH]ncc23)c(NC(=O)OC(C)(C)C)c1]

To a solution of Int-3 (785.9 mg, 2.7 mmol) in THE (5 mL) was added n-BuLi (2.3 mL, 5.8 mmol) at −78° C. dropwise. The mixture was stirred at −78° C. for 30 minutes. Then a solution of 1H-indazole-4-carbaldehyde (200.0 mg, 1.4 mmol) in THE (5 mL) was added dropwise to the above mixture at −78° C. and the mixture was stirred at −78° C. for another 30 minutes. After completion, the reaction was quenched with saturated ammonium chloride (5 mL) and extracted with EtOAc (30 mL×3). The combined organic layer was washed with brine (50 mL), dried over anhydrous sodium sulfate and concentrated in vacuo to give a residue, which was purified by silica-gel chromatography (elute with EtOAc:PE=0-30%) to give compound 033a (238.0 mg, 24.7% yield) as a yellow solid. MS (ESI⁺) obsd. [(M+H)⁺]: 355.2.

### Step 2: tert-Butyl N-[2-(1H-indazole-4-carbonyl)-5-methyl-3-pyridyl]carbamate

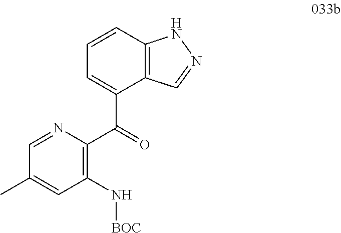 [SMILES: Cc1cnc(C(=O)c2cccc3[nH]ncc23)c(NC(=O)OC(C)(C)C)c1]

To a solution of compound 033a (630.0 mg, 1.7 mmol) in DCM (3 mL) was added DMP (904.8 mg, 2.1 mmol). The mixture was stirred at 25° C. for 1 hour. After completion, the mixture was quenched with saturated sodium sulfite (10 m), exacted with EtOAc (10 mL×3). The combined organic layer was washed with brine (10 mL), dried over anhydrous sodium sulfate and concentrated in vacuo to give a residue, which was purified by silica-gel chromatography (elute with EtOAc:PE=0-50%) to give compound 033b (605.0 mg, 96.0% yield) as a yellow solid. MS (ESI⁺) obsd. [(M+H)⁺]: 353.2.

### Step 3: (3-Amino-5-methylpyridin-2-yl)(1H-indazol-4-yl)methanone

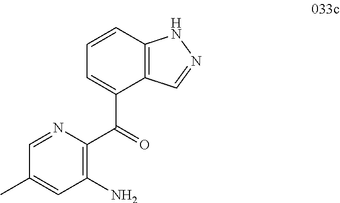 [SMILES: Cc1cnc(C(=O)c2cccc3[nH]ncc23)c(N)c1]

The title compound was prepared in analogy to the preparation of compound 001b, replacing compound 001a with compound 033b. Compound 033c (380.0 mg) was obtained as a yellow solid. MS (ESI⁺) obsd. [(M+H)⁺]: 253.1.

### Step 4: 2-Chloro-N-[2-(1H-indazole-4-carbonyl)-5-methyl-3-pyridyl]acetamide

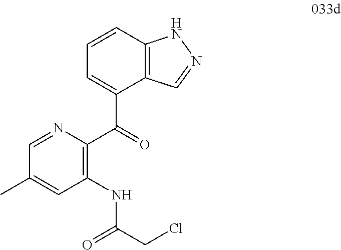 [SMILES: Cc1cnc(C(=O)c2cccc3[nH]ncc23)c(NC(=O)CCl)c1]

The title compound was prepared in analogy to the preparation of compound 001c, replacing compound 001b with compound 033c. Compound 033d (350.0 mg, crude) was obtained as a yellow solid. MS (ESI⁺) obsd. [(M+H)⁺]: 329.1.

### Step 5: 4-(1H-Indazol-4-yl)-7-methyl-3-pyridin-1-ium-1-yl-1H-1,5-naphthyridin-2-one;chloride

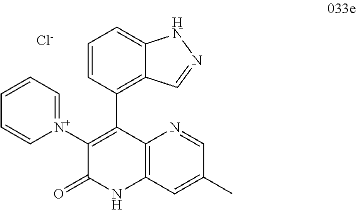 [SMILES: Cc1cnc2c(-c3cccc4[nH]ncc34)c(-[n+]3ccccc3)c(=O)[nH]c2c1 ; [Cl-]]

The title compound was prepared in analogy to the preparation of compound 001d, replacing compound 001c with compound 033d. Compound 033e (345.0 mg, crude) was obtained as a black solid. MS (ESI⁺) obsd. [(M-Cl)⁺]: 354.1.

### Step 6: 3-Amino-4-(1H-indazol-4-yl)-7-methyl-1H-1,5-naphthyridin-2-one

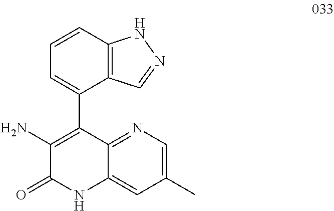 [SMILES: Cc1cnc2c(-c3cccc4[nH]ncc34)c(N)c(=O)[nH]c2c1]

The title compound was prepared in analogy to the preparation of Example 001, replacing compound 001d with compound 033e in step 5. Example 033 (68.7 mg) was obtained as a white solid. MS (ESI⁺) obsd. [(M+H)⁺]: 292.1.

Example 033: ¹H NMR (400 MHz, DMSO-d₆) δ ppm 7.98 (s, 1H), 7.57-7.55 (d, J=8.0 Hz, 1H), 7.50 (s, 1H), 7.47-7.43 (m, 1H), 7.41 (s, 1H), 7.05-7.03 (m, 1H), 5.03 (s, 2H), 2.30 (s, 3H).

### Example 034: 3-Amino-7-cyclopropyl-4-(1H-indazol-4-yl)-1H-1,5-naphthyridin-2-one

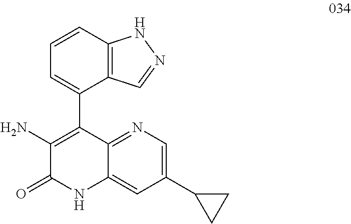 [SMILES: Nc1c(-c2cccc3[nH]ncc23)c2ncc(C3CC3)cc2[nH]c1=O]

### Step 1: tert-Butyl N-[5-chloro-2-[hydroxy(1H-indazol-4-yl)methyl]-3-pyridyl]carbamate

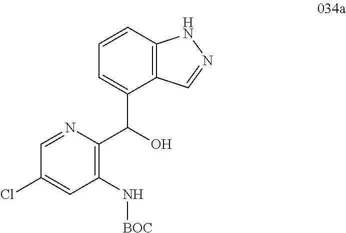 [SMILES: CC(C)(C)OC(=O)Nc1cc(Cl)cnc1C(O)c1cccc2[nH]ncc12]

The title compound was prepared in analogy to the preparation of 033a, replacing compound Int-3 with compound Int-2. Compound 034a (3.0 g) was obtained as a white solid. MS (ESI⁺) obsd. [(M+H)⁺]: 375.1.

### Step 2: Tert-butyl N-[5-chloro-2-(1H-indazole-4-carbonyl)-3-pyridyl]carbamate

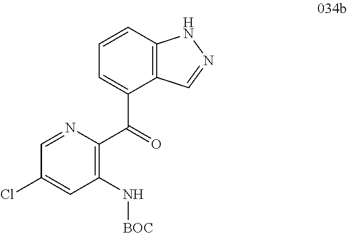 [SMILES: CC(C)(C)OC(=O)Nc1cc(Cl)cnc1C(=O)c1cccc2[nH]ncc12]

The title compound was prepared in analogy to the preparation of compound 033b, replacing compound 033a with compound 034a. Compound 034b (940.0 mg) was obtained as a white solid. MS (ESI⁺) obsd. [(M+H)⁺]: 373.1.

### Step 3: tert-Butyl (5-chloro-2-(1-(tetrahydro-2H-pyran-2-yl)-1H-indazole-4-carbonyl)pyridin-3-yl)carbamate

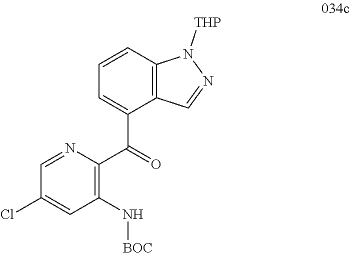

A mixture of compound 034b (200.0 mg, 0.54 mmol), dihydropyran (0.98 mL, 10.7 mmol) and TsOH (9.2 mg, 0.05 mmol) in DCM (2.5 mL) and THE (2.5 mL) was stirred at 25° C. for 12 hours. After completion, the mixture was concentrated in vacuo to give a residue, which was purified by silica-gel chromatography (elute with EtOAc:PE=0-10%) to give compound 034c (200.0 mg, 81.6% yield) as a yellow solid. MS (ESI⁺) obsd. [(M+H)⁺]: 457.2.

### Step 4: tert-Butyl N-[5-cyclopropyl-2-(1-tetrahydropyran-2-ylindazole-4-carbonyl)-3-pyridyl]carbamate

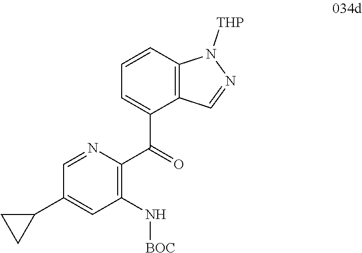

The mixture of compound 034c (100.0 mg, 0.22 mmol), cyclopropylboronic acid (75.2 mg, 0.88 mmol), Pd(dppf)Cl₂ (16.01 mg, 0.02 mmol) and K₂CO₃ (120.99 mg, 0.88 mmol, 4.0 eq) in 1,4-dioxane (1 mL) was stirred at 100° C. for 12 hours under N₂ protection. After completion, the mixture was poured into water (10 mL) and extracted with EtOAc(3×10 mL). The combined organic layer was washed with brine (10 mL), dried over anhydrous sodium sulfate and concentrated in vacuo to give a crude residue, which was purified by silica-gel chromatography (elute with EtOAc:PE=0-10%) to give compound 034d (80.0 mg, 79% yield) as a yellow solid. MS (ESI⁺) obsd. [(M+H)⁺]: 463.3.

### Step 5: (3-Amino-5-cyclopropyl-2-pyridyl)-(1H-indazol-4-yl)methanone

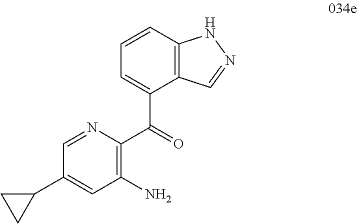 [SMILES: Nc1cc(C2CC2)cnc1C(=O)c1cccc2[nH]ncc12]

The title compound was prepare in analogy to the preparation of compound 001b, replacing compound 001a with compound 034d. Compound 034e (380.0 mg) was obtained as a yellow solid. MS (ESI⁺) obsd. [(M+H)⁺]: 279.1.

### Step 6: 2-Chloro-N-[5-cyclopropyl-2-(1H-indazole-4-carbonyl)-3-pyridyl]acetamide

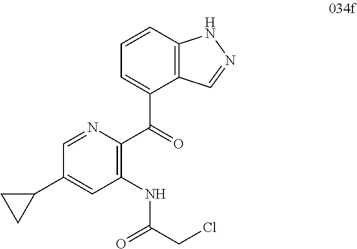 [SMILES: O=C(CCl)Nc1cc(C2CC2)cnc1C(=O)c1cccc2[nH]ncc12]

The title compound was prepared in analogy to the preparation of compound 001c, replacing compound 001b with compound 034e. Compound 034f (120.0 mg, crude) was obtained as a yellow solid. MS (ESI⁺) obsd. [(M+H)⁺]: 355.1.

### Step 7: 7-Cyclopropyl-4-(1H-indazol-4-yl)-3-pyridin-1-ium-1-yl-1H-1,5-naphthyridin-2-one;chloride

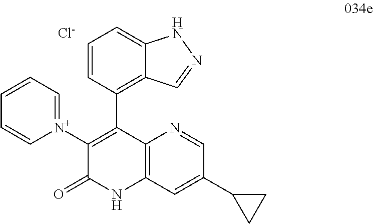 [SMILES: O=c1[nH]c2cc(C3CC3)cnc2c(-c2cccc3[nH]ncc23)c1-[n+]1ccccc1 ; [Cl-]]

The title compound was prepared in analogy to the preparation of compound 001d, replacing compound 001c with compound 034f. Compound 034e (345.0 mg, crude) was obtained as a black solid. MS (ESI⁺) obsd. [(M-Cl)⁺]: 380.2.

### Step 8: 3-Amino-7-cyclopropyl-4-(1H-indazol-4-yl)-1H-1,5-naphthyridin-2-one

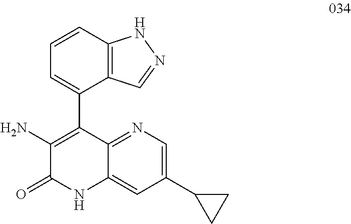 [SMILES: Nc1c(-c2cccc3[nH]ncc23)c2ncc(C3CC3)cc2[nH]c1=O]

The title compound was prepared in analogy to the preparation of Example 001, replacing compound 001d with compound 034e in step 5. Example 034 (68.7 mg) was obtained as a white solid. MS (ESI⁺) obsd. [(M+H)⁺]: 318.1.

Example 034: ¹H NMR (400 MHz, DMSO-d₆) δ ppm 13.06 (s, 1H), 11.99 (s, 1H), 7.98 (d, J=2.0 Hz, 1H), 7.56 (d, J=8.4 Hz, 1H), 7.52-7.41 (m, 2H), 7.25-7.21 (m, 1H), 7.04 (d, J=6.8 Hz, 1H), 2.02-1.89 (s, 1H), 1.04-0.95 (m, 2H), 0.71-0.64 (s, 2H).

### Example 035: 3-Amino-4-(1H-indazol-4-yl)-7-thiazol-2-yl-1H-1,5-naphthyridin-2-one

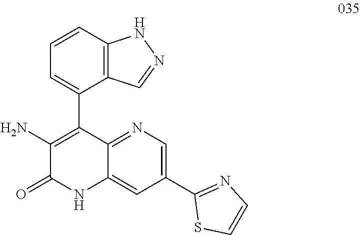 [SMILES: Nc1c(-c2cccc3[nH]ncc23)c2ncc(-c3nccs3)cc2[nH]c1=O]

The title compound was prepared in analogy to the preparation of Example 034, replacing cyclopropylboronic acid with tetrabutyl(thiazol-2-yl)tin in step 4. Example 035 (9.3 mg) was obtained as a yellow solid. MS (ESI⁺) obsd. [(M+H)⁺]: 361.1.

Example 035: ¹H NMR (400 MHz, DMSO-d₆) δ ppm 12.87 (s, 1H), 8.52 (d, J=1.6 Hz, 1H), 8.43 (s, 1H), 8.00 (d, J=3.2 Hz, 1H), 7.90 (d, J=2.8 Hz, 1H), 7.76-7.67 (m, 2H), 7.58-7.51 (m, 1H), 7.15 (d, J=6.8 Hz, 1H).

### Example 036: 3-Amino-7-chloro-4-(6-fluoro-1H-indazol-4-yl)-1H-1,5-naphthyridin-2-one

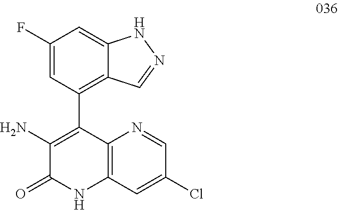 [SMILES: Nc1c(-c2cc(F)cc3[nH]ncc23)c2ncc(Cl)cc2[nH]c1=O]

### Step 1: 6-Fluoro-1-tetrahydropyran-2-yl-4-(4,4,5,5-tetramethyl-1,3,2-dioxaborolan-2-yl)indazole

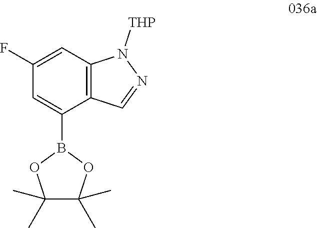

The mixture of 4-bromo-6-fluoro-1-tetrahydropyran-2-yl-indazole (500 mg, 1.7 mmol), bis(pinacolato)diboron (488.0 mg, 1.9 mmol), AcOK (492 mg, 5.0 mmol) and 1,1′-bis(diphenylphosphino)ferrocenedichloro palladium(II) dichloromethane complex (124.0 mg, 0.17 mmol) in 1,4-dioxane (8 mL) was stirred at 100° C. for 4 hours under N₂ protection. After completion, the mixture was diluted with EtOAc (50 mL) and washed with water and brine. The organic layer was concentrated in vacuo to give a crude residue, which was purified by silica-gel chromatography (elute with EtOAc:PE=0-12%) to afford compound 036a (350.0 mg, 60% yield) as a yellow oil. MS (ESI⁺) obsd. [(M+H)⁺]: 347.1.

### Step 2: 3-Amino-7-chloro-4-(6-fluoro-1-tetrahydropyran-2-yl-indazol-4-yl)-1H-1,5-naphthyridin-2-one

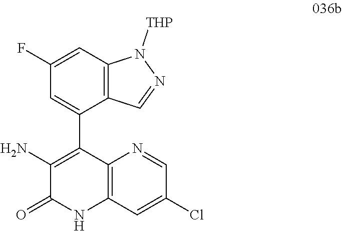

The mixture of compound 036a (37.8 mg, 109.3 umol), Cs₂CO₃ (89.0 mg, 273.2 umol), Pd(dtbpf)Cl₂ (7.1 mg, 10.9 umol) and compound Int-10 (30.0 mg, 109.3 umol) in 1,4-dioxane (4 mL) and water was stirred at 90° C. for 1 hour under N₂ atmosphere. After completion, the resulting mixture was quenched with water and extracted with DCM/MeOH (9/1, 20 mL×3). The combined organic layers were concentrated in vacuo to afford crude compound 036b (50 mg, 49% purify, 54% yield), which was used in next step without further purification. MS (ESI⁺) obsd. [(M+H)⁺]: 414.0.

### Step 3: 3-Amino-7-chloro-4-(6-fluoro-1H-indazol-4-yl)-1H-1,5-naphthyridin-2-one

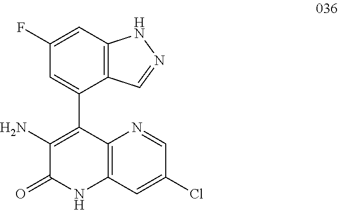 [SMILES: Nc1c(-c2cc(F)cc3[nH]ncc23)c2ncc(Cl)cc2[nH]c1=O]

To a solution of compound 036b (50 mg, 49% purify, 59.3 umol) in DCM (10 mL) was added TFA (1.0 mL). The mixture was stirred at rt for 4 hours. After completion, the mixture was concentrated in vacuo to give crude residue, which was purified by pre-HPLC (NH₃ as additive) to afford Example 036 (1.0 mg, 2.4% yield) as a white powder. MS (ESI⁺) obsd. [(M+H)⁺]: 330.0.

Example 036: ¹H NMR (500 MHz, DMSO-d₆) δ ppm 13.13 (s, 1H), 12.29 (br s, 1H), 8.16 (d, J=2.3 Hz, 1H), 7.63 (d, J=2.3 Hz, 1H), 7.56 (s, 1H), 7.36-7.32 (m, 1H), 6.93 (dd, J=2.1, 9.9 Hz, 1H), 5.59 (s, 2H). ¹⁹F NMR (471 MHz, DMSO-d₆) δ ppm −115.97 (s, 1F).

The following Examples 037 to 043 were prepared in analogy to the procedure described for the preparation of Example 036, replacing 4-bromo-6-fluoro-1-tetrahydropyran-2-yl-indazole with HEAD indicated in Table 5 in step1.

TABLE 5
Compound synthesis and characterization

| | Compound Names and | | |
|---|---|---|---|
| Ex. | Structures | HEAD | NMR and (ESI⁺) |
| 037 | 3-Amino-7-chloro-4-(7- | 4-Bromo-7-chloro- | ¹H NMR (500 MHz, DMSO- |
| | chloro-1H-indazol-4- | 1-tetrahydropyran- | d₆) δ ppm 13.58 (s, 1H), |
| | yl)-1H-1,5- | 2-yl-indazole | 12.28 (s, 1H), 8.13 (d, J = |
| | naphthyridin-2-one | | 2.3 Hz, 1H), 7.67 (s, 1H), |
| | 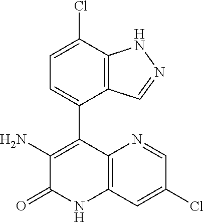 [SMILES: Nc1c(-c2ccc(Cl)c3[nH]ncc23)c2ncc(Cl)cc2[nH]c1=O] | | 7.63 (d, J = 2.3 Hz, 1H), 7.54 (d, J = 7.5 Hz, 1H), 7.04 (d, J = 7.6 Hz, 1H), 5.53 (s, 2H). MS (ESI⁺) obsd. [(M + H)⁺]: 346.0. |
| 038 | 3-Amino-7-chloro-4-(7- | 4-Bromo-7-methyl- | ¹H NMR (500 MHz, DMSO- |
| | methyl-1H-indazol-4- | 1-tetrahydropyran- | d₆) δ ppm 13.02 (br s, 1H), |
| | yl)-1H-1,5- | 2-yl-indazole | 7.87 (s, 1H), 7.44 (s, 2H), |
| | naphthyridin-2-one | | 7.19 (dd, J = 0.8, 7.1 Hz, |
| | 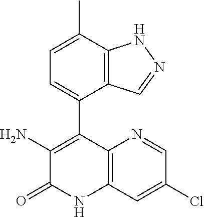 [SMILES: Cc1ccc(-c2c(N)c(=O)[nH]c3cc(Cl)cnc23)c2cn[nH]c12] | | 1H), 6.92 (d, J = 7.0 Hz, 1H), 4.99 (br s, 2H), 2.57 (s, 3H). MS (ESI⁺) obsd. [(M + H)⁺]: 326.1. |
| 039 | 3-Amino-7-chloro-4- | 4-Bromo-6,7- | ¹H NMR (500 MHz, DMSO- |
| | (6,7-difluoro-1H- | difluoro-1- | d₆) δ ppm 7.88 (s, 1H), 7.54 |
| | indazol-4-yl)-1H-1,5- | tetrahydropyran-2- | (d, J = 3.2 Hz, 1H), 7.41 (s, |
| | naphthyridin-2-one | yl-indazole | 1H), 6.97 (dd, J = 6.3, 10.9 |
| | 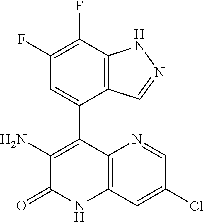 [SMILES: Nc1c(-c2cc(F)c(F)c3[nH]ncc23)c2ncc(Cl)cc2[nH]c1=O] | | Hz, 1H), 5.25 (br s, 2H). MS (ESI⁺) obsd. [(M + H)⁺]: 348.0. |
| 040 | 3-Amino-7-chloro-4-(5- | 4-Bromo-5-fluoro- | ¹H NMR (500 MHz, DMSO- |
| | fluoro-1H-indazol-4- | 1H-indazole | d₆) δ ppm 13.18 (br s, 1H), |
| | yl)-1H-1,5- | | 12.05 (br s, 1H), 8.13 (br d, |
| | naphthyridin-2-one | | J = 7.3 Hz, 1H), 7.66-7.56 |
| | 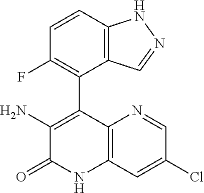 [SMILES: Nc1c(-c2c(F)ccc3[nH]ncc23)c2ncc(Cl)cc2[nH]c1=O] | | (m, 3H), 7.33 (t, J = 9.2 Hz, 1H), 5.59 (br s, 2H). ¹⁹F NMR (471 MHz, DMSO-d₆) δ ppm −123.23 (s, 1F). MS (ESI⁺) obsd. [(M + H)⁺]: 330.0. |
| 041 | 3-Amino-7-chloro-4-[7- | 4-Bromo-7- | ¹H NMR (400 MHz, DMSO- |
| | (trifluoromethyl)-1H- | (Trifluoromethyl)- | d₆) δ ppm 13.62 (s, 1H), |
| | indazol-4-yl]-1H-1,5- | 1H-indazole | 12.30 (s, 1H), 8.12 (d, J = |
| | naphthyridin-2-one | | 2.3 Hz, 1H), 7.83 (d, J = 7.6 |
| | 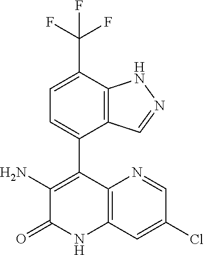 [SMILES: Nc1c(-c2ccc(C(F)(F)F)c3[nH]ncc23)c2ncc(Cl)cc2[nH]c1=O] | | Hz, 1H), 7.77 (s, 1H), 7.64 (d, J = 2.1 Hz, 1H), 7.21 (d, J = 7.4 Hz, 1H), 5.63 (s, 2H). ¹⁹F NMR (376 MHz, DMSO-d₆) δ ppm −59.95 (s, 3F). MS (ESI⁺) obsd. [(M + H)⁺]: 380.0. |
| 042 | 3-Amino-7-chloro-4-(7- | Compound Int-11 | ¹H NMR (400 MHz, DMSO- |
| | fluoro-5-methyl-1H- | | d₆) δ ppm 13.46 (br s, 1H), |
| | indazol-4-yl)-1H-1,5- | | 12.25 (s, 1H), 8.12 (d, J = |
| | naphthyridin-2-one | | 2.3 Hz, 1H), 7.63 (d, J = 2.3 |
| | 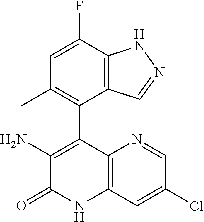 [SMILES: Cc1cc(F)c2[nH]ncc2c1-c1c(N)c(=O)[nH]c2cc(Cl)cnc12] | | Hz, 1H), 7.50 (s, 1H), 7.19 (d, J = 12.1 Hz, 1H), 5.36 (br s, 2H), 2.06 (s, 3H). ¹⁹F NMR (376 MHz, DMSO-d₆) δ ppm −133.11 (s, 1F). MS (ESI⁺) obsd. [(M + H)⁺]: 344.0. |
| 043 | 3-Amino-7-chloro-4-(5- | Compound Int-12 | ¹H NMR (400 MHz, DMSO- |
| | chloro-7-fluoro-1H- | | d₆) δ ppm 13.80 (s, 1H), |
| | indazol-4-yl)-1H-1,5- | | 12.28 (s, 1H), 8.12 (d, J = |
| | naphthyridin-2-one | | 2.3 Hz, 1H), 7.66 (s, 1H), |
| | 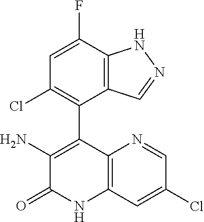 [SMILES: Nc1c(-c2c(Cl)cc(F)c3[nH]ncc23)c2ncc(Cl)cc2[nH]c1=O] | | 7.62 (d, J = 2.3 Hz, 1H), 7.48 (d, J = 10.4 Hz, 1H), 5.66 (s, 2H). ¹⁹F NMR (376 MHz, DMSO-d₆) δ ppm −129.93 (s, 1F). MS (ESI⁺) obsd. [(M + H)⁺]: 363.9. |

### Example 044: 3-Amino-4-(7-fluoro-1H-indazol-4-yl)-7-methyl-6-piperazin-1-yl-1H-1,5-naphthyridin-2-one

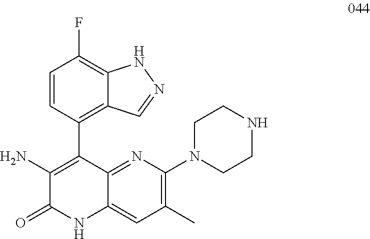 [SMILES: Cc1cc2[nH]c(=O)c(N)c(-c3ccc(F)c4[nH]ncc34)c2nc1N1CCNCC1]

### Step 1: tert-Butyl 4-(7-amino-8-(7-fluoro-1H-indazol-4-yl)-3-methyl-6-oxo-5,6-dihydro-1,5-naphthyridin-2-yl)piperazine-1-carboxylate

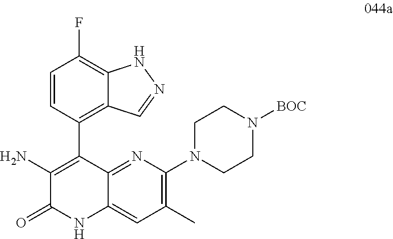 [SMILES: Cc1cc2[nH]c(=O)c(N)c(-c3ccc(F)c4[nH]ncc34)c2nc1N1CCN(C(=O)OC(C)(C)C)CC1]

The title compound was prepared in analogy to the preparation of Example 022, replacing compound 1-methylpiperazine with 1-Boc-piperazine to give compound 044a (20.0 mg, crude) as a yellow oil. MS (ESI⁺) obsd. [(M+H)⁺]: 494.4

### Step 2: 3-Amino-4-(7-fluoro-1H-indazol-4-yl)-7-methyl-6-(piperazin-1-yl)-1,5-naphthyridin-2(1H)-one

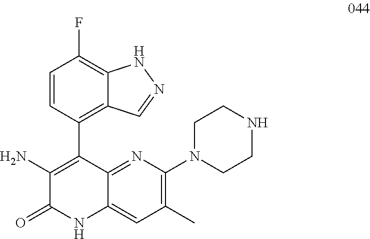 [SMILES: Cc1cc2[nH]c(=O)c(N)c(-c3ccc(F)c4[nH]ncc34)c2nc1N1CCNCC1]

A mixture of compound 044a (20.0 mg, 0.04 mmol) in HCl/EtOAc (1.0 mL, 4 M) was stirred at 25° C. for 1 hour. After completion, the mixture was concentrated in vacuo to give a residue, which was purified by prep-HPLC (NH₃ as additive) and lyophilized to give Example 044 (2.0 mg, 12.2% yield) as a yellow solid. MS (ESI⁺) obsd. [(M+H)⁺]: 394.3.

Example 044: ¹H NMR (400 MHz, DMSO-d₆) δ ppm 13.57 (s, 1H), 11.92 (s, 1H), 7.60 (d, J=3.2 Hz, 1H), 7.35 (s, 1H), 7.27 (dd, J=8.0, 11.2 Hz, 1H), 7.08 (dd, J=4.0, 7.6 Hz, 1H), 5.12 (s, 2H), 2.62-2.54 (m, 8H), 2.23 (s, 3H). ¹⁹F NMR (376 MHz, DMSO-d₆) δ ppm −132.96 (s, 1F).

### Example 045: 3-Amino-7-chloro-4-(3-methyl-1H-indazol-4-yl)-1H-1,5-naphthyridin-2-one

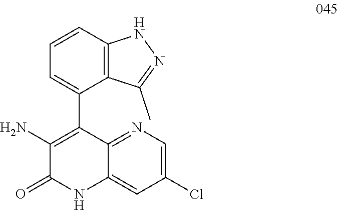 [SMILES: Cc1n[nH]c2cccc(-c3c(N)c(=O)[nH]c4cc(Cl)cnc34)c12]

### Step 1: 3-Methyl-1-(tetrahydro-2H-pyran-2-yl)-1H-indazole-4-carbaldehyde

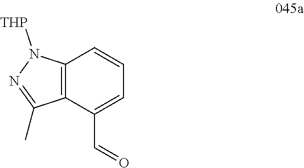

To a mixture of compound 4-bromo-3-methyl-1-tetrahydropyran-2-yl-indazole (2.0 g, 6.8 mmol) in THE (20 mL) was added n-BuLi (3.0 mL, 7.5 mmol) at −78° C. and the mixture was stirred at −78° C. for 0.5 hour. Then DMF (2.6 mL, 33.9 mmol) was added to the mixture and the mixture was stirred at 25° C. for another 1 hour. After completion, the mixture was quenched with sat. NH₄Cl aq. (40 mL), extracted with EtOAc (100 mL×2). The combined organic layer was washed with brine (100 mL), dried over anhydrous sodium sulfate and concentrated under reduced pressure to give a residue, which was purified by silica-gel chromatography (elute with EtOAc:PE=5-50%) to give compound 045a (1.4 g, 84.3% yield) as a white solid. MS (ESI⁺) obsd. [(M+H)⁺]: 245.1.

### Step 2: tert-Butyl (5-chloro-2-(hydroxy(3-methyl-1-(tetrahydro-2H-pyran-2-yl)-1H-indazol-4-yl)methyl)pyridin-3-yl)carbamate

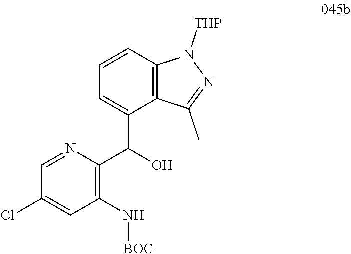

To a solution of Int-2 (475.0 mg, 1.5 mmol) in tetrahydrofuran (5 mL) was added NaH (67.5 mg, 1.7 mmol) at 0° C. The reaction mixture was stirred at 25° C. for 30 minutes. Then n-BuLi (0.68 mL, 1.7 mmol) was added dropwise at −78° C. and stirred for another 30 minutes. Then a solution of 045a (300.0 mg, 1.2 mmol) in tetrahydrofuran (2 mL) was added to the mixture and the mixture was stirred at −78° C. for another 30 minutes. After completion, the mixture was quenched with saturated aqueous ammonium chloride (50 mL) and extracted with ethyl acetate (80 mL×3). The combined organic layer was washed with brine (80 mL), dried over anhydrous sodium sulfate and concentrated in vacuo to give the residue, which was purified by silica gel-chromatography (elute with EtOAc:PE=0-20%) to give 045b (550.0 mg, 1.16 mmol, 77.3% yield) as a yellow oil. MS (ESI⁺) obsd. [(M+H)⁺]: 473.2.

### Step 3: tert-Butyl (5-chloro-2-(3-methyl-1-(tetrahydro-2H-pyran-2-yl)-1H-indazole-4-carbonyl)pyridin-3-yl)carbamate

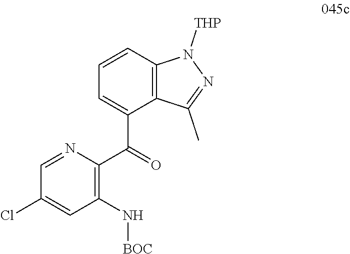

The mixture of compound 045b (850.0 mg, 1.8 mmol) and DMP (1.0 g, 5.39 mmol) in DCM (15 mL) was stirred at 25° C. for 6 hours. After completion, the mixture was quenched with saturated sodium sulfite aq. (30 mL) and saturated Na₂CO₃ aq. (30 mL), and extracted with EtOAc (30 mL×3). The combined organic layer was washed with brine (40 mL), dried over anhydrous sodium sulfate and concentrated in vacuo to give a residue, which was purified by silica-gel chromatography (elute with EtOAc:PE=5-40%) to give compound 045c (730.0 mg, 86.1% yield) as a yellow oil. MS (ESI⁺) obsd. [(M+H)⁺]: 471.2.

### Step 4: (3-Amino-5-chloropyridin-2-yl)(3-methyl-1H-indazol-4-yl)methanone

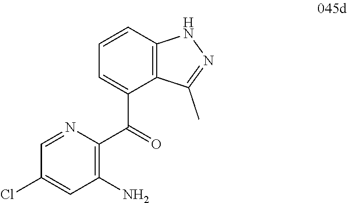 [SMILES: Cc1n[nH]c2cccc(C(=O)c3ncc(Cl)cc3N)c12]

The title compound was prepared in analogy to the preparation of compound 001b, replacing 001a with compound 045c. Compound 045d (420.0 mg, crude) was obtained as a yellow oil. MS (ESI⁺) obsd. [(M+H)⁺]: 287.0.

### Step 5: 2-Chloro-N-(5-chloro-2-(3-methyl-1H-indazole-4-carbonyl)pyridin-3-yl)acetamide

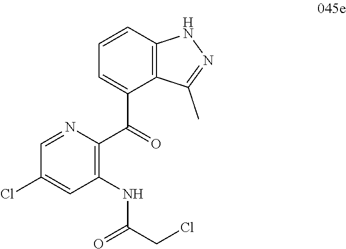 [SMILES: Cc1n[nH]c2cccc(C(=O)c3ncc(Cl)cc3NC(=O)CCl)c12]

The title compound was prepared in analogy to the preparation of compound 001c, replacing compound 001b with compound 045d. Compound 045e (540 mg, crude) was obtained as a brown oil. MS (ESI⁺) obsd. [(M+H)⁺]: 363.0.

### Step 6: 1-(7-Chloro-4-(3-methyl-1H-indazol-4-yl)-2-oxo-1,2-dihydro-1,5-naphthyridin-3-yl)pyridin-1-ium chloride

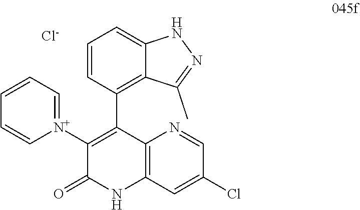 [SMILES: Cc1n[nH]c2cccc(-c3c(-[n+]4ccccc4)c(=O)[nH]c4cc(Cl)cnc34)c12 ; [Cl-]]

The title compound was prepared in analogy to the preparation of compound 001d, replacing compound 001c with compound 045e. Compound 045f (440.0 mg, crude) was obtained as a brown oil. MS (ESI⁺) obsd. [(M-Cl)⁺]: 388.1.

### Step 7: 3-Amino-7-chloro-4-(3-methyl-1H-indazol-4-yl)-1,5-naphthyridin-2(1H)-one

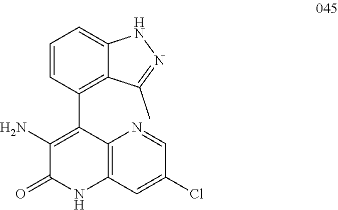 [SMILES: Cc1n[nH]c2cccc(-c3c(N)c(=O)[nH]c4cc(Cl)cnc34)c12]

The title compound was prepared in analogy to the preparation of Example 001 in step 5, replacing compound 001d with compound 045f. Compound 045 (270.0 mg) was obtained as a brown solid. MS (ESI⁺) obsd. [(M+H)⁺]: 326.1.

Example 045: ¹H NMR (400 MHz, DMSO+D₂O) δ ppm 8.12 (d, J=2.4 Hz, 1H),

7.70 (d, J=2.0 Hz, 1H), 7.55-7.49 (m, 1H), 7.48-7.39 (m, 1H), 6.88 (d, J=6.4 Hz, 1H), 1.79 (s, 3H).

The following Examples 046-057 were prepared in analogy to the procedure described for the preparation of Example 045, replacing Int-2 with HALOCPD indicated in Table 6 and compound 045a with 7-Fluoro-1-tetrahydropyran-2-yl-indazole-4-carbaldehyde in step 2.

TABLE 6
Compound synthesis and characterization

| | Compound Names and | | |
|---|---|---|---|
| Ex. | Structures | HALOCPD | NMR and (ESI⁺) |
| 046 | 3-Amino-6-(cyclobutoxy)- | Compound | ¹H NMR (400 MHz, DMSO-d₆) δ ppm |
| | 4-(7-fluoro-1H-indazol-4- | Int-7 | 13.57 (s, 1H), 11.96 (s, 1H), 7.62 (d, J = |
| | yl)-7-methyl-1H-1,5- | | 0.8 Hz, 1H), 7.37 (s, 1H), 7.29 (dd, J = |
| | naphthyridin-2-one | | 8.0, 10.8 Hz, 1H), 7.04 (dd, J = 4.0, 8.0 |
| | 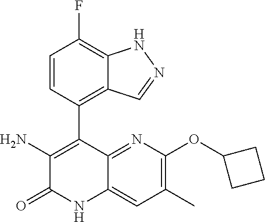 [SMILES: Cc1cc2[nH]c(=O)c(N)c(-c3ccc(F)c4[nH]ncc34)c2nc1OC1CCC1] | | Hz, 1H), 5.19-5.11 (m, 2H), 4.35 (m, 1H), 2.12 (s, 3H), 1.79-1.66 (m, 3H), 1.63-1.49 (m, 2H), 1.23-1.14 (m, 1H). ¹⁹F NMR (376 MHz, DMSO-d₆) δ ppm −133.00 (s, 1F). MS (ESI⁺) obsd. [(M + H)⁺]: 380.2. |
| 047 | 3-Amino-6- | Compound | ¹H NMR (400 MHz, DMSO-d₆) δ ppm |
| | (cyclopropylmethoxy)-4-(7- | Int-8 | 13.56 (s, 1H), 11.97 (s, 1H), 7.62 (s, |
| | fluoro-1H-indazol-4-yl)-7- | | 1H), 7.38 (s, 1H), 7.27 (dd, J = 8.1, |
| | methyl-1H-1,5- | | 11.2 Hz, 1H), 7.06 (dd, J = 4.3, 7.9 Hz, |
| | naphthyridin-2-one | | 1H), 5.17 (s, 2H), 3.57 (d, J = 6.8 Hz, |
| | 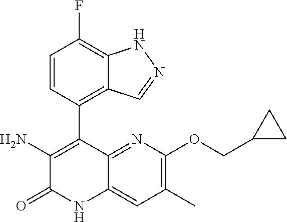 [SMILES: Cc1cc2[nH]c(=O)c(N)c(-c3ccc(F)c4[nH]ncc34)c2nc1OCC1CC1] | | 2H), 2.14 (s, 3H), 0.87-0.80 (m, 1H), 0.25-0.19 (m, 2H), −0.15-−0.19 (m, 2H). ¹⁹F NMR (400 MHz, DMSO-d₆) δ ppm −132.96 (s, 1F). MS (ESI⁺) obsd. [(M + H)⁺]: 380.2. |
| 048 | 3-Amino-4-(7-fluoro-1H- | tert-Butyl N- | ¹H NMR (400 MHz, DMSO-d₆) δ ppm |
| | indazol-4-yl)-6,7-dimethyl- | (2-bromo- | 13.77 (br s, 1H), 11.90 (s, 1H), 7.65 (s, |
| | 1H-quinolin-2-one | 4,5-dimethyl- | 1H), 7.36 (dd, J = 8.0, 11.2 Hz, 1H), |
| | 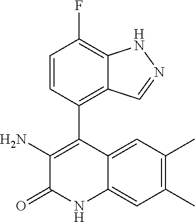 [SMILES: Cc1cc2[nH]c(=O)c(N)c(-c3ccc(F)c4[nH]ncc34)c2cc1C] | phenyl) carbamate | 7.09 (s, 1H), 6.99 (dd, J = 4.0, 7.6 Hz, 1H), 6.49 (s, 1H), 4.70 (s, 2H), 2.22 (s, 3H), 2.01 (s, 3H). ¹⁹F NMR (376 MHz, DMSO-d₆) δ ppm −132.08 (s, 1F). MS (ESI⁺) obsd. [(M + H)⁺]: 323.1. |
| 049 | 3-Amino-6-chloro-4-(7- | Compound | ¹H NMR (400 MHz, DMSO-d₆) δ ppm |
| | fluoro-1H-indazol-4-yl)-7,8- | Int-14 | 13.63 (br s, 1H), 11.49 (br s, 1H), 7.63 |
| | dimethyl-1H-1,5- | | (br s, 1 H), 7.29 (dd, J = 11.07, 7.94 Hz, |
| | naphthyridin-2-one | | 1H), 6.99 (dd, J = 7.75, 4.13 Hz, 1H), |
| | 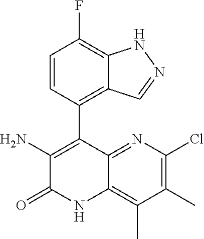 [SMILES: Cc1c(Cl)nc2c(-c3ccc(F)c4[nH]ncc34)c(N)c(=O)[nH]c2c1C] | | 5.33 (s, 2 H), 2.47 (s, 3 H), 2.32 (s, 3H). ¹⁹F NMR (376 MHz, DMSO-d₆) δ ppm −132.61 (s, 1F). MS (ESI⁺) obsd. [(M + H)⁺]: 358.0. |
| 050 | 3-Amino-8-chloro-4-(7- | tert-Butyl N- | ¹H NMR (400 MHz, MeOD-d₄) δ ppm |
| | fluoro-1H-indazol-4-yl)-1H- | (2-bromo-4- | 8.06 (d, J = 5.2 Hz, 1H), 7.66 (d, J = 4.0 |
| | 1,5-naphthyridin-2-one | chloro-3- | Hz, 1H), 7.34 (d, J = 5.2 Hz, 1H), 7.30- |
| | 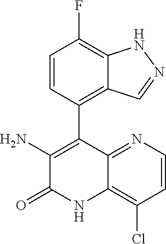 [SMILES: Nc1c(-c2ccc(F)c3[nH]ncc23)c2nccc(Cl)c2[nH]c1=O] | pyridyl) carbamate | 7.24 (m, 1H), 7.11 (d, J = 12 Hz, 1H), 4.59 (s, 2H). ¹⁹F NMR (376 MHz, MeOD-d₄) δ ppm −134.42 (s, 1F). MS (ESI⁺) obsd. [(M + H)⁺]: 330.2. |
| 051 | 3-Amino-4-(7-fluoro-1H- | Compound | ¹H NMR (400 MHz, DMSO-d₆) δ ppm |
| | indazol-4-yl)-8-methoxy-7- | Int-15 | 13.60 (s, 1H), 11.68 (s, 1H), 7.96 (s, |
| | methyl-1H-1,5- | | 1H), 7.61 (s, 1H), 7.27 (dd, J = 11.2, 7.6 |
| | naphthyridin-2-one | | Hz, 1H) 6.98 (dd, J = 8.0, 4.4 Hz, 1H) |
| | 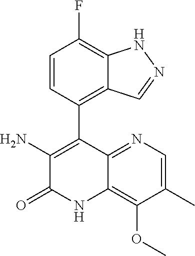 [SMILES: COc1c(C)cnc2c(-c3ccc(F)c4[nH]ncc34)c(N)c(=O)[nH]c12] | | 5.18 (s, 2H) 3.86 (s, 3 H) 2.25 (s, 3H). ¹⁹F NMR (377 MHz, DMSO-d₆) δ ppm −132.93 (s, 1 F). MS (ESI⁺) obsd. [(M + H)⁺]: 340.2. |
| 052 | 3-Amino-7-chloro-4-(7- | Compound | ¹H NMR (400 MHz, DMSO-d₆) δ ppm |
| | fluoro-1H-indazol-4-yl)-6- | Int-16 | 13.62 (s, 1H), 12.14 (s, 1H), 7.64 (s, |
| | methyl-1H-1,5- | | 1H), 7.59 (s, 1H), 7.28 (d, J = 19.2 Hz, |
| | naphthyridin-2-one | | 1H), 7.01 (d, J = 12.0 Hz, 1H), 5.37 (s, |
| | 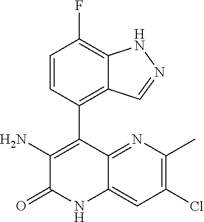 [SMILES: Cc1nc2c(-c3ccc(F)c4[nH]ncc34)c(N)c(=O)[nH]c2cc1Cl] | | 2H), 2.26 (s, 3H). ¹⁹F NMR (376 MHz, DMSO-d₆) δ ppm −132.658 (s, 1F). MS (ESI⁺) obsd. [(M + H)⁺]: 344.1. |
| 053 | 3-Amino-8-chloro-4-(7- | tert-Butyl N- | ¹H NMR (500 MHz, DMSO-d₆) δ ppm |
| | fluoro-1H-indazol-4-yl)-7- | (2-bromo-4- | 13.61 (s, 1H), 11.60 (s, 1H), 8.07 (s, |
| | methyl-1H-1,5- | chloro-5- | 1H), 7.63 (s, 1H), 7.28 (dd, J = 8.0, 10.9 |
| | naphthyridin-2-one | methyl-3- | Hz, 1H), 6.99 (dd, J = 4.1, 7.6 Hz, 1H), |
| | 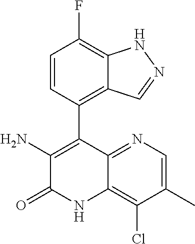 [SMILES: Cc1cnc2c(-c3ccc(F)c4[nH]ncc34)c(N)c(=O)[nH]c2c1Cl] | pyridyl) carbamate | 5.39 (br s, 2H), 2.34 (s, 3H). ¹⁹F NMR (471 MHz, DMSO-d₆) δ ppm −132.71 (s, 1F). MS (ESI⁺) obsd. [(M + H)⁺]: 343.9. |
| 054 | 3-Amino-4-(7-fluoro-1H- | Compound | ¹H NMR (400 MHz, MeOD-d₄) δ ppm |
| | indazol-4-yl)-7,8-dimethyl- | Int-17 | 7.96 (s, 1H), 7.61 (d, J = 3.2 Hz, 1H), |
| | 1H-1,5-naphthyridin-2-one | | 7.28 (dd, J = 11.2, 7.6 Hz, 1H), 7.10 |
| | 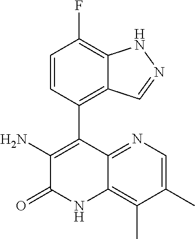 [SMILES: Cc1cnc2c(-c3ccc(F)c4[nH]ncc34)c(N)c(=O)[nH]c2c1C] | | (dd, J = 7.6, 4.0 Hz, 1H), 2.50 (s, 3H), 2.36 (s, 3H). ¹⁹F NMR (376 MHz, MeOD-d₄) δ ppm −134.69 (s, 1F). MS (ESI⁺) obsd. [(M + H)⁺]: 324.3. |
| 055 | 3-Amino-7-chloro-6- | Compound | ¹H NMR (400 MHz, DMSO-d₆) δ ppm |
| | cyclopropyl-4-(7-fluoro-1H- | Int-18 | 13.58 (s, 1H), 12.11 (s, 1H), 7.57 (s, |
| | indazol-4-yl)-1H-1,5- | | 1H), 7.56 (s, 1H), 7.27 (dd, J = 8.0, 11.2 |
| | naphthyridin-2-one | | Hz, 1H), 7.01 (dd, J = 4.4, 7.6 Hz, 1H), |
| | 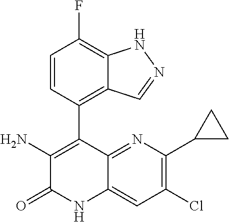 [SMILES: Nc1c(-c2ccc(F)c3[nH]ncc23)c2nc(C3CC3)c(Cl)cc2[nH]c1=O] | | 5.45 (s, 2H), 2.26-2.21 (m, 1H), 0.65- 0.62 (m, 2H), 0.39-0.35 (m, 1H), 0.25- 0.21 (m, 1H). ¹⁹F NMR (376 MHz, DMSO-d₆) δ ppm −132.75 (s, 1F). MS (ESI⁺) obsd. [(M + H)⁺]: 370.1. |
| 056 | 3-Amino-7-chloro-4-(7- | Compound | ¹H NMR (400 MHz, DMSO-d₆) δ ppm |
| | fluoro-1H-indazol-4-yl)-6,8- | Int-19 | 13.60 (s, 1H), 11.47 (s, 1H), 7.62 (s, |
| | dimethyl-1H-1,5- | | 1H), 7.28 (dd, J = 3.6, 11.6 Hz, 1H), |
| | naphthyridin-2-one | | 6.99 (dd, J = 4.0, 7.6 Hz, 1H), 5.29 (s, |
| | 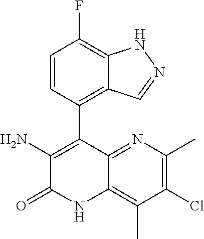 [SMILES: Cc1nc2c(-c3ccc(F)c4[nH]ncc34)c(N)c(=O)[nH]c2c(C)c1Cl] | | 2H), 2.55 (s, 3H), 2.26 (s, 3H). ¹⁹F NMR (376 MHz, DMSO-d₆) δ ppm −132.82 (s, 1F). MS (ESI⁺) obsd. [(M + H)⁺]: 358.2. |
| 057 | 3-Amino-8-chloro-4-(7- | Compound | ¹H NMR (400 MHz, DMSO-d₆) δ ppm |
| | fluoro-1H-indazol-4-yl)-6,7- | Int-20 | 13.79 (s, 1H), 11.28 (s, 1H), 7.79 (s, |
| | dimethyl-1H-1,5- | | 1H), 7.32-7.27 (m, 1H), 6.97-6.94 (m, |
| | naphthyridin-2-one | | 1H), 5.44 (s, 2H), 2.26 (s, 3H), 1.38 (s, |
| | 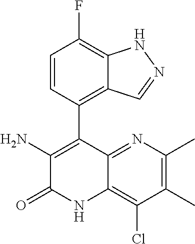 [SMILES: Cc1nc2c(-c3ccc(F)c4[nH]ncc34)c(N)c(=O)[nH]c2c(Cl)c1C] | | 3H). ¹⁹F NMR (376 MHz, DMSO-d₆) δ ppm −131.74 (s, 1F). MS (ESI⁺) obsd. [(M + H)⁺]: 358.2. |

### Example 058: 1-[[7-Amino-8-(7-fluoro-1H-indazol-4-yl)-3-methyl-6-oxo-5H-1,5-naphthyridin-2-yl]oxymethyl]cyclopropanecarbonitrile

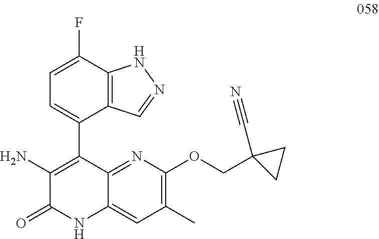 [SMILES: Cc1cc2[nH]c(=O)c(N)c(-c3ccc(F)c4[nH]ncc34)c2nc1OCC1(C#N)CC1]

### Step 1: tert-Butyl (6-((1-cyanocyclopropyl)methoxy)-2-(7-fluoro-1-(tetrahydro-2H-pyran-2-yl)-1H-indazole-4-carbonyl)-5-methylpyridin-3-yl)carbamate

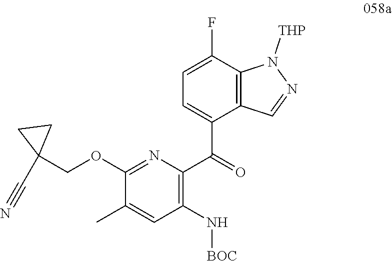

The mixture of Int-13 (50.0 mg, 0.11 mmol), 1-(bromomethyl)cyclopropanecarbonitrile (34.01 mg, 0.21 mmol) and K₂CO₃ (44.32 mg, 0.32 mmol) in DMF (2 mL) was stirred at 80° C. for 2 hours. After completion, the mixture was poured into water (10 mL) and the suspension was collected by filtration. The filter cake was washed with water (30 mL), concentrated under reduced pressure to afford compound 058a (50.0 mg, 81.8% yield) as a light yellow oil. MS (ESI⁺) obsd. [(M+H)⁺]: 550.2.

### Step 2: 1-(((5-Amino-6-(7-fluoro-1H-indazole-4-carbonyl)-3-methylpyridin-2-yl)oxy)methyl)cyclopropane-1-carbonitrile

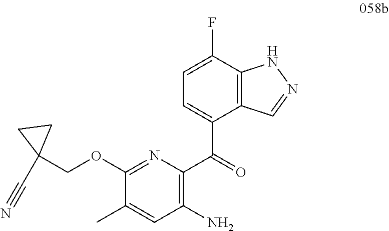 [SMILES: Cc1cc(N)c(C(=O)c2ccc(F)c3[nH]ncc23)nc1OCC1(C#N)CC1]

The title compound was prepared in analogy to the preparation of compound 001b, replacing compound 001a with compound 058a. Compound 058b (30.0 mg) was obtained as a yellow solid. MS (ESI⁺) obsd. [(M+H)⁺]: 366.2.

### Step 3: 2-Chloro-N-(6-((1-cyanocyclopropyl)methoxy)-2-(7-fluoro-1H-indazole-4-carbonyl)-5-methylpyridin-3-yl)acetamide

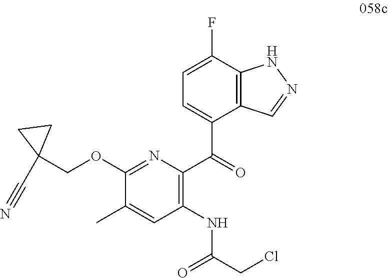 [SMILES: Cc1cc(NC(=O)CCl)c(C(=O)c2ccc(F)c3[nH]ncc23)nc1OCC1(C#N)CC1]

The title compound was prepared in analogy to the preparation of compound 001c, replacing compound 001b with compound 058b. Compound 058c (35.0 mg, crude) was obtained as a black solid. MS (ESI⁺) obsd. [(M+H)⁺]: 442.1.

### Step 4: 1-[[8-(7-Fluoro-1H-indazol-4-yl)-3-methyl-6-oxo-7-pyridin-1-ium-1-yl-5H-1,5-naphthyridin-2-yl]oxymethyl]cyclopropanecarbonitrile;chloride

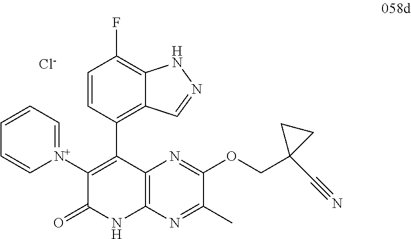 [SMILES: Cc1nc2[nH]c(=O)c(-[n+]3ccccc3)c(-c3ccc(F)c4[nH]ncc34)c2nc1OCC1(C#N)CC1 ; [Cl-]]

The title compound was prepared in analogy to the preparation of compound 001d, replacing compound 001c with compound 058c. Compound 058d (30.0 mg) was obtained as a brown solid. MS (ESI⁺) obsd. [(M-Cl)⁺]: 467.3.

### Step 5: 1-[[7-Amino-8-(7-fluoro-1H-indazol-4-yl)-3-methyl-6-oxo-5H-1,5-naphthyridin-2-yl]oxymethyl]cyclopropanecarbonitrile

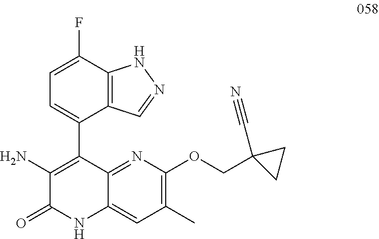 [SMILES: Cc1cc2[nH]c(=O)c(N)c(-c3ccc(F)c4[nH]ncc34)c2nc1OCC1(C#N)CC1]

The title compound was prepared in analogy to the preparation of Example 001 in step 5, replacing compound 001d with compound 058d. Example 058 (5.0 mg) was obtained as a yellow solid. MS (ESI⁺) obsd. [(M+H)⁺]: 405.2.

Example 058: ¹H NMR (400 MHz, DMSO-d₆) δ ppm 13.61 (s, 1H), 12.04 (s, 1H), 7.65 (d, J=2.4 Hz, 1H), 7.44 (s, 1H), 7.29 (dd, J=8.4, 10.8 Hz, 1H), 7.07 (dd, J=4.4, 8.0 Hz, 1H), 5.25 (s, 2H), 3.84-3.76 (m, 2H), 2.19 (s, 3H), 1.05-0.98 (m, 2H), 0.57-0.53 (m, 1H), 0.42-0.38 (m, 1H). ¹⁹F NMR (400 MHz, DMSO-d₆) δ ppm −132.66 (s, 1F).

The following Examples 059 to 068 were prepared in analogy to the procedure described for the preparation of Example 058, replacing 1-(bromomethyl)cyclopropanecarbonitrile with BRANCH indicated in Table 7 in step 1.

TABLE 7
Compound synthesis and characterization

| | Compound Names and | | |
|---|---|---|---|
| Ex. | Structures | BRANCH | NMR and (ESI⁺) |
| 059 | 3-Amino-6-(2,2- | 1,1-Difluoro-2- | ¹H NMR (400 MHz, DMSO- |
| | difluoroethoxy)-4-(7- | iodoethane | d₆) δ ppm 13.62 (s, 1H), |
| | fluoro-1H-indazol-4- | | 12.07 (s, 1H), 7.67 (d, J = |
| | yl)-7-methyl-1H-1,5- | | 3.2 Hz, 1H), 7.45 (s, 1H), |
| | naphthyridin-2-one | | 7.28 (dd, J = 8.0, 11.2 Hz, |
| | 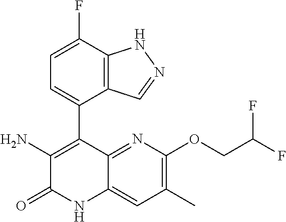 [SMILES: Cc1cc2[nH]c(=O)c(N)c(-c3ccc(F)c4[nH]ncc34)c2nc1OCC(F)F] | | 1H), 7.09 (dd, J = 4.4, 8.0 Hz, 1H), 5.86 (tt, J = 4.0, 55.6 Hz, 1H), 4.01-3.88 (m, 2H), 2.16 (s, 3H). ¹⁹F NMR (376 MHz, DMSO-d₆) δ = −125.636 (s, 2F), −132.720 (s, 1F). MS (ESI⁺) obsd. [(M + H)⁺]: 390.1. |
| 060 | 3-Amino-4-(7-fluoro- | Iodomethane | ¹H NMR (400 MHz, DMSO- |
| | 1H-indazol-4-yl)-6- | | d₆) δ ppm 7.63 (s, 1H), 7.41 |
| | methoxy-7-methyl-1H- | | (s, 1H), 7.28 (dd, J = 8.0, |
| | 1,5-naphthyridin-2-one | | 11.2 Hz, 1H), 7.11 (dd, J = |
| | 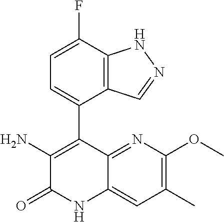 [SMILES: COc1nc2c(-c3ccc(F)c4[nH]ncc34)c(N)c(=O)[nH]c2cc1C] | | 4.2, 7.8 Hz, 1H), 3.32 (s, 3H), 2.13 (s, 3H) ¹⁹F NMR (376 MHz, DMSO-d₆) δ ppm −132.76 (s, 1F) MS (ESI⁺) obsd. [(M + H)⁺]: 340.2. |
| 061 | Ethyl 2-[[7-amino-8-(7- | Ethyl 2- | ¹H NMR (400 MHz, DMSO- |
| | fluoro-1H-indazol-4- | bromoacetate | d₆) δ ppm 13.57 (s, 1H), |
| | yl)-3-methyl-6-oxo-5H- | | 12.03 (s, 1H), 7.57 (d, J = |
| | 1,5-naphthyridin-2- | | 3.2 Hz, 1H), 7.43 (s, 1H), |
| | yl]oxy]acetate | | 7.25 (dd, J = 8.0, 10.8 Hz, |
| | 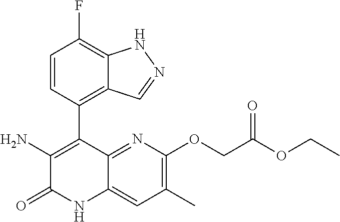 [SMILES: CCOC(=O)COc1nc2c(-c3ccc(F)c4[nH]ncc34)c(N)c(=O)[nH]c2cc1C] | | 1H), 6.97 (dd, J = 4.0, 7.6 Hz, 1H), 5.18-5.16 (m, 2H), 4.44-4.25 (m, 2H), 3.61-3.56 (m, 2H), 2.19 (s, 3H), 0.95 (t, J = 6.8 Hz, 3H). ¹⁹F NMR (400 MHz, DMSO-d₆) δ ppm −133.09 (s, 1F). MS (ESI⁺) obsd. [(M + H)⁺]: 412.1. |
| 062 | 3-Amino-6- | Sodium | ¹H NMR (400 MHz, MeOD- |
| | (difluoromethoxy)-4-(7- | chlorodifluoro- | d₄) δ ppm 7.68 (s, 1H), 7.57 |
| | fluoro-1H-indazol-4- | acetate | (s, 1H), 7.30-7.25 (m, 1H), |
| | yl)-7-methyl-1H-1,5- | | 7.18 (dd, J = 4.4, 8.0 Hz, |
| | naphthyridin-2-one | | 1H), 6.80 (t, J = 74.0 Hz, |
| | 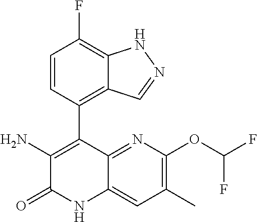 [SMILES: Cc1cc2[nH]c(=O)c(N)c(-c3ccc(F)c4[nH]ncc34)c2nc1OC(F)F] | | 1H), 2.28 (s, 3H) ¹⁹F NMR (376 MHz, MeOD-d₄) δ ppm −90.50 (dd, J = 174.0, 78.0 Hz, 2F), −134.60 (s, 1F). MS (ESI⁺) obsd. [(M + H)⁺]: 376.3. |
| 063 | 3-Amino-6-(1- | 1- | ¹H NMR (400 MHz, DMSO- |
| | bicyclo[1.1.1]pentanyl | (Bromomethyl) | d₆) δ ppm 13.57 (s, 1H), |
| | methoxy)-4-(7-fluoro- | bicyclo[1.1.1] | 11.96 (s, 1H), 7.64 (d, J = |
| | 1H-indazol-4-yl)-7- | pentane | 2.4 Hz, 1H), 7.38 (s, 1H), |
| | methyl-1H-1,5- | | 7.29 (dd, J = 8.0, 11.2 Hz, |
| | naphthyridin-2-one | | 1H), 7.06 (dd, J = 4.0, 7.6 |
| | 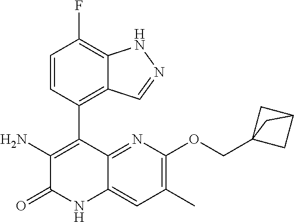 [SMILES: Cc1cc2[nH]c(=O)c(N)c(-c3ccc(F)c4[nH]ncc34)c2nc1OCC12CC(C1)C2] | | Hz, 1H), 5.16 (s, 2H), 3.76- 3.67 (m, 2H), 2.28 (s, 1H), 2.14 (s, 3H), 1.34 (s, 6H) ¹⁹F NMR (374 MHz, DMSO-d₆) δ ppm −133.05 (s, 1F). MS (ESI⁺) obsd. [(M + H)⁺]: 406.1. |
| 064 | 3-Amino-4-(7-fluoro- | 2-Iodopropane | ¹H NMR (400 MHz, MeOD- |
| | 1H-indazol-4-yl)-6- | | d₄) δ ppm 7.67 (d, J = 3.2 |
| | isopropoxy-7-methyl- | | Hz, 1H), 7.43 (s, 1H), 7.26 |
| | 1H-1,5-naphthyridin-2- | | (dd, J = 7.6, 10.8 Hz, 1H), |
| | one | | 7.15 (dd, J = 4.0, 7.6 Hz, |
| | 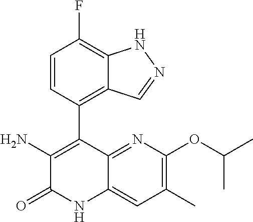 [SMILES: Cc1cc2[nH]c(=O)c(N)c(-c3ccc(F)c4[nH]ncc34)c2nc1OC(C)C] | | 1H), 4.56 (dd, J = 6.0, 12.4 Hz, 1H), 2.18 (s, 3H), 1.03 (d, J = 6.4 Hz, 3H), 0.92 (d, J = 6.4 Hz, 3H). ¹⁹F NMR (376 MHz, DMSO-d₆) δ ppm −134.89 (s, 1F). MS (ESI⁺) obsd. [(M + H)⁺]: 368.1. |
| 065 | 3-Amino-4-(7-fluoro- | 1-Bromopropane | ¹H NMR (400 MHz, DMSO- |
| | 1H-indazol-4-yl)-7- | | d₆) δ ppm 13.56 (s, 1H), |
| | methyl-6-propoxy-1H- | | 11.98 (s, 1H), 7.63 (s, 1H), |
| | 1,5-naphthyridin-2-one | | 7.38 (s, 1H), 7.28 (dd, J = |
| | 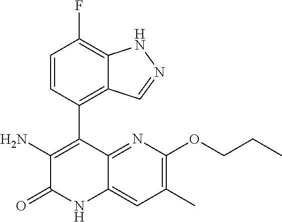 [SMILES: CCCOc1nc2c(-c3ccc(F)c4[nH]ncc34)c(N)c(=O)[nH]c2cc1C] | | 8.0, 10.8 Hz, 1H), 7.06 (dd, J = 4.4, 8.0 Hz, 1H), 5.16 (s, 2H), 3.67-3.62 (m, 2H), 2.13 (s, 3H), 1.37-1.30 (m, 2H), 0.61 (t, J = 7.6 Hz, 3H) ¹⁹F NMR (376 MHz, DMSO-d₆) δ ppm −133.04 (s, 1F). MS (ESI⁺) obsd. [(M + H)⁺]: 368.2. |
| 066 | 3-Amino-4-(7-fluoro- | 1,1,1-Trifluoro-2- | ¹H NMR (400 MHz, DMSO- |
| | 1H-indazol-4-yl)-7- | iodoethane | d₆) δ ppm 13.60 (d, J = 1.6 |
| | methyl-6-(2,2,2- | | Hz, 1H), 12.10 (s, 1H), 7.65 |
| | trifluoroethoxy)-1H- | | (d, J = 2.0 Hz, 1H), 7.48 (s, |
| | 1,5-naphthyridin-2-one | | 1H), 7.33-7.24 (m, 1H), 7.09 |
| | 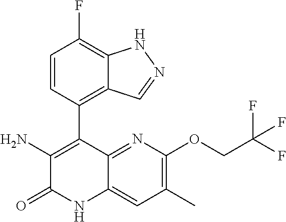 [SMILES: Cc1cc2[nH]c(=O)c(N)c(-c3ccc(F)c4[nH]ncc34)c2nc1OCC(F)(F)F] | | (dd, J = 4.0, 8.0 Hz, 1H), 5.32 (d, J = 5.6 Hz, 2H), 4.46-4.30 (m, 2H), 2.18 (s, 3H). ¹⁹F NMR (376 MHz, DMSO-d₆) δ ppm −72.80 (s, 3F), −132.66 (s, 1F). MS (ESI⁺) obsd. [(M + H)⁺]: 408.2. |
| 067 | Methyl 3-[[7-amino-8- | Methyl 3- | ¹H NMR (400 MHz, MeOD- |
| | (7-fluoro-1H-indazol-4- | bromocyclo- | d₄) δ ppm 7.66 (d, J = 3.6 |
| | yl)-3-methyl-6-oxo-5H- | butane- | Hz, 1H), 7.44 (s, 1H), 7.28 |
| | 1,5-naphthyridin-2- | carboxylate | (dd, J = 10.4, 7.6 Hz, 1H), |
| | yl]oxy]cyclobutane- | | 7.14 (dd, J = 7.6, 4.0 Hz, |
| | carboxylate | | 1H), 4.39 (t, J = 7.44 Hz, |
| | 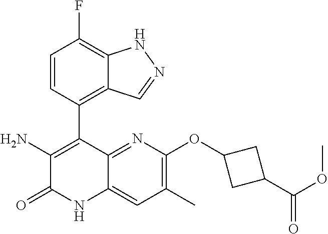 [SMILES: COC(=O)C1CC(Oc2nc3c(-c4ccc(F)c5[nH]ncc45)c(N)c(=O)[nH]c3cc2C)C1] | | 1H), 3.62 (s, 3H), 2.41-2.49 (m, 1H), 2.21 (s, 3H), 1.92- 2.10 (m, 4H). ¹⁹F NMR (376 MHz, MeOD-d₄) δ ppm −134.72 (s, 1F). MS (ESI⁺) obsd. [(M + H)⁺]: 438.3. |
| 068 | 3-Amino-6-[2- | 2-Iodo-N,N- | ¹H NMR (400 MHz, DMSO- |
| | (dimethylamino)ethoxy]- | dimethyl- | d₆) δ = 7.65 (d, J = 3.2 Hz, |
| | 4-(7-fluoro-1H- | ethanamine | 1H), 7.49 (s, 1H), 7.30 (dd, |
| | indazol-4-yl)-7-methyl- | | J = 8.0, 11.6 Hz, 1H), 7.10 |
| | 1H-1,5-naphthyridin-2- | | (dd, J = 4.0, 7.6 Hz, 1H), |
| | one | | 4.10-4.03 (m, 1H), 4.01-3.96 |
| | 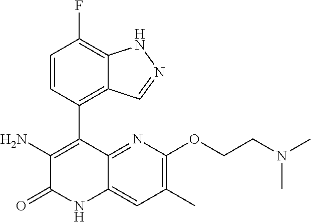 [SMILES: Cc1cc2[nH]c(=O)c(N)c(-c3ccc(F)c4[nH]ncc34)c2nc1OCCN(C)C] | | (m, 1H), 3.10 (t, J = 5.2 Hz, 2H), 2.61 (d, J = 5.2 Hz, 6H), 2.17 (s, 3H) ¹⁹F NMR (376 MHz, DMSO-d₆) δ ppm −132.24 (s, 1F). MS (ESI⁺) obsd. [(M + H)⁺]: 397.2. |

### Example 069: 3-Amino-4-(7-fluoro-1H-indazol-4-yl)-7-methyl-6-(4-piperidyl)-1H-1,5-naphthyridin-2-one

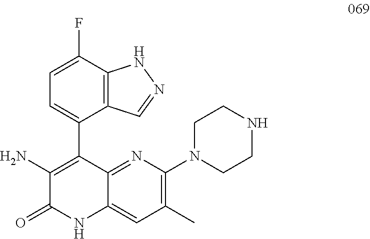 [SMILES: Cc1cc2[nH]c(=O)c(N)c(-c3ccc(F)c4[nH]ncc34)c2nc1N1CCNCC1]

### Step 1: tert-Butyl 4-[7-amino-8-(7-fluoro-1H-indazol-4-yl)-3-methyl-6-oxo-5H-1,5-naphthyridin-2-yl]-3,6-dihydro-2H-pyridine-1-carboxylate

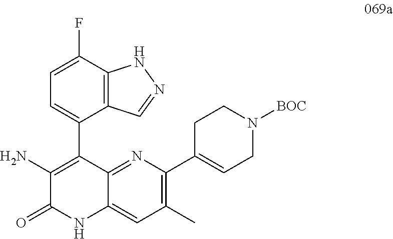 [SMILES: Cc1cc2[nH]c(=O)c(N)c(-c3ccc(F)c4[nH]ncc34)c2nc1C1=CCN(C(=O)OC(C)(C)C)CC1]

A mixture of Example 021 (50.0 mg, 0.13 mmol), Pd(dppf)Cl₂ (9.4 mg, 0.01 mmol), K₂CO₃ (35.6 mg, 0.26 mmol) and 1-N-Boc-4-(4,4,5,5-tetramethyl-[1,3,2]dioxaborolan-2-yl)-3,6-dihydro-2H-pyridine (59.7 mg, 0.19 mmol) in 1,4-dioxane (1 mL) and water (0.3 mL) was stirred at 80° C. for 2 hours under N₂ protection. After completion, the mixture was concentrated in vacuo to give a crude, which was purified by silica-gel chromatography (elute with EtOAc:PE=0-20%) to give compound 069a (30.0 mg, 47.5% yield) as a yellow solid. MS (ESI⁺) obsd. [(M+H)⁺]: 491.4.

### Step 2: tert-Butyl 4-[7-amino-8-(7-fluoro-1H-indazol-4-yl)-3-methyl-6-oxo-5H-1,5-naphthyridin-2-yl]piperidine-1-carboxylate

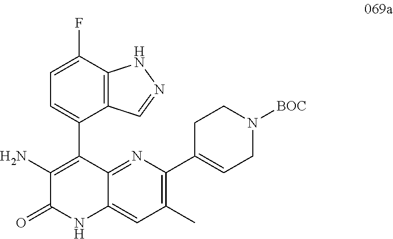 [SMILES: Cc1cc2[nH]c(=O)c(N)c(-c3ccc(F)c4[nH]ncc34)c2nc1C1=CCN(C(=O)OC(C)(C)C)CC1]

The mixture of compound 069a (30.0 mg, 0.06 mmol) and Pd/C (289.72 mg, 10% wt) in EtOAc (3 mL) was stirred under H₂(15 psi) at 25° C. for 1 hour. After completion, the mixture was filtered and the filtrate was concentrated in vacuo to give compound 069b (30.0 mg, 100% yield) as a yellow oil. MS (ESI⁺) obsd. [(M+H)⁺]: 493.2.

### Step 3: 3-Amino-4-(7-fluoro-1H-indazol-4-yl)-7-methyl-6-(4-piperidyl)-1H-1,5-naphthyridin-2-one

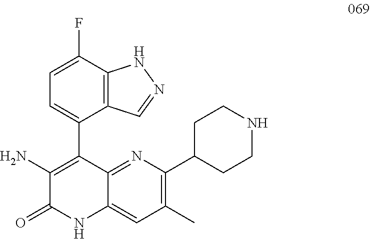 [SMILES: Cc1cc2[nH]c(=O)c(N)c(-c3ccc(F)c4[nH]ncc34)c2nc1C1CCNCC1]

To a solution of compound 069b (23.0 mg, 0.05 mmol) in EtOAc (0.5 mL) was added EtOAc/HCl (4.0 mL, 16.0 mmol). The mixture was stirred at 25° C. for 3 hours. After completion, solvent was evaporated and the residue was purified by pre-HPLC (NH₃ as additive) to afford Example 069 (12.5 mg, 52.3% yield) as white solid. MS (ESI⁺) obsd. [(M+H)⁺]: 393.1.

Example 069: ¹H NMR (400 MHz, DMSO-d₆) δ ppm 7.57 (d, J=3.2 Hz, 1H), 7.33-7.31 (m, 1H), 7.30-7.22 (m, 1H), 7.07 (dd, J=4.4, 8.0 Hz, 1H), 5.12 (s, 2H), 2.83-2.72 (m, 3H), 2.45-2.38 (m, 2H), 2.30 (s, 3H), 1.42-1.38 (m, 1H), 1.32-1.21 (m, 2H), 1.15-1.05 (m, 1H). ¹⁹F NMR (376 MHz, DMSO-d₆) δ ppm −132.91 (s, 1F).

### Example 070: 3-Amino-4-(7-fluoro-1H-indazol-4-yl)-6-(3-hydroxy-3-methyl-but-1-ynyl)-7-methyl-1H-1,5-naphthyridin-2-one

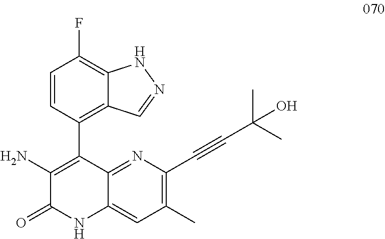 [SMILES: Cc1cc2[nH]c(=O)c(N)c(-c3ccc(F)c4[nH]ncc34)c2nc1C#CC(C)(C)O]

The mixture of Example 021 (20.0 mg, 0.05 mmol), 2-methyl-3-butyn-2-ol (17.34 mg, 0.21 mmol), TEA (1.46 mL, 7.19 mmol), Pd(PPh₃)₂Cl₂ (1.81 mg) and CuI (0.98 mg, 0.01 mmol) in THE (0.5 mL) was stirred at 60° C. for 12 hours under N₂ protection. After completion, the mixture was poured into water (10 mL) and extracted with EtOAc (10 mL×3). The combined organic layer was washed with brine (20 mL), dried over anhydrous sodium sulfate and concentrated under reduced pressure to give a residue, which was purified by prep-HPLC (NH₄HCO₃ as additive) and lyophilized to give Example 070 (6.1 mg, 28.8% yield) as a yellow solid. MS (ESI⁺) obsd. [(M+H)⁺]: 392.1.

Example 070: ¹H NMR (400 MHz, DMSO-d₆) δ ppm 13.66 (s, 1H), 12.12 (s, 1H), 7.64 (s, 1H), 7.45 (s, 1H), 7.30 (dd, J=8.0, 11.2 Hz, 1H), 6.97 (dd, J=4.0, 7.6 Hz, 1H), 5.43 (s, 1H), 5.18 (s, 2H), 2.36 (s, 3H), 1.38 (s, 6H). ¹⁹F NMR (376 MHz, DMSO-d₆) δ ppm −132.65 (s, 1F).

The following Examples 071 to 077 were prepared in analogy to the procedure described for the preparation of Example 071, replacing 2-methyl-3-butyn-2-ol with ALKYNE indicated in Table 8.

TABLE 8
Compound synthesis and characterization

| | Compound Names and | | |
|---|---|---|---|
| Ex. | Structures | ALKYNE | NMR and (ESI⁺) |
| 071 | 3-Amino-4-(7-fluoro-1H- indazol-4-yl)-6-(3- methoxyprop-1-ynyl)- 7-methyl-1H-1,5- naphthyridin-2-one 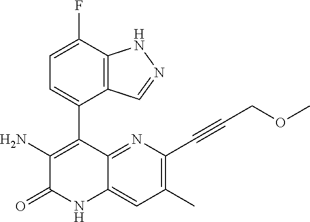 [SMILES: COCC#Cc1nc2c(-c3ccc(F)c4[nH]ncc34)c(N)c(=O)[nH]c2cc1C] | Methyl propargyl ether | ¹H NMR (400 MHz, MeOD- d₄) δ ppm 7.63 (d, J = 2.40 Hz, 1H), 7.53 (s, 1H), 7.22- 7.31 (m, 1H), 7.08-7.16 (m, 1H), 4.26 (s, 2H), 3.35 (s, 3H), 2.47 (s, 3H). ¹⁹F NMR (376 MHz, MeOD-d₄) δ ppm −134.74 (s, 1 F). MS (ESI⁺) obsd. [(M + H)⁺]: 378.1. |
| 072 | 3-Amino-4-(7-fluoro- 1H-indazol-4-yl)-6-(3- methoxy-3-methyl-but- 1-ynyl)-7-methyl-1H- 1,5-naphthyridin-2-one 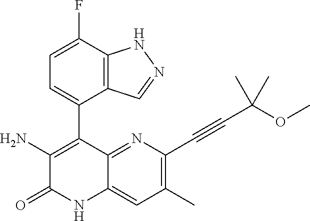 [SMILES: COC(C)(C)C#Cc1nc2c(-c3ccc(F)c4[nH]ncc34)c(N)c(=O)[nH]c2cc1C] | 3-Methoxy-3- methyl-but-1-yne | ¹H NMR (400 MHz, DMSO- d₆) δ ppm 13.63 (s, 1H), 12.21 (s, 1H), 7.64 (s, 1H), 7.46 (s, 1H), 7.30-7.27 (m, 1H), 7.00 (dd, J = 4.0, 7.6 Hz, 1H), 5.22 (s, 2H), 3.21 (s, 3H), 2.37 (s, 3H), 1.39 (s, 6H) ¹⁹F NMR (376 MHz, DMSO-d₆) δ ppm −132.59 (s, 1F). MS (ESI⁺) obsd. [(M + H)⁺]: 406.3. |
| 073 | 3-Amino-4-(7-fluoro- 1H-indazol-4-yl)-6-[2- (4- hydroxytetrahydrofuran- 3-yl)ethynyl]-7- methyl-1H-1,5- naphthyridin-2-one 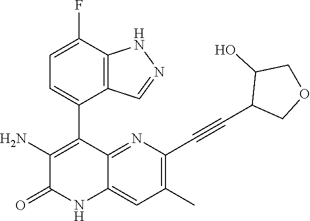 [SMILES: Cc1cc2[nH]c(=O)c(N)c(-c3ccc(F)c4[nH]ncc34)c2nc1C#CC1COCC1O] | 4- Ethynyltetrahydro- furan-3-ol | ¹H NMR (400 MHz, DMSO- d₆) δ ppm 13.63 (m, 1H), 12.19 (d, J = 2.8 Hz, 1H), 7.62 (s, 1H), 7.43 (s, 1H), 7.27-7.35 (m, 1H), 6.97 (dd, J = 7.20, 4.0 Hz, 1H), 5.44 (d, J = 4.0 Hz, 1H), 5.20 (s, 2 H), 4.24-4.29 (m, 1H), 3.98 (t, J = 7.60 Hz, 1H), 3.81-3.87 (m, 1H), 3.60-3.65 (m, 1H), 3.50 (dd, J = 9.2, 2.4 Hz, 1H), 2.97 (td, J = 4.40 Hz, 2.44 Hz, 1H), 2.33 (s, 3 H). ¹⁹F NMR (377 MHz, DMSO-d₆) δ ppm −132.71 (s, 1 F). MS (ESI⁺) obsd. [(M + H)⁺]: 420.3. |
| 074 | 3-Amino-4-(7-fluoro- 1H-indazol-4-yl)-6-(3- hydroxybut-1-ynyl)-7- methyl-1H-1,5- naphthyridin-2-one 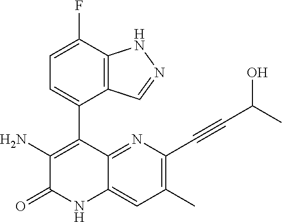 [SMILES: Cc1cc2[nH]c(=O)c(N)c(-c3ccc(F)c4[nH]ncc34)c2nc1C#CC(C)O] | But-3-yn-2-ol | ¹H NMR (400 MHz, DMSO- d₆) δ ppm 13.64 (s, 1H), 12.19 (s, 1H), 7.63 (s, 1H), 7.45 (s, 1H), 7.30 (dd, J = 8.0, 11.2 Hz, 1H), 6.98 (dd, J = 4.4, 8.0 Hz, 1H), 5.40 (d, J = 5.2 Hz, 1H), 5.20 (s, 2H), 4.53-4.47 (m 1H), 2.36 (s, 3H), 1.30 (d, J = 6.8 Hz, 3H). ¹⁹F NMR (376 MHz, DMSO-d₆) δ ppm −132.72 (s, 1F). MS (ESI⁺) obsd. [(M + H)⁺]: 378.1. |
| 075 | 3-Amino-6-[3- (dimethylamino)prop-1- ynyl]-4-(7-fluoro-1H- indazol-4-yl)-7-methyl- 1H-1,5-naphthyridin-2- one 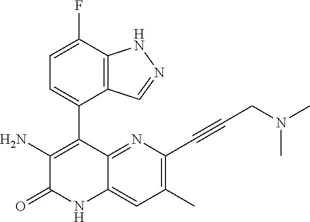 [SMILES: Cc1cc2[nH]c(=O)c(N)c(-c3ccc(F)c4[nH]ncc34)c2nc1C#CCN(C)C] | N,N-dimethylprop- 2-yn-1-amine | ¹H NMR (400 MHz, DMSO- d₆) δ ppm 13.63 (s, 1H), 12.21 (s, 1H), 7.62 (s, 1H), 7.45 (s, 1H), 7.30 (dd, J = 8.0, 18.8 Hz, 1H), 7.00 (dd, J = 4.0, 8.0 Hz, 1H), 5.23 (s, 2H), 3.41 (s, 2H), 2.40 (s, 3H), 2.20 (s, 6H). ¹⁹F NMR (400 MHz, DMSO-d₆) δ ppm −132.769 (s, 1F) MS (ESI⁺) obsd. [(M + H)⁺]: 391.3. |
| 076 | 3-Amino-4-(7-fluoro- 1H-indazol-4-yl)-7- methyl-6-(3- morpholinoprop-1- ynyl)-1H-1,5- naphthyridin-2-one 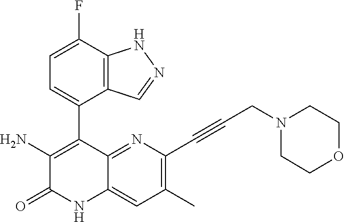 [SMILES: Cc1cc2[nH]c(=O)c(N)c(-c3ccc(F)c4[nH]ncc34)c2nc1C#CCN1CCOCC1] | 4-Prop-2- ynylmorpholine | ¹H NMR (400 MHz, DMSO- d₆) δ ppm 12.35 (s, 1H), 11.54 (br s, 1H), 7.65 (d, J = 3.2 Hz, 1H), 7.51 (s, 1H), 7.30 (dd, J = 8.0, 11.2 Hz, 1H), 6.99 (dd, J = 4.0, 8.0 Hz, 1H), 4.30 (s, 2H), 3.71 (br s, 3H), 3.37 (br s, 2H), 3.07 (br s, 2H), 2.43 (s, 3H). ¹⁹F NMR (376 MHz, DMSO-d₆) δ ppm −132.60 (s, 1F). MS (ESI⁺) obsd. [(M + H)⁺]: 433.2. |
| 077 | 3-Amino-6-(3-amino-3- methyl-but-1-ynyl)-4- (7-fluoro-1H-indazol-4- yl)-7-methyl-1H-1,5-naphthyridin-2-one 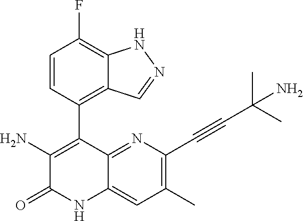 [SMILES: Cc1cc2[nH]c(=O)c(N)c(-c3ccc(F)c4[nH]ncc34)c2nc1C#CC(C)(C)N] | 1,1- Dimethylpropargyl amine | ¹H NMR (400 MHz, DMSO- d₆) δ ppm 13.68 (s, 1H), 7.64 (d, J = 3.2 Hz, 1H), 7.44 (s, 1H), 7.30 (dd, J = 7.8, 11.2 Hz, 1H), 6.98 (d, J = 12 Hz, 1H), 5.16 (s, 2H), 2.35 (s, 3H), 1.31 (s, 6H) ¹⁹F NMR (376 MHz, DMSO- d₆) δ ppm −132.674 (s, 1F). MS (ESI⁺) obsd. [(M + H)⁺]: 391.2. |

### Example 078: 3-Amino-4-(7-fluoro-1H-indazol-4-yl)-6-(2-hydroxy-2-methyl-propoxy)-7-methyl-1H-1,5-naphthyridin-2-one

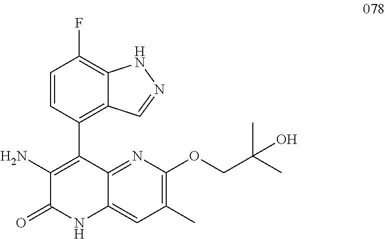 [SMILES: Cc1cc2[nH]c(=O)c(N)c(-c3ccc(F)c4[nH]ncc34)c2nc1OCC(C)(C)O]

To a solution of Example 061 (20.0 mg, 0.05 mmol) in THE (1 mL) was added methyl magnesium bromide (0.16 mL, 0.49 mmol) at 0° C. under N₂ atmosphere and the mixture was stirred at 0° C. for 2 hours. After completion, the reaction mixture was quenched with saturated ammonium chloride (5 mL) and extracted with EtOAc (5 mL×3). The combined organic layer was washed with brine (15 mL), dried over anhydrous sodium sulfate and concentrated under reduced pressure to give a residue, which was purified by prep-HPLC (NH₄HCO₃ as additive) and lyophilized to give Example 078 (8.0 mg, 40.5% yield) as a white solid. MS (ESI⁺) obsd. [(M+H)⁺]: 398.3.

Example 078: ¹H NMR (400 MHz, DMSO-d₆) δ ppm 13.57 (s, 1H), 11.98 (s, 1H), 7.63 (d, J=2.0 Hz, 1H), 7.39 (s, 1H), 7.28 (dd, J=7.6, 10.4 Hz, 1H), 7.07 (dd, J=4.0, 7.6 Hz, 1H), 5.16-5.15 (m, 2H), 4.31 (s, 1H), 3.54-3.41 (m, 2H), 2.17 (s, 3H), 0.97 (s, 3H), 0.94 (s, 3H). ¹⁹F NMR (376 MHz, DMSO-d₆) δ ppm −132.90 (s, 1F).

### Example 079: 3-[[7-Amino-8-(7-fluoro-1H-indazol-4-yl)-3-methyl-6-oxo-5H-1,5-naphthyridin-2-yl]oxy]cyclobutanecarbonitrile

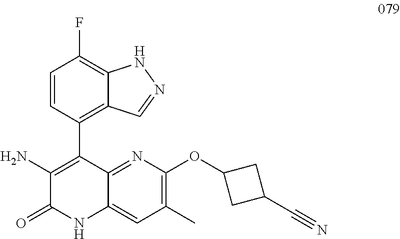 [SMILES: Cc1cc2[nH]c(=O)c(N)c(-c3ccc(F)c4[nH]ncc34)c2nc1OC1CC(C#N)C1]

A mixture of Example 021 (100.0 mg, 0.26 mmol), 3-hydroxycyclobutanecarbonitrile (50.0 mg, 0.52 mmol), BNMO (9.49 mg, 0.03 mmol), CuI (4.9 mg, 0.03 mmol) and t-BuONa (1.3 mL, 2.6 mmol) in 1,4-dioxane (1 mL) was stirred at 60° C. for 12 hours under N₂ protection. After completion, the reaction mixture was quenched with saturated aqueous ammonium chloride (5 mL) and extracted with EtOAc (10 mL×3). The combined organic layer was washed with brine (10 mL), dried over anhydrous sodium sulfate and concentrated under reduced pressure to give a residue, which was purified by prep-HPLC (NH₄HCO₃ as additive) and lyophilized to give Example 079 (15.2 mg, 13.9% yield) as a yellow solid. MS (ESI⁺) obsd. [(M+H)⁺]: 405.1.

Example 079: ¹H NMR (400 MHz, DMSO-d₆) δ ppm 13.62 (br s, 1H), 12.00 (br s, 1H), 7.63 (t, J=3.2 Hz, 1H), 7.40 (d, J=3.6 Hz, 1H), 7.33-7.26 (m, 1H), 7.08-7.01 (m, 1H), 5.19 (d, J=11.6 Hz, 2H), 4.65-4.61 (m, 0.5H), 4.37-4.29 (m, 0.5H), 3.15-3.07 (m, 0.5H), 2.81-2.72 (m, 0.5H), 2.37-2.19 (m, 1.5H), 2.16-2.09 (m, 4H), 2.02-1.96 (m, 1H), 1.83-1.76 (m, 0.5H). ¹⁹F NMR (400 MHz, DMSO-d₆) δ ppm −132.59 (s, 0.5F), −132.77 (s, 0.5F).

### Example 080: trans-3-Amino-6-(3-fluorocyclobutoxy)-4-(7-fluoro-1H-indazol-4-yl)-7-methyl-1H-1,5-naphthyridin-2-one

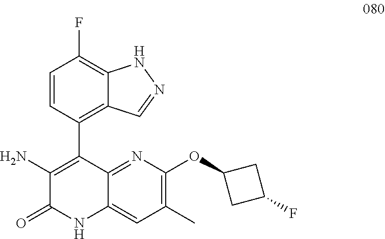 [SMILES: Cc1cc2[nH]c(=O)c(N)c(-c3ccc(F)c4[nH]ncc34)c2nc1O[C@H]1C[C@H](F)C1]

### Step 1: 3-Amino-4-(7-fluoro-1H-indazol-4-yl)-6-iodo-7-methyl-1H-1,5-naphthyridin-2-one

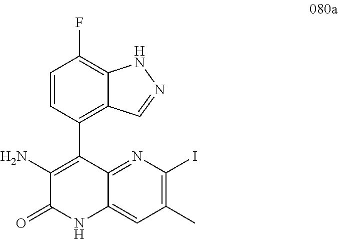 [SMILES: Cc1cc2[nH]c(=O)c(N)c(-c3ccc(F)c4[nH]ncc34)c2nc1I]

A mixture of Example 021 (600.0 mg, 1.55 mmol), NaI (463.4 mg, 3.1 mmol), N1,N2-dimethylethane-1,2-diamine (68.1 mg, 0.77 mmol) and CuI (147.2 mg, 0.77 mmol) in 1,4-dioxane (10 mL) was stirred at 110° C. for 3 hours under N₂ protection. After completion, the mixture was poured into water (50 mL) and extracted with EtOAc (50 mL×3). The combined organic layer was washed with brine (80 mL), dried over anhydrous sodium sulfate and concentrated under reduced pressure to give a residue, which was purified by silica-gel chromatography (elute with EtOAc:PE=25-70%) to give compound 080a (300.0 mg, 44.6% yield) as a yellow solid. MS (ESI⁺) obsd. [(M+H)⁺]: 436.1.

### Step 2: trans-3-Amino-6-(3-fluorocyclobutoxy)-4-(7-fluoro-1H-indazol-4-yl)-7-methyl-1H-1,5-naphthyridin-2-one

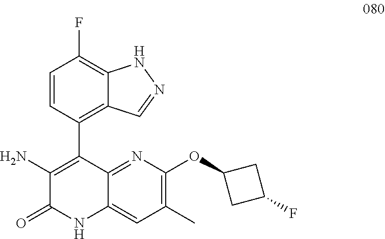 [SMILES: Cc1cc2[nH]c(=O)c(N)c(-c3ccc(F)c4[nH]ncc34)c2nc1O[C@H]1C[C@H](F)C1]

The mixture of compound 080a (50.0 mg, 0.11 mmol), 3-fluorocyclobutanol (20.7 mg, 0.23 mmol), BNMO (4.23 mg, 0.01 mmol), t-BuONa (0.57 mL, 1.15 mmol) and CuI (2.19 mg, 0.01 mmol) in 1,4-dioxane (1 mL) was stirred at 60° C. for 12 hours under N₂ protection. After completion, the reaction mixture was quenched with saturated aqueous ammonium chloride (5 mL) and extracted with ethyl acetate (10 mL×3). The combined organic layer was washed with brine (10 mL), dried over anhydrous sodium sulfate and concentrated in vacuo to give a residue, which was purified by prep-HPLC (NH₄HCO₃ as additive) and lyophilized to give Example 080 (1.6 mg, 3.2% yield) as an off-white solid. MS (ESI⁺) obsd. [(M+H)⁺]: 398.2.

Example 080: ¹H NMR (400 MHz, DMSO-d₆) δ ppm 13.58 (s, 1H), 11.99 (s, 1H), 7.61 (s, 1H), 7.39 (s, 1H), 7.29 (dd, J=8.0, 11.2 Hz, 1H), 7.03 (dd, J=4.0, 7.6 Hz, 1H), 5.22-5.09 (m, 2.5H), 5.02-4.97 (m, 0.5H), 4.66-4.56 (m, 1H), 2.13 (s, 3H), 2.09-1.95 (m, 4H). ¹⁹F NMR (400 MHz, DMSO-d₆) δ ppm −132.97 (s, 1F), −176.79 (s, 1F).

### Example 081: 3-Amino-6-(3,3-difluoropropoxy)-4-(7-fluoro-1H-indazol-4-yl)-7-methyl-1H-1,5-naphthyridin-2-one

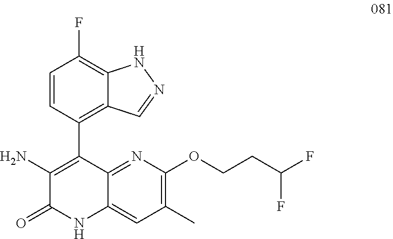 [SMILES: Cc1cc2[nH]c(=O)c(N)c(-c3ccc(F)c4[nH]ncc34)c2nc1OCCC(F)F]

### Step 1: tert-Butyl N-[2-(7-fluoro-1-tetrahydropyran-2-yl-indazole-4-carbonyl)-6-(3-hydroxypropoxy)-5-methyl-3-pyridyl]carbamate

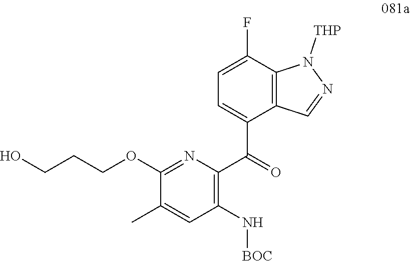

A mixture of Int-13 (500.0 mg, 1.06 mmol), 3-iodopropanol (790.6 mg, 4.3 mmol) and K₂CO₃ (587.5 mg, 4.3 mmol) in DMF (5 mL) was stirred at 100° C. for 2 hours. After completion, the mixture was poured into water (10 mL) and extracted with ethyl acetate (20 mL×3). The combined organic layer was washed with brine (20 mL), dried over anhydrous sodium sulfate and concentrated under reduced pressure to give crude compound 081a (560.0 mg, 100% yield) as a light yellow oil. MS (ESI⁺) obsd. [(M+H)⁺]: 529.2.

### Step 2: tert-Butyl N-[2-(7-fluoro-1-tetrahydropyran-2-yl-indazole-4-carbonyl)-5-methyl-6-(3-oxopropoxy)-3-pyridyl]carbamate

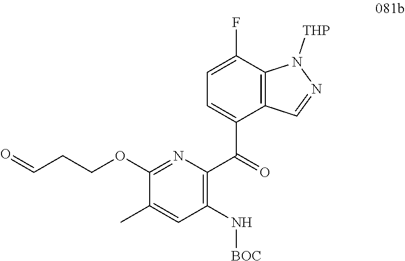

A mixture of compound 081a (560.0 mg, 1.06 mmol) and DMP (674.06 mg, 1.59 mmol) in DCM (6 mL) was stirred at 25° C. for 1 hour. After completion, the reaction mixture was quenched with saturated sodium sulfite (10 mL) and extracted with EtOAc (20 mL×3). The combined organic layer was washed with brine (20 mL), dried over anhydrous sodium sulfate and concentrated under reduced pressure to give a residue, which was purified by silica-gel column chromatography (elute with EtOAc:PE=0-15%) to give compound 081b (150.0 mg, 26.9% yield) as a yellow solid. MS (ESI⁺) obsd. [(M+H)⁺]: 527.3.

### Step 3: tert-Butyl N-[6-(3,3-difluoropropoxy)-2-(7-fluoro-1-tetrahydropyran-2-yl-indazole-4-carbonyl)-5-methyl-3-pyridyl]carbamate

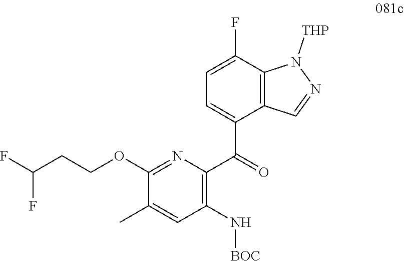

A mixture of compound 081b (170.0 mg, 0.32 mmol) and BAST (0.6 mL, 3.23 mmol) in DCM (5 mL) was stirred at 0° C. for 1 hour. After completion, the reaction mixture was quenched with saturated sodium bicarbonate (10 mL) and extracted with EtOAc (20 mL×3). The combined organic layer was washed with brine (10 mL), dried over anhydrous sodium sulfate and concentrated under reduced pressure to give a residue, which was purified by column chromatography (elute with EtOAc:PE=0-10%) to give compound 081c (50.0 mg, 28.2% yield) as a yellow solid. MS (ESI⁺) obsd. [(M+H)⁺]: 549.4.

### Step 4: [3-Amino-6-(3,3-difluoropropoxy)-5-methyl-2-pyridyl]-(7-fluoro-1H-indazol-4-yl)methanone

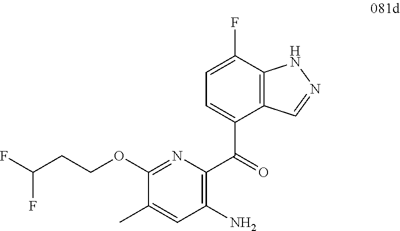 [SMILES: Cc1cc(N)c(C(=O)c2ccc(F)c3[nH]ncc23)nc1OCCC(F)F]

The title compound was prepared in analogy to the preparation of compound 001b, replacing compound 001a with compound 081c. Compound 081d (28.0 mg) was obtained as a yellow solid. MS (ESI⁺) obsd. [(M+H)⁺]: 365.2.

### Step 5: 2-Chloro-N-[6-(3,3-difluoropropoxy)-2-(7-fluoro-1H-indazole-4-carbonyl)-5-methyl-3-pyridyl]acetamide

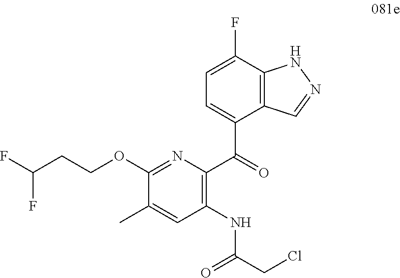 [SMILES: Cc1cc(NC(=O)CCl)c(C(=O)c2ccc(F)c3[nH]ncc23)nc1OCCC(F)F]

The title compound was prepared in analogy to the preparation of compound 001c, replacing compound 001b with compound 081d. Compound 081e (27.0 mg, crude) was obtained as a black solid. MS (ESI⁺) obsd. [(M+H)⁺]: 441.2.

### Step 6: 6-(3,3-Difluoropropoxy)-4-(7-fluoro-1H-indazol-4-yl)-7-methyl-3-pyridin-1-ium-1-yl-1H-1,5-naphthyridin-2-one;chloride

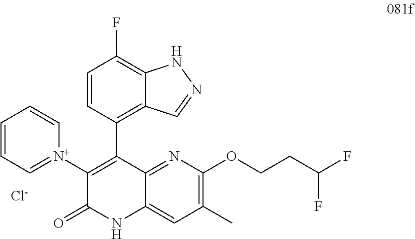 [SMILES: Cc1cc2[nH]c(=O)c(-[n+]3ccccc3)c(-c3ccc(F)c4[nH]ncc34)c2nc1OCCC(F)F ; [Cl-]]

The title compound was prepared in analogy to the preparation of compound 001d, replacing compound 001c with compound 081e. Compound 081f (30 mg) was obtained as a brown solid. MS (ESI⁺) obsd. [(M-Cl)⁺]: 466.3.

### Step 7: 3-Amino-6-(3,3-difluoropropoxy)-4-(7-fluoro-1H-indazol-4-yl)-7-methyl-1H-1,5-naphthyridin-2-one

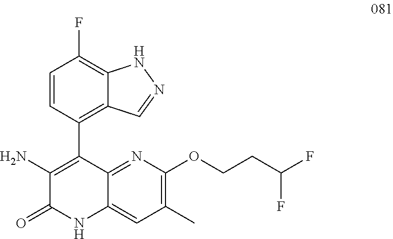 [SMILES: Cc1cc2[nH]c(=O)c(N)c(-c3ccc(F)c4[nH]ncc34)c2nc1OCCC(F)F]

The title compound was prepared in analogy to the preparation of Example 001 in step 5, replacing compound 001d with compound 081f. Example 081 (4.9 mg) was obtained as a yellow solid. MS (ESI⁺) obsd. [(M+H)⁺]: 404.0.

Example 081: ¹H NMR (400 MHz, DMSO-d₆) δ ppm 13.58 (s, 1H), 12.01 (s, 1H), 7.64 (s, 1H), 7.41 (s, 1H), 7.27 (dd, J=8.0, 10.8 Hz, 1H), 7.07 (dd, J=4.0, 8.0 Hz, 1H), 6.07-5.66 (m, 1H), 5.19 (s, 2H), 3.94-3.72 (m, 2H), 2.14 (s, 3H), 1.99-1.88 (m, 2H). ¹⁹F NMR (400 MHz, DMSO-d₆) δ ppm −115.78 (s, 2F), −132.87 (s, 1F).

### Example 082: 3-Amino-4-(7-fluoro-1H-indazol-4-yl)-7-methyl-6-(1-methyl-4-piperidyl)-1H-1,5-naphthyridin-2-one

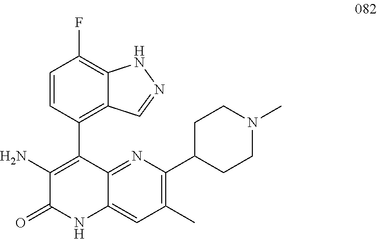 [SMILES: Cc1cc2[nH]c(=O)c(N)c(-c3ccc(F)c4[nH]ncc34)c2nc1C1CCN(C)CC1]

### Step 1: 3-Amino-4-(7-fluoro-1H-indazol-4-yl)-7-methyl-6-(1-methyl-3,6-dihydro-2H-pyridin-4-yl)-1H-1,5-naphthyridin-2-one

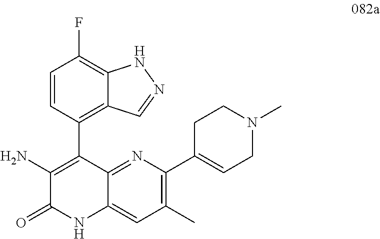 [SMILES: Cc1cc2[nH]c(=O)c(N)c(-c3ccc(F)c4[nH]ncc34)c2nc1C1=CCN(C)CC1]

A mixture of 1-methyl-4-(4,4,5,5-tetramethyl-1,3,2-dioxaborolan-2-yl)-3,6-dihydro-2H-pyridine (43.1 mg, 0.19 mmol), K₂CO₃ (35.6 mg, 0.26 mmol), Example 021 (50.0 mg, 0.13 mmol) and Pd(dppf)Cl₂ (9.4 mg, 0.01 mmol) in 1,4-dioxane (1 mL) and water (0.30 mL) was stirred at 80° C. for 2 hours under N₂ protection. After completion, the reaction mixture was evaporated in vacuo to give a residue, which was purified by prep-HPLC (NH₄HCO₃ as additive) and lyophilized to give compound 082a (35.0 mg, 67.2% yield) as a yellow solid. MS (ESI⁺) obsd. [(M+H)⁺]: 405.4.

### Step 2: 3-Amino-4-(7-fluoro-1H-indazol-4-yl)-7-methyl-6-(1-methyl-4-piperidyl)-1H-1,5-naphthyridin-2-one

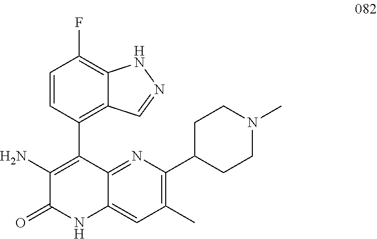 [SMILES: Cc1cc2[nH]c(=O)c(N)c(-c3ccc(F)c4[nH]ncc34)c2nc1C1CCN(C)CC1]

A mixture of compound 082a (25.0 mg, 0.06 mmol) and Pd/C (30.0 mg, 10% weight on carbon) in ethyl acetate (1 mL) was stirred under H₂ (15 psi) at 25° C. for 12 hours. After completion, the suspension was filtered and the filtrate was concentrated in vacuo to give a residue, which was purified by prep-HPLC (NH₄HCO₃ as additive) and lyophilized to give Example 082 (10.4 mg, 39.8% yield) as a white solid. MS (ESI⁺) obsd. [(M+H)⁺]: 407.2.

Example 082: ¹H NMR (400 MHz, DMSO-d₆) δ ppm 13.57 (s, 1H), 11.99 (s, 1H), 7.55 (d, J=3.2 Hz, 1H), 7.32 (s, 1H), 7.28 (dd, J=7.6, 11.2 Hz, 1H), 7.06 (dd, J=4.4, 8.0 Hz, 1H), 5.13 (s, 2H), 2.64-2.58 (m, 2H), 2.30 (s, 3H), 2.03 (s, 3H), 1.86-1.75 (m, 2H), 1.40-1.21 (m, 5H). ¹⁹F NMR (400 MHz, DMSO-d₆) δ ppm −132.95 (s, 1F).

### Example 083: 7-Amino-8-(7-fluoro-1H-indazol-4-yl)-2,3-dimethyl-5H-pyrido[2,3-b]pyrazin-6-one

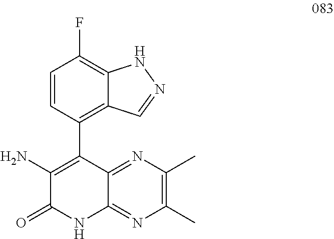 [SMILES: Cc1nc2[nH]c(=O)c(N)c(-c3ccc(F)c4[nH]ncc34)c2nc1C]

### Step 1: 3-Amino-N-methoxy-N,5,6-trimethylpyrazine-2-carboxamide

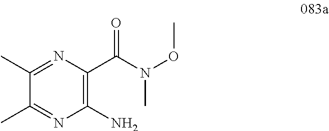

To a solution of N,O-dimethylhydroxylamine hydrochloride (2.5 g, 25.4 mmol) in THE (30 mL) was added LiHMDS (50.8 mL, 50.8 mmol) dropwise at 0° C. Half hour later, methyl 3-amino-5,6-dimethylpyrazine-2-carboxylate (2.3 g, 12.7 mmol) in THE (20 mL) was added dropwise to the above mixture at 0° C. And then, the mixture was warmed up to 20° C. and stirred for 0.5 hour. After completion, the mixture was quenched by slowly adding saturated ammonium chloride (30 mL) and extracted with EtOAc (15 mL×3). The combined organic layer was washed with brine (30 mL), dried over anhydrous sodium sulfate and concentrated under reduced pressure to give a residue, which was purified by silica-gel chromatography (elute with EtOAc:PE=0-50%) to give compound 083a (1.07 g, 40.1% yield) as an off-white solid. MS (ESI⁺) obsd. [(M+H)⁺]: 211.2.

### Step 2: (3-Amino-5,6-dimethylpyrazin-2-yl)(7-fluoro-1-(tetrahydro-2H-pyran-2-yl)-1H-indazol-4-yl)methanone

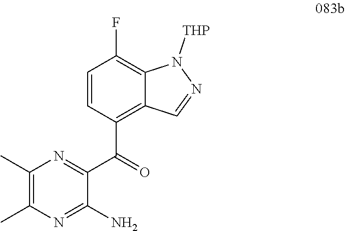

To a solution of 4-bromo-7-fluoro-1-(tetrahydro-2H-pyran-2-yl)-1H-indazole (1.8 g, 6.02 mmol) in THE (20 mL) was added n-BuLi (2.89 mL, 7.22 mmol) dropwise at −78° C. under N₂ atmosphere. And the mixture was stirred at −78° C. for 0.5 hour. And then, a solution of compound 083a (1.01 g, 4.8 mmol) in THE (10 mL) was added dropwise to the above mixture at −78° C., and stirred at −78° C. for another 0.5 hour. After completion, the reaction mixture was quenched with saturated ammonium chloride (30 mL) and extracted with EtOAc (15 mL×3). The combined organic layer was washed with brine (40 mL), dried over anhydrous sodium sulfate and concentrated under reduced pressure to give a residue, which was purified by silica-gel chromatography (elute with EtOAc:PE=30-50%) to give compound 083b (145.0 mg, 6.5% yield) as a yellow solid. MS (ESI⁺) obsd. [(M+H)⁺]: 370.2.

### Step 3: 2-Chloro-N-(3-(7-fluoro-1-(tetrahydro-2H-pyran-2-yl)-1H-indazole-4-carbonyl)-5,6-dimethylpyrazin-2-yl)acetamide

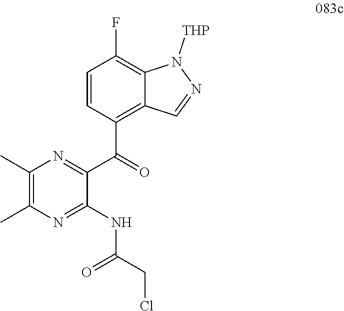

To a solution of compound 083b (50.0 mg, 0.14 mmol) and TEA (0.08 mL, 0.54 mmol) in chloroform (1.5 mL) was added dropwise chloroacetyl chloride (0.02 mL, 0.27 mmol) at 0° C. Then the mixture was stirred at 20° C. for 1 hour. After completion, the mixture was concentrated in vacuo to give compound 083c (50.0 mg, crude) as a light grey solid, which was used in the next step directly. MS (ESI⁺) obsd. [(M-THP+H)⁺]: 362.0.

### Step 4: 8-(7-Fluoro-1-tetrahydropyran-2-yl-indazol-4-yl)-2,3-dimethyl-7-pyridin-1-ium-1-yl-5H-pyrido[2,3-b]pyrazin-6-one;chloride

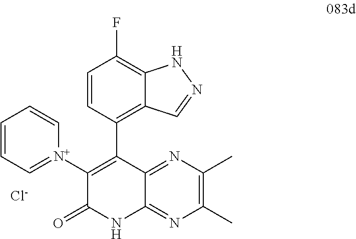 [SMILES: Cc1nc2[nH]c(=O)c(-[n+]3ccccc3)c(-c3ccc(F)c4[nH]ncc34)c2nc1C ; [Cl-]]

A solution of compound 083c (50.0 mg, 0.11 mmol) in pyridine (2.5 mL) was stirred at 70° C. for 12 hours. The mixture was concentrated under reduced pressure to give compound 083d (40.0 mg, 70.4% yield) as a black solid, which was used in the next step directly. MS (ESI⁺) obsd. [(M-Cl)⁺]:471.4.

### Step 5: 7-Amino-8-(7-fluoro-1-(tetrahydro-2H-pyran-2-yl)-1H-indazol-4-yl)-2,3-dimethylpyrido[2,3-b]pyrazin-6(5H)-one

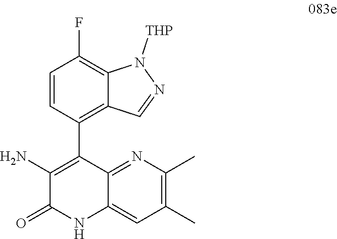

A solution of compound 083d (90.0 mg, 0.18 mmol) and hydrazine hydrate (0.5 mL, 85% purity) in ethanol (1 mL) was stirred at 50° C. for 12 hours. After completion, the reaction mixture was poured into water (4 mL) and extracted with EtOAc (3 mL×3). The combined organic layer was washed with brine (10 mL), dried over anhydrous sodium sulfate and concentrated under reduced pressure to give a residue, which was purified by prep-HPLC (NH₄HCO₃ as additive) and lyophilized to give compound 083e (30.0 mg, 41.4% yield) as a light brown solid. MS (ESI⁺) obsd. [(M+H)⁺]:409.3.

### Step 6: 7-Amino-8-(7-fluoro-1H-indazol-4-yl)-2,3-dimethyl-5H-pyrido[2,3-b]pyrazin-6-one

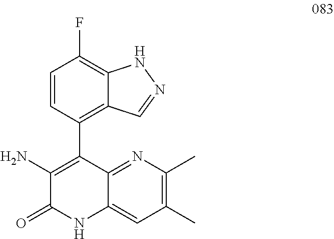 [SMILES: Cc1cc2[nH]c(=O)c(N)c(-c3ccc(F)c4[nH]ncc34)c2nc1C]

A solution of compound 083e (30.0 mg, 0.07 mmol) in HCl/EA (2.0 mL, 4M) was stirred at 25° C. for 1 hour. After completion, the mixture was concentrated under reduced pressure to give a residue, which was purified by prep-HPLC (NH₄HCO₃ as additive) and lyophilized to give Example 083 (2.1 mg, 8.2% yield) as a white solid. MS (ESI⁺) obsd. [(M+H)⁺]: 325.0.

Example 083: ¹H NMR (400 MHz, DMSO-d₆) δ ppm 13.63 (s, 1H), 7.66 (d, J=0.8 Hz, 1H), 7.29 (dd, J=7.6, 10.8 Hz, 1H), 7.02 (dd, J=4.0, 7.6 Hz, 1H), 5.38 (s, 2H), 2.44 (s, 3H), 2.23 (s, 3H). ¹⁹F NMR (376 MHz, DMSO-d₆) δ ppm −132.60 (s, 1F).

### Example 084: 7-Amino-8-(7-fluoro-1H-indazol-4-yl)-2,4-dimethyl-5H-pyrido[3,2-d]pyrimidin-6-one

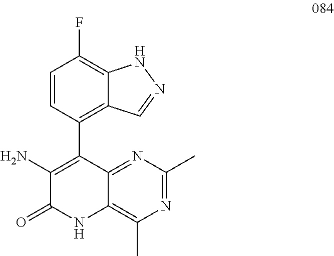 [SMILES: Cc1nc(C)c2[nH]c(=O)c(N)c(-c3ccc(F)c4[nH]ncc34)c2n1]

The title compound was prepared in analogy to the preparation of Example 083, replacing methyl 3-amino-5,6-dimethylpyrazine-2-carboxylate with methyl 5-amino-2,6-dimethyl-pyrimidine-4-carboxylate in step 1. Example 084 (4.9 mg) was obtained as a yellow solid. MS (ESI⁺) obsd. [(M+H)⁺]: 325.2.

Example 084: ¹H NMR (400 MHz, DMSO-d₆) δ ppm 13.62 (s, 1H), 11.70 (s, 1H), 7.66 (d, J=2.4 Hz, 1H), 7.28 (dd, J=11.2, 7.6 Hz, 1H), 6.99 (dd, J=7.6, 4.0 Hz, 1H), 5.82 (s, 2H), 2.60 (s, 3H), 2.26 (s, 3H). ¹⁹F NMR (376 MHz, DMSO-d₆) δ ppm −132.69 (s, 1F).

### Example 085: 3-Amino-7-cyclopropyl-4-(7-fluoro-1H-indazol-4-yl)-6-methyl-1H-1,5-naphthyridin-2-one

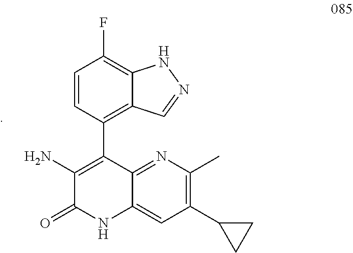 [SMILES: Cc1nc2c(-c3ccc(F)c4[nH]ncc34)c(N)c(=O)[nH]c2cc1C1CC1]

### Step 1: (3-Amino-6-bromo-5-cyclopropylpyridin-2-yl)(7-fluoro-1H-indazol-4-yl)methanone

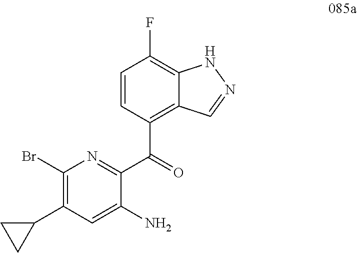 [SMILES: Nc1cc(C2CC2)c(Br)nc1C(=O)c1ccc(F)c2[nH]ncc12]

A mixture of compound 015b (340.0 mg, 1.2 mmol) and NBS (214.0 mg, 1.2 mmol) in DMF (3 mL) was stirred at 25° C. for 1 hour. After completion, the reaction mixture was quenched with saturated sodium sulfite (20 mL) and extracted with ethyl acetate (20 mL×3). The combined organic layer was washed with brine (40 mL), dried over anhydrous sodium sulfate and concentrated under reduced pressure to give a residue, which was purified by prep-HPLC (NH₄HCO₃ as additive) and lyophilized to give compound 085a (210.0 mg, 48.8% yield) as a yellow solid. MS (ESI⁺) obsd. [(M+H)⁺]: 375.0.

### Step 2: N-(6-bromo-5-cyclopropyl-2-(7-fluoro-1H-indazole-4-carbonyl)pyridin-3-yl)-2-chloroacetamide

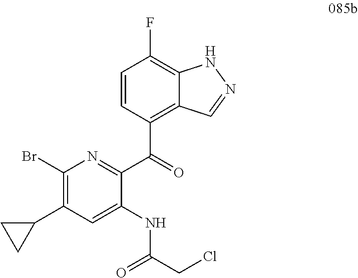 [SMILES: O=C(CCl)Nc1cc(C2CC2)c(Br)nc1C(=O)c1ccc(F)c2[nH]ncc12]

The title compound was prepared in analogy to the preparation of compound 001c, replacing compound 001b with compound 085a. Compound 085b (220.0 mg) was obtained as a brown oil. MS (ESI⁺) obsd. [(M+H)⁺]: 451.0.

### Step 3: 6-Bromo-7-cyclopropyl-4-(7-fluoro-1H-indazol-4-yl)-3-pyridin-1-ium-1-yl-1H-1,5-naphthyridin-2-one;chloride

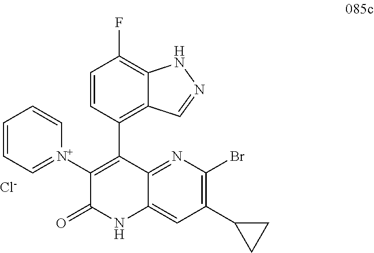 [SMILES: O=c1[nH]c2cc(C3CC3)c(Br)nc2c(-c2ccc(F)c3[nH]ncc23)c1-[n+]1ccccc1 ; [Cl-]]

The title compound was prepared in analogy to the preparation of compound 001d, replacing compound 001c with compound 085b. Compound 085c (200 mg, crude) was obtained as a brown oil. MS (ESI⁺) obsd. [(M-Cl)⁺]: 478.0.

### Step 4: 3-Amino-6-bromo-7-cyclopropyl-4-(7-fluoro-1H-indazol-4-yl)-1,5-naphthyridin-2(1H)-one

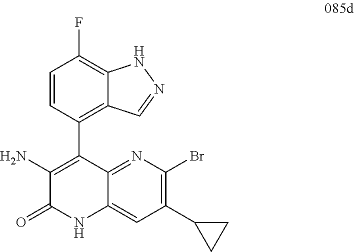 [SMILES: Nc1c(-c2ccc(F)c3[nH]ncc23)c2nc(Br)c(C3CC3)cc2[nH]c1=O]

The title compound was prepared in analogy to the preparation of Example 001 in step 5, replacing compound 001d with compound 085c. Compound 085d (200.0 mg, crude) was obtained as a brown oil. MS (ESI⁺) obsd. MS (ESI⁺) obsd. [(M+H)⁺]: 414.2.

### Step 5: 3-Amino-7-cyclopropyl-4-(7-fluoro-1H-indazol-4-yl)-6-methyl-1,5-naphthyridin-2(1H)-one

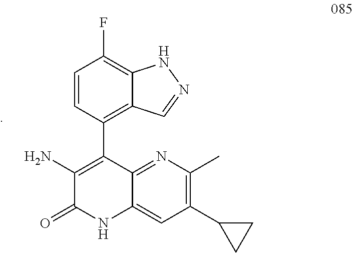 [SMILES: Cc1nc2c(-c3ccc(F)c4[nH]ncc34)c(N)c(=O)[nH]c2cc1C1CC1]

A mixture of compound 085d (60.0 mg, 0.14 mmol), trimethylboroxine (0.05 mL, 0.16 mmol), Pd(dppf)Cl₂ (10.6 mg, 0.01 mmol) and K₂CO₃ (40.04 mg, 0.29 mmol) in 1,4-dioxane (3 mL) was stirred at 100° C. for 12 hours under N₂ protection. After completion, the mixture was filtered and concentrated under reduced pressure to give a residue, which was purified by prep-HPL (NH₄HCO₃ as additive) and lyophilized to give Example 085 (28.3 mg, 54.1% yield) as a yellow solid. MS (ESI⁺) obsd. [(M+H)⁺]: 350.3.

Example 085: ¹H NMR (400 MHz, DMSO-d₆) δ ppm 13.61 (s, 1H) 11.95 (s, 1H), 7.62 (s, 1H), 7.29 (dd, J=11.2, 8.0 Hz, 1H), 7.21 (s, 1H), 7.01 (dd, J=7.6, 4.0 Hz, 1H), 4.49-5.57 (m, 2H), 2.33 (s, 3H) 1.91-1.95 (m, 1H) 0.97 (dd, J=8.4, 1.8 Hz, 2H), 0.57 (d, J=3.6 Hz, 2H). ¹⁹F NMR (377 MHz, DMSO-d₆) δ ppm −132.91 (m, 1 F).

### Example 086: 3-Amino-6-[3-(dimethylamino)propyl]-4-(7-fluoro-1H-indazol-4-yl)-7-methyl-1H-1,5-naphthyridin-2-one

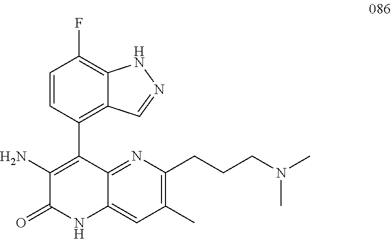 [SMILES: Cc1cc2[nH]c(=O)c(N)c(-c3ccc(F)c4[nH]ncc34)c2nc1CCCN(C)C]

A mixture of Example 075 (50.0 mg, 0.13 mmol) and Pd/C (24. mg, 10% purity) in methanol (4 mL) was stirred at rt under H₂ (15 psi) atmosphere for 0.5 hour. After completion, the mixture was filtered through a pad of celite and the filtrate was concentrated under reduced pressure to give a residue, which was purified by prep-HPLC (NH₄HCO₃ as additive) and lyophilized to give Example 086 (40.0 mg, 79.2% yield) as a yellow solid. MS (ESI⁺) obsd. [(M+H)⁺]: 395.2.

Example 086: ¹H NMR (400 MHz, DMSO-d₆) δ ppm 13.57 (br s, 1H), 11.99 (br s, 1H), 7.56 (d, J=3.4 Hz, 1H), 7.33 (s, 1H), 7.26 (d, J=19.2 Hz, 1H), 7.03 (dd, J=4.0, 8.0 Hz, 1H), 5.11 (s, 2H), 2.46-2.39 (m, 1H), 2.26 (s, 3H), 2.00-1.95 (m, 2H), 1.90 (s, 6H), 1.46-1.38 (m, 2H). ¹⁹F NMR (376 MHz, DMSO-d₆) δ ppm −133.086 (s, 1 F).

### Example 087: Methyl 7-amino-8-(7-fluoro-1H-indazol-4-yl)-3-methyl-6-oxo-5H-1,5-naphthyridine-2-carboxylate

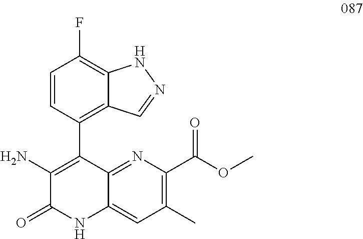 [SMILES: COC(=O)c1nc2c(-c3ccc(F)c4[nH]ncc34)c(N)c(=O)[nH]c2cc1C]

A mixture of Example 021 (100.0 mg, 0.26 mmol), Pd(dppf)Cl₂ (37.7 mg, 0.05 mmol) and TEA (0.07 mL, 0.52 mmol) in methanol (2 mL) was stirred at 80° C. for 12 hours under CO (50 psi) atmosphere. After completion, the mixture was poured into water (10 mL) and extracted with EtOAc (10 mL×3). The combined organic layer was washed with brine (20 mL), dried over anhydrous sodium sulfate and concentrated under reduced pressure to give a residue, which was purified by prep-HPLC (HCl as additive) and lyophilized to give Example 087 (47.0 mg, 44.1% yield) as a yellow solid. MS (ESI⁺) obsd. [(M+H)⁺]: 368.2.

Example 087: ¹H NMR (400 MHz, DMSO-d₆) δ ppm 12.32 (s, 1H), 7.65 (d, J=3.2 Hz, 1H), 7.49 (s, 1H), 7.29 (dd, J=8.0, 11.2 Hz, 1H), 7.03 (dd, J=4.4, 8.0 Hz, 1H), 3.65 (s, 3H), 2.41 (s, 3H). ¹⁹F NMR (376 MHz, DMSO-d₆) δ ppm −132.57 (s, 1F).

### Example 088: 3-Amino-4-(7-fluoro-1H-indazol-4-yl)-6-(1-hydroxy-1-methyl-ethyl)-7-methyl-1H-1,5-naphthyridin-2-one

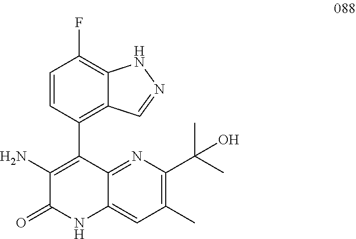 [SMILES: Cc1cc2[nH]c(=O)c(N)c(-c3ccc(F)c4[nH]ncc34)c2nc1C(C)(C)O]

The title compound was prepared in analogy to the preparation of Example 078, replacing Example 061 with Example 087. Example 088 (10.0 mg) was obtained as a yellow solid. MS (ESI⁺) obsd. [(M+H)⁺]: 368.3.

Example 089: ¹H NMR (400 MHz, MeOD-d₄) δ ppm 7.95 (s, 1H), 7.93 (d, J=3.2 Hz, 1H), 7.31 (dd, J=8.0, 10.8 Hz, 1H), 7.07 (dd, J=4.0, 7.6 Hz, 1H), 2.59 (s, 3H), 1.62 (s, 3H), 1.53 (s, 3H). ¹⁹F NMR (376 MHz, MeOD-d₄) δ ppm −130.91 (s, 1F).

### Example 089: 7-Amino-8-(7-fluoro-1H-indazol-4-yl)-3-methyl-6-oxo-5H-1,5-naphthyridine-2-carboxylic acid

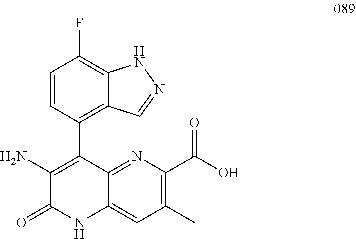 [SMILES: Cc1cc2[nH]c(=O)c(N)c(-c3ccc(F)c4[nH]ncc34)c2nc1C(=O)O]

A mixture of Example 087 (6.0 mg, 0.01 mmol) and NaOH (0.5 mL, 2.0 mmol) in methanol (1 mL) and water (1 ml) was stirred at 25° C. for 1 hour. After completion, the mixture was acidified with HCl (1 M) to 3 (PH value). Then the mixture was extracted with EtOAc (10 mL×3). The combined organic layer was washed with brine (20 mL), dried over anhydrous sodium sulfate and concentrated under reduced pressure to give a residue, which was purified by prep-HPLC (HCl as additive) and lyophilized to give Example 089 (5.2 mg, 91.2% yield) as a yellow solid. MS (ESI⁺) obsd. [(M+H)⁺]: 354.2.

Example 089: ¹H NMR (400 MHz, DMSO-d₆) δ ppm 12.39 (s, 1H), 7.68 (d, J=3.2 Hz, 1H), 7.52 (s, 1H), 7.31 (dd, J=8.0, 11.2 Hz, 1H), 7.07 (dd, J=4.0, 8.0 Hz, 1H), 2.48 (s, 3H). ¹⁹F NMR (376 MHz, DMSO-d₆) δ ppm −132.24 (s, 1F).

### Example 090: 3-Amino-8-chloro-4-(7-fluoro-1H-indazol-4-yl)-6-methyl-1H-1,5-naphthyridin-2-one

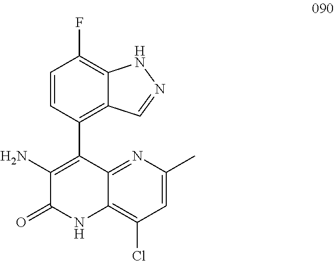 [SMILES: Cc1cc(Cl)c2[nH]c(=O)c(N)c(-c3ccc(F)c4[nH]ncc34)c2n1]

### Step 1: (3-Amino-4-chloropyridin-2-yl)(7-fluoro-1-tosyl-1H-indazol-4-yl)methanone

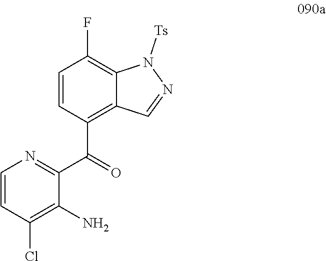 [SMILES: Cc1ccc(S(=O)(=O)n2ncc3c(C(=O)c4nccc(Cl)c4N)ccc(F)c32)cc1]

To a solution of (3-amino-4-chloro-2-pyridyl)-(7-fluoro-1H-indazol-4-yl)methanone (150.0 mg, 0.52 mmol) in THE (10 mL) was added NaH (22.7 mg, 0.57 mmol) at 0° C. After stirred at 0° C. for 30 minutes, a solution of p-toluenesulfonyl chloride (103.3 mg, 0.54 mmol) in THE (2 mL) was added dropwise to the mixture at 0° C. And then, the mixture was warmed up to 25° C. and stirred for another 1 hour. After completion, the reaction mixture was quenched with saturated sodium bicarbonate (10 mL) and extracted with EtOAc (10 mL×3). The combined organic layer was washed with brine (10 mL), dried over anhydrous sodium sulfate and concentrated under reduced pressure to give a residue, which was purified by silica-gel column chromatography (elute with EtOAc:PE=0-50%) to give compound 090a (150.0 mg, 65.34% yield) as a yellow solid. MS (ESI⁺) obsd. [(M+H)⁺]: 445.1.

### Step 2: (3-Amino-6-bromo-4-chloropyridin-2-yl)(7-fluoro-1-tosyl-1H-indazol-4-yl)methanone

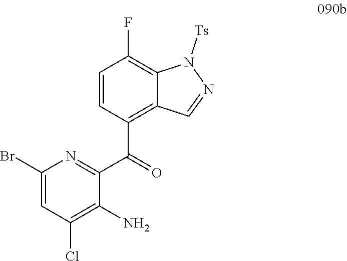 [SMILES: Cc1ccc(S(=O)(=O)n2ncc3c(C(=O)c4nc(Br)cc(Cl)c4N)ccc(F)c32)cc1]

A mixture of compound 090a (140.0 mg, 0.31 mmol) and NBS (67.21 mg, 0.38 mmol) in DMF (1 mL) was stirred at 50° C. for 4 hours. After completion, the reaction mixture was concentrated under reduced pressure to give a residue, which was purified by silica-gel chromatography (elute with EtOAc:PE=0-80%) to give compound 090b (160.0 mg, 97.8% yield) as a yellow solid. MS (ESI⁺) obsd. [(M+H)⁺]: 523.2.

### Step 3: (3-Amino-4-chloro-6-methylpyridin-2-yl)(7-fluoro-1-tosyl-1H-indazol-4-yl)methanone

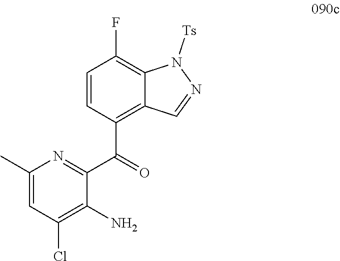 [SMILES: Cc1ccc(S(=O)(=O)n2ncc3c(C(=O)c4nc(C)cc(Cl)c4N)ccc(F)c32)cc1]

A mixture of compound 090b (145.0 mg, 0.28 mmol), trimethylboroxine (0.12 mL, 0.42 mmol), Pd(dppf)Cl₂ (20.26 mg, 0.03 mmol) and K₂CO₃ (76.52 mg, 0.55 mmol) in 1,4-dioxane (10 mL) was stirred at 80° C. for 12 hours. After completion, the suspension was filtered and the filtrate was concentrated under reduced pressure to give a residue, which was purified by silica-gel chromatography (elute with EtOAc:PE=0-80%) to give compound 090c (120.0 mg, 94.5% yield) as a yellow solid. MS (ESI⁺) obsd. [(M+H)⁺]: 459.2

### Step 4: 2-Chloro-N-(4-chloro-2-(7-fluoro-1-tosyl-1H-indazole-4-carbonyl)-6-methylpyridin-3-yl)acetamide

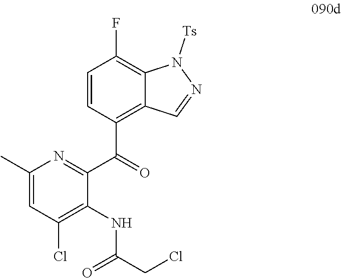 [SMILES: Cc1ccc(S(=O)(=O)n2ncc3c(C(=O)c4nc(C)cc(Cl)c4NC(=O)CCl)ccc(F)c32)cc1]

The title compound was prepared in analogy to the preparation of compound 001c, replacing compound 001b with compound 090c. Compound 090d (110 mg, crude) was obtained as a black solid. MS (ESI⁺) obsd. [(M+H)⁺]: 535.1.

### Step 5: 8-Chloro-4-[7-fluoro-1-(p-tolylsulfonyl)indazol-4-yl]-6-methyl-3-pyridin-1-ium-1-yl-1H-1,5-naphthyridin-2-one;chloride

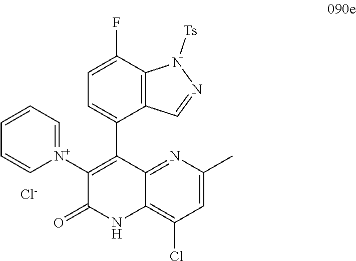 [SMILES: Cc1ccc(S(=O)(=O)n2ncc3c(-c4c(-[n+]5ccccc5)c(=O)[nH]c5c(Cl)cc(C)nc45)ccc(F)c32)cc1 ; [Cl-]]

The title compound was prepared in analogy to the preparation of compound 001d, replacing compound 001c with compound 090d. Compound 090e (120 mg, crude) was obtained as a black solid. MS (ESI⁺) obsd. [(M-Cl)⁺]: 560.1.

### Step 6: 3-Amino-8-chloro-4-(7-fluoro-1H-indazol-4-yl)-6-methyl-1H-1,5-naphthyridin-2-one

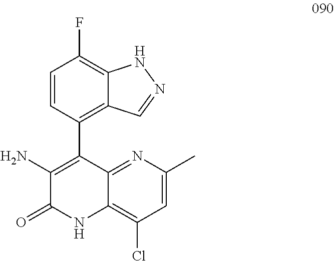 [SMILES: Cc1cc(Cl)c2[nH]c(=O)c(N)c(-c3ccc(F)c4[nH]ncc34)c2n1]

A mixture of compound 090e (120.0 mg, 0.2 mmol) and hydrazine monohydrate (1.0 mL) in ethanol (1 mL) was stirred at 50° C. for 1 hour. After completion, the reaction mixture was concentrated under reduced pressure to give a residue, which was purified by prep-HPLC (NH₄HCO₃ as additive) and lyophilized to give Example 090 (27.0 mg, 37.8% yield) as a brown solid. MS (ESI⁺) obsd. [(M+H)⁺]: 344.2.

Example 090: ¹H NMR (400 MHz, DMSO-d₆) δ ppm 13.60 (br s, 1H), 11.61 (br s, 1H), 7.64 (s, 1H), 7.28 (dd, J=8.0, 10.8 Hz, 1H), 7.22 (s, 1H), 7.00 (dd, J=4.0, 7.6 Hz, 1H), 5.43 (s, 2H), 2.19 (s, 3H). ¹⁹F NMR (400 MHz, DMSO-d₆) δ ppm −132.76 (s, 1F).

### Example 091: 3-Amino-6-chloro-4-(7-fluoro-1H-indazol-4-yl)-8-methyl-1H-1,5-naphthyridin-2-one

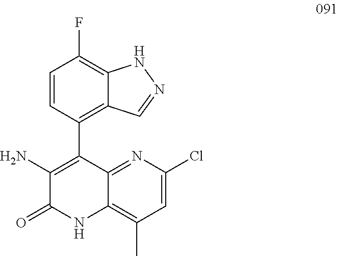 [SMILES: Cc1cc(Cl)nc2c(-c3ccc(F)c4[nH]ncc34)c(N)c(=O)[nH]c12]

### Step 1: (3-Amino-4-methylpyridin-2-yl)(7-fluoro-1H-indazol-4-yl)methanone

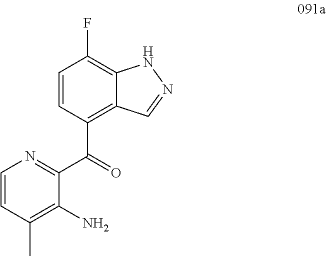 [SMILES: Cc1ccnc(C(=O)c2ccc(F)c3[nH]ncc23)c1N]

A solution of compound Int-21 (400.0 mg, 0.88 mmol) in HCl/EA (6.0 mL, 4 M) was stirred at 25° C. for 1 hour. After completion, the reaction mixture was concentrated under reduced pressure to give compound 091a (210.0 mg, 88.0% yield) as a yellow solid. MS (ESI⁺) obsd. [(M+H)⁺]: 271.2.

### Step 2: (3-Amino-4-methylpyridin-2-yl)(7-fluoro-1-tosyl-1H-indazol-4-yl)methanone

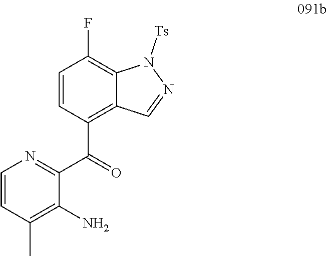 [SMILES: Cc1ccc(S(=O)(=O)n2ncc3c(C(=O)c4nccc(C)c4N)ccc(F)c32)cc1]

The title compound was prepared in analogy to the preparation of compound 090a, replacing (3-amino-4-chloro-2-pyridyl)-(7-fluoro-1H-indazol-4-yl)methanone with compound 091a. Compound 091b (100.0 mg) was obtained as a yellow solid. MS (ESI⁺) obsd. [(M+H)⁺]: 425.2.

### Step 3: (3-Amino-6-chloro-4-methylpyridin-2-yl)(7-fluoro-1-tosyl-1H-indazol-4-yl)methanone

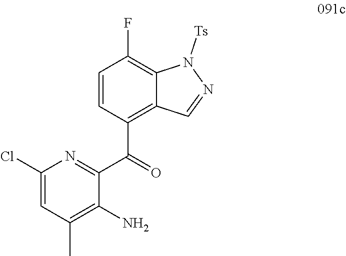 [SMILES: Cc1ccc(S(=O)(=O)n2ncc3c(C(=O)c4nc(Cl)cc(C)c4N)ccc(F)c32)cc1]

A mixture of compound 091b (50.0 mg, 0.12 mmol) and NCS (17.3 mg, 0.13 mmol) in DMF (1 mL) was stirred at 80° C. for 0.5 hour. After completion, the reaction mixture was quenched with saturated sodium sulfite (5 mL) and extracted with EtOAc (5 mL×3). The combined organic layer was washed with brine (6 mL), dried over anhydrous sodium sulfate and concentrated under reduced pressure to give a residue, which was purified by prep-HPLC (NH₄HCO₃ as additive) and lyophilized to give compound 091c (20.0 mg, 37.0% yield) as a yellow solid. MS (ESI⁺) obsd. [(M+H)⁺]: 459.3.

### Step 4: 2-Chloro-N-(6-chloro-2-(7-fluoro-1-tosyl-1H-indazole-4-carbonyl)-4-methylpyridin-3-yl)acetamide

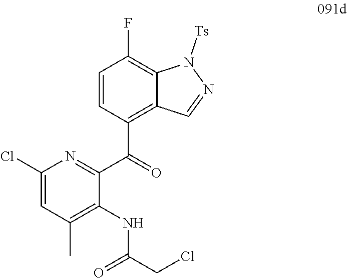 [SMILES: Cc1ccc(S(=O)(=O)n2ncc3c(C(=O)c4nc(Cl)cc(C)c4NC(=O)CCl)ccc(F)c32)cc1]

The title compound was prepared in analogy to the preparation of compound 001c, replacing compound 001b with compound 091c. Compound 091d (15.0 mg, crude) was obtained as a brown solid. MS (ESI⁺) obsd. [(M+H)⁺]: 535.2.

### Step 5: 6-Chloro-4-[7-fluoro-1-(p-tolylsulfonyl)indazol-4-yl]-8-methyl-3-pyridin-1-ium-1-yl-1H-1,5-naphthyridin-2-one;chlorid

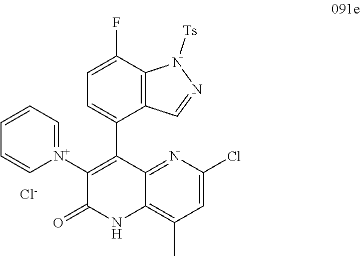 [SMILES: Cc1ccc(S(=O)(=O)n2ncc3c(-c4c(-[n+]5ccccc5)c(=O)[nH]c5c(C)cc(Cl)nc45)ccc(F)c32)cc1 ; [Cl-]]

The title compound was prepared in analogy to the preparation of compound 001d, replacing compound 001c with compound 091d. Compound 091e (16.0 mg, crude) was obtained as a brown solid. MS (ESI⁺) obsd. [(M-Cl)⁺]: 560.2.

### Step 6: 3-Amino-6-chloro-4-(7-fluoro-1H-indazol-4-yl)-8-methyl-1H-1,5-naphthyridin-2-one

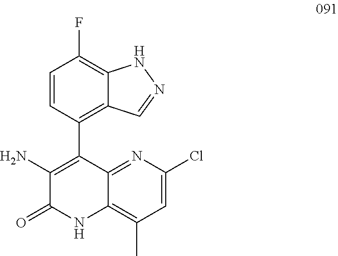 [SMILES: Cc1cc(Cl)nc2c(-c3ccc(F)c4[nH]ncc34)c(N)c(=O)[nH]c12]

The title compound was prepared in analogy to the preparation of Example 090, replacing compound 090e with compound 091e in step 6. Example 091 (6.4 mg) was obtained as a yellow solid. MS (ESI⁺) obsd. [(M+H)⁺]: 344.1.

Example 091: ¹H NMR (400 MHz, DMSO-d₆) δ ppm 13.64 (s, 1H), 11.63 (br s, 1H), 7.66 (d, J=1.2 Hz, 1H), 7.26-7.33 (m, 1H), 7.11 (s, 1H), 6.99 (dd, J=7.6, 4.4 Hz, 1H) 5.47 (s, 2H) 2.48 (s, 3H). ¹⁹F NMR (377 MHz, DMSO-d₆) δ ppm −132.57 (s, 1 F).

### Example 092: 7-Amino-8-(7-fluoro-1H-indazol-4-yl)-3-methyl-6-oxo-5H-1,5-naphthyridine-2-carbonitrile

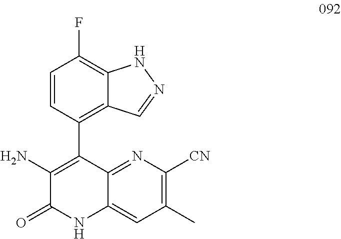 [SMILES: Cc1cc2[nH]c(=O)c(N)c(-c3ccc(F)c4[nH]ncc34)c2nc1C#N]

### Step 1: (3-Amino-6-bromo-5-methyl-2-pyridyl)-[7-fluoro-1-(p-tolylsulfonyl)indazol-4-yl]methanone

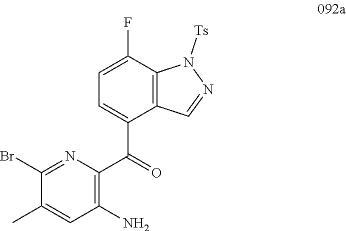 [SMILES: Cc1ccc(S(=O)(=O)n2ncc3c(C(=O)c4nc(Br)c(C)cc4N)ccc(F)c32)cc1]

The title compound was prepared in analogy to the preparation of compound 090a, replacing (3-amino-4-chloro-2-pyridyl)-(7-fluoro-1H-indazol-4-yl)methanone with compound 021c. Compound 092a (500.0 mg) was obtained as a yellow solid. MS (ESI⁺) obsd. [(M+H)⁺]: 503.2.

### Step 2: 5-Amino-6-[7-fluoro-1-(p-tolylsulfonyl)indazole-4-carbonyl]-3-methyl-pyridine-2-carbonitrile

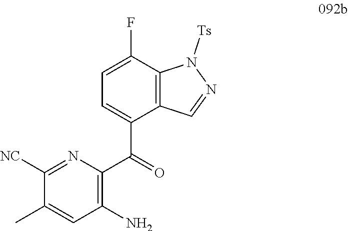 [SMILES: Cc1ccc(S(=O)(=O)n2ncc3c(C(=O)c4nc(C#N)c(C)cc4N)ccc(F)c32)cc1]

A mixture of compound 092a (500.0 mg, 0.99 mmol), DPPF (110.1 mg, 0.2 mmol), Zn(CN)₂ (350.0 mg, 3.0 mmol), Pd(PPh₃)₄ (229.6 mg, 0.2 mmol) and Zn (130.0 mg, 2.0 mmol) in DMA (10 mL) was stirred at 100° C. for 2 hours under N₂ protection. After completion, the reaction mixture was quenched with saturated sodium sulfite (20 mL) and extracted with ethyl acetate (20 mL×3). The combined organic layer was washed with brine (40 mL), dried over anhydrous sodium sulfate and concentrated under reduced pressure to give a residue, which was purified by silica-gel chromatography (elute with EtOAc:PE=0-100%) to give compound 092b (100.0 mg, 22.4% yield) as a yellow solid. MS (ESI⁺) obsd. [(M+H)⁺]: 450.1.

### Step 3: 2-Chloro-N-[6-cyano-2-[7-fluoro-1-(p-tolylsulfonyl)indazole-4-carbonyl]-5-methyl-3-pyridyl]acetamide

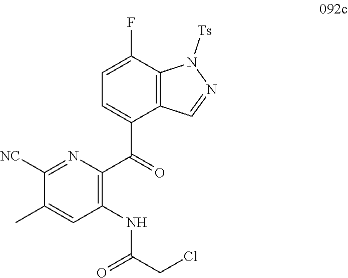 [SMILES: Cc1ccc(S(=O)(=O)n2ncc3c(C(=O)c4nc(C#N)c(C)cc4NC(=O)CCl)ccc(F)c32)cc1]

The title compound was prepared in analogy to the preparation of compound 001c, replacing compound 001b with compound 092b. Compound 092c (110.0 mg, crude) was obtained as a yellow solid. MS (ESI⁺) obsd. [(M+H)⁺]: 526.3.

### Step 4: 8-(7-Fluoro-1H-indazol-4-yl)-3-methyl-6-oxo-7-pyridin-1-ium-1-yl-5H-1,5-naphthyridine-2-carbonitrile;chloride

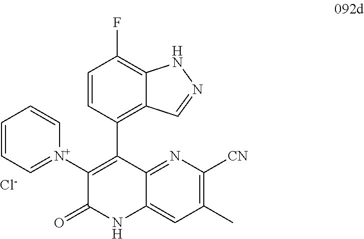 [SMILES: Cc1cc2[nH]c(=O)c(-[n+]3ccccc3)c(-c3ccc(F)c4[nH]ncc34)c2nc1C#N ; [Cl-]]

A mixture of compound 092c (110.0 mg, 0.21 mmol) in pyridine (3.0 mL) was stirred at 50° C. for 12 hours. After completion, the reaction mixture was concentrated under reduced pressure to give compound 092d (90.0 mg, crude) as a brown solid. MS (ESI⁺) obsd. [(M-Cl)⁺]: 397.2.

### Step 5: 7-Amino-8-(7-fluoro-1H-indazol-4-yl)-3-methyl-6-oxo-5H-1,5-naphthyridine-2-carbonitrile

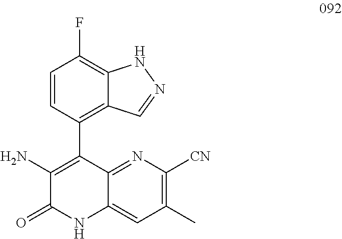 [SMILES: Cc1cc2[nH]c(=O)c(N)c(-c3ccc(F)c4[nH]ncc34)c2nc1C#N]

A mixture of compound 092d (90.0 mg, 0.21 mmol) and hydrazine monohydrate (0.1 mL, 98% purity) in ethanol (1 mL) was stirred at 50° C. for 1 hour. After completion, the mixture was poured into water (15 mL) and extracted with ethyl acetate (10 mL×3). The combined organic layer was washed with brine (30 mL), dried over anhydrous sodium sulfate and concentrated under reduced pressure to give a residue, which was purified by prep-HPLC (HCl as additive) and lyophilized to give Example 092 (11.2 mg, 13.4% yield) as a yellow solid. MS (ESI⁺) obsd. [(M+H)⁺]: 335.0.

Example 092: ¹H NMR (400 MHz, DMSO-d₆) δ ppm 12.52 (s, 1H), 7.67 (d, J=3.2 Hz, 1H), 7.56 (s, 1H), 7.31 (dd, J=7.6, 10.8 Hz, 1H), 7.01 (dd, J=4.0, 7.6 Hz, 1H), 2.46 (s, 3H). ¹⁹F NMR (376 MHz, DMSO-d₆) δ ppm −132.20 (s, 1F).

### Example 093: 3-Amino-4-(7-fluoro-1H-indazol-4-yl)-7-methyl-6-(oxetan-3-yl)-1H-1,5-naphthyridin-2-one

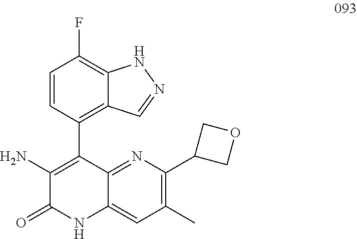 [SMILES: Cc1cc2[nH]c(=O)c(N)c(-c3ccc(F)c4[nH]ncc34)c2nc1C1COC1]

To a solution of pyridine-2,6-bis(carboximidamide);hydrogen chloride (257.1 mg, 1.3 mmol), NiCl₂ (66.8 mg, 0.52 mmol), NaI (193.1 mg, 1.3 mmol), 3-bromooxetane (176.4 mg, 1.3 mmol), Example 021 (100.0 mg, 0.26 mmol), Zn (168.4 mg, 2.6 mmol) in DMA (3 mL) was added TFA (25.0 mg, 0.26 mmol) at 25° C. And then, the mixture was stirred at 60° C. for 16 hours. After completion, the mixture was poured into water (40 mL) and extracted with EtOAc (40 mL×3). The combined organic layer was washed with brine (80 mL), dried over anhydrous sodium sulfate and concentrated under reduced pressure to give a residue, which was purified by prep-HPLC (NH₄HCO₃ as additive) and lyophilized to give Example 093 (2.7 mg, 2.6% yield) as a white solid. MS (ESI⁺) obsd. [(M+H)⁺]: 366.1.

Example 093: ¹H NMR (400 MHz, DMSO-d₆) δ ppm 13.57 (m, 1H), 12.08 (s, 1H), 7.62 (s, 1H), 7.37 (s, 1H), 7.30 (dd, J=8.0, 11.2 Hz, 1H), 7.10 (dd, J=4.4, 8.0 Hz, 1H), 5.23 (s, 2H), 4.61-4.55 (m, 2H), 4.49-4.47 (m, 1H), 4.45-4.42 (m, 1H), 4.41-4.35 (m, 1H), 2.10 (s, 3H). ¹⁹F NMR (376 MHz, DMSO-d₆) δ ppm −132.88 (s, 1F).

### Example 094: 3-Amino-4-(7-fluoro-1H-indazol-4-yl)-6,8-dimethyl-1H-1,5-naphthyridin-2-one

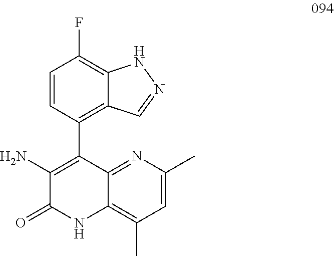 [SMILES: Cc1cc(C)c2[nH]c(=O)c(N)c(-c3ccc(F)c4[nH]ncc34)c2n1]

A mixture of Example 090 (15.0 mg, 0.04 mmol), trimethylboroxine (0.02 mL, 0.07 mmol), Pd(dppf)Cl₂ (3.19 mg) and K₂CO₃ (12.06 mg, 0.09 mmol) in 1,4-dioxane (2 mL) was stirred at 100° C. for 2 hours under N₂ protection. After completion, the suspension was filtered and the filtrate was concentrated in vacuo to give a residue, which was purified by prep-HPLC (NH₄HCO₃ as additive) and lyophilized to give Example 094 (3.0 mg, 14.7% yield) as a yellow solid. MS (ESI⁺) obsd. [(M+H)⁺]: 324.1.

Example 094: ¹H NMR (400 MHz, DMSO-d₆) δ=13.60 (s, 1H), 11.42 (s, 1H), 7.59 (d, J=3.2 Hz, 1H), 7.34-7.22 (m, 1H), 7.01-6.97 (m, 1H), 6.90 (s, 1H), 5.14 (s, 2H), 2.44 (s, 3H), 2.16 (s, 3H). ¹⁹F NMR (376 MHz, DMSO-d₆) δ ppm −133.068 (s, 1F).

### Example 095: 3-Amino-6-chloro-7-cyclopropyl-4-(7-fluoro-1H-indazol-4-yl)-1H-1,5-naphthyridin-2-one

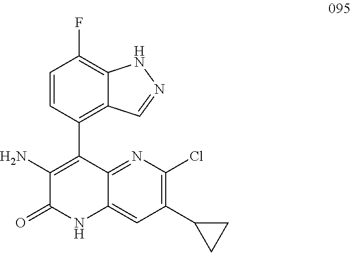 [SMILES: Nc1c(-c2ccc(F)c3[nH]ncc23)c2nc(Cl)c(C3CC3)cc2[nH]c1=O]

### Step 1: (3-Amino-6-chloro-5-cyclopropylpyridin-2-yl)(7-fluoro-1H-indazol-4-yl)methanone

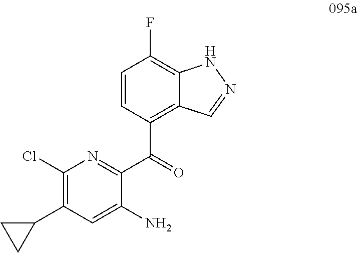 [SMILES: Nc1cc(C2CC2)c(Cl)nc1C(=O)c1ccc(F)c2[nH]ncc12]

To a mixture of compound 015b (350.0 mg, 1.2 mmol) in DMF (2.5 mL) was added NCS (165.6 mg, 1.2 mmol) portionwise at 0° C. The mixture was slowly warmed up to 50° C. and stirred for 1 hour. After completion, the reaction mixture was quenched with saturated sodium sulfite (20 mL) and extracted with EtOAc (20 mL×3). The combined organic layer was washed with brine (40 mL), dried over anhydrous sodium sulfate and concentrated under reduced pressure to give a residue, which was purified by prep-HPLC (NH₄HCO₃ as additive) and lyophilized to give compound 095a (210.0 mg, 53.8% yield) as a yellow solid. MS (ESI⁺) obsd. [(M+H)⁺]: 331.0.

### Step 2: 2-Chloro-N-(6-chloro-5-cyclopropyl-2-(7-fluoro-1H-indazole-4-carbonyl)pyridin-3-yl)acetamide

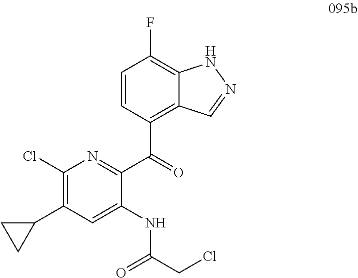 [SMILES: O=C(CCl)Nc1cc(C2CC2)c(Cl)nc1C(=O)c1ccc(F)c2[nH]ncc12]

The title compound was prepared in analogy to the preparation of compound 001c, replacing compound 001b with compound 095a. Compound 095b (80.0 mg, crude) was obtained as a yellow gum. MS (ESI⁺) obsd. [(M+H)⁺]: 407.2.

### Step 3: 6-Chloro-7-cyclopropyl-4-(7-fluoro-1H-indazol-4-yl)-3-pyridin-1-ium-1-yl-1H-1,5-naphthyridin-2-one;chloride

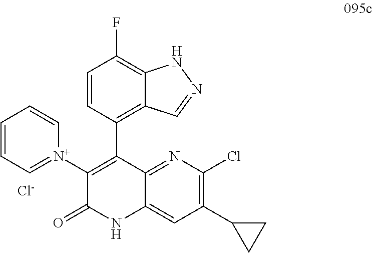 [SMILES: O=c1[nH]c2cc(C3CC3)c(Cl)nc2c(-c2ccc(F)c3[nH]ncc23)c1-[n+]1ccccc1 ; [Cl-]]

The title compound was prepared in analogy to the preparation of compound 001d, replacing compound 001c with compound 095b. Compound 095c (70.0 mg, crude) was obtained as a brown solid. MS (ESI⁺) obsd. [(M-Cl)⁺]: 432.2.

### Step 4: 3-Amino-6-chloro-7-cyclopropyl-4-(7-fluoro-1H-indazol-4-yl)-1H-1,5-naphthyridin-2-one

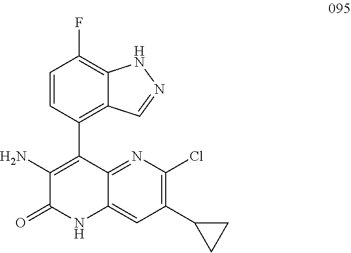 [SMILES: Nc1c(-c2ccc(F)c3[nH]ncc23)c2nc(Cl)c(C3CC3)cc2[nH]c1=O]

The title compound was prepared in analogy to the preparation of Example 001 in step 5, replacing compound 001d with compound 095c. Example 095 (29.3 mg) was obtained as a yellow solid. MS (ESI⁺) obsd. [(M)⁺]: 370.0.

Example 095: ¹H NMR (400 MHz, DMSO-d₆) δ ppm 13.64 (s, 1H) 12.10 (s, H) 7.66 (s, 1H) 7.27-7.34 (m, 1H) 7.26 (s, 1H) 7.01 (dd, J=7.6, 4.0 Hz, 1H) 5.39 (s, 2H) 2.02-2.09 (m, 1H) 1.04 (d, J=6.8 Hz, 2H) 0.64 (d, J=4.0 Hz, 2H). ¹⁹F NMR (377 MHz, DMSO-d₆) δ ppm −132.46 (s, 1 F).

### Example 096: 3-Amino-4-(7-fluoro-1H-indazol-4-yl)-6-[3-(1-hydroxy-1-methyl-ethyl)cyclobutoxy]-7-methyl-1H-1,5-naphthyridin-2-one

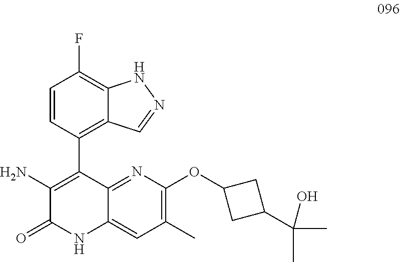 [SMILES: Cc1cc2[nH]c(=O)c(N)c(-c3ccc(F)c4[nH]ncc34)c2nc1OC1CC(C(C)(C)O)C1]

The title compound was prepared in analogy to the preparation of Example 078, replacing Example 061 with Example 067. Example 096 (20.1 mg) was obtained as a yellow solid. MS (ESI⁺) obsd. [(M+H)⁺]: 438.3.

Example 096: ¹H NMR (400 MHz, DMSO-d₆) δ ppm 13.55 (s, 1H), 11.94 (s, 1H), 77.61 (s, 1H), 7.36 (s, 1H), 7.27 (dd, J=11.2, 7.6 Hz, 1H), 7.03 (dd, J=7.6, 4.4 Hz, 1H), 5.14 (s, 2H) 4.14-4.09 (m, 1H) 3.99 (s, 1H) 2.11 (s, 3H) 1.64-1.55 (m, 3H), 1.48-1.36 (m, 2H), 0.86 (d, J=4.4 Hz, 6H). ¹⁹F NMR (376 MHz, DMSO-d₆) δ ppm −133.23 (s, 1 F).

### Example 097: 3-Amino-4-(7-fluoro-1H-indazol-4-yl)-7-methyl-6-(trifluoromethyl)-1H-1,5-naphthyridin-2-one

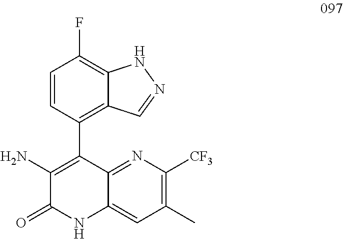 [SMILES: Cc1cc2[nH]c(=O)c(N)c(-c3ccc(F)c4[nH]ncc34)c2nc1C(F)(F)F]

A mixture of compound 080a (130.0 mg, 0.3 mmol) and (1,10-phenanthroline-κN¹,κN¹⁰)(trifluoromethyl)copper (184.5 mg, 0.6 mmol) in DMF (3 mL) was stirred at 80° C. for 3 hours. After completion, the reaction mixture was poured into water (40 mL) and extracted with EtOAc (30 mL×3). The combined organic layer was washed with brine (30 mL), dried over anhydrous sodium sulfate and concentrated under reduced pressure to give a residue, which was purified by prep-HPLC (NH₄HCO₃ as additive) and lyophilized to give Example 097 (23.3 mg, 18.6% yield) as a grey solid. MS (ESI⁺) obsd. [(M+H)⁺]: 378.0.

Example 097: ¹H NMR (400 MHz, DMSO-d₆) δ ppm 13.60 (s, 1H), 7.65 (d, J=1.2 Hz, 1H), 7.57 (s, 1H), 7.32-7.26 (m, 1H), 7.05 (dd, J=4.0, 8.0 Hz, 1H), 5.50 (s, 2H), 2.43 (s, 3H). ¹⁹F NMR (376 MHz, DMSO-d₆) δ ppm −62.83 (s, 1F), −132.54 (s, 1F).

### Example 098: 3-Amino-4-(7-fluoro-1H-indazol-4-yl)-7-methyl-6-(1-methylazetidin-3-yl)-1H-1,5-naphthyridin-2-one

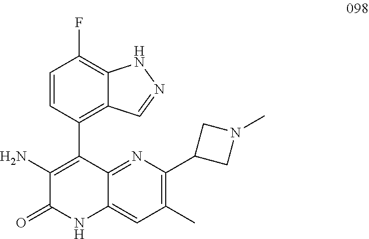 [SMILES: Cc1cc2[nH]c(=O)c(N)c(-c3ccc(F)c4[nH]ncc34)c2nc1C1CN(C)C1]

### Step 1: tert-Butyl 3-(7-amino-8-(7-fluoro-1H-indazol-4-yl)-3-methyl-6-oxo-5,6-dihydro-1,5-naphthyridin-2-yl)azetidine-1-carboxylate

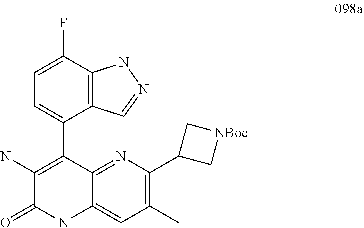 [SMILES: Cc1cc2[nH]c(=O)c(N)c(-c3ccc(F)c4[nH]ncc34)c2nc1C1CN(C(=O)OC(C)(C)C)C1]

The title compound was prepared in analogy to the preparation of Example 093, replacing 3-bromooxetane with tert-butyl 3-bromoazetidine-1-carboxylate. Compound 098a (60.1 mg) was obtained as a yellow solid. MS (ESI⁺) obsd. [(M+H)⁺]: 465.2.

### Step 2: 3-Amino-6-(azetidin-3-yl)-4-(7-fluoro-1H-indazol-4-yl)-7-methyl-1,5-naphthyridin-2(1H)-one

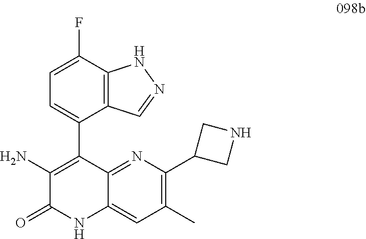 [SMILES: Cc1cc2[nH]c(=O)c(N)c(-c3ccc(F)c4[nH]ncc34)c2nc1C1CNC1]

A solution of compound 098a (60.1 mg, 0.12 mmol) in TFA (2 mL) was stirred at rt for 1 hour. After completion, solvent was evaporated in vacuo to give a residue, which was purified by prep-HPLC (NH₄HCO₃ as additive) to afford compound 098b (30.0 mg, 68.4% yield) as yellow solid. MS (ESI⁺) obsd. [(M+H)⁺]: 365.1.

### Step 3: 3-Amino-4-(7-fluoro-1H-indazol-4-yl)-7-methyl-6-(1-methylazetidin-3-yl)-1H-1,5-naphthyridin-2-one

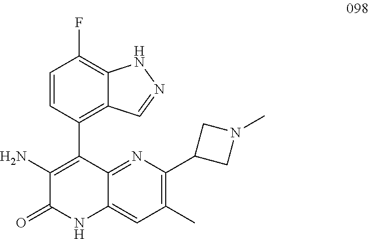 [SMILES: Cc1cc2[nH]c(=O)c(N)c(-c3ccc(F)c4[nH]ncc34)c2nc1C1CN(C)C1]

To a solution of compound 098b (30.0 mg, 0.08 mmol), formaldehyde (12.36 mg, 0.41 mmol) in Methanol (3 mL) was added NaBH₃CN (26.28 mg, 0.41 mmol) at 20° C. and stirred for 7 hours. After completion, the mixture was filtered and the filtrate was concentrated under reduced pressure to give a residue, which was purified by prep-HPLC (NH₄HCO₃ as additive) and lyophilized to give Example 098 (10.0 mg, 31.2% yield) as a white solid. MS (ESI⁺) obsd. [(M+H)⁺]: 379.1.

Example 098: ¹H NMR (400 MHz, DMSO-d₆) δ ppm 7.61 (d, J=3.6 Hz, 1H), 7.31 (s, 1H), 7.30-7.24 (m, 1H), 7.10 (dd, J=4.0, 8.0 Hz, 1H), 5.22 (s, 2H), 3.72-3.64 (m, 1H), 3.40-3.37 (m, 2H), 2.78-2.70 (m, 2H), 2.18 (s, 3H), 1.87 (s, 3H). ¹⁹F NMR (376 MHz, DMSO-d₆) δ ppm −73.43 (s, 3F), −132.96 (s, 1F).

### Example 099: 3-Amino-7-chloro-4-(7-fluoro-1H-indazol-4-yl)-8-methyl-1H-1,5-naphthyridin-2-one

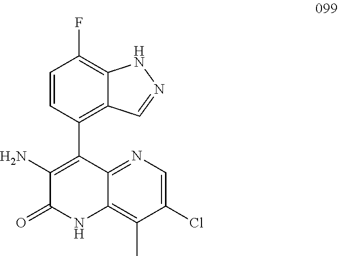 [SMILES: Cc1c(Cl)cnc2c(-c3ccc(F)c4[nH]ncc34)c(N)c(=O)[nH]c12]

### Step 1: 5-Chloro-2-iodo-4-methyl-3-nitropyridine

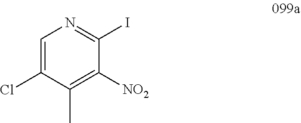

A mixture of 5-chloro-4-methyl-3-nitro-pyridin-2-amine (10.0 g, 53.3 mmol), CuI (3.1 g, 16.0 mmol) and KI (17.7 g, 106.6 mmol) in DME (60 mL) and toluene (24 mL) was heated to 65° C. under N₂ protection. To the above mixture, tert-butyl nitrite (17.3 mL, 143.9 mmol) was added at 65° C. in portions over 10 minutes and the mixture was stirred at 65° C. for another 12 hours. After completion, the reaction mixture was quenched with saturated sodium sulfite (50 mL) and the resulting mixture was extracted with ethyl acetate (25 mL×3). The combined organic layer was washed with brine (70 mL), dried over anhydrous sodium sulfate and concentrated in vacuo to give a residue, which was purified by silica gel chromatography (elute with EtOAc:PE=0-20%) to give compound 099a (6.5 g, 40.9% yield) as a light yellow solid. MS (ESI⁺) obsd. [(M+H)⁺]: 299.1.

### Step 2: (5-Chloro-4-methyl-3-nitropyridin-2-yl)(7-fluoro-1-(tetrahydro-2H-pyran-2-yl)-1H-indazol-4-yl)methanol

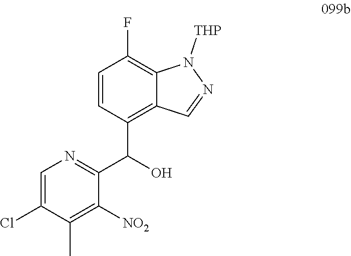

To a solution of compound 099a (4.5 g, 15.1 mmol) in THE (20 mL) was added PhMgBr (3 M, 5.53 mL) at −40° C. under nitrogen atmosphere. And then, a solution of 7-fluoro-1-(tetrahydro-2H-pyran-2-yl)-1H-indazole-4-carbaldehyde (5.2 g, 21.1 mmol) in THE (20 mL) was added to the above mixture at −40° C. The mixture was slowly warmed up to rt and stirred for 30 minutes. After completion, the reaction mixture was quenched with saturated ammonium chloride (30 mL) and extracted with EtOAc (15 mL×3). The combined organic layer was washed with brine (45 mL), dried over anhydrous sodium sulfate and concentrated in vacuo to give a residue, which was purified by silica-gel chromatography (elute with THF:PE=0-30%) to give compound 099b (1.6 g, 45.4% yield) as a light yellow solid. MS (ESI⁺) obsd. [(M+H)⁺]: 421.1.

### Step 3: (5-Chloro-4-methyl-3-nitropyridin-2-yl)(7-fluoro-1-(tetrahydro-2H-pyran-2-yl)-1H-indazol-4-yl)methanone

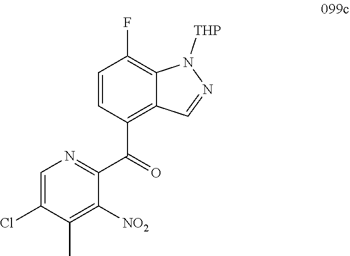

A mixture of compound 099b (300.0 mg, 0.71 mmol) and MnO₂ (1.24 g, 14.26 mmol) in chloroform (4 mL) was stirred at 50° C. for 2 hours. After completion, the mixture was filtered and the filtrate was concentrated under in vacuo to give compound 099c (290.0 mg, crude) as an orange solid. MS (ESI⁺) obsd. [(M-THP+H)⁺]: 335.1.

### Step 4: (3-Amino-5-chloro-4-methylpyridin-2-yl)(7-fluoro-1-(tetrahydro-2H-pyran-2-yl)-1H-indazol-4-yl)methanone

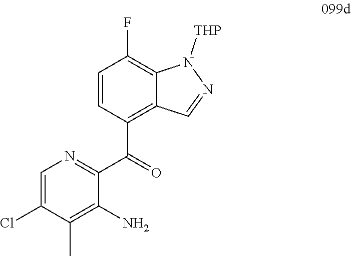

A mixture of compound 099c (290.0 mg, 0.69 mmol), Fe (386.7 mg, 6.9 mmol) and NH₄Cl (370.4 mg, 6.9 mmol) in ethanol (1.5 mL) and water (0.5 mL) was stirred at 80° C. for 2 h. The mixture was filtered and the filtrate was concentrated in vacuo to give a residue, which was purified by silica-gel chromatography (elute with THF:PE=0-30%) to give compound 099d (130.0 mg, 48.3% yield) as a yellow solid. MS (ESI⁺) obsd. [(M+H)⁺]: 389.1

### Step 5: (3-Amino-5-chloro-4-methylpyridin-2-yl)(7-fluoro-1H-indazol-4-yl)methanone

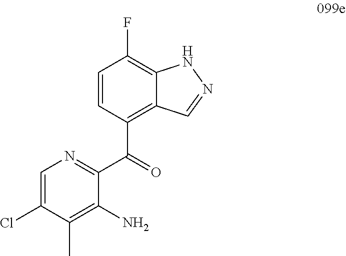 [SMILES: Cc1c(Cl)cnc(C(=O)c2ccc(F)c3[nH]ncc23)c1N]

A solution of compound 099d (130.0 mg, 0.33 mmol) in HCl/EA (4 M, 4.0 mL) was stirred at 20° C. for 1 hour. After completion, the reaction mixture was concentrated under reduced pressure to give compound 099e (110.0 mg, crude) as a yellow solid. MS (ESI⁺) obsd. [(M+H)⁺]: 305.2.

### Step 6: 2-Chloro-N-(5-chloro-2-(7-fluoro-1H-indazole-4-carbonyl)-4-methylpyridin-3-yl)acetamide

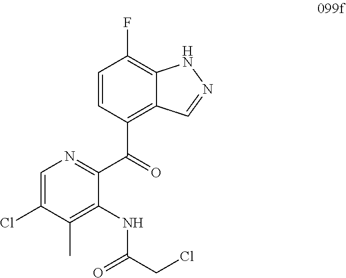 [SMILES: Cc1c(Cl)cnc(C(=O)c2ccc(F)c3[nH]ncc23)c1NC(=O)CCl]

The title compound was prepared in analogy to the preparation of compound 001c, replacing compound 001b with compound 099e. Compound 099f (150.0 mg, crude) was obtained as a light grey solid. MS (ESI⁺) obsd. [(M+H)⁺]: 381.0.

### Step 7: 1-(7-Chloro-4-(7-fluoro-1H-indazol-4-yl)-8-methyl-2-oxo-1,2-dihydro-1,5-naphthyridin-3-yl)pyridin-1-ium chloride

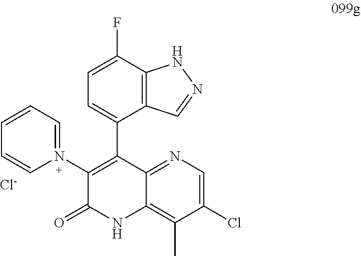

The title compound was prepared in analogy to the preparation of compound 001d, replacing compound 001c with compound 099f. Compound 099g (150.0 mg, crude) was obtained as a black solid. MS (ESI⁺) obsd. [(M-Cl)⁺]: 406.1.

### Step 8: 3-Amino-7-chloro-4-(7-fluoro-1H-indazol-4-yl)-8-methyl-1,5-naphthyridin-2(1H)-one

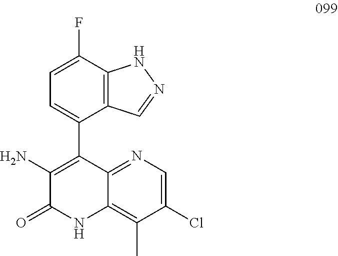 [SMILES: Cc1c(Cl)cnc2c(-c3ccc(F)c4[nH]ncc34)c(N)c(=O)[nH]c12]

The title compound was prepared in analogy to the preparation of Example 001 in step 5, replacing compound 001d with compound 099g. Example 099 (15.3 mg) was obtained as a light red solid. MS (ESI⁺) obsd. [(M+H)⁺]: 344.0.

Example 099: ¹H NMR (400 MHz, DMSO-d₆) δ ppm 13.64 (s, 1H), 11.67 (s, 1H), 8.12 (s, 1H), 7.63 (d, J=2.4 Hz, 1H), 7.28 (dd, J=8.0, 11.2 Hz, 1H), 6.98 (dd, J=4.4, 8.0 Hz, 1H), 2.56 (s, 3H). ¹⁹F NMR (376 MHz, DMSO-d₆) δ ppm −132.66 (s, 1F).

### Example 100: 3-Amino-7-cyclopropyl-4-(7-fluoro-1H-indazol-4-yl)-8-methyl-1H-1,5-naphthyridin-2-one

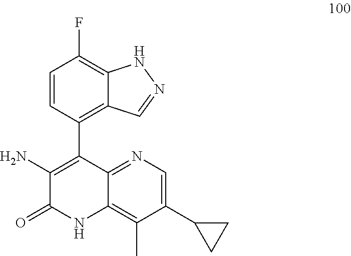 [SMILES: Cc1c(C2CC2)cnc2c(-c3ccc(F)c4[nH]ncc34)c(N)c(=O)[nH]c12]

The title compound was prepare in analogy to the preparation of Example 099, replacing compound 099c with compound Int-22 in step 4. Example 100 (15.3 mg) was obtained as a yellow solid. MS (ESI⁺) obsd. [(M+H)⁺]: 350.0.

Example 100: ¹H NMR (400 MHz, DMSO-d₆) δ ppm 13.05 (s, 1H), 12.21 (s, 1H), 7.85 (d, J=3.2 Hz, 1H), 7.60 (s, 1H), 7.39 (dd, J=7.6, 11.2 Hz, 1H), 7.09 (dd, J=4.4, 8.0 Hz, 1H), 6.71 (s, 2H), 2.76 (s, 3H), 2.07-1.99 (m, 1H), 1.03-0.99 (m, 2H), 0.69-0.65 (m, 2H). ¹⁹F NMR (376 MHz, DMSO-d₆) δ ppm −130.42 (s, 1F).

### Example 101: 3-Amino-4-(7-fluoro-1H-indazol-4-yl)-8-methyl-7-(trideuteriomethyl)-1H-1,5-naphthyridin-2-one

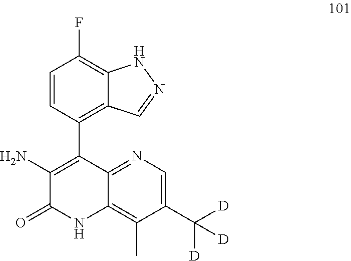 [SMILES: [2H]C([2H])([2H])c1cnc2c(-c3ccc(F)c4[nH]ncc34)c(N)c(=O)[nH]c2c1C]

The title compound was prepared in analogy to the preparation of Example 099, replacing compound 099d with compound Int-23 in step 5. Example 101 (6.3 mg) was obtained as a yellow solid. MS (ESI⁺) obsd. [(M+H)⁺]: 326.9.

Example 101: ¹H NMR (400 MHz, MeOD-d₄) δ ppm 7.87 (s, 1H), 7.86 (d, J=3.2 Hz, 1H), 7.40 (dd, J=7.6, 10.8 Hz, 1H), 7.23 (dd, J=4.0, 8.0 Hz, 1H), 2.67 (s, 3H). ¹⁹F NMR (376 MHz, MeOD-d₄) δ ppm −131.47 (s, 1F).

### Example 102: 3-Amino-4-(7-fluoro-1H-indazol-4-yl)-5-methoxy-7-methyl-1H-quinolin-2-one

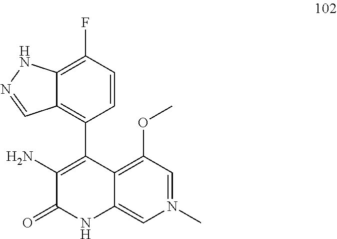

The title compound was prepared in analogy to the preparation of Example 045, replacing Int-2 with tert-butyl N-(3-methoxy-5-methyl-phenyl)carbamate and compound 045a with 7-fluoro-1-tetrahydropyran-2-yl-indazole-4-carbaldehyde in step 2. Example 102 (7.7 mg) was obtained as a yellow solid. MS (ESI⁺) obsd. [(M+H)⁺]: 339.1.

Example 102: ¹H NMR (400 MHz, DMSO-d₆) δ ppm 13.54 (br s, 1H), 11.96 (s, 1H), 7 60 (d, J=3.6 Hz, 1H), 7.21 (dd. J=8.0, 11.2 Hz, 1H), 6.84 (dd. J=4.0, 8.0 Hz, 1H), 6.76 (s, 1H), 6.37 (s, 1H). 2.98 (s, 31H), 2.28 (s, 3H). ¹⁹F NMR (377 MHz, DMSO-d₆ ppm −133.94 (s, 1F).

### Example 103: 3-Amino-5-chloro-4-(7-fluoro-1H-indazol-4-yl)-7-methyl-1H-quinolin-2-one

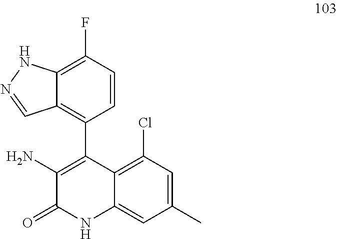 [SMILES: Cc1cc(Cl)c2c(-c3ccc(F)c4[nH]ncc34)c(N)c(=O)[nH]c2c1]

The title compound was prepared in analogy to the preparation of Example 045, replacing Int-2 with tert-butyl N-(3-chloro-5-methyl-phenyl)carbamate and compound 045a with 7-fluoro-1-tetrahydropyran-2-yl-indazole-4-carbaldehyde in step 2. Example 103 (19.8 mg) was obtained as a yellow solid. MS (ESI⁺) obsd. [(M+H)⁺]: 343.1.

Example 103: ¹H NMR (400 MHz, DMSO)-d) δ ppm 13.65 (s, 1H), 12.21 (s, 1H), 7.68 (s, 1H)), 7.28-7.23 (m, 1H), 7.12 (s, 1H), 6.91 (dd, J=4.4 Hz, J=7.6 Hz. 1H), 6.87 (d, J=1.6 Hz, 1_H), 4.78 (s, 2H), 2.28 (s, 3H). ¹⁹F NMR (377 MHz, DMSO-d₆) δ ppm −132.740 (s, 1F).

### Example 104: 3-Amino-6-bromo-4-(7-fluoro-1H-indazol-4-yl)-7-methyl-1H-quinolin-2-one

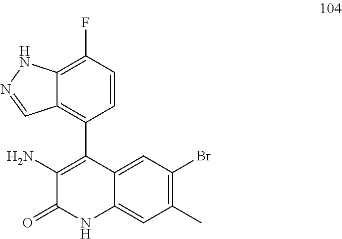 [SMILES: Cc1cc2[nH]c(=O)c(N)c(-c3ccc(F)c4[nH]ncc34)c2cc1Br]

The title compound was prepared in analogy to the preparation of Example 021, replacing Int-3 with tert-butyl N-(2-bromo-5-methyl-phenyl)carbamate in step 1. Example 104 (69.5 mg) was obtained as a yellow solid. MS (ESI⁺) obsd. [(M+H)⁺]: 387.0.

Example 104: ¹H NMR (400 MHz, DMSO-d₆) δ ppm 7.71 (d, J=3.2 Hz, 1H), 7.36 (dd, J=7.6, 11.2 Hz, 1H), 7.24 (s, 1H), 7.02 (dd, J=4.0, 7.6 Hz, 1H), 6.81 (s, 1H), 4.99 (s, 2H), 2.32 (s, 3H). ¹⁹F NMR (376 MHz, DMSO-d₆) δ ppm −131.43 (s, 1F).

### Example 105: 3-Amino-4-(7-fluoro-1H-indazol-4-yl)-7-methyl-6-(2-oxa-6-azaspiro[3.3]heptan-6-yl)-1H-quinolin-2-one

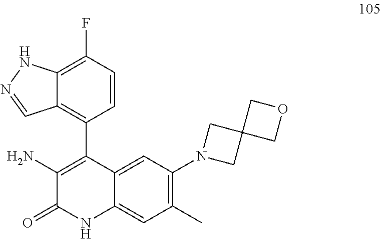 [SMILES: Cc1cc2[nH]c(=O)c(N)c(-c3ccc(F)c4[nH]ncc34)c2cc1N1CC2(COC2)C1]

The mixture of Example 104 (50.0 mg, 0.13 mmol), 2-oxa-6-azaspiro[3.3]heptane (60.0 mg, 0.61 mmol), t-BuONa (0.8 mL, 0.8 mmol) and CPhos-Pd-G3 (5.2 mg, 0.01 mmol) in degassed 1,4-dioxane (4 mL) was stirred at 100° C. for 12 h under N₂ protection. After completion, the suspension was filtered and the filtrate was concentrated under reduced pressure to give a residue, which was purified by prep-HPLC (NH₄HCO₃ as additive) to give Example 105 (12.7 mg, 25.2% yield) as a yellow solid. MS (ESI⁺) obsd. [(M+H)⁺]: 406.2.

Example 105: ¹H NMR (400 MHz, DMSO-d₆) δ ppm 13.78 (s, 1H), 11.80 (s, 1H), 7.66 (d, J=3.2 Hz, 1H), 7.36 (dd, J=7.6, 11.2 Hz, 1H), 7.00 (dd, J=4.0, 7.6 Hz, 1H), 6.97 (s, 1H), 5.80 (s, 1H), 4.72 (s, 2H), 4.59-4.55 (m, 4H), 3.68 (d, J=7.2 Hz, 2H), 3.62 (d, J=7.2 Hz, 2H), 2.14 (s, 3H).

### Example 106: 3-Amino-4-(7-fluoro-1H-indazol-4-yl)-7-methyl-6-[1-(2,2,2-trifluoroethyl)-4-piperidyl]-1H-quinolin-2-one

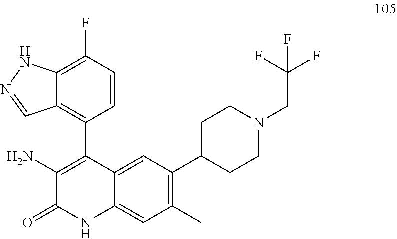 [SMILES: Cc1cc2[nH]c(=O)c(N)c(-c3ccc(F)c4[nH]ncc34)c2cc1C1CCN(CC(F)(F)F)CC1]

### Step 1: 3-Amino-4-(7-fluoro-1H-indazol-4-yl)-7-methyl-6-[1-(2,2,2-trifluoroethyl)-3,6-dihydro-2H-pyridin-4-yl]-1H-quinolin-2-one

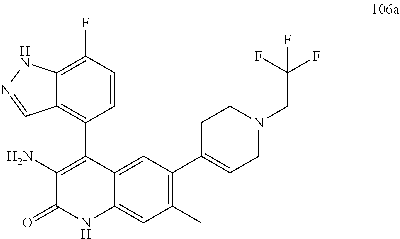 [SMILES: Cc1cc2[nH]c(=O)c(N)c(-c3ccc(F)c4[nH]ncc34)c2cc1C1=CCN(CC(F)(F)F)CC1]

A mixture of Example 104 (30.0 mg, 0.077 mmol), 4-(4,4,5,5-tetramethyl-1,3,2-dioxaborolan-2-yl)-1-(2,2,2-trifluoroethyl)-3,6-dihydro-2H-pyridine (27.1 mg, 0.093 mmol), Pd(dppf)Cl₂ (6.3 mg, 7.8 umol) and K₂CO₃ (32.1 mg, 0.23 mmol) in 1,4-dioxane (3 mL) and water (600 uL) was stirred in sealed tube under N₂ atmosphere at 100° C. for 2 h. After completion, the resulting mixture was concentrated to give a residue, which was suspended in EtOAc and filtered. The filtrate was concentrated to give compound 106a (36.0 mg, 98.6% yield) as a yellow solid, which was used in the next step without purification. MS (ESI⁺) obsd. [(M+H)⁺]: 472.4.

### Step 2: 3-Amino-4-(7-fluoro-1H-indazol-4-yl)-7-methyl-6-[1-(2,2,2-trifluoroethyl)-4-piperidyl]-1H-quinolin-2-one

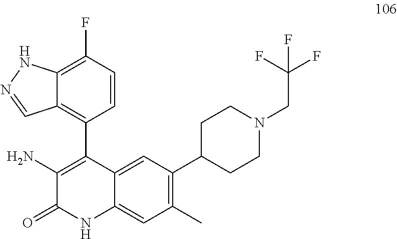 [SMILES: Cc1cc2[nH]c(=O)c(N)c(-c3ccc(F)c4[nH]ncc34)c2cc1C1CCN(CC(F)(F)F)CC1]

A mixture of compound 106a (36.0 mg, 0.076 mmol) and Pd/C (9 mg) in MeOH (10 mL) was stirred under H₂ balloon at rt for 16 h. After completion, the resulting mixture was filtered and the filtrate was concentrated under reduced pressure to give a residue, which was purified by pre-HPLC (FA as additive) to afford Example 106 (4.3 mg, 11.1% yield) as a white solid. MS (ESI⁺) obsd. [(M+H)⁺]: 474.7.

Example 106: ¹H NMR (400 MHz, DMSO-d6) δ ppm 13.76 (s, 1H), 11.89 (s, 1H),

7.63 (br d, J=1.9 Hz, 1H), 7.38 (dd, J=8.0, 11.0 Hz, 1H), 7.08 (s, 1H), 7.03 (dd, J=4.0, 7.6 Hz, 1H), 6.62 (s, 1H), 4.74 (br s, 2H), 3.09 (q, J=10.4 Hz, 2H), 2.92-2.79 (m, 2H), 2.41-2.31 (m, 2H), 2.29 (s, 3H), 2.04-1.94 (m, 1H), 1.58-1.50 (m, 1H), 1.48-1.40 (m, 1H), 1.20-0.98 (m, 2H).

### Example 107: 3-Amino-4-(7-fluoro-1H-indazol-4-yl)-6,7-dimethyl-1H-1,8-naphthyridin-2-one

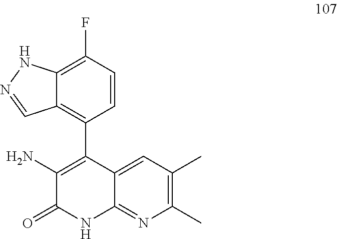 [SMILES: Cc1cc2c(-c3ccc(F)c4[nH]ncc34)c(N)c(=O)[nH]c2nc1C]

The title compound was prepared in analogy to the preparation of Example 045, replacing Int-2 with 3-bromo-5,6-dimethyl-pyridin-2-amine and compound 045a with 7-fluoro-1-tetrahydropyran-2-yl-indazole-4-carbaldehyde in step 2. Example 107 (44.7 mg) was obtained as a yellow solid. MS (ESI⁺) obsd. [(M+H)⁺]: 324.3.

Example 107: ¹H NMR (400 MHz, MeOD-d₄) δ ppm 7.75 (d, J=3.2 Hz, 1H), 7.42 (s, 1H), 7.37 (dd, J=7.6, 10.8 Hz, 1H), 7.16 (dd, J=4.0, 7.6 Hz, 1H), 2.66 (s, 3H), 2.25 (s, 3H). ¹⁹F NMR (376 MHz, MeOD-d₄) δ ppm −132.82 (s, 1F).

### Example 108: 3-Amino-6-bromo-8-chloro-4-(7-fluoro-1H-indazol-4-yl)-7-methyl-1H-1,5-naphthyridin-2-one

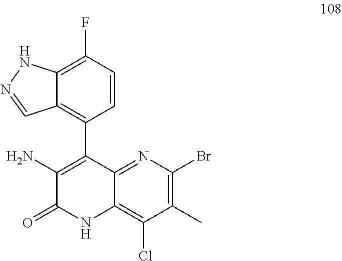 [SMILES: Cc1c(Br)nc2c(-c3ccc(F)c4[nH]ncc34)c(N)c(=O)[nH]c2c1Cl]

The title compound was prepared in analogy to the preparation of Example 021, replacing Int-3 with tert-butyl N-(2-bromo-4-chloro-5-methyl-3-pyridyl)carbamate in step 1. Example 108 (10.5 mg) was obtained as a yellow solid. MS (ESI⁺) obsd. [(M+H)⁺]: 421.9.

Example 108: ¹H NMR (400 MHz, DMSO-d₆) δ ppm 13.64 (br s, 1H), 11.67-11.80 (m, 1H), 7.69 (br s, 1H), 7.30 (dd, J=11.13, 7.63 Hz, 1H), 7.01 (dd, J=7.82, 3.94 Hz, 1H), 5.63 (s, 2H), 2.45 (s, 3H). ¹⁹F NMR (376 MHz, DMSO-d₆) δ ppm −132.28 (s, 1F).

### Example 109: 3-Amino-8-chloro-6-cyclopropyl-4-(7-fluoro-1H-indazol-4-yl)-7-methyl-1H-1,5-naphthyridin-2-one

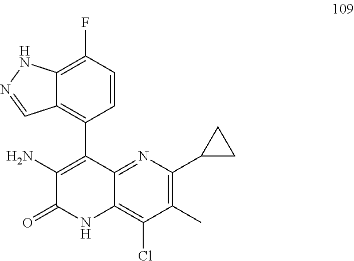 [SMILES: Cc1c(C2CC2)nc2c(-c3ccc(F)c4[nH]ncc34)c(N)c(=O)[nH]c2c1Cl]

A mixture of compound Example 108 (100.0 mg, 0.23 mmol), cyclopropylboronic acid (30.5 mg, 0.35 mmol), potassium carbonate (81.7 mg, 0.59 mmol) and 1,1′-bis(diphenylphosphino)ferrocene palladium dichloride (38.6 mg, 0.047 mmol) in degassed 1,4-dioxane (4 mL) was stirred at 80° C. for 3 h. After completion, the mixture was filtered and concentrated under vacuum to give a residue, which was purified by prep-HPLC (FA as additive) to give Example 109 (18.3 mg, 20.7% yield) as a yellow solid. MS (ESI⁺) obsd. [(M+H)⁺]: 384.0.

Example 109: ¹H NMR (400 MHz, DMSO-d₆) δ ppm 13.55 (br s, 1H), 11.26-11.41

(m, 1H), 7.55 (br s, 1H), 7.27 (dd, J=7.9, 11.1 Hz, 1H), 7.00 (dd, J=4.1, 7.7 Hz, 1H), 5.35 (br s, 2H), 2.47 (s, 3H), 2.00-2.09 (m, 1H), 0.58 (dd, J=3.6, 8.1 Hz, 2H), 0.35 (dt, J=4.2, 8.9 Hz, 1H), 0.23 (dt, J=4.3, 8.7 Hz, 1H). ¹⁹F NMR (376 MHz, DMSO-d₆) δ ppm −132.84 (s, 1F).

The following Examples 110 to 114 were prepared in analogy to the procedure described for the preparation of Example 109, replacing cyclopropylboronic acid with CLREAG indicated in Table 9.

TABLE 9
Compound synthesis and characterization

| | Compound Name and | | |
|---|---|---|---|
| Ex. | Structures | CLREAG | NMR and (ESI⁺) |
| 110 | 3-Amino-4-(7-fluoro- 1H-indazol-4-yl)-6,7,8- trimethyl-1H-1,5- naphthyridin-2-one 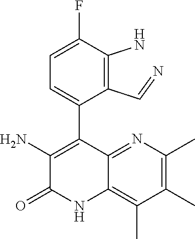 | 2,4,6-Trimethyl- 1,3,5,2,4,6- trioxatriborinane Notes: 105° C. (reaction temperature) | ¹H NMR (400 MHz, MeOD- d₄) δ ppm 13.58 (br s, 1H), 11.25 (s, 1H), 7.58 (br s, 1H), 7.28 (br dd, J = 8.1, 11.1 Hz, 1H), 6.99 (dd, J = 4.2, 7.7 Hz, 1H), 5.01 (br s, 2H), 2.40 (s, 3H), 2.20 (s, 3H), 2.18 (s, 3H). ¹⁹F NMR (376 MHz, DMSO-d₆) δ ppm −133.11 (s, 1F). MS (ESI⁺) obsd. [(M + H)⁺]: 338.0. |
| 111 | 3-Amino-8-chloro-6- ethynyl-4-(7-fluoro-1H- indazol-4-yl)-7-methyl- 1H-1,5-naphthyridin-2- one 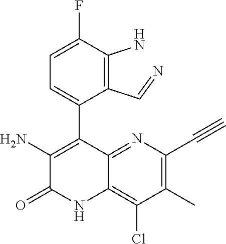 | Tributyl(ethynyl) stannane Notes: without water and base (potassium carbonate) | ¹H NMR (400 MHz, MeOH- d4) δ ppm 7.55 (d, J = 3.3 Hz, 1H), 7.17 (dd, J = 7.9, 10.9 Hz, 1H), 7.02 (dd, J = 4.1, 7.8 Hz, 1H), 3.57 (s, 1H), 2.49 (s, 3H) ¹⁹F NMR (376 MHz, DMSO-d6) δ ppm −134.61 (s, 1F). MS (ESI⁺) obsd. [(M + H)⁺]: 368.3. |
| 112 | 6-Acetyl-3-amino-8- chloro-4-(7-fluoro-1H- indazol-4-yl)-7-methyl- 1H-1,5-naphthyridin-2- one 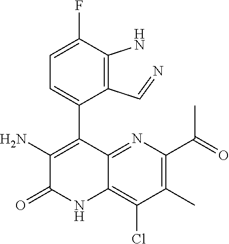 | Tributyl(1- ethoxyvinyl) stannane Notes: without water and base (potassium carbonate) | ¹H NMR (400 MHz, MeOD- d₄) δ ppm 7.59 (d, J = 3.1 Hz, 1H), 7.18 (br d, J = 10.9 Hz, 1H), 7.13-7.09 (m, 1H), 2.55 (s, 3H), 1.97 (s, 3H). ¹⁹F NMR (377 MHz, MeOD-d₄) δ ppm −134.51 (s, 1F). MS (ESI⁺) obsd. [(M + H)⁺]: 386.1. |
| 113 | 3-Amino-8-chloro-4-(7- fluoro-1H-indazol-4- yl)-7-methyl-6-vinyl- 1H-1,5-naphthyridin-2- one 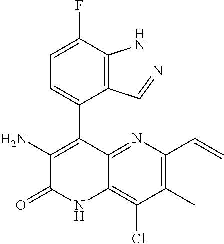 | Tributyl(vinyl) stannane Notes: without water and base (potassium carbonate) | ¹H NMR (400 MHz, MeOD- d₄) δ ppm 7.57 (d, J = 3.1 Hz, 1H), 7.21-7.14 (m, 1H), 7.12-7.07 (m, 1H), 6.85 (dd, J = 10.5, 16.6 Hz, 1H), 5.49 (dd, J = 2.6, 16.8 Hz, 1H), 5.06 (dd, J = 2.6, 10.6 Hz, 1H), 2.40 (s, 3H) ¹⁹F NMR (376 MHz, MeOD-d₄) δ ppm −134.89 (s, 1F). MS (ESI⁺) obsd. [(M + H)⁺]: 370.1. |
| 114 | 3-Amino-6,8- dicyclopropyl-4-(7- fluoro-1H-indazol-4- yl)-7-methyl-1H-1,5- naphthyridin-2-one 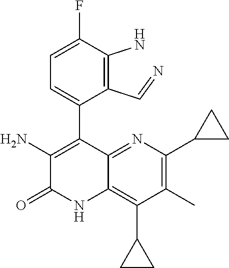 | Cyclopropylboronic acid Notes: 105° C. (reaction temperature) | ¹H NMR (400 MHz, DMSO- d₆) δ ppm 13.53 (br s, 1H), 10.69 (s, 1H), 7.51 (br s, 1H), 7.26 (dd, J = 7.9, 11.0 Hz, 1H), 6.98 (dd, J = 4.2, 7.7 Hz, 1H), 5.11 (br s, 2H), 2.48 (s, 3H), 1.93-2.02 (m, 1 H), 1.83-1.93 (m, 1 H), 1.19-1.32 (m, 3 H), 0.50- 0.57 (m, 3 H), 0.34 (dt, J = 8.63, 4.44 Hz, 1 H), 0.21 (dt, J = 8.60, 4.39 Hz, 1 H). ¹⁹F NMR (376 MHz, DMSO-d₆) δ ppm −133.14 (s, 1F). MS (ESI⁺) obsd. [(M + H)⁺]: 390.2. |

### Example 115: 3-Amino-7-bromo-4-(7-fluoro-1H-indazol-4-yl)-8-methyl-1H-1,5-naphthyridin-2-one

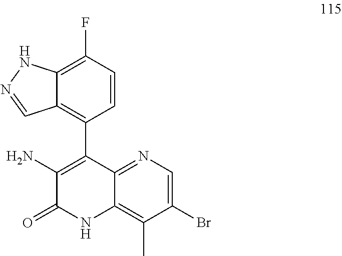 [SMILES: Cc1c(Br)cnc2c(-c3ccc(F)c4[nH]ncc34)c(N)c(=O)[nH]c12]

The title compound was prepared in analogy to the preparation of Example 045, replacing Int-2 with tert-butyl N-(5-bromo-2-iodo-4-methyl-3-pyridyl)carbamate and compound 045a with 7-fluoro-1-tetrahydropyran-2-yl-indazole-4-carbaldehyde in step 2. Example 115 (30.5 mg) was obtained as a yellow solid. MS (ESI⁺) obsd. [(M+H)⁺]: 388.0.

Example 115: ¹H NMR (400 MHz, DMSO-d₆) δ ppm 13.64 (s, 1H), 11.67 (s, 1H), 8.12 (s, 1H), 7.63 (d, J=2.4 Hz, 1H), 7.28 (dd, J=8.0, 11.2 Hz, 1H), 6.98 (dd, J=4.4, 8.0 Hz, 1H), 2.56 (s, 3H).

### Example 116: 3-Amino-4-(7-fluoro-1H-indazol-4-yl)-7-iodo-8-methyl-1H-1,5-naphthyridin-2-one

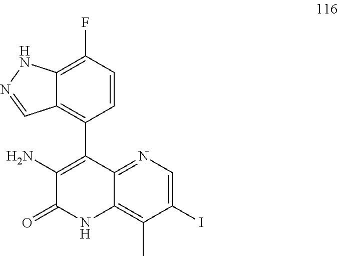 [SMILES: Cc1c(I)cnc2c(-c3ccc(F)c4[nH]ncc34)c(N)c(=O)[nH]c12]

A mixture of Example 115 (20.0 mg, 0.05 mmol), CuI (4.91 mg, 0.03 mmol), NaI (77.23 mg, 0.52 mmol), N,N-dimethylethylenediamine (6.81 mg, 0.08 mmol) in 1,4-dioxane (2 mL) was stirred at 110° C. for 12 h. After completion, the mixture was poured into water (3 mL) and extracted with EtOAc (3×5 mL). The combined organic layer was dried over anhydrous sodium sulfate and concentrated under reduced pressure to give a residue, which was purified by prep-HPLC (NH₄HCO₃ as additive) to give Example 116 (1.3 mg, 5.7% yield) as a white solid. MS (ESI⁺) obsd. [(M+H)⁺]: 436.2.

Example 116: ¹H NMR (400 MHz, DMSO-d₆) δ ppm 13.62 (s, 1H), 11.43 (s, 1H), 8.35 (s, 1H), 7.62 (d, J=1.6 Hz, 1H), 7.27 (d, J=8.4, 11.8 Hz, 1H), 6.97-6.94 (m, 1H), 5.37 (s, 2H), 2.62 (s, 3H). ¹⁹F NMR (376 MHz, DMSO-d₆) δ ppm −132.78 (s, 1F).

### Example 117: 3-Amino-7-(azetidin-1-yl)-4-(7-fluoro-1H-indazol-4-yl)-8-methyl-1H-1,5-naphthyridin-2-one

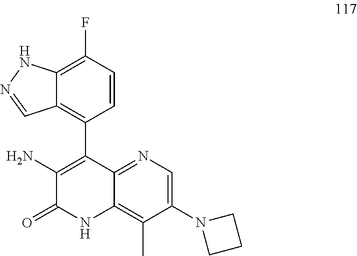 [SMILES: Cc1c(N2CCC2)cnc2c(-c3ccc(F)c4[nH]ncc34)c(N)c(=O)[nH]c12]

A mixture of Example 115 (60.0 mg, 0.15 mmol), azetidine (13.24 mg, 0.23 mmol), t-BuONa (1.55 mL, 3.09 mmol) and CPhos-Pd-G3 (12.46 mg, 0.02 mmol) in degassed 1,4-dioxane (2 mL) was stirred at 100° C. for 2 h. After completion, the reaction mixture was quenched with saturated sodium bicarbonate (5 mL) and extracted with EtOAc (5 mL×3). The combined organic phase was dried over anhydrous sodium sulfate and concentrated under reduce pressure to give a residue, which was purified by prep-HPLC (NH₄HCO₃ as additive) to give Example 117 (13.9 mg, 24.2% yield) as a yellow solid. MS (ESI⁺) obsd. [(M+H)⁺]: 365.2.

Example 117: ¹H NMR (400 MHz, DMSO-d6) δ ppm 13.54 (s, 1H), 11.22 (d, J=2.4 Hz, 1H), 7.58 (s, 1H), 7.52 (s, 1H), 7.28-7.24 (m, 1H), 6.96-6.93 (m, 1H), 4.82 (s, 2H), 3.90 (t, J=7.2 Hz, 4H), 2.25 (s, 3H), 2.24-2.17 (m, 2H). ¹⁹F NMR (400 MHz, DMSO-d6) δ ppm −133.127 (s, 1F).

### Example 118: 7-Amino-8-(7-fluoro-1H-indazol-4-yl)-4-methyl-6-oxo-5H-1,5-naphthyridine-3-carbonitrile

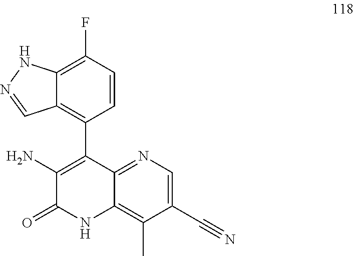 [SMILES: Cc1c(C#N)cnc2c(-c3ccc(F)c4[nH]ncc34)c(N)c(=O)[nH]c12]

A mixture of Example 115 (20.0 mg, 0.06 mmol), Zn (0.84 mg, 0.01 mmol), Zn(CN)₂ (4.54 mg, 0.04 mmol) and Pd(PPh₃)₄ (5.95 mg, 0.01 mmol) in degassed DMF (2 mL) was stirred at 100° C. for 3 h. After completion, the mixture was diluted with water (5 mL) and extracted with EtOAc (5 mL×3). The combined organic layer was concentrated under vacuum to give crude, which was purified by prep-HPLC (NH₄HCO₃ as additive) to give Example 118 as yellow solid (3.5 mg, 17.5% yield). MS (ESI⁺) obsd. [(M+H)⁺]: 335.2.

Example 118: ¹H NMR (400 MHz, DMSO-d₆) δ ppm 13.32 (s, 1H), 11.33 (s, 1H), 8.31 (s, 1H), 7.58 (d, J=1.6 Hz, 1H), 7.23 (d, J=8.4, 11.8 Hz, 1H), 6.97-6.94 (m, 1H), 5.34 (s, 2H), 2.32 (s, 3H). ¹⁹F NMR (376 MHz, DMSO-d₆) δ ppm −132.18 (s, 1F).

### Example 119: 3-Amino-7-cyclobutyl-4-(7-fluoro-1H-indazol-4-yl)-8-methyl-1H-1,5-naphthyridin-2-one

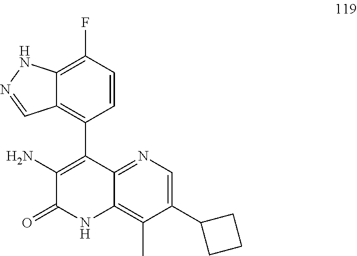 [SMILES: Cc1c(C2CCC2)cnc2c(-c3ccc(F)c4[nH]ncc34)c(N)c(=O)[nH]c12]

A mixture of Example 115 (50.0 mg, 0.13 mmol), 2,6-lutidine (27.6 mg, 0.26 mmol), bis[3,5-difluoro-2-[5-(trifluoromethyl)-2-pyridyl]phenyl]iridium(1+);4-tert-butyl-2-(4-tert-butyl-2-pyridyl)pyridine;hexafluorophosphate (14.5 mg, 0.013 mmol), potassium cyclobutyltrifluoroborate (31.3 mg, 0.19 mmol) and [4,4′-bis(1,1-dimethylethyl)-2,2′-bipyridine]nickel(II) dichloride (10.3 mg, 0.026 mmol) in 1,4-dioxane (4 mL) was stirred in sealed tube under N₂ atmosphere with 450 nm light (16 W) at rt for 16 h. The resulting mixture was filtered and then purified by pre-HPLC (FA as additive) to afford Example 119 (2.2 mg, 4.5% yield) as a yellow solid. MS (ESI⁺) obsd. [(M+H)⁺]: 364.4.

Example 119: ¹H NMR (400 MHz, DMSO-d6) δ ppm 13.54 (br s, 1H), 11.14 (s, 1H), 7.95 (s, 1H), 7.53 (br d, J=2.8 Hz, 1H), 7.21 (dd, J=7.8, 11.2 Hz, 1H), 6.90 (dd, J=4.1, 7.7 Hz, 1H), 5.05 (s, 2H), 3.62 (quin, J=8.7 Hz, 1H), 2.28 (s, 3H), 2.26-2.20 (m, 2H), 2.11-1.97 (m, 1H), 1.97-1.85 (m, 1H), 1.76-1.62 (m, 1H). ¹⁹F NMR (376 MHz, DMSO-d6) δ ppm −131.75 (s, 1F).

### Example 120: 3-Amino-4-(7-fluoro-1H-indazol-4-yl)-8-methyl-7-pyridazin-4-yl-1H-1,5-naphthyridin-2-one

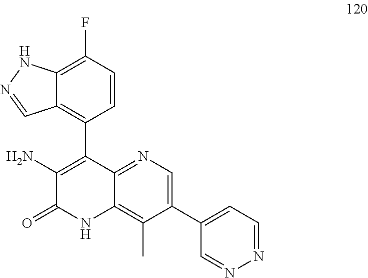 [SMILES: Cc1c(-c2ccnnc2)cnc2c(-c3ccc(F)c4[nH]ncc34)c(N)c(=O)[nH]c12]

A mixture of Example 115 (20.0 mg, 0.06 mmol), tributyl(pyridazin-4-yl)stannane (40.0 mg, 0.10 mmol) and Pd(PPh₃)₄ (18.0 mg, 0.03 mmol) in degassed dioxane (4 mL) was stirred at 100° C. for 3 h under N₂ protection. After completion, the mixture was diluted with water (5 mL), extracted with EtOAc (5 mL×3). The combined organic layer was concentrated under vacuum to give crude, which was purified by prep-HPLC (NH₄HCO₃ as additive) to give Example 120 as yellow solid (3.5 mg, 15.0% yield). MS (ESI⁺) obsd. [(M+H)⁺]: 388.2.

Example 120: ¹H NMR (400 MHz, DMSO-d₆) δ ppm 13.62 (br s, 1H), 11.64 (s, 1H), 9.42-9.14 (m, 2H), 8.09 (s, 1H), 7.78 (dd, J=2.4, 5.4 Hz, 1H), 7.66 (br s, 1H), 7.29 (br dd, J=7.9, 11.0 Hz, 1H), 7.00 (dd, J=4.1, 7.7 Hz, 1H), 5.47 (br s, 2H), 2.46 (s, 3H). ¹⁹F NMR (376 MHz, DMSO-d6) δ ppm −132.83 (s, 1F).

The following Examples 121, 123 to 125 were prepared in analogy to the procedure described for the preparation of Example 120, replacing tributyl(pyridazin-4-yl)stannane with TINCPD indicated in Table 10.

TABLE 10
Compound synthesis and characterization

| | Compound Names and | | |
|---|---|---|---|
| Ex. | Structures | TINCPD | NMR and (ESI⁺) |
| 121 | 3-Amino-4-(7-fluoro- 1H-indazol-4-yl)-8- methyl-7-thiazol-2-yl- 1H-1,5-naphthyridin-2- one 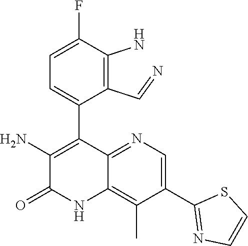 | Tributyl(thiazol-2- yl)stannane | ¹H NMR (400 MHz, DMSO- d₆) δ ppm 13.62 (br s, 1H), 11.59 (s, 1H), 8.37 (s, 1H), 8.02 (d, J = 3.3 Hz, 1H), 7.88 (d, J = 3.3 Hz, 1H), 7.66 (br s, 1H), 7.30 (dd, J = 7.9, 11.2 Hz, 1H), 7.01 (dd, J = 4.1, 7.8 Hz, 1H), 5.51 (s, 2H), 2.70 (s, 3H). ¹⁹F NMR (376 MHz, DMSO-d₆) δ ppm −132.87 (s, 1F). MS (ESI⁺) obsd. [(M + H)⁺]: 393.2. |
| 123 | 3-Amino-4-(7-fluoro- 1H-indazol-4-yl)-8- methyl-7-vinyl-1H-1,5- naphthyridin-2-one 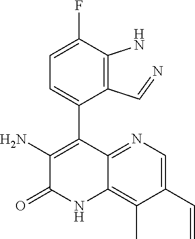 | Tributyl(vinyl) stannane | ¹H NMR (400 MHz, MeOD- d₄) δ ppm 13.60 (br s, 1H), 11.43 (s, 1H), 8.27 (s, 1H), 7.60 (br s, 1H), 7.28 (dd, J = 7.9, 11.1 Hz, 1H), 7.07- 6.91 (m, 2H), 5.70 (dd, J = 1.3, 17.5 Hz, 1H), 5.38- 5.33 (m, 1H), 5.27 (s, 2H), 2.47 (s, 3H). ¹⁹F NMR (376 MHz, DMSO-d₆) δ ppm −133.00 (s, 1F). MS (ESI⁺) obsd. [(M + H)⁺]: 336.1. |
| 124 | 3-Amino-7-ethynyl-4- (7-fluoro-1H-indazol-4- yl)-8-methyl-1H-1,5- naphthyridin-2-one 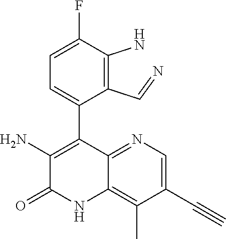 | Tributyl(ethynyl) stannane | ¹H NMR (400 MHz, MeOD- d₄) δ ppm 8.07 (s, 1H), 7.54 (br d, J = 2.5 Hz, 1H), 7.18 (dd, J = 7.8, 10.9 Hz, 1H), 7.00 (dd, J = 4.1, 7.8 Hz, 1H), 3.82 (s, 1H), 2.58 (s, 3H). ¹⁹F NMR (376 MHz, MeOD-d₄) δ ppm −134.60 (s, 1F). MS (ESI⁺) obsd. [(M + H)⁺]: 334.1. |
| 125 | 3-Amino-4-(7-fluoro- 1H-indazol-4-yl)-7- isopropenyl-8-methyl- 1H-1,5-naphthyridin-2- one 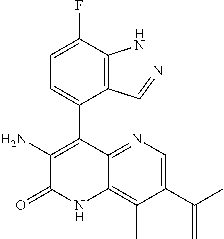 | Tributyl(isopropenyl) stannane | ¹H NMR (400 MHz, DMSO- d₆) δ ppm 12.22 (s, 1H), 7.88 (d, J = 2.8 Hz, 1H), 7.80 (s, 1H), 7.40 (dd, J = 7.6, 11.2 Hz, 1H), 7.10 (dd, J = 4.0, 7.6 Hz, 1H), 6.75 (s, 2H), 5.45 (s, 1H), 5.03 (s, 1H), 2.60 (s, 3H), 2.04 (s, 3H). ¹⁹F NMR (376 MHz, DMSO-d₆) δ ppm −130.52 (s, 1F). MS (ESI⁺) obsd. [(M + H)⁺]: 350.1. |

### Example 122: 3-Amino-4-(7-fluoro-1H-indazol-4-yl)-8-methyl-7-(oxetan-3-yl)-1H-1,5-naphthyridin-2-one

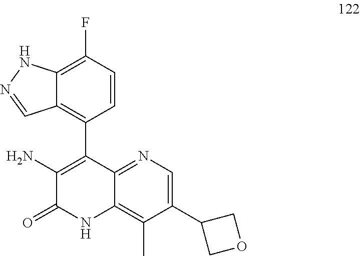 [SMILES: Cc1c(C2COC2)cnc2c(-c3ccc(F)c4[nH]ncc34)c(N)c(=O)[nH]c12]

A mixture of Example 115 (50.0 mg, 0.13 mmol), 2,6-lutidine (27.6 mg, 0.26 mmol), bis[3,5-difluoro-2-[5-(trifluoromethyl)-2-pyridyl]phenyl]iridium(1+);4-tert-butyl-2-(4-tert-butyl-2-pyridyl)pyridine;hexafluorophosphate (14.5 mg, 0.013 mmol), 3-bromooxetane (26.5 mg, 0.19 mmol), tris(trimethylsilyl)silane (32.0 mg, 0.13 mmol) and [4,4′-bis(1,1-dimethylethyl)-2,2′-bipyridine]nickel(II) dichloride (10.3 mg, 0.026 mmol) in 1,4-dioxane (4 mL) was stirred in sealed tube under N₂ atmosphere with 450 nm light (16 W) at rt for 16 h. The resulting mixture was filtered and then purified by pre-HPLC (FA as additive) to afford Example 122 (1.6 mg, 3.4% yield) as a white solid. MS (ESI⁺) obsd. [(M+H)⁺]: 366.1.

Example 122: ¹H NMR (500 MHz, DMSO-d6) δ ppm 13.54 (br s, 1H), 11.32 (s, 1H), 8.04 (s, 1H), 7.53 (br s, 1H), 7.22 (br dd, J=7.9, 11.1 Hz, 1H), 6.91 (dd, J=4.0, 7.7 Hz, 1H), 5.13 (s, 2H), 4.85 (ddd, J=2.1, 5.9, 8.3 Hz, 2H), 4.67 (ddd, J=5.9, 7.6, 11.9 Hz, 2H), 4.50 (quin, J=8.0 Hz, 1H), 2.20 (s, 3H).

### Example 126: 3-Amino-7-cyclopentyl-4-(7-fluoro-1H-indazol-4-yl)-8-methyl-1H-1,5-naphthyridin-2-one

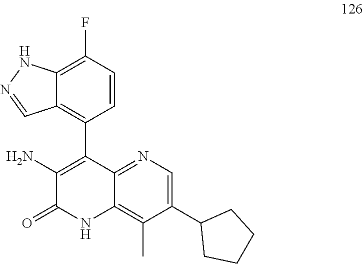 [SMILES: Cc1c(C2CCCC2)cnc2c(-c3ccc(F)c4[nH]ncc34)c(N)c(=O)[nH]c12]

### Step 1: 3-Amino-7-(cyclopent-1-en-1-yl)-4-(7-fluoro-1H-indazol-4-yl)-8-methyl-1,5-naphthyridin-2(1H)-one

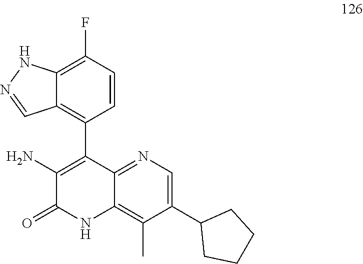 [SMILES: Cc1c(C2CCCC2)cnc2c(-c3ccc(F)c4[nH]ncc34)c(N)c(=O)[nH]c12]

A mixture of Example 115 (100.0 mg, 0.26 mmol), cyclopenten-1-ylboronic acid (86.5 mg, 0.77 mmol), Pd(dppf)Cl₂ (18.85 mg, 0.03 mmol) and Cs₂CO₃ (167.87 mg, 0.52 mmol) in degassed 1,4-dioxane (2 mL) and water (0.2 mL) was stirred at 100° C. for 6 h. After completion, the reaction mixture was quenched with saturated ammonium chloride (4 mL) and extracted with ethyl acetate (4 mL×3). The combined organic layer was dried over anhydrous sodium sulfate and concentrated under reduced pressure to give a residue, which was purified by silica-gel chromatography (elute with EtOAC:PE=0 to 20%) to give compound 126a (80.0 mg, 82.7% yield) as a light brown solid. MS (ESI⁺) obsd. [(M+H)⁺]: 376.2

### Step 2: 3-Amino-7-cyclopentyl-4-(7-fluoro-1H-indazol-4-yl)-8-methyl-1H-1,5-naphthyridin-2-one

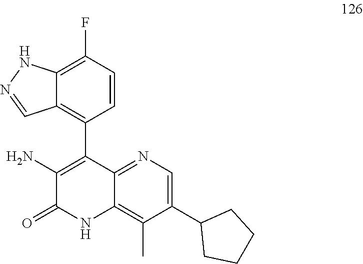 [SMILES: Cc1c(C2CCCC2)cnc2c(-c3ccc(F)c4[nH]ncc34)c(N)c(=O)[nH]c12]

A mixture of compound 126a (80.0 mg, 0.21 mmol), Pd/C (68.04 mg, 0.06 mmol) in methanol (2 mL) was stirred under H₂ balloon at 25° C. for 1 h. The mixture was filtered and the filtrate was concentrated under reduced pressure to give a residue, which was purified by prep-HPLC (HCl as additive) to give Example 126 (22.6 mg, 27.3% yield) as a yellow solid. MS (ESI⁺) obsd. [(M+H)⁺]: 378.1.

Example 126: ¹H NMR (400 MHz, DMSO-d₆) δ ppm 13.75 (s, 1H), 11.78 (s, 1H), 7.86 (s, 1H), 7.75 (s, 1H), 7.35 (dd, J=7.7, 10.1 Hz, 1H), 7.04 (dd, J=3.9, 7.6 Hz, 1H), 3.28-3.24 (m, 1H), 2.57 (s, 3H), 2.04-1.97 (m, 2H), 1.76-1.63 (m, 4H), 1.57-1.49 (m, 2H). ¹⁹FNMR (376 MHz, DMSO-d₆) δ ppm −130.870 (s, 1F).

### Example 127: 3-Amino-4-(7-fluoro-1H-indazol-4-yl)-7-isopropyl-8-methyl-1H-1,5-naphthyridin-2-one

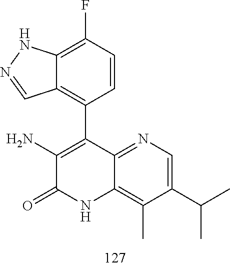 [SMILES: Cc1c(C(C)C)cnc2c(-c3ccc(F)c4[nH]ncc34)c(N)c(=O)[nH]c12]

The title compound was prepared in analogy to the preparation of Example 126, replacing compound 126a with Example 125 in step 2. Example 127 (27.4 mg) was obtained as a yellow solid. MS (ESI⁺) obsd. [(M+H)⁺]: 352.1.

Example 127: ¹H NMR (400 MHz, DMSO-d₆) δ ppm 7.81 (d, J=3.6 Hz, 1H), 7.78 (s, 1H), 7.37 (dd, J=7.6, 11.2 Hz, 1H), 7.09 (dd, J=4.0, 8.0 Hz, 1H), 3.31-3.25 (m, 1H), 2.61 (s, 3H), 1.20 (s, 3H), 1.19 (s, 3H). ¹⁹F NMR (376 MHz, DMSO-d₆) δ ppm −130.24 (s, 1F).

### Example 128: 3-Amino-7-ethyl-4-(7-fluoro-1H-indazol-4-yl)-8-methyl-1H-1,5-naphthyridin-2-one

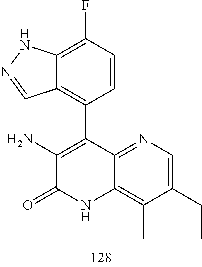 [SMILES: CCc1cnc2c(-c3ccc(F)c4[nH]ncc34)c(N)c(=O)[nH]c2c1C]

The title compound was prepared in analogy to the preparation of Example 126, replacing compound 126a with Example 123 in step 2. Example 128 (27.4 mg) was obtained as a yellow solid. MS (ESI⁺) obsd. [(M+H)⁺]: 338.1.

Example 128: ¹H NMR (400 MHz, MeOD-d₄) δ ppm 7.86 (d, J=3.2 Hz, 1H), 7.84 (s, 1H), 7.40 (dd, J=7.6, 10.8 Hz, 1H), 7.23 (dd, J=4.0, 7.6 Hz, 1H), 2.88 (q, J=7.6 Hz, 2H), 2.70 (s, 3H), 1.27 (t, J=7.6 Hz, 3H). ¹⁹F NMR (400 MHz, MeOD-d₄) δ ppm −131.48 (s, 1F).

### Example 129: 3-Amino-4-(7-fluoro-1H-indazol-4-yl)-8-methyl-7-(1-methylcyclopropyl)-1H-1,5-naphthyridin-2-one

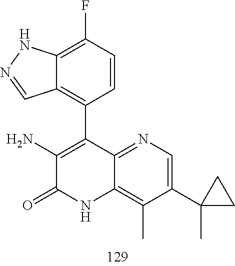 [SMILES: Cc1c(C2(C)CC2)cnc2c(-c3ccc(F)c4[nH]ncc34)c(N)c(=O)[nH]c12]

### Step 1: tert-Butyl N-[5-bromo-2-(7-fluoro-1-tetrahydropyran-2-yl-indazole-4-carbonyl)-4-methyl-3-pyridyl]carbamate

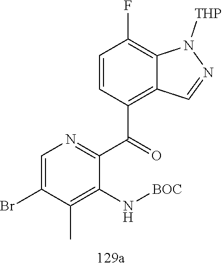

A mixture of Int-23a (1.0 g, 2.3 mmol), Boc anhydride (0.6 g, 2.8 mmol), DIPEA (0.5 g, 3.8 mmol) and DMAP (0.1 g, 0.83 mmol) in DCM (20 mL) was stirred at rt for 10 h. After completion, the mixture was washed with saturated NH₄Cl aqueous solution (20 mL×3). The organic layer was dried over anhydrous Na₂SO₄, concentrated under vacuum to give crude, which was purified by silica-gel chromatography (elute with EtOAc:PE=0 to 20%) to give compound 129a (1.0 g, 81.5% yield) as a light brown oil. MS (ESI⁺) obsd. [(M+H)⁺]: 533.3.

### Step 2: tert-Butyl (2-(7-fluoro-1-(tetrahydro-2H-pyran-2-yl)-1H-indazole-4-carbonyl)-4-methyl-5-(1-methylcyclopropyl)pyridin-3-yl)carbamate

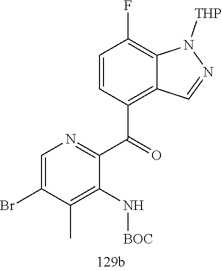

To a solution of compound 129a (300.0 mg, 0.56 mmol), 4,4,5,5-tetramethyl-2-(1-methylcyclopropyl)-1,3,2-dioxaborolane (307.21 mg, 1.69 mmol), Pd(dppf)Cl₂ (41.15 mg, 0.06 mmol) and Cs₂CO₃ (549.76 mg, 1.69 mmol) in degassed 1,4-dioxane (5 mL) and water (0.5 mL) was stirred at 100° C. for 12 h. After completion, the mixture was filtered and the filtrate was poured into water (10 mL) and extracted with EtOAc (10 mL×3). The combined organic layer was washed with brine (15 mL), dried over Na₂SO₄ and concentrated under reduced pressure to give a residue, which was purified by silica-gel chromatography (elute with EtOAC:PE=0 to 30%) to give compound 129b (190.0 mg, 66.4% yield) as a yellow solid. MS (ESI⁺) obsd. [(M+H)⁺]: 509.3.

### Step 3: (3-Amino-4-methyl-5-(1-methylcyclopropyl)pyridin-2-yl)(7-fluoro-1H-indazol-4-yl)methanone

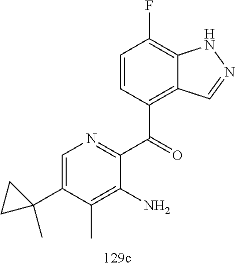 [SMILES: Cc1c(C2(C)CC2)cnc(C(=O)c2ccc(F)c3[nH]ncc23)c1N]

A solution of compound 129b (190.0 mg, 0.37 mmol) in HCl/EtOAc (3.0 mL, 4M) was stirred at 25° C. for 2 h. The reaction mixture was concentrated under reduced pressure to give crude compound 129c (120.0 mg, crude) as a yellow solid. MS (ESI⁺) obsd. [(M+H)⁺]: 325.2.

### Step 4: 2-Chloro-N-(2-(7-fluoro-1H-indazole-4-carbonyl)-4-methyl-5-(1-methylcyclopropyl)pyridin-3-yl)acetamide

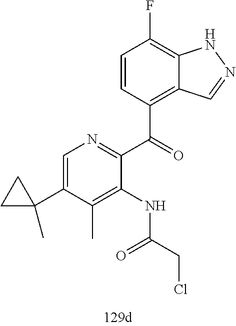 [SMILES: Cc1c(C2(C)CC2)cnc(C(=O)c2ccc(F)c3[nH]ncc23)c1NC(=O)CCl]

The title compound was prepared in analogy to the preparation of compound 001c, replacing compound 001b with compound 129c. Compound 129d (140.0 mg, crude) was obtained as a yellow solid. MS (ESI⁺) obsd. [(M+H)⁺]: 401.0.

### Step 5: 1-(4-(7-Fluoro-1H-indazol-4-yl)-8-methyl-7-(1-methylcyclopropyl)-2-oxo-1,2-dihydro-1,5-naphthyridin-3-yl)pyridin-1-ium chloride

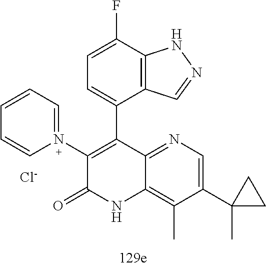

The title compound was prepared in analogy to the preparation of compound 001d, replacing compound 001c with compound 129d. Compound 129e (160.0 mg, crude) was obtained as a yellow solid. MS (ESI⁺) obsd. [(M-Cl)⁺]: 426.3.

### Step 6: 3-Amino-4-(7-fluoro-1H-indazol-4-yl)-8-methyl-7-(1-methylcyclopropyl)-1H-1,5-naphthyridin-2-one

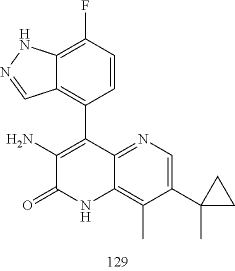 [SMILES: Cc1c(C2(C)CC2)cnc2c(-c3ccc(F)c4[nH]ncc34)c(N)c(=O)[nH]c12]

The title compound was prepared in analogy to the preparation of compound 001, replacing compound 001d with compound 129e. Example 129 (140.0 mg, crude) was obtained as a yellow solid. MS (ESI⁺) obsd. [(M+H)⁺]: 364.1.

Example 129: ¹H NMR (400 MHz, DMSO-d₆) δ ppm 13.09 (s, 1H), 12.21 (s, 1H), 7.87 (d, J=3.6 Hz, 1H), 7.82 (s, 1H), 7.40 (dd, J=7.6, 11.2 Hz, 1H), 7.08 (dd, J=4.0, 7.6 Hz, 1H), 6.75 (s, 2H), 2.79 (s, 3H), 1.31 (s, 3H), 0.87-0.84 (m, 2H), 0.81-0.78 (m, 2H). ¹⁹F NMR (376 MHz, DMSO-d₆) δ=−130.40 (s, 1F).

The following Examples 130 to 132 and 144 were prepared in analogy to the procedure described for the preparation of Example 129, replacing 4,4,5,5-tetramethyl-2-(1-methylcyclopropyl)-1,3,2-dioxaborolane with BOROCPD indicated in Table 11 in step 2.

TABLE 11
Compound synthesis and characterization

| | Compound Names and | | |
|---|---|---|---|
| Ex. | Structures | BOROCPD | NMR and (ESI⁺) |
| 130 | 3-Amino-7-(2,2- dimethylcyclopropyl)- 4-(7-fluoro-1H-indazol- 4-yl)-8-methyl-1H-1,5- naphthyridin-2-one 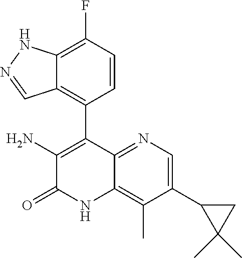 [SMILES: Cc1c(C2CC2(C)C)cnc2c(-c3ccc(F)c4[nH]ncc34)c(N)c(=O)[nH]c12] | 2-(2,2- Dimethylcyclopropyl)- 4,4,5,5- tetramethyl-1,3,2- dioxaborolane | ¹H NMR (400 MHz, MeOH- d₄) δ ppm 7.87 (d, J = 2.4 Hz, 1H), 7.65 (s, 1H), 7.42- 7.37 (m, 1H), 7.24-7.21 (m, 1H), 2.75 (s, 3H), 1.97-1.93 (m, 1H), 1.38 (s, 3H), 1.01- 0.98 (m, 1H), 0.89-0.87 (m, 1H), 0.79 (d, J = 1.2 Hz, 3H). ¹⁹F NMR (376 MHz, MeOH- d₄) δ ppm −131.44 (s, 1F). MS (ESI⁺) obsd. [(M + H)⁺]: 378.1. |
| 131 | 3-Amino-7- (cyclopropylmethyl)-4- (7-fluoro-1H-indazol-4- yl)-8-methyl-1H-1,5- naphthyridin-2-one 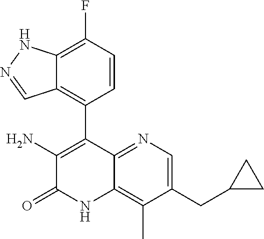 [SMILES: Cc1c(CC2CC2)cnc2c(-c3ccc(F)c4[nH]ncc34)c(N)c(=O)[nH]c12] | Cyclopropylmethyl (trifluoro)boranuide | ¹H NMR (400 MHz, MeOD- d₄) δ ppm = 7.97 (s, 1H), 7.87 (d, J = 3.2 Hz, 1H), 7.40 (dd, J = 7.6 Hz, J = 10.8 Hz, 1H), 7.24 (dd, J = 4 Hz, J = 8.0 Hz, 1H), 2.75 (d, J = 6.8 Hz, 2H), 2.69 (s, 3H), 1.06- 1.02 (m, 1H), 0.62-0.60 (m, 2H), 0.28-0.24 (m, 2H). ¹⁹F NMR (377 MHz, MeOD- d₄) δ ppm −131.39 (s, 1F). MS (ESI⁺) obsd. [(M + H)⁺]: 364.3. |
| 132 | 3-Amino-7-(2- fluorocyclopropyl)-4- (7-fluoro-1H-indazol-4- yl)-8-methyl-1H-1,5- naphthyridin-2-one 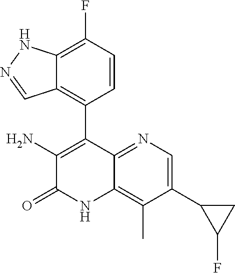 [SMILES: Cc1c(C2CC2F)cnc2c(-c3ccc(F)c4[nH]ncc34)c(N)c(=O)[nH]c12] | 2-(2- Fluorocyclopropyl)- 4,4,5,5- tetramethyl-1,3,2- dioxaborolane | ¹H NMR (400 MHz, MeOD- d₄) δ ppm 7.84 (d, J = 7.6 Hz, 1H), 7.63 (s, 1H), 7.42-7.37 (m, 1H), 7.23-7.19 (m, 1H), 4.81-4.65 (m, 1H), 2.84 (s, 3H), 2.64-2.54 (m, 1H), 1.70-1.60 (m, 1H), 1.25-1.21 (m, 1H). ¹⁹F NMR (377 MHz, MeOD- d₄) δ = −131.375 (s, 1F), −207.093 (s, 1F). MS (ESI⁺) obsd. [(M + H)⁺]: 368.1. |
| 144 | 3-Amino-4-(7-fluoro- 1H-indazol-4-yl)-8- methyl-7-[rac-(1R,2R)- 2-methylcyclopropyl]- 1H-1,5-naphthyridin-2- one 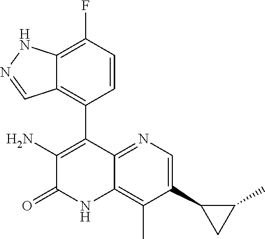 [SMILES: Cc1c([C@@H]2C[C@H]2C)cnc2c(-c3ccc(F)c4[nH]ncc34)c(N)c(=O)[nH]c12] | 4,4,5,5- Tetramethyl-2- [(1R,2R)-2- methylcyclopropyl]- 1,3,2- dioxaborolane | ¹H NMR (400 MHz, MeOD- d₄) δ = 7.81 (s, 1H), 7.66 (s, 1H), 7.40-7.35 (m, 1H), 7.21-7.18 (m, 1H), 2.79 (s, 3H), 1.78-1.73 (m, 1H), 1.30 (d, J = 5.6 Hz, 3H), 1.04-0.99 (m, 1H), 0.99- 0.93 (m, 1H), 0.88-0.83 (m, 1H). ¹⁹F NMR (400 MHz, MeOD- d₄) δ ppm −131.947 (s, 1F). MS (ESI⁺) obsd. [(M + H)⁺]: 364.1. |

### Example 133: 3-Amino-4-(7-fluoro-1H-indazol-4-yl)-8-methyl-7-(2,2,2-trifluoroethyl)-1H-1,5-naphthyridin-2-one

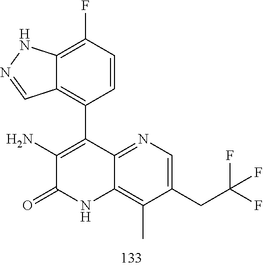 [SMILES: Cc1c(CC(F)(F)F)cnc2c(-c3ccc(F)c4[nH]ncc34)c(N)c(=O)[nH]c12]

The title compound was prepared in analogy to the preparation of Example 101, replacing iodomethane-d₃ with 1,1,1-trifluoro-2-iodo-ethane in step 3 of the synthesis of Int-23. Example 133 (14.7 mg, crude) was obtained as a yellow solid. MS (ESI⁺) obsd. [(M+H)⁺]: 392.1.

Example 133: ¹H NMR (400 MHz, DMSO-d₆) δ ppm 13.81 (s, 1H), 12.06 (s, 1H), 8.07 (s, 1H), 7.83 (s, 1H), 7.37 (dd, J=8.0, 11.6 Hz, 1H), 7.08 (dd, J=4.0, 7.6 Hz, 1H), 4.01-3.93 (m, 2H), 2.62 (s, 3H). ¹⁹F NMR (376 MHz, DMSO-d₆) δ=−63.92 (s, 3F), −130.98 (s, 1F).

### Example 134: 3-Amino-7-(1,1-difluoroethyl)-4-(7-fluoro-1H-indazol-4-yl)-8-methyl-1H-1,5-naphthyridin-2-one

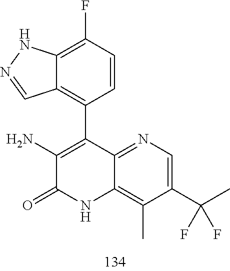 [SMILES: Cc1c(C(C)(F)F)cnc2c(-c3ccc(F)c4[nH]ncc34)c(N)c(=O)[nH]c12]

### Step 1: 3-Amino-7-(1-ethoxyvinyl)-4-(7-fluoro-1H-indazol-4-yl)-8-methyl-1,5-naphthyridin-2(1H)-one

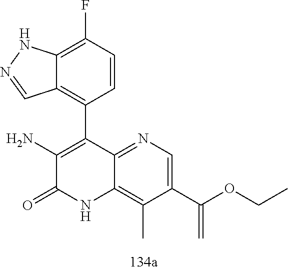 [SMILES: C=C(OCC)c1cnc2c(-c3ccc(F)c4[nH]ncc34)c(N)c(=O)[nH]c2c1C]

A mixture of Example 115 (150.0 mg, 0.39 mmol), Pd(PPh₃)₄ (44.65 mg, 0.04 mmol) and tributyl(1-ethoxyvinyl)tin (230.0 mg. 0.64 mmol) in degassed 1,4-dioxane (5 mL) was stirred at 100° C. for 4 h The reaction mixture was filtered and the filtrate was concentrated under reduced pressure to give compound 134a (150.0 mg, crude) as a dark brown oil, which was used in the next step directly. MS (ESI⁺) obsd. [(M+H)⁺]: 380.0

### Step 2: 7-Acetyl-3-amino-4-(7-fluoro-1H-indazol-4-yl)-8-methyl-1,5-naphthyridin-2(1H)-one

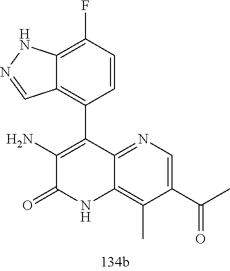 [SMILES: CC(=O)c1cnc2c(-c3ccc(F)c4[nH]ncc34)c(N)c(=O)[nH]c2c1C]

A solution of compound 134a (150 0 mg, 0.4 mmol) in HCl/EA (4M. 2.0 mL) and THF (2 mL) was stirred at 25° C. for 2 h. After completion, the reaction mixture was concentrated under reduced pressure to give a residue, which was purified by prep-HPLC (aqueous ammonia hydroxide as additive) to give compound 134b (50.0 mg, 36.0% yield) as a yellow solid. MS (ESI⁺) obsd. [(M+H)⁺]: 352.0.

### Step 3: 3-Amino-4-(7-fluoro-1H-indazol-4-yl)-8-methyl-7-(2-methyl-1,3-dithiolan-2-yl)-1,5-naphthyridin-2(1H)-one

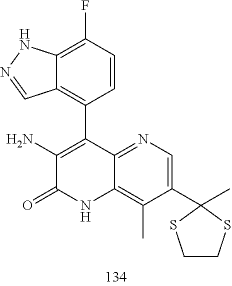 [SMILES: Cc1c(C2(C)SCCS2)cnc2c(-c3ccc(F)c4[nH]ncc34)c(N)c(=O)[nH]c12]

A mixture of compound 134b (20.0 mg, 0.06 mmol). BF₃·Et₂O (0.03 mL, 0.23 mmol) and 1,2-ethanedithiol (21.45 mg, 0.23 mmol) in DCM (2 mL) was stirred at 40° C. for 10 h. After completion, the mixture was concentrated under reduced pressure to give a crude residue, which was purified by prep-HPLC (aqueous ammonia hydroxide as additive) to give compound 134c (9.0 mg, 37.0% yield) as a yellow solid. MS (ESI⁺) obsd. [(M+H)⁺]: 428.1.

### Step 4: 3-Amino-7-(1,1-difluoroethyl)-4-(7-fluoro-1H-indazol-4-yl)-8-methyl-1H-1,5-naphthyridin-2-one

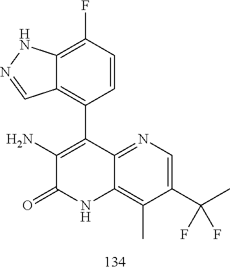 [SMILES: Cc1c(C(C)(F)F)cnc2c(-c3ccc(F)c4[nH]ncc34)c(N)c(=O)[nH]c12]

To a solution of NIS (18.42 mg, 0.08 mmol) in DCM (2.0 mL) was added drop wise pyridine hydrofluoride (0.02 mL, 0.16 mmol) at −60° C., and the reaction mixture was stirred at −60° C. for 1 h. Compound 134c (7.0 mg, 0.02 mmol) dissolved in DCM (0.1 mL) was added dropwise to the above solution at −60° C. Then the reaction mixture was stirred at −60° C. for 2 h. After completion, the reaction mixture was concentrated under reduced pressure to give a crude residue, which was purified by prep-HPLC (HCl as additive) to give Example 134 (2.33 mg, 34.0% yield) as a yellow solid. MS (ESI⁺) obsd. [(M+H)⁺]: 374.1.

Example 134: ¹H NMR (400 MHz., MeOD-d₄) δ ppm 8.04 (s, 1H), 7.86 (d, J=3.2 Hz, 1H), 7.41-7.36 (m, 1H), 7.24-7.21 (m, 1H), 2.80 (s, 3H), 2.08 (t, J=18.8 Hz, 3H). ¹⁹F NMR (376 MHz, MeOD-d₄) δ ppm −85.378 (m, 2F), −131.65 (s, 1F).

### Example 135: 3-Amino-7-fluoro-4-(7-fluoro-1H-indazol-4-yl)-8-methyl-1H-1,5-naphthyridin-2-one

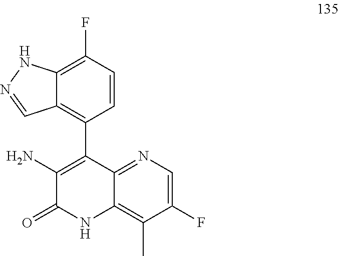 [SMILES: Cc1c(F)cnc2c(-c3ccc(F)c4[nH]ncc34)c(N)c(=O)[nH]c12]

The title compound was prepared in analogy to the preparation of Example 045, replacing Int-2 with tert-butyl N-(2-bromo-5-fluoro-4-methyl-3-pyridyl)carbamate and compound 045a with 7-fluoro-1-tetrahydropyran-2-yl-indazole-4-carbaldehyde in step 2. Example 135 (26.5 mg) was obtained as a yellow solid. MS (ESI⁺) obsd. [(M+H)⁺]: 328.1.

Example 135: ¹H NMR (400 MHz, MeOD-d₄) δ ppm 8.08 (d, J=2.4 Hz, 1H), 7.80 (d, J=3.2 Hz, 1H), 7.36 (dd, J=7.6, 10.8 Hz, 1H), 7.19 (dd, J=4.0, 7.6 Hz, 1H), 2.62 (d, J=1.6 Hz, 3H). ¹⁹F NMR (376 MHz, MeOD-d₄) δ ppm −132.27 (s, 1F), −135.80 (s, 1F).

### Example 136: 3-Amino-4-(7-fluoro-1H-indazol-4-yl)-7-methoxy-8-methyl-1H-1,5-naphthyridin-2-one

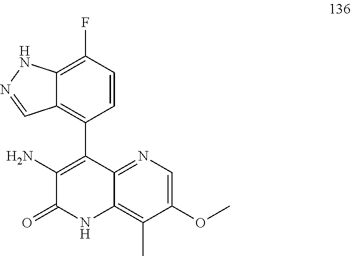 [SMILES: COc1cnc2c(-c3ccc(F)c4[nH]ncc34)c(N)c(=O)[nH]c2c1C]

A mixture of Example 116 (100.0 mg, 0.23 mmol), MeONa (0.13 mL, 0.69 mmol), CuI (8.75 mg, 0.05 mmol) and 1,10-phenanthroline (8.28 mg, 0.05 mmol) in degassed DMF (2 mL) was stirred at 100° C. for 16 h. After completion, the mixture was filtered and the filtrate was purified by prep-HPLC (NH₄HCO₃ as additive) to give Example 136 (5.93 mg, 7.6% yield) as a yellow solid. MS (ESI⁺) obsd. [(M+H)⁺]: 340.1.

Example 136: ¹H NMR (400 MHz, DMSO-d6) δ ppm 13.60 (s, 1H), 11.35 (s, 1H), 8.00 (s, 1H), 7.60 (s, 1H), 7.28 (dd, J=7.6, 11.2 Hz, 1H), 6.98 (dd, J=4.0, 7.6 Hz, 1H), 4.98 (s, 2H), 3.85 (s, 3H), 2.34 (s, 3H). ¹⁹F NMR (376 MHz, DMSO-d6) δ ppm −133.01 (s, 1F).

### Example 137: 3-Amino-4-(7-fluoro-1H-indazol-4-yl)-8-methyl-7-(2,2,2-trifluoroethoxy)-1H-1,5-naphthyridin-2-one

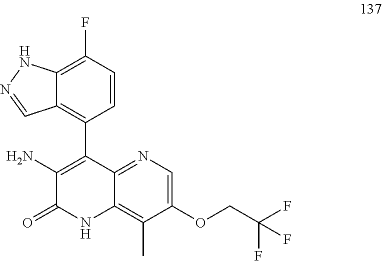 [SMILES: Cc1c(OCC(F)(F)F)cnc2c(-c3ccc(F)c4[nH]ncc34)c(N)c(=O)[nH]c12]

### Step 1: tert-Butyl (2-(7-fluoro-1-(tetrahydro-2H-pyran-2-yl)-1H-indazole-4-carbonyl)-4-methyl-5-(4,4,5,5-tetramethyl-1,3,2-dioxaborolan-2-yl)pyridin-3-yl)carbamate

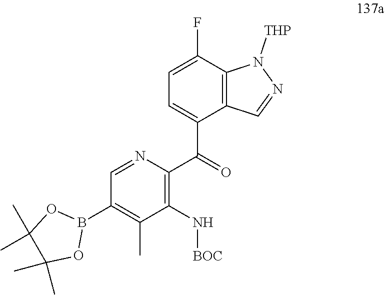

A mixture of compound 129a (1.6 g, 3.0 mmol), bis(pinacolato)diboron (1.52 g, 6.0 mmol), Pd(dppf)Cl₂ (219 mg, 0.3 mmol) and KOAc (1.0 g, 10.5 mmol) in degassed 1,4-dioxane (20 mL) was stirred at 110° C. for 12 h under N₂ protection. After completion, the reaction mixture was quenched with saturated ammonium chloride (20 mL) and extracted with EtOAc (50 mL×3). The combined organic layer was dried over anhydrous sodium sulfate and concentrated under reduced pressure to give compound 137a (1.7 g, 97.6% yield) as a yellow solid. MS (ESI⁺) obsd. [(M+H)⁺]: 581.4.

### Step 2: tert-Butyl (2-(7-fluoro-1-(tetrahydro-2H-pyran-2-yl)-1H-indazole-4-carbonyl)-5-hydroxy-4-methylpyridin-3-yl) carbamate

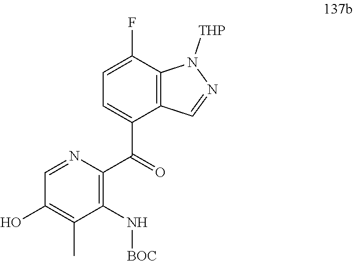

To a solution of compound 137a (1.7 g, 2.9 mmol) in THE (20 mL) and water (3 mL), was added H₂O₂ (7.0 g, 61.7 mmol) dropwise at rt and stirred at 25° C. for 2 h. After completion, the reaction mixture was quenched with saturated Na₂SO₃ (40 mL) and extracted with EtOAc (50 mL×3). The combined organic layer was washed with brine (60 mL), dried over anhydrous sodium sulfate and concentrated under vacuum to give a residue, which was purified by silica-gel chromatography (elute with EtOAc:PE=0 to 20%) to compound 137b (1.3 g, 97.8% yield) as a yellow gum. MS (ESI⁺) obsd. [(M+H)⁺]: 471.3.

### Step 3: tert-Butyl N-[2-(7-fluoro-1-tetrahydropyran-2-yl-indazole-4-carbonyl)-4-methyl-5-(2,2,2-trifluoroethoxy)-3-pyridyl]carbamate

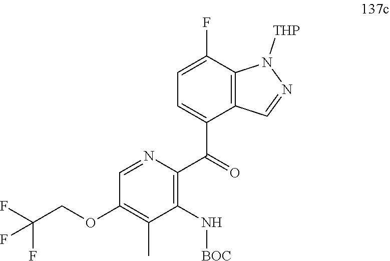

A mixture of compound 137b (200 mg, 0.43 mmol), K₂CO₃ (117 mg, 0.85 mmol), 1,1,1-trifluoro-2-iodo-ethane (394 mg, 1.9 mmol) and NaI (23.0 mg, 0.13 mmol) in DMF (3 mL) was stirred at 25° C. for 2 h. The reaction mixture was quenched with saturated aqueous ammonium chloride (10 mL) and extracted with EtOAc (10 mL×3). The combined organic layer was dried over anhydrous Na₂SO₄, concentrated under vacuum to give reduce, which was purified by silica-gel chromatography (elute with EtOAc:PE=0 to 20%) to compound 137c (200.0 mg, 85.1% yield) as a yellow oil. MS (ESI⁺) obsd. [(M+H)⁺]: 553.3.

### Step 4: (3-Amino-4-methyl-5-(2,2,2-trifluoroethoxy)pyridin-2-yl)(7-fluoro-1H-indazol-4-yl)methanone

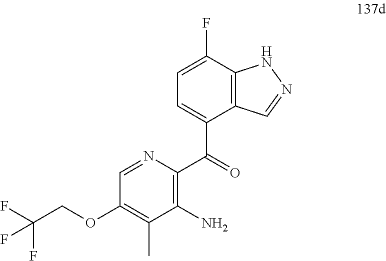 [SMILES: Cc1c(OCC(F)(F)F)cnc(C(=O)c2ccc(F)c3[nH]ncc23)c1N]

A solution of compound 137c (200 mg, 0.36 mmol) in HCl/EA (4.0 mL, 4 M) was stirred at 25° C. for 3 h. The reaction mixture was concentrated under reduced pressure to give a residue, which was purified by prep-TLC (EtOAc/PE=1:2) to give compound 137d (130.0 mg, 97.5% yield) as a yellow solid. MS (ESI⁺) obsd. [(M+H)⁺]: 369.3.

### Step 5: 2-Chloro-N-(2-(7-fluoro-1H-indazole-4-carbonyl)-4-methyl-5-(2,2,2-trifluoroethoxy)pyridin-3-yl)acetamide

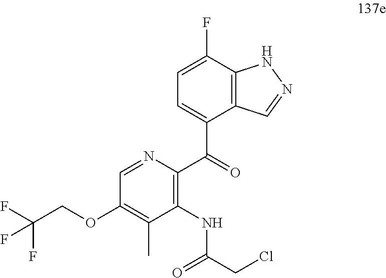 [SMILES: Cc1c(OCC(F)(F)F)cnc(C(=O)c2ccc(F)c3[nH]ncc23)c1NC(=O)CCl]

The title compound was prepared in analogy to the preparation of compound 001c, replacing compound 001b with compound 137d. Compound 137e (140.0 mg, crude) was obtained as a yellow solid. MS (ESI⁺) obsd. [(M+H)⁺]: 445.2.

### Step 6: 1-(4-(7-Fluoro-1H-indazol-4-yl)-8-methyl-2-oxo-7-(2,2,2-trifluoroethoxy)-1,2-dihydro-1,5-naphthyridin-3-yl)pyridin-1-ium chloride

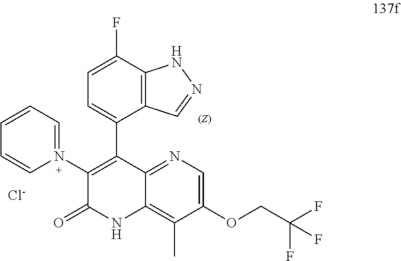

The title compound was prepared in analogy to the preparation of compound 001d, replacing compound 001c with compound 137e. Compound 137f (160.0 mg, crude) was obtained as a yellow solid. MS (ESI⁺) obsd. [(M-Cl)⁺]: 470.2.

### Step 7: 3-Amino-4-(7-fluoro-1H-indazol-4-yl)-8-methyl-7-(2,2,2-trifluoroethoxy)-1H-1,5-naphthyridin-2-one

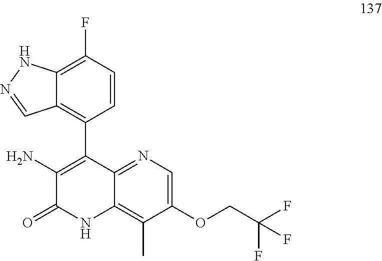 [SMILES: Cc1c(OCC(F)(F)F)cnc2c(-c3ccc(F)c4[nH]ncc34)c(N)c(=O)[nH]c12]

The title compound was prepared in analogy to the preparation of compound 001, replacing compound 001d with compound 137f. Example 137 (140.0 mg, crude) was obtained as a yellow solid. MS (ESI⁺) obsd. [(M+H)⁺]: 408.3.

Example 137: ¹H NMR (400 MHz, DMSO-d₆) δ ppm 13.61 (s, 1H), 11.45 (s, 1H), 8.05 (s, 1H), 7.61 (d, J=3.2 Hz, 1H), 7.28 (dd, J=7.6, 11.2 Hz, 1H), 6.96 (dd, J=4.0, 7.6 Hz, 1H), 5.09 (s, 2H), 4.82 (q, J=8.8 Hz, 2H), 2.38 (s, 3H). ¹⁹F NMR (376 MHz, DMSO-d₆) δ ppm −72.98 (s, 1F), −132.89 (s, 1F).

The following Examples 138 to 140 were prepared in analogy to the procedure described for the preparation of Example 137, replacing 1,1,1-trifluoro-2-iodo-ethane with IODOCPD indicated in Table 12 in step 3.

TABLE 12
Compound synthesis and characterization

| | Compound Names and | | |
|---|---|---|---|
| Ex. | Structures | IODOCPD | NMR and (ESI⁺) |
| 138 | 3-Amino-4-(7-fluoro- 1H-indazol-4-yl)-7- isopropoxy-8-methyl- 1H-1,5-naphthyridin-2- one 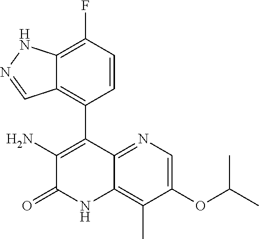 [SMILES: Cc1c(OC(C)C)cnc2c(-c3ccc(F)c4[nH]ncc34)c(N)c(=O)[nH]c12] | 2-Iodopropane | ¹H NMR (400 MHz, MeOH- d₄) δ ppm 7.87 (d, J = 2.4 Hz, 1H), 7.65 (s, 1H), 7.42- 7.37 (m, 1H), 7.24-7.21 (m, 1H), 2.75 (s, 3H), 1.97-1.93 (m, 1H), 1.38 (s, 3H), 1.01- 0.98 (m, 1H), 0.89-0.87 (m, 1H), 0.79 (d, J = 1.2 Hz, 3H). ¹⁹F NMR (376 MHz, MeOH- d₄) δ ppm −131.44 (s, 1F). MS (ESI⁺) obsd. [(M + H)⁺]: 368.1. |
| 139 | 3-Amino-7- (cyclobutoxy)-4-(7- fluoro-1H-indazol-4- yl)-8-methyl-1H-1,5- naphthyridin-2-one 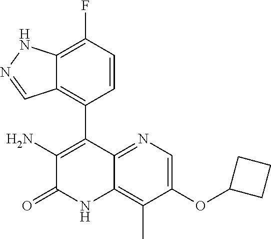 [SMILES: Cc1c(OC2CCC2)cnc2c(-c3ccc(F)c4[nH]ncc34)c(N)c(=O)[nH]c12] | Iodocyclobutane | ¹H NMR (400 MHz, MeOD- d₄) δ ppm 7.85 (d, J = 3.2 Hz, 1H), 7.45 (s, 1H), 7.39 (dd, J = 8.0, 10.8 Hz, 1H), 7.22 (dd, J = 4.0, 8.0 Hz, 1H), 4.78-4.76 (m, 1H), 2.59 (s, 3H), 2.51-2.43 (m, 2H), 2.27-2.17 (m, 2H), 1.95-1.86 (m, 1H), 1.80-1.68 (m, 1H). ¹⁹F NMR (376 MHz, MeOD-d₄) δ ppm −131.56 (s, 1F). MS (ESI⁺) obsd. [(M + H)⁺]: 380.1. |
| 140 | 3-Amino-7-ethoxy-4- (7-fluoro-1H-indazol-4- yl)-8-methyl-1H-1,5- naphthyridin-2-one 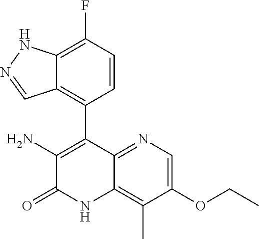 [SMILES: CCOc1cnc2c(-c3ccc(F)c4[nH]ncc34)c(N)c(=O)[nH]c2c1C] | Iodoethane | ¹H NMR (400 MHz, DMSO- d₆) δ ppm 13.79 (s, 1H), 11.90 (s, 1H), 7.76 (s, 2H), 7.35 (dd, J = 8.0, 11.2 Hz, 1H), 7.06 (dd, J = 4.4, 8.0 Hz, 1H), 4.13 (q, J = 7.2 Hz, 2H), 2.46 (s, 3H), 1.36 (t, J = 6.8 Hz, 3H). ¹⁹FNMR (376 MHz, DMSO- d₆) δ ppm −134.42 (s, 1F). MS (ESI⁺) obsd. [(M + H)⁺]: 354.1. |

### Example 141: 3-Amino-4-(7-fluoro-1H-indazol-4-yl)-8-methyl-7-methylsulfanyl-1H-1,5-naphthyridin-2-one

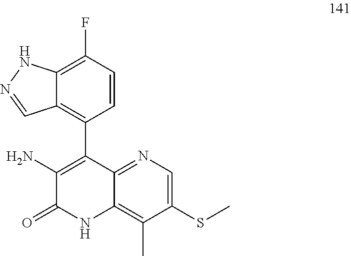 [SMILES: CSc1cnc2c(-c3ccc(F)c4[nH]ncc34)c(N)c(=O)[nH]c2c1C]

The mixture of Example 115 (100.0 mg, 0.26 mmol), sodium thiomethoxide (72.22 mg, 1.03 mmol), t-BuONa (1.29 mL, 2.58 mmol) and Pd(PPh₃)₄ (29.77 mg, 0.03 mmol) in degassed DMSO (2 mL) was stirred at 100° C. for 12 h under N₂ protection. After completion, the reaction mixture was quenched with aqueous ammonium chloride (10 mL) and extracted with ethyl acetate (20 mL×3). The combined organic layer was dried over anhydrous Na₂SO₄ and concentrated under reduced pressure to give a residue, which was purified by prep-HPLC (HCl as additive) to give Example 141 (93.0 mg, 91.7% yield) as a yellow solid. MS (ESI⁺) obsd. [(M+H)⁺]: 356.1.

Example 141: ¹H NMR (400 MHz, MeOD-d₄) δ ppm 7.88 (d, J=3.2 Hz, 1H), 7.81 (s, 1H), 7.40 (dd, J=7.6, 10.8 Hz, 1H), 7.24 (dd, J=4.0, 8.0 Hz, 1H), 2.76 (s, 3H), 2.54 (s, 3H). ¹⁹F NMR (376 MHz, MeOD-d₄) δ ppm −131.39 (s, 1F).

### Example 142 & 143: 3-Amino-4-(7-fluoro-1H-indazol-4-yl)-8-methyl-7-methylsulfinyl-1H-1,5-naphthyridin-2-one (142) & 3-Amino-4-(7-fluoro-1H-indazol-4-yl)-8-methyl-7-methylsulfonyl-1H-1,5-naphthyridin-2-one (143)

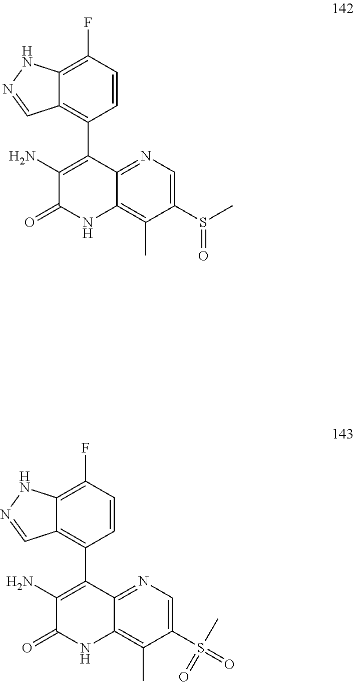 [SMILES: Cc1c(S(C)=O)cnc2c(-c3ccc(F)c4[nH]ncc34)c(N)c(=O)[nH]c12 ; Cc1c(S(C)(=O)=O)cnc2c(-c3ccc(F)c4[nH]ncc34)c(N)c(=O)[nH]c12]

A mixture of Example 141 (18.0 mg, 45.9 umol) and 3-chloroperoxybenzoic acid (25.7 mg, 114.8 umol) in DCM (5.0 mL) and DMF (2.0 mL) was stirred at rt for 3 h. After completion, the reaction mixture was quenched with sat. Na₂S₂O₃-solution and extracted four times with EtOAc (5 mL×3). The organic layers were dried over anhydrous Na₂SO₄ and concentrated under vacuum to give crude residue, which was purified by pre-HPLC (FA as additive) to give example Example 142 (5.9 mg, 34% yield) as a white powder and Example 143 (1.8 mg, 10% yield) as a white powder.

Example 142: ¹H NMR (500 MHz, DMSO-d6) δ ppm 13.63 (br s, 1H), 11.69 (s, 1H), 8.40 (s, 1H), 7.63 (br s, 1H), 7.34-7.25 (m, 1H), 7.03-6.94 (m, 1H), 5.57 (br s, 2H), 2.79 (d, J=4.6 Hz, 3H), 2.52 (s, 3H). MS (ESI⁺) obsd. [(M+H)⁺]: 372.1.

Example 143: ¹H NMR (500 MHz, DMSO-d6) δ ppm 13.63 (br s, 1H), 11.75 (s, 1H), 8.48 (s, 1H), 7.64 (br s, 1H), 7.29 (br dd, J=8.0, 10.9 Hz, 1H), 6.97 (dd, J=4.0, 7.6 Hz, 1H), 5.86 (br s, 2H), 3.26 (s, 3H), 2.81 (s, 3H). MS (ESI⁺) obsd. [(M+H)⁺]: 388.1.

### Example 145: 3-Amino-8-(difluoromethyl)-4-(7-fluoro-1H-indazol-4-yl)-7-methyl-1H-1,5-naphthyridin-2-one

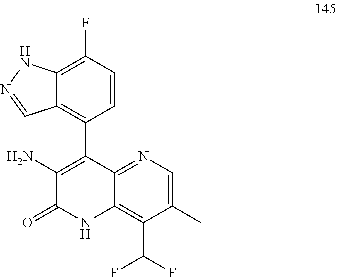 [SMILES: Cc1cnc2c(-c3ccc(F)c4[nH]ncc34)c(N)c(=O)[nH]c2c1C(F)F]

The title compound was prepared in analogy to the preparation of Example 045, replacing Int-2 with tert-butyl N-[4-(difluoromethyl)-2-iodo-5-methyl-3-pyridyl]carbamate and compound 045a with 7-fluoro-1-tetrahydropyran-2-yl-indazole-4-carbaldehyde in step 2. Example 145 (46.1 mg) was obtained as a yellow solid. MS (ESI⁺) obsd. [(M+H)⁺]: 360.3.

Example 145: ¹H NMR (400 MHz, MeOD-d₄) δ ppm 7.98 (s, 1H), 7.90 (d, J=3.2 Hz, 1H), 7.85-7.52 (m, 1H), 7.40 (dd, J=7.6, 10.8 Hz, 1H), 7.24 (dd, J=4.0, 7.6 Hz, 1H), 2.60 (s, 3H). ¹⁹F NMR (376 MHz, MeOD-d₄) δ ppm −118.09 (s, 2F), −131.14 (s, 1F).

### Example 146: 3-Amino-4-(7-chloro-1H-indazol-4-yl)-7,8-dimethyl-1H-1,5-naphthyridin-2-one

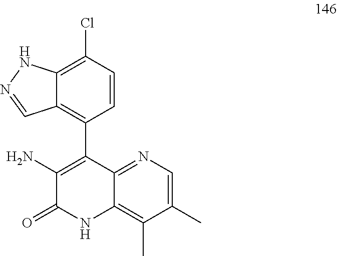 [SMILES: Cc1cnc2c(-c3ccc(Cl)c4[nH]ncc34)c(N)c(=O)[nH]c2c1C]

The title compound was prepared in analogy to the preparation of Example 045, replacing Int-2 with tert-butyl N-(2-bromo-4,5-dimethyl-3-pyridyl)carbamate and compound 045a with 7-chloro-1-tetrahydropyran-2-yl-indazole-4-carbaldehyde in step 2. Example 146 (38.1 mg) was obtained as a yellow solid. MS (ESI⁺) obsd. [(M+H)⁺]: 340.8.

Example 146: ¹H NMR (500 MHz, DMSO-d6) δ ppm 13.55 (s, 1H), 11.38 (s, 1H), 7.92 (s, 1H), 7.60 (s, 1H), 7.53 (d, J=7.5 Hz, 1H), 7.02 (d, J=7.5 Hz, 1H), 5.17 (s, 2H), 2.40 (s, 3H), 2.24 (s, 3H).

### Example 147: 3-Amino-6-bromo-4-(7-fluoro-1H-indazol-4-yl)-8-methyl-1H-1,5-naphthyridin-2-one

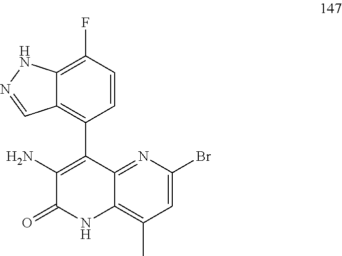 [SMILES: Cc1cc(Br)nc2c(-c3ccc(F)c4[nH]ncc34)c(N)c(=O)[nH]c12]

The title compound was prepared in analogy to the preparation of Example 091, replacing NCS with NBS in step 4. Example 147 (44.7 mg) was obtained as a yellow solid. MS (ESI⁺) obsd. [(M+H)⁺]: 388.0.

Example 147: ¹H NMR (400 MHz, DMSO-d₆) δ ppm 13.71 (s, 1H), 11.56 (br s, 1H), 7.59 (d, J=1.2 Hz, 1H), 7.21-7.36 (m, 1H), 7.07 (s, 1H), 6.87 (dd, J=7.6, 4.4 Hz, 1H) 5.45 (s, 2H) 2.43 (s, 3H).

### Example 148: 3-Amino-4-(7-fluoro-1H-indazol-4-yl)-6-(3-hydroxy-3-methyl-but-1-ynyl)-8-methyl-1H-1,5-naphthyridin-2-one

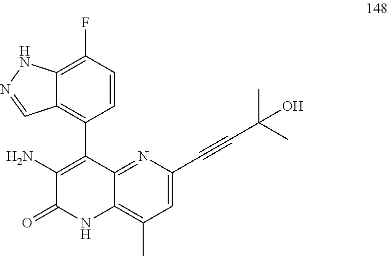 [SMILES: Cc1cc(C#CC(C)(C)O)nc2c(-c3ccc(F)c4[nH]ncc34)c(N)c(=O)[nH]c12]

A mixture of Example 147 (60.0 mg, 0.15 mmol), 2-methyl-3-butyn-2-ol (26.0 mg, 0.31 mmol), diisopropylamine (4.01 mL, 23.09 mmol), Pd(PPh₃)₂Cl₂ (5.42 mg, 0.01 mmol) and CuI (2.94 mg, 0.02 mmol) in degassed 1,4-dioxane (1 mL) was stirred at 80° C. for 3 h under N₂ protection. The reaction mixture was filtered, and the filtrate was concentrated under reduced pressure to give a residue, which was purified by prep-HPLC (NH₄HCO₃ as additive) to afford Example 148 (45.3 mg, 71.0% yield) as a yellow solid. MS (ESI⁺) obsd. [(M+H)⁺]: 392.2.

Example 148: ¹H NMR (400 MHz, DMSO-d₆) δ ppm 13.65 (s, 1H), 7.65 (s, 1H), 7.30 (dd, J=8.0, 11.2 Hz, 1H), 7.13 (s, 1H), 6.95 (dd, J=4.0, 8.0 Hz, 1H), 5.42 (s, 1H), 5.27 (s, 2H), 2.46 (s, 3H), 1.35 (s, 6H). ¹⁹F NMR (376 MHz, DMSO-d₆) δ ppm −132.78 (s, 1F).

The following Examples 149 and 150 were prepared in analogy to the procedure described for the preparation of Example 148, replacing 2-methyl-3-butyn-2-ol with ALKYNE indicated in Table 13.

TABLE 13
Compound synthesis and characterization

| | Compound Names and | | |
|---|---|---|---|
| Ex. | Structures | ALKYNE | NMR and (ESI⁺) |
| 149 | 3-Amino-4-(7-fluoro- 1H-indazol-4-yl)-6-(3- methoxy-3-methyl-but- 1-ynyl)-8-methyl-1H- 1,5-naphthyridin-2-one 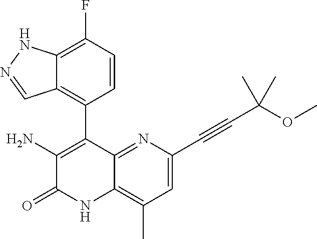 [SMILES: COC(C)(C)C#Cc1cc(C)c2[nH]c(=O)c(N)c(-c3ccc(F)c4[nH]ncc34)c2n1] | 3-Methoxy-3- methyl-but-1- yne | ¹H NMR (400 MHz, DMSO-d₆) δ ppm 13.65 (s, 1H), 11.61 (d, J = 1.6 Hz, 1H), 7.65 (s, 1H), 7.29 (dd, J = 8.0, 11.2 Hz, 1H), 7.20 (s, 1H), 6.98 (dd, J = 4.0, 8.0 Hz, 1H), 5.31 (s, 2H), 3.19 (s, 3H), 2.47 (s, 3H), 1.36 (d, J = 2.0 Hz, 6H). ¹⁹F NMR (376 MHz, DMSO-d₆) δ ppm −132.70 (s, 1F). MS (ESI⁺) obsd. [(M + H)⁺]: 406.2. |
| 150 | 3-Amino-4-(7-fluoro- 1H-indazol-4-yl)-8- methyl-6-[2-(oxetan-3- yl)ethynyl]-1H-1,5- naphthyridin-2-one 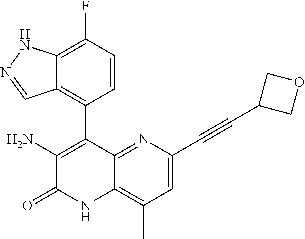 [SMILES: Cc1cc(C#CC2COC2)nc2c(-c3ccc(F)c4[nH]ncc34)c(N)c(=O)[nH]c12] | 3-Ethynyloxetane | ¹H NMR (400 MHz, DMSO-d₆) δ ppm 13.62 (br s, 1H), 11.65 (br s, 1H), 7.65 (d, J = 3.2 Hz, 1H), 7.30 (dd, J = 8.0, 11.2 Hz, 1H), 7.22 (s, 1H), 6.96 (dd, J = 4.0, 8.0 Hz, 1H), 4.69 (dd, J = 5.6, 8.4 Hz, 2H), 4.51 (dd, J = 5.6, 6.8 Hz, 2H), 4.05-4.01 (m, 1H), 2.47 (s, 3H). ¹⁹F NMR (376 MHz, DMSO-d₆) δ ppm −132.70 (s, 1F). MS (ESI⁺) obsd. [(M + H)⁺]: 390.1. |

### Example 151: 3-Amino-4-(7-fluoro-1H-indazol-4-yl)-7-methyl-6-(2-thienyl)-1H-1,5-naphthyridin-2-one

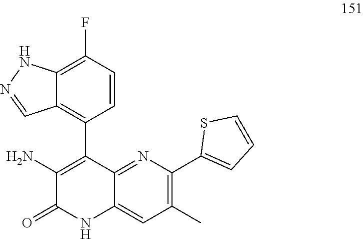 [SMILES: Cc1cc2[nH]c(=O)c(N)c(-c3ccc(F)c4[nH]ncc34)c2nc1-c1cccs1]

The mixture of Example 021 (20.0 mg, 0.05 mmol), tributyl(2-thienyl)stannane (40.0 mg, 0.10 mmol) and Pd(PPh₃)₄ (4.0 mg) in degassed 1,4-dioxane (2 mL) was stirred at 100° C. for 2 h under N₂ protection. After completion, the mixture was concentrated under vacuum to give crude, which was purified by prep-HPLC (NH₄HCO₃ as additive) to give Example 151 (6.1 mg, 31.6% yield) as a yellow solid. MS (ESI⁺) obsd. [(M+H)⁺]: 392.1.

Example 151: ¹H NMR (400 MHz, MeOH-d₄) a ppm 9.15 (d, J=2.1 Hz, 1H), 8.53 (br d, J=2.8 Hz, 1H), 8.39 (s, 1H), 8.26 (d, J=2.1 Hz, 1H), 8.14-8.03 (m, 3H), 3.65 (s, 3H).

The following Examples 152 to 155 were prepared in analogy to the procedure described for the preparation of Example 151, replacing tributyl(2-thienyl)stannane with TINCPD indicated in Table 14.

TABLE 14
Compound synthesis and characterization

| | Compound Names and | | |
|---|---|---|---|
| Ex. | Structures | TINCPD | NMR and (ESI⁺) |
| 152 | 3-Amino-4-(7-fluoro- 1H-indazol-4-yl)-7- methyl-6-thiazol-4-yl- 1H-1,5-naphthyridin-2- one 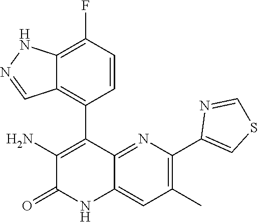 [SMILES: Cc1cc2[nH]c(=O)c(N)c(-c3ccc(F)c4[nH]ncc34)c2nc1-c1cscn1] | Tributyl (thiazol-4-yl) stannane | ¹H NMR (400 MHz, MeOD-d₄) δ ppm 10.17 (d, J = 2.1 Hz, 1H), 8.98 (br d, J = 2.8 Hz, 1H), 8.79 (s, 1H), 8.58 (d, J = 2.1 Hz, 1H), 8.54-8.43 (m, 2H), 3.85 (s, 3H). ¹⁹F NMR (376 MHz, MeOH-d₄) δ ppm −133.61 (s, 1F). MS (ESI⁺) obsd. [(M + H)⁺]: 393.2. |
| 153 | 3-Amino-4-(7-fluoro- 1H-indazol-4-yl)-7- methyl-6-thiazol-2-yl- 1H-1,5-naphthyridin-2- one 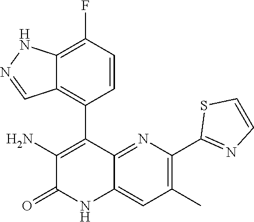 [SMILES: Cc1cc2[nH]c(=O)c(N)c(-c3ccc(F)c4[nH]ncc34)c2nc1-c1nccs1] | Tributyl(thiazol- 2-yl)stannane | ¹H NMR (400 MHz, DMSO-d₆) δ ppm 13.59 (br s, 1H), 12.25 (br s, 1H), 7.82 (d, J = 3.3 Hz, 1H), 7.67 (br d, J = 2.5 Hz, 1H), 7.56- 7.44 (m, 2H), 7.33 (dd, J = 7.8, 11.2 Hz, 1H), 7.14 (dd, J = 4.2, 7.8 Hz, 1H), 5.46 (s, 2H), 2.74 (s, 3H). ¹⁹F NMR (376 MHz, DMSO-d₆) δ ppm −132.73 (s, 1F). MS (ESI⁺) obsd. [(M + H)⁺]: 393.2. |
| 154 | 3-Amino-4-(7-fluoro- 1H-indazol-4-yl)-7- methyl-6-oxazol-2-yl- 1H-1,5-naphthyridin-2- one 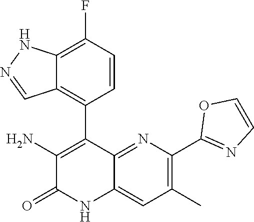 [SMILES: Cc1cc2[nH]c(=O)c(N)c(-c3ccc(F)c4[nH]ncc34)c2nc1-c1ncco1] | Tributyl(oxazol- 2-yl)stannane | ¹H NMR (400 MHz, DMSO-d₆) δ ppm 13.61 (br s, 1H), 12.27 (br d, J = 1.5 Hz, 1H), 8.02 (s, 1H), 7.68 (br s, 1H), 7.55 (s, 1H), 7.38- 7.24 (m, 2H), 7.08 (dd, J = 4.2, 7.8 Hz, 1H), 5.32 (s, 2H), 2.61 (s, 3H). ¹⁹F NMR (376 MHz, DMSO-d₆) δ ppm −132.75 (s, 1F). MS (ESI⁺) obsd. [(M + H)⁺]: 377.1. |
| 155 | 3-Amino-4-(7-fluoro- 1H-indazol-4-yl)-7- methyl-6-(2-pyridyl)- 1H-1,5-naphthyridin-2- one 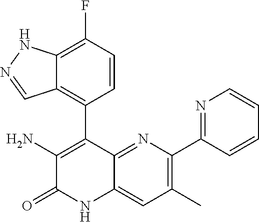 [SMILES: Cc1cc2[nH]c(=O)c(N)c(-c3ccc(F)c4[nH]ncc34)c2nc1-c1ccccn1] | Tributyl (2-pyridyl)stannane | ¹H NMR (400 MHz, DMSO-d₆) δ ppm 13.53 (br s, 1H), 12.13 (s, 1H), 8.45 (d, J = 4.0 Hz, 1H), 7.68-7.54 (m, 2H), 7.44 (s, 1H), 7.33-7.14 (m, 3H), 7.04 (dd, J = 4.2, 7.8 Hz, 1H), 5.21 (s, 2H), 2.46 (s, 3H) ¹⁹F NMR (376 MHz, DMSO-d₆) δ ppm −132.71 (s, 1F). MS (ESI⁺) obsd. [(M + H)⁺]: 387.2. |

### Example 156: 3-Amino-4-(7-fluoro-1H-indazol-4-yl)-7-methyl-6-(3-methylbut-1-ynyl)-1H-1,5-naphthyridin-2-one

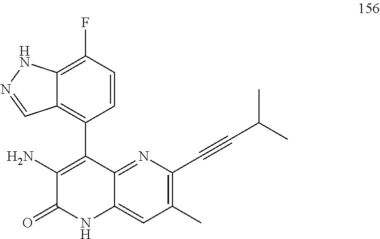 [SMILES: Cc1cc2[nH]c(=O)c(N)c(-c3ccc(F)c4[nH]ncc34)c2nc1C#CC(C)C]

The title compound was prepared in analogy to the preparation of Example 070, replacing 2-methyl-3-butyn-2-ol with 3-methylbut-1-yne. Example 156 (11.7 mg) was obtained as a yellow solid. MS (ESI⁺) obsd. [(M+H)⁺]: 376.2.

Example 156: ¹H NMR (400 MHz, DMSO-d₆) δ ppm 13.65 (br s, 1H), 12.21 (s, 1H), 7.64 (d, J=3.2 Hz, 1H), 7.45 (s, 1H), 7.30 (dd, J=8.0, 11.2 Hz, 1H), 6.98 (dd, J=4.0, 8.0 Hz, 1H), 2.76-2.69 (m, 1H), 2.34 (s, 3H), 1.14 (d, J=6.8 Hz, 6H). ¹⁹F NMR (376 MHz, DMSO-d₆) δ ppm −132.64 (s, 1F).

### Example 157: 3-Amino-6-ethyl-4-(7-fluoro-1H-indazol-4-yl)-7-methyl-1H-1,5-naphthyridin-2-one

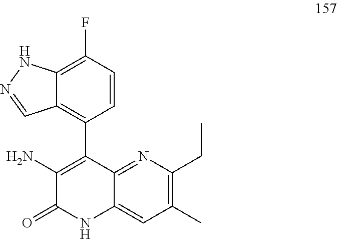 [SMILES: CCc1nc2c(-c3ccc(F)c4[nH]ncc34)c(N)c(=O)[nH]c2cc1C]

### Step 1: 3-Amino-4-(7-fluoro-1H-indazol-4-yl)-7-methyl-6-vinyl-1,5-naphthyridin-2(1H)-one

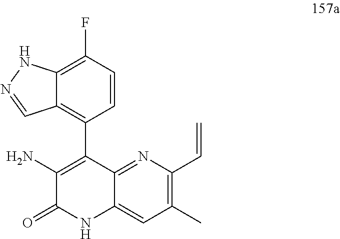 [SMILES: C=Cc1nc2c(-c3ccc(F)c4[nH]ncc34)c(N)c(=O)[nH]c2cc1C]

A mixture of Example 021 (200.0 mg, 0.52 mmol), vinylboronic acid pinacol ester (119.03 mg, 0.77 mmol), Pd(dppf)Cl₂ (37.7 mg, 0.05 mmol) and Cs₂CO₃ (335.72 mg, 1.03 mmol) in degassed 1,4-dioxane (3 mL) and water (0.300 mL) was stirred at 100° C. for 12 h under N₂ protection. After completion, the mixture was poured into water (5 mL) and extracted with EtOAc (5 mL×3). The combined organic layer was dried over anhydrous Na₂SO₄ and concentrated under reduced pressure to give a residue, which was purified by silica-gel chromatograpy (elute with EtOAc:PE=0 to 20%) to give compound 157a (100.0 mg, 57.8% yield) as a yellow solid. MS (ESI⁺) obsd. [(M+H)⁺]: 336.2.

### Step 2: 3-Amino-6-ethyl-4-(7-fluoro-1H-indazol-4-yl)-7-methyl-1H-1,5-naphthyridin-2-one

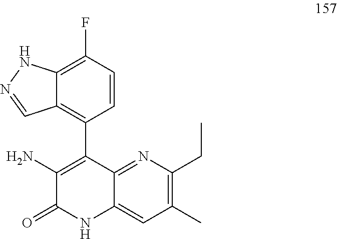 [SMILES: CCc1nc2c(-c3ccc(F)c4[nH]ncc34)c(N)c(=O)[nH]c2cc1C]

A suspension of Pd/C (100.0 mg, 0.09 mmol) and compound 157a (100.0 mg, 0.3 mmol) in methanol (3 mL) was stirred at 25° C. for 0.5 h under H₂ (15 psi) atmosphere. After completion, the mixture was filtered and the filtrate was concentrated under reduced pressure to give a residue, which was purified by prep-HPLC (HCl as additive) to give Example 157 (14.7 mg, 14.6% yield) as a yellow solid. MS (ESI⁺) obsd. [(M+H)⁺]: 338.1.

Example 157: ¹H NMR (400 MHz, MeOD-d₄) δ ppm 7.95 (s, 1H), 7.86 (d, J=3.2 Hz, 1H), 7.39 (dd, J=7.6, 10.8 Hz, 1H), 7.25 (dd, J=4.0, 7.6 Hz, 1H), 2.87 (q, J=7.6 Hz, 2H), 2.49 (s, 3H), 1.11 (t, J=7.6 Hz, 3H). ¹⁹F NMR (376 MHz, MeOD-d₄) δ ppm −131.64 (s, 1F).

### Example 158: 3-Amino-7-ethyl-4-(7-fluoro-1H-indazol-4-yl)-1H-1,5-naphthyridin-2-one

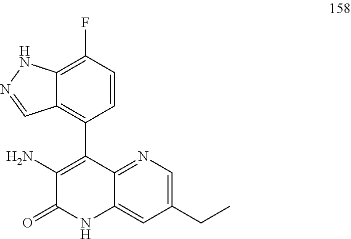 [SMILES: CCc1cnc2c(-c3ccc(F)c4[nH]ncc34)c(N)c(=O)[nH]c2c1]

The title compound was prepared in analogy to the preparation of Example 157, replacing Example 021 with Example 001 in step 1. Example 158 (48.9 mg) was obtained as a yellow solid. MS (ESI⁺) obsd. [(M+H)⁺]: 324.1.

Example 158: ¹H NMR (400 MHz, MeOD-d₄) δ ppm 7.98 (d, J=1.6 Hz, 1H), 7.92 (d, J=1.6 Hz, 1H), 7.86 (d, J=3.2 Hz, 1H), 7.39 (dd, J=7.6, 10.8 Hz, 1H), 7.23 (dd, J=3.6, 7.6 Hz, 1H), 2.82 (q, J=7.6 Hz, 2H), 1.32 (t, J=7.6 Hz, 3H). ¹⁹F NMR (377 MHz, MeOD-d₄) δ ppm −131.73 (s, 1F).

### Example 159: 3-Amino-4-(7-chloro-1H-indazol-4-yl)-6,7-dimethyl-1H-1,5-naphthyridin-2-one

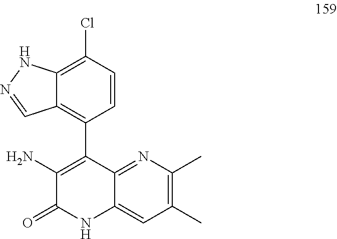 [SMILES: Cc1cc2[nH]c(=O)c(N)c(-c3ccc(Cl)c4[nH]ncc34)c2nc1C]

The title compound was prepared in analogy to the preparation of Example 045, replacing Int-2 with tert-butyl N-(2-bromo-5,6-dimethyl-3-pyridyl)carbamate and compound 045a with 7-chloro-1-tetrahydropyran-2-yl-indazole-4-carbaldehyde in step 2. Example 159 (17.1 mg) was obtained as a yellow solid. MS (ESI⁺) obsd. [(M+H)⁺]: 324.3.

Example 159: ¹H NMR (400 MHz, MeOD-d₄) δ ppm 7.95 (s, 1H), 7.88 (s, 1H), 7.68 (d, J=7.6 Hz, 1H), 7.24 (d, J=7.6 Hz, 1H), 2.50 (s, 3H), 2.45 (s, 3H).

### Example 160: 3-Amino-5-chloro-4-(7-fluoro-1H-indazol-4-yl)-7,8-dimethyl-1H-1,6-naphthyridin-2-one

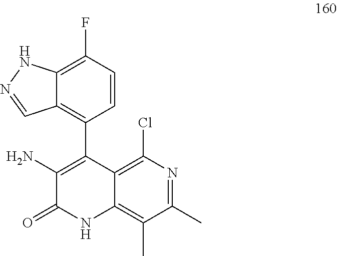 [SMILES: Cc1nc(Cl)c2c(-c3ccc(F)c4[nH]ncc34)c(N)c(=O)[nH]c2c1C]

The title compound was prepared in analogy to the preparation of Example 045, replacing Int-2 with Int-24 and compound 045a with 7-Fluoro-1-tetrahydropyran-2-yl-indazole-4-carbaldehyde in step 2. Example 160 (9.9 mg) was obtained as a yellow solid. MS (ESI⁺) obsd. [(M+H)⁺]: 358.1.

Example 160: ¹H NMR (400 MHz, DMSO-d₆) δ ppm 11.52 (s, 1H), 7.74 (d, J=3.6 Hz, 1H), 7.26 (dd, J=8.0, 11.2 Hz, 1H), 6.91 (dd, J=4.0, 7.6 Hz, 1H), 2.40 (s, 3H), 2.37 (s, 3H). ¹⁹F NMR (376 MHz, DMSO-d₆) δ ppm −132.41 (s, 1F).

### Example 161: 3-Amino-4-(7-fluoro-1H-indazol-4-yl)-7,8-dimethyl-2-oxo-1H-1,6-naphthyridine-5-carbonitrile

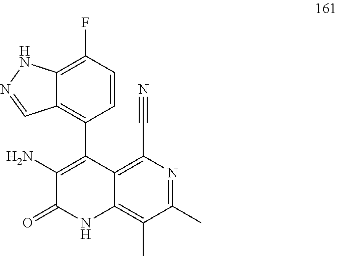 [SMILES: Cc1nc(C#N)c2c(-c3ccc(F)c4[nH]ncc34)c(N)c(=O)[nH]c2c1C]

A mixture of Example 160 (40.0 mg, 0.11 mmol), Zn (14.62 mg, 0.22 mmol), Pd(dppf)Cl₂ (16.36 mg, 0.02 mmol) and Zn(CN)₂ (39.39 mg, 0.34 mmol) in DMF (3 mL) was stirred at 100° C. for 12 h under N₂ protection. After completion, the reaction mixture was quenched with saturated sodium bicarbonate (10 mL) and extracted with ethyl acetate (15 mL×3). The combined organic layer was dried over anhydrous sodium sulfate and concentrated under reduced pressure to give a residue, which was purified by prep-HPLC (NH₄CO₃ as additive) give Example 161 (13.9 mg, 34.3% yield) as an off-white solid. MS (ESI⁺) obsd. [(M+H)⁺]: 349.1.

Example 161: ¹H NMR (400 MHz, DMSO-d₆) δ ppm 13.71 (s, 1H), 11.69 (s, 1H), 7.75 (s, 1H), 7.29 (dd, J=8.0, 10.8 Hz, 1H), 7.03 (dd, J=4.0, 7.6 Hz, 1H), 5.43 (s, 2H), 2.46 (s, 3H), 2.45 (s, 3H). ¹⁹F NMR (376 MHz, DMSO-d₆) δ ppm −131.10 (s, 1F).

### Example 162: 3-Amino-5-chloro-7-cyclopropyl-4-(7-fluoro-1H-indazol-4-yl)-8-methyl-1H-1,6-naphthyridin-2-one

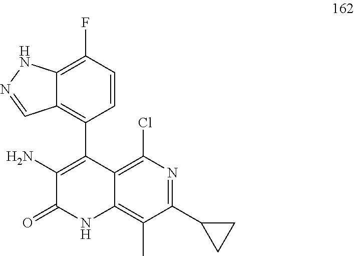 [SMILES: Cc1c(C2CC2)nc(Cl)c2c(-c3ccc(F)c4[nH]ncc34)c(N)c(=O)[nH]c12]

The title compound was prepared in analogy to the preparation of Example 160, replacing 2-chloro-6-methyl-pyridin-4-amine with 2-chloro-6-cyclopropyl-pyridin-4-amine in step 1 of the synthesis of Int-24. Example 162 (11.9 mg) was obtained as a yellow solid. MS (ESI⁺) obsd. [(M+H)⁺]: 384.3.

Example 162: ¹H NMR (400 MHz, MeOD-d₄) δ ppm 7.72 (d, J=3.2 Hz, 1H), 7.24 (dd, J=7.6, 11.2 Hz, 1H), 7.00 (dd, J=4.0, 7.6 Hz, 1H), 2.57 (s, 3H), 2.28-2.18 (m, 1H), 1.00-0.90 (m, 4H). ¹⁹F NMR (376 MHz, MeOD-d₄) δ ppm −134.49 (s, 1F).

### Example 163: 3-Amino-5-chloro-7-cyclopropyl-4-(7-fluoro-1H-indazol-4-yl)-1H-1,6-naphthyridin-2-one

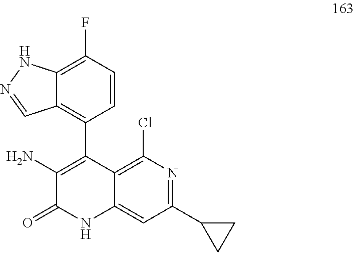 [SMILES: Nc1c(-c2ccc(F)c3[nH]ncc23)c2c(Cl)nc(C3CC3)cc2[nH]c1=O]

The title compound was prepared in analogy to the preparation of Example 045, replacing Int-2 with Int-25 and compound 045a with 7-fluoro-1-tetrahydropyran-2-yl-indazole-4-carbaldehyde in step 2. Example 163 (4.3 mg) was obtained as a yellow solid. MS (ESI⁺) obsd. [(M+H)⁺]: 370.2.

Example 163: ¹H NMR (400 MHz, MeOD-d₄) δ ppm 7.79 (d, J=3.2 Hz, 1H), 7.26 (dd, J=7.6, 11.2 Hz, 1H), 7.05-7.02 (m, 2H), 2.11 (d, J=16.4 Hz, 1H), 1.13-1.09 (m, 2H), 1.00-0.97 (m, 2H). ¹⁹F NMR (376 MHz, MeOD-d₄) δ ppm −133.84 (s, 1F).

### Example 164: 3-Amino-5-chloro-4-(7-fluoro-1H-indazol-4-yl)-8-methyl-1H-1,6-naphthyridin-2-one

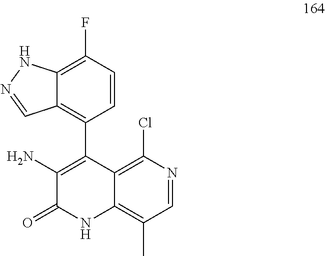 [SMILES: Cc1cnc(Cl)c2c(-c3ccc(F)c4[nH]ncc34)c(N)c(=O)[nH]c12]

The title compound was prepared in analogy to the preparation of Example 163, replacing 2-chloro-6-cyclopropyl-pyridin-4-amine with 2-chloro-5-methyl-pyridin-4-amine in step 1 of the synthesis of Int-25. Example 164 (4.3 mg) was obtained as a yellow solid. MS (ESI⁺) obsd. [(M+H)⁺]: 344.1.

Example 164: ¹H NMR (400 MHz, DMSO-d₆) δ=13.67 (s, 1H), 11.70 (s, 1H), 7.89 (d, J=0.4 Hz, 1H), 7.75 (s, 1H), 7.29-7.24 (m, 1H), 6.94-6.91 (m, 1H), 5.12 (s, 2H), 2.41 (s, 3H). ¹⁹F NMR (377 MHz, DMSO-d₆) δ ppm −131.44 (s, 1F).

### Example 165: 3-amino-7-cyclopropyl-4-(7-fluoro-1H-indazol-4-yl)-8-methyl-1H-1,6-naphthyridin-2-one

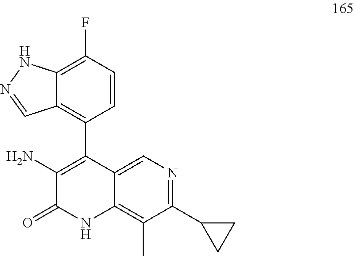 [SMILES: Cc1c(C2CC2)ncc2c(-c3ccc(F)c4[nH]ncc34)c(N)c(=O)[nH]c12]

A mixture of Example 162 (60.0 mg, 0.16 mmol) and Pd/C (60 mg, 0.16 mmol) in methanol (2 mL) was stirred at 25° C. for 3 h under H₂ balloon. The mixture was filtered and the filtrate was concentrated under reduced pressure to give a residue, which was purified by prep-HPLC (HCl as additive) to give Example 165 (3.53 mg, 6.4% yield) as a white solid. MS (ESI⁺) obsd. [(M+H)⁺]: 350.3.

Example 165: ¹H NMR (400 MHz, MeOD-d₄) δ ppm 7.80 (d, J=2.4 Hz, 1H), 7.63 (s, 1H), 7.36 (dd, J=8.0, 10.8 Hz, 1H), 7.17 (dd, J=4.0, 7.8 Hz, 1H), 2.70 (s, 3H), 2.38-2.34 (m, 1H), 1.26-1.23 (m, 2H), 1.00-0.97 (m, 2H). ¹⁹F NMR (376 MHz, MeOD-d₄) δ ppm −134.49 (s, 1F).

### Example 166: 3-Amino-7-cyclopropyl-4-(7-fluoro-1H-indazol-4-yl)-5-methyl-1H-1,6-naphthyridin-2-one

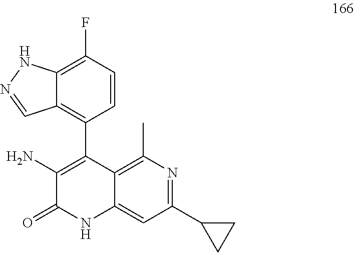 [SMILES: Cc1nc(C2CC2)cc2[nH]c(=O)c(N)c(-c3ccc(F)c4[nH]ncc34)c12]

A mixture of Example 163 (200 mg, 0.54 mmol), trimethylboroxine (0.31 mL, 1.08 mmol), Pd(dppf)Cl₂ (39.5 mg, 0.05 mmol) and Cs₂CO₃ (440 mg, 1.35 mmol) in 1,4-dioxane (2 mL) was stirred at 100° C. for 12 h under N₂ protection. After completion, the reaction mixture was poured into saturated ammonium chloride (5 mL) and extracted with ethyl acetate (10 mL×3). The combined organic layer was dried over anhydrous sodium sulfate and concentrated under reduced pressure to give a residue, which was purified by prep-HPLC (HCl as additive) to give Example 166 (122.6 mg, 56.4% yield) as a yellow solid. MS (ESI⁺) obsd. [(M+H)⁺]: 350.3.

Example 166: ¹H NMR (400 MHz, MeOD-d₄) δ ppm 7.88-7.87 (m, 1H), 7.32 (dd, J=7.6, 10.8 Hz, 1H), 7.13 (dd, J=3.9, 7.8 Hz, 1H), 7.02 (s, 1H), 2.27-2.23 (m, 1H), 1.94 (s, 3H), 1.35-1.29 (m, 2H), 1.08-1.04 (m, 2H). ¹⁹F NMR (376 MHz, MeOD-d₄) δ ppm −132.34 (s, 1F).

### Example 167: 3-Amino-7-cyclopropyl-4-(7-fluoro-1H-indazol-4-yl)-2-oxo-1H-1,6-naphthyridine-5-carbonitrile

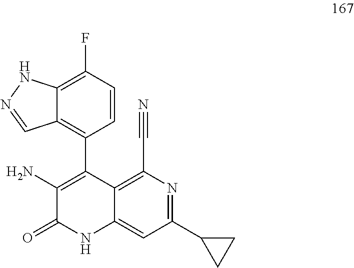 [SMILES: N#Cc1nc(C2CC2)cc2[nH]c(=O)c(N)c(-c3ccc(F)c4[nH]ncc34)c12]

A mixture of Example 163 (100 mg, 0.27 mmol), Zn(CN)₂ (94.9 mg, 0.81 mmol), Pd(dppf)Cl₂ (19.7 mg, 0.03 mmol) and Zn (43.9 mg, 0.68 mmol) in DMF (3 mL) was stirred at 130° C. for 12 h under N₂ protection. After completion, the reaction mixture was poured into saturated ammonium chloride (5 mL) and extracted with EtOAc (10 mL×3). The combined organic layer was dried over anhydrous sodium sulfate and concentrated under reduced pressure to give a residue, which was purified by prep-HPLC (NH₄HCO₃ as additive) to give Example 167 (22.1 mg, 22.4% yield) as a yellow solid. MS (ESI⁺) obsd. [(M+H)⁺]: 361.3.

Example 167: ¹H NMR (400 MHz, MeOD-d₄) δ ppm 7.74 (d, J=3.2 Hz, 1H), 7.28-7.26 (m, 1H), 7.24 (s, 1H), 7.12 (dd, J=4.0, 7.6 Hz, 1H), 2.09-2.05 (m, 1H), 1.00-0.94 (m, 4H). ¹⁹F NMR (376 MHz, MeOD-d₄) δ ppm −132.74 (s, 1F).

### Example 168: 3-Amino-7-cyclopropyl-5-(difluoromethoxy)-4-(7-fluoro-1H-indazol-4-yl)-1H-1,6-naphthyridin-2-one

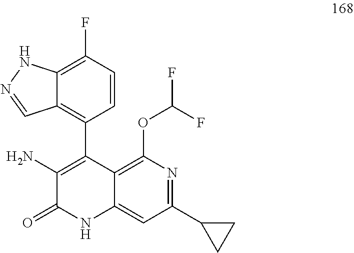 [SMILES: Nc1c(-c2ccc(F)c3[nH]ncc23)c2c(OC(F)F)nc(C3CC3)cc2[nH]c1=O]

### Step 1: 3-Amino-5-benzyloxy-7-cyclopropyl-4-(7-fluoro-1H-indazol-4-yl)-1H-1,6-naphthyridin-2-one

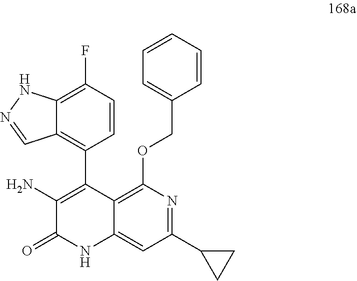 [SMILES: Nc1c(-c2ccc(F)c3[nH]ncc23)c2c(OCc3ccccc3)nc(C3CC3)cc2[nH]c1=O]

A mixture of Example 163 (110 mg, 0.30 mmol) and benzyloxysodium (50 mg, 0.38 mmol) in toluene (2 mL) was stirred at 120° C. for 12 h. After completion, the mixture was poured to saturated NH₄Cl solution (5 mL) and extracted with EtOAc (10 mL×3). The combined organic layer was dried over anhydrous Na₂SO₄ and concentrated under reduced pressure to give a residue, which was purified by prep-HPLC (HCl as additive) to give compound 168a (30.8 mg, 23.2% yield) as a yellow solid. MS (ESI⁺) obsd. [(M+H)⁺]: 442.2.

### Step 2: 3-Amino-7-cyclopropyl-4-(7-fluoro-1H-indazol-4-yl)-5-hydroxy-1H-1,6-naphthyridin-2-one

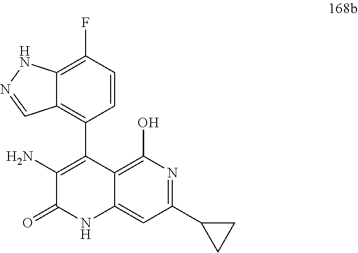 [SMILES: Nc1c(-c2ccc(F)c3[nH]ncc23)c2c(O)nc(C3CC3)cc2[nH]c1=O]

A mixture of compound 168a (30.8 mg) and Pd/C (20 mg) in EtOAc (10 mL) was stirred at 25° C. under H₂ balloon for 0.5 h. After completion, the mixture was filtered and the filtrate was concentrated under vacuum to give a crude compound 168b (30.0 mg, 100% yield), which was used in the next step directly. MS (ESI⁺) obsd. [(M+H)⁺]: 352.1

### Step 3: 3-Amino-7-cyclopropyl-5-(difluoromethoxy)-4-(7-fluoro-1H-indazol-4-yl)-1H-1,6-naphthyridin-2-one

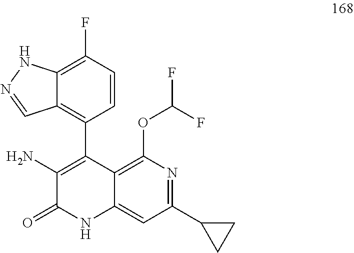 [SMILES: Nc1c(-c2ccc(F)c3[nH]ncc23)c2c(OC(F)F)nc(C3CC3)cc2[nH]c1=O]

A mixture of compound 168b (30.0 mg crude, 0.085 mmol), Na₂SO₄ (20.2 mg, 0.16 mmol) and 2,2-difluoro-2-(fluorosulfonyl)acetic acid (118.35 mg, 0.66 mmol) in MeCN (2 mL) was stirred at 25° C. for 1 h. After completion, the reaction mixture was quenched with saturated sodium bicarbonate (5 mL) and extracted with ethyl acetate (8 mL×3). The combined organic layer was dried over anhydrous sodium sulfate and concentrated under reduced pressure to give Example 168 (5.4 mg, 15.8% yield) as a yellow solid. MS (ESI⁺) obsd. [(M+H)⁺]: 402.2.

Example 168: ¹H NMR (400 MHz, MeOD-d₄) δ ppm 7.69 (d, J=2.4 Hz, 1H), 7.23-7.18 (m, 1H), 7.02 (t, J=73.2 Hz, 1H), 7.00-6.98 (m, 2H), 2.01-1.95 (m, 1H), 0.97-0.86 (m, 4H). ¹⁹F NMR (376 MHz, MeOD-d₄) δ ppm −91.59 (dd, J=176, 564 Hz, 2F), −135.37 (s, 1F).

### Example 169: 3-Amino-7-cyclopropyl-4-(7-fluoro-1H-indazol-4-yl)-5-methoxy-1H-1,6-naphthyridin-2-one

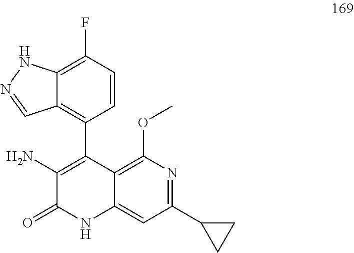 [SMILES: COc1nc(C2CC2)cc2[nH]c(=O)c(N)c(-c3ccc(F)c4[nH]ncc34)c12]

The title compound was prepared in analogy to the preparation of compound 168a, replacing benzyloxysodium with sodium methylate. Example 169 (41.9 mg) was obtained as a yellow solid. MS (ESI⁺) obsd. [(M+H)⁺]: 366.3.

Example 169: ¹H NMR (400 MHz, MeOD-d₄) δ ppm 7.68 (d, J=3.3 Hz, 1H), 7.21 (dd, J=7.6, 11.2 Hz, 1H), 6.96 (dd, J=4.0, 7.6 Hz, 1H), 6.79 (s, 1H), 3.23 (s, 3H), 2.00-1.93 (m, 1H), 0.99-0.95 (m, 2H), 0.92-0.87 (m, 2H). ¹⁹F NMR (376 MHz, MeOD-d₄) δ ppm −135.44 (s, 1F).

### Example 170: 3-amino-8-fluoro-4-(7-fluoro-1H-indazol-4-yl)-5-methoxy-1H-1,6-naphthyridin-2-one

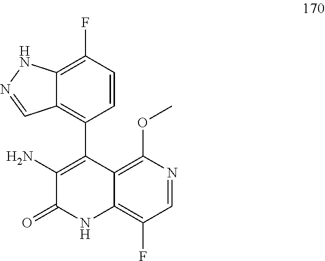 [SMILES: COc1ncc(F)c2[nH]c(=O)c(N)c(-c3ccc(F)c4[nH]ncc34)c12]

The title compound was prepared in analogy to the preparation of Example 163, replacing 2-chloro-6-cyclopropyl-pyridin-4-amine with 5-fluoro-2-methoxy-pyridin-4-amine in step 1 of the synthesis of Int-25. Example 170 (11.2 mg) was obtained as a yellow solid. MS (ESI⁺) obsd. [(M+H)⁺]: 344.1.

Example 170: ¹H NMR (400 MHz, MeOD-d₄) δ ppm 7.76 (d, J=1.6 Hz, 1H), 7.71 (d, J=3.6 Hz, 1H), 7.22 (dd, J=8.0, 11.2 Hz, 1H), 6.98 (dd, J=4.0, 8.0 Hz, 1H), 3.25 (s, 3H). ¹⁹F NMR (376 MHz, MeOD-d₄) δ ppm −135.39 (s, 1F), −159.76 (s, 1F).

### Example 171: 3-Amino-8-chloro-4-(7-fluoro-1H-indazol-4-yl)-5-methoxy-1H-1,6-naphthyridin-2-one

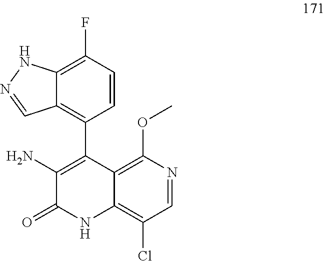 [SMILES: COc1ncc(Cl)c2[nH]c(=O)c(N)c(-c3ccc(F)c4[nH]ncc34)c12]

The title compound was prepared in analogy to the preparation of Example 163, replacing 2-chloro-6-cyclopropyl-pyridin-4-amine with 5-chloro-2-methoxy-pyridin-4-amine in step 1 of the synthesis of Int-25. Example 171 (60.0 mg) was obtained as a yellow solid. MS (ESI⁺) obsd. [(M+H)⁺]: 360.0.

Example 171: ¹H NMR (400 MHz, MeOD-d₄) δ ppm 7.90 (s, 1H), 7.70 (d, J=3.2 Hz, 1H), 7.23 (dd, J=8.0, 11.2 Hz, 1H), 6.98 (dd, J=4.0, 7.6 Hz, 1H), 3.26 (s, 3H). ¹⁹F NMR (376 MHz, MeOD-d₄) δ ppm −135.45 (s, 1F).

### Example 172: 3-Amino-4-(7-fluoro-1H-indazol-4-yl)-5-methoxy-8-methyl-1H-1,6-naphthyridin-2-one

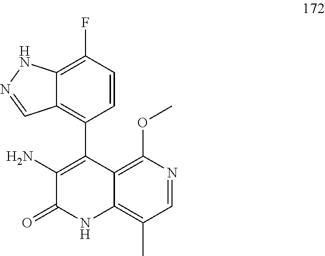 [SMILES: COc1ncc(C)c2[nH]c(=O)c(N)c(-c3ccc(F)c4[nH]ncc34)c12]

A mixture of Example 171 (100.0 mg, 0.28 mmol), catacxium A Pd G2 (92.93 mg, 0.14 mmol), K₃PO₄ (271.71 mg, 0.83 mmol), methylboronic acid (133.11 mg, 2.22 mmol) in 1,4-dioxane (2 mL) and water (0.40 mL) was stirred at 100° C. for 12 h under N₂ protection. After completion, the mixture was filtered and the filtrate was concentrated under reduced pressure to give a residue, which was purified by prep-HPLC (NH₄HCO₃ as additive) to give Example 172 (51.1 mg, 52.4% yield) as a white solid. MS (ESI⁺) obsd. [(M+H)⁺]: 340.1.

Example 172: ¹H NMR (400 MHz, DMSO-d₆) δ ppm 13.81-13.31 (m, 1H), 11.43 (br s, 1H), 7.67-7.64 (m, 2H), 7.22 (dd, J=8.0, 11.2 Hz, 1H), 6.86 (dd, J=4.0, 7.6 Hz, 1H), 4.74 (s, 2H), 3.12 (s, 3H), 2.33 (s, 3H). ¹⁹F NMR (376 MHz, DMSO-d₆) δ ppm −133.55 (s, 1F).

### Example 173: 3-Amino-4-(7-fluoro-1H-indazol-4-yl)-5-methoxy-8-methyl-1H-1,7-naphthyridin-2-one

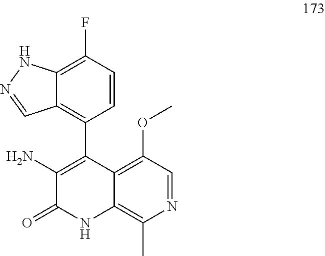 [SMILES: COc1cnc(C)c2[nH]c(=O)c(N)c(-c3ccc(F)c4[nH]ncc34)c12]

The title compound was prepare in analogy to the preparation of Example 163, replacing 2-chloro-6-cyclopropyl-pyridin-4-amine with 5-methoxy-2-methyl-pyridin-3-amine in step 1 of the synthesis of Int-25. Example 173 (2.4 mg) was obtained as a yellow solid. MS (ESI⁺) obsd. [(M+H)⁺]: 340.0.

Example 173: ¹H NMR (400 MHz, MeOD-d₄) δ ppm 7.73 (d, J=2.8 Hz, 1H), 7.68 (s, 1H), 7.23 (dd, J=7.6, 10.8 Hz, 1H), 7.00 (dd, J=4.0, 7.6 Hz, 1H), 3.25 (s, 3H), 2.82 (s, 3H). ¹⁹F NMR (376 MHz, MeOD-d₄) δ=−134.82 (s, 1F).

### Example 174: 3-Amino-4-(7-fluoro-1H-indazol-4-yl)-5-methoxy-6,8-dimethyl-1H-1,7-naphthyridin-2-one

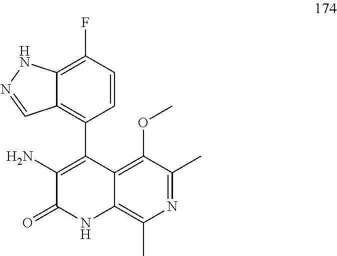 [SMILES: COc1c(C)nc(C)c2[nH]c(=O)c(N)c(-c3ccc(F)c4[nH]ncc34)c12]

### Step 1: tert-Butyl (4-((7-fluoro-1-(tetrahydro-2H-pyran-2-yl)-1H-indazol-4-yl)(hydroxy)methyl)-5-methoxypyridin-3-yl)carbamate

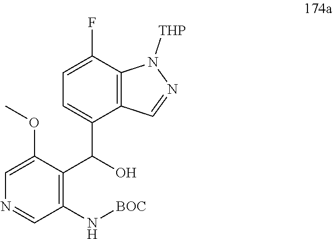

The title compound was prepared in analogy to the preparation of compound 045b, replacing Int-2 with tert-butyl N-(5-methoxy-3-pyridyl)carbamate and compound 045a with 7-fluoro-1-tetrahydropyran-2-yl-indazole-4-carbaldehyde. Compound 174a (8.4 g) was obtained as a yellow solid. MS (ESI⁺) obsd. [(M+H)⁺]: 473.2.

### Step 2: tert-Butyl (4-(7-fluoro-1-(tetrahydro-2H-pyran-2-yl)-1H-indazole-4-carbonyl)-5-methoxypyridin-3-yl)carbamate

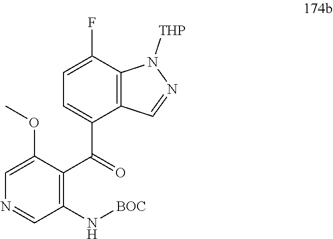

The title compound was prepared in analogy to the preparation of compound 045c, replacing compound 045b with compound 174a. Compound 174b (0.9 g) was obtained as a yellow solid. MS (ESI⁺) obsd. [(M+H)⁺]: 471.3.

### Step 3: (3-Amino-5-methoxypyridin-4-yl)(7-fluoro-1H-indazol-4-yl)methanone

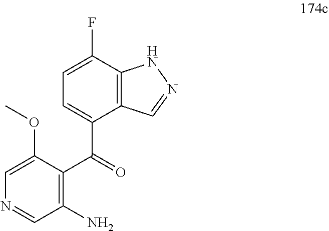 [SMILES: COc1cncc(N)c1C(=O)c1ccc(F)c2[nH]ncc12]

The title compound was prepared in analogy to the preparation of compound 045d, replacing compound 045c with compound 174b. Compound 174c (0.54 g) was obtained as a yellow solid. MS (ESI⁺) obsd. [(M+H)⁺]: 287.1.

### Step 4: (3-Amino-2,6-dibromo-5-methoxypyridin-4-yl)(7-fluoro-1H-indazol-4-yl)methanone

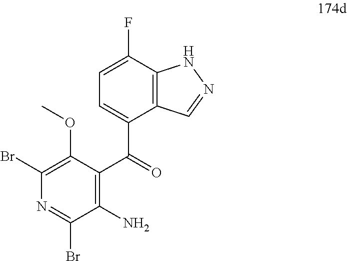 [SMILES: COc1c(Br)nc(Br)c(N)c1C(=O)c1ccc(F)c2[nH]ncc12]

To a solution of compound 174 (1.3 g, 4.54 mmol) in MeCN (15 mL) was added NBS (1.21 g, 6.81 mmol) at −20° C. The mixture was stirred at −20° C. for 0.5 h. After completion, the mixture was concentrated under reduced pressure to give a residue, which was purified by silica-gel chromatography (elute with EtOAc:PE=0 to 100%) to give compound 174d (600.0 mg, 29.8% yield) as a yellow solid. MS (ESI⁺) obsd. [(M+H)⁺]: 445.0.

### Step 5: 2-Chloro-N-(2,6-dibromo-4-(7-fluoro-1H-indazole-4-carbonyl)-5-methoxypyridin-3-yl)acetamide

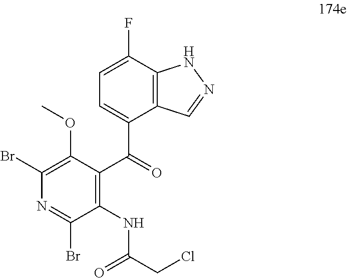 [SMILES: COc1c(Br)nc(Br)c(NC(=O)CCl)c1C(=O)c1ccc(F)c2[nH]ncc12]

To a solution of compound 174d (100.0 mg, 0.23 mmol) in toluene (3 mL) was added chloroacetyl chloride (0.05 mL, 0.68 mmol). The mixture was stirred at 100° C. for 1 h. After completion, the reaction mixture was concentrated under reduced pressure to give compound 174e (115.0 mg, crude) as a yellow solid. MS (ESI⁺) obsd. [(M+H)⁺]: 521.0.

### Step 6: 1-(6,8-Dibromo-4-(7-fluoro-1H-indazol-4-yl)-5-methoxy-2-oxo-1,2-dihydro-1,7-naphthyridin-3-yl)pyridin-1-ium chloride

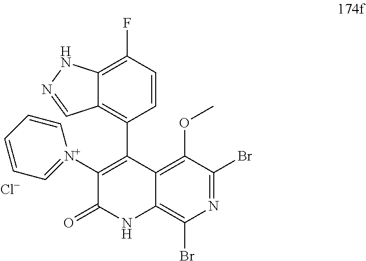 [SMILES: COc1c(Br)nc(Br)c2[nH]c(=O)c(-[n+]3ccccc3)c(-c3ccc(F)c4[nH]ncc34)c12 ; [Cl-]]

A solution of compound 174e (115.0 mg, 0.22 mmol) in pyridine (3.0 mL) was stirred at 50° C. for 3 h. The mixture was concentrated under reduced pressure to give compound 174f (120.0 mg, crude) as a brown solid. MS (ESI⁺) obsd. [(M-Cl)⁺]: 546.0.

### Step 7: 3-Amino-6,8-dibromo-4-(7-fluoro-1H-indazol-4-yl)-5-methoxy-1,7-naphthyridin-2(1H)-one

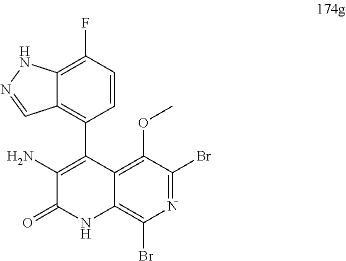 [SMILES: COc1c(Br)nc(Br)c2[nH]c(=O)c(N)c(-c3ccc(F)c4[nH]ncc34)c12]

A mixture of compound 174f (120.0 mg, 0.21 mmol) and hydrazine hydrate (0.1 mL, 2.06 mmol) in ethanol (5 mL) was stirred at 70° C. for 1 h. After completion, the mixture was concentrated under reduced pressure to give a residue, which was purified by silica-gel chromatography (elute with EtOAc:PE=0 to 100%) to give compound 174g (90.0 mg, 90.3% yield) as a yellow solid. MS (ESI⁺) obsd. [(M+H)⁺]: 484.1.

### Step 8: 3-Amino-4-(7-fluoro-1H-indazol-4-yl)-5-methoxy-6,8-dimethyl-1H-1,7-naphthyridin-2-one

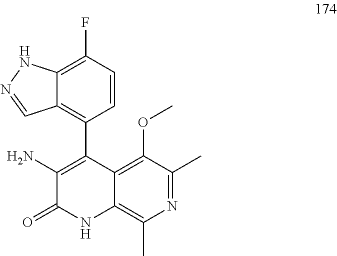 [SMILES: COc1c(C)nc(C)c2[nH]c(=O)c(N)c(-c3ccc(F)c4[nH]ncc34)c12]

A mixture of compound 174g (30.0 mg, 0.06 mmol), trimethylboroxine (0.09 mL, 0.31 mmol), Pd(dppf)Cl₂ (4.54 mg, 0.01 mmol) and Cs₂CO₃ (40.47 mg, 0.12 mmol) in 1,4-dioxane (2 mL) was stirred at 100° C. for 12 h under N₂ protection. After completion, the mixture was filtered and the filtrate was concentrated under reduced pressure to give a residue, which was purified by prep-HPLC (HCl as additive) to give Example 174 (6.8 mg, 28.0% yield) as a white solid. MS (ESI⁺) obsd. [(M+H)⁺]: 354.2.

Example 174: ¹H NMR (400 MHz, MeOD-d₄) δ ppm 7.72 (d, J=2.8 Hz, 1H), 7.27 (dd, J=7.6, 10.8 Hz, 1H), 7.13 (dd, J=4.0, 7.6 Hz, 1H), 2.93 (s, 3H), 2.83 (s, 3H), 2.43 (s, 3H). ¹⁹F NMR (376 MHz, MeOD-d₄) δ=−134.22 (s, 1F).

### Example 175: 3-Amino-6-chloro-4-(7-fluoro-1H-indazol-4-yl)-8-methyl-1H-1,7-naphthyridin-2-one

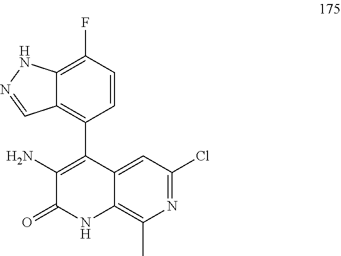 [SMILES: Cc1nc(Cl)cc2c(-c3ccc(F)c4[nH]ncc34)c(N)c(=O)[nH]c12]

The title compound was prepared in analogy to the preparation of Example 163, replacing 2-chloro-6-cyclopropyl-pyridin-4-amine with 6-chloro-2-methyl-pyridin-3-amine in step 1 of the synthesis of Int-25. Example 175 (60.0 mg) was obtained as a yellow solid. MS (ESI⁺) obsd. [(M+H)⁺]: 344.1.

Example 175: ¹H NMR (400 MHz, MeOD-d₄) δ ppm 7.77 (d, J=3.2 Hz, 1H), 7.35 (dd, J=7.6, 10.8 Hz, 1H), 7.12 (dd, J=3.6, 7.6 Hz, 1H), 6.77 (s, 1H), 2.80 (s, 3H). ¹⁹F NMR (376 MHz, MeOD-d₄) δ ppm −132.47 (s, 1F).

### Example 176: 3-Amino-5-chloro-4-(7-fluoro-1H-indazol-4-yl)-8-methyl-1H-1,7-naphthyridin-2-one

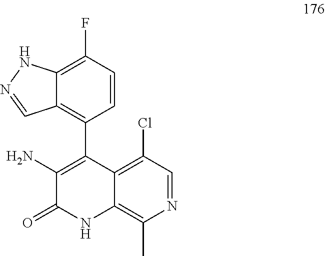 [SMILES: Cc1ncc(Cl)c2c(-c3ccc(F)c4[nH]ncc34)c(N)c(=O)[nH]c12]

The title compound was prepared in analogy to the preparation of Example 163, replacing 2-chloro-6-cyclopropyl-pyridin-4-amine with 5-chloro-2-methyl-pyridin-3-amine in step 1 of the synthesis of Int-25. Example 176 (60.0 mg) was obtained as a yellow solid. MS (ESI⁺) obsd. [(M+H)⁺]: 344.0.

Example 176: ¹H NMR (400 MHz, DMSO-d₆,) δ ppm 13.67 (br s, 1H), 11.17 (br s, 1H), 7.89 (s, 1H), 7.76 (d, J=3.2 Hz, 1H), 7.25 (dd, J=8.0, 11.2 Hz, 1H), 6.93 (dd, J=4.0, 8.0 Hz, 1H), 5.54 (s, 2H), 2.67 (s, 3H) ¹⁹F NMR (376 MHz, DMSO-d₆) δ ppm −132.25.

### Example 177: 3-Amino-4-(7-fluoro-1H-indazol-4-yl)-5,8-dimethyl-1H-1,7-naphthyridin-2-one

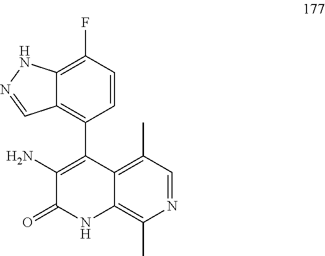 [SMILES: Cc1ncc(C)c2c(-c3ccc(F)c4[nH]ncc34)c(N)c(=O)[nH]c12]

The title compound was prepared in analogy to the preparation of Example 163, replacing 2-chloro-6-cyclopropyl-pyridin-4-amine with 2,5-dimethylpyridin-3-amine in step 1 of the synthesis of Int-25. Example 177 (19.1 mg) was obtained as a yellow solid. MS (ESI⁺) obsd. [(M+H)⁺]: 324.1.

Example 177: ¹H NMR (400 MHz, MeOD-d₄) δ ppm 7.83 (d, J=3.6 Hz, 1H), 7.79 (s, 1H), 7.30 (dd. J=8.0, 11.2 Hz, 1H), 7.12 (dd, J=4.0, 8.0 Hz. 1H). 2.88 (s. 3H). 1.63 (s, 311). ¹⁹F NMR (376 MHz, MeOD-d₄) δ ppm −132.57 (s, 1F),

### Example 178: 3-Amino-5-ethyl-4-(7-fluoro-1H-indazol-4-yl)-8-methyl-1H-1,7-naphthyridin-2-one

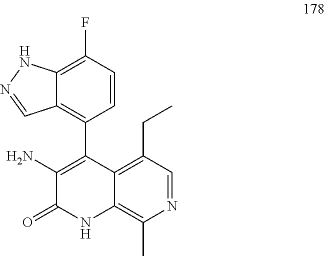 [SMILES: CCc1cnc(C)c2[nH]c(=O)c(N)c(-c3ccc(F)c4[nH]ncc34)c12]

The title compound was prepared in analogy to the preparation of Example 158, replacing Example 001 with Example 176 in step 1. Example 178 (9.2 mg) was obtained as a yellow solid. MS (ESI⁺) obsd. [(M+H)⁺]: 338.0.

Example 178: ¹H NMR (400 MHz, MeOD-d₄) δ ppm 7.82 (s, 1H), 7.77 (s, 1H), 7.34-7.29 (m, 1H), 7.17-7.14 (m, 1H), 2.87 (s, 3H), 2.03-1.90 (m, 2H), 0.75 (t, J=7.6 Hz, 3H). ¹⁹F NMR (376 MHz, MeOD-d₄) δ ppm −132.54 (s, 1F).

### Example 179: 3-Amino-5-fluoro-4-(7-fluoro-1H-indazol-4-yl)-8-methyl-1H-1,7-naphthyridin-2-one

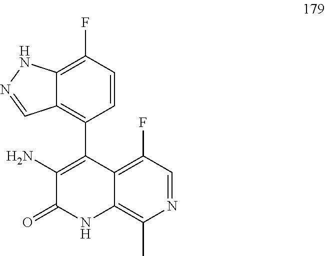 [SMILES: Cc1ncc(F)c2c(-c3ccc(F)c4[nH]ncc34)c(N)c(=O)[nH]c12]

The title compound was prepared in analogy to the preparation of Example 163, replacing 2-chloro-6-cyclopropyl-pyridin-4-amine with 5-fluoro-2-methyl-3-pyridinamine in step 1 of the synthesis of Int-25. Example 179 (10.1 mg) was obtained as a yellow solid. MS (ESI⁺) obsd. [(M+H)⁺]: 328.1.

Example 179: ¹H NMR (400 MHz, MeOD-d₄) δ ppm 8.10 (d, J=6.0 Hz, 1H), 7.84 (d, J=3.2 Hz, 1H), 7.27 (dd, J=8.0, 10.8 Hz, 1H), 7.11 (dd, J=4.0, 8.0 Hz, 1H), 2.87 (s, 3H). ¹⁹F NMR (376 MHz, MeOD-d₄) δ ppm −130.82 (s, 1F), −133.50 (s, 1F).

### Example 180: 3-Amino-5-benzyloxy-8-chloro-4-(7-fluoro-1H-indazol-4-yl)-1H-1,7-naphthyridin-2-one

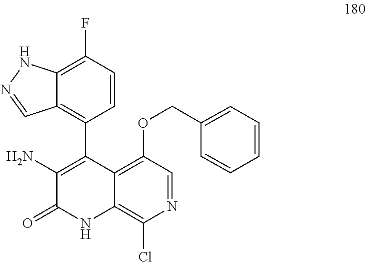 [SMILES: Nc1c(-c2ccc(F)c3[nH]ncc23)c2c(OCc3ccccc3)cnc(Cl)c2[nH]c1=O]

The title compound was prepared in analogy to the preparation of Example 001, replacing compound 001a with compound Int-26d in step 2. Example 180 (21.8 mg) was obtained as a yellow solid. MS (ESI⁺) obsd. [(M+H)⁺]: 436.0.

Example 180: ¹H NMR (400 MHz, DMSO-d₆) δ ppm 13.41 (s, 1H), 11.44 (s, 1H), 7.74 (s, 1H), 7.70 (d, J=2.4 Hz, 1H), 7.24-7.18 (m, 1H), 7.14-7.07 (m, 2H), 6.90 (dd, J=7.6, 11.2 Hz, 1H), 6.77 (dd, J=4.0, 7.6 Hz, 1H), 6.43 (d, J=7.2 Hz, 2H), 5.35 (br s, 2H), 4.76-4.46 (m, 2H). ¹⁹F NMR (400 MHz, DMSO-d₆) δ ppm −132.86 (s, 1F).

### Example 181: 3-Amino-5-benzyloxy-4-(7-fluoro-1H-indazol-4-yl)-8-methyl-1H-1,7-naphthyridin-2-one

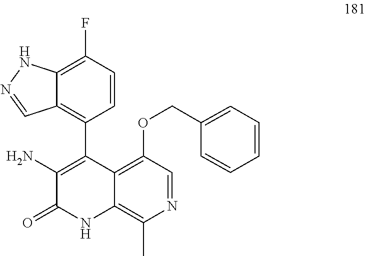 [SMILES: Cc1ncc(OCc2ccccc2)c2c(-c3ccc(F)c4[nH]ncc34)c(N)c(=O)[nH]c12]

The title compound was prepared in analogy to the preparation of Example 001, replacing compound 001a with compound Int-26e in step 2. Example 181 (16.5 mg) was obtained as a yellow solid. MS (ESI⁺) obsd. [(M+H)⁺]: 416.1.

Example 181: ¹H NMR (400 MHz, DMSO-d₆) δ ppm 13.43 (br s, 1H), 7.78 (s, 1H), 7.64 (br s, 1H), 7.22-7.15 (m, 1H), 7.14-7.05 (m, 2H), 6.92 (dd, J=7.2, 11.2 Hz, 1H), 6.77 (dd, J=4.4, 7.2 Hz, 1H), 6.42 (d, J=7.2 Hz, 2H), 5.07 (s, 2H), 4.72-4.31 (m, 2H), 2.61 (s, 3H). ¹⁹F NMR (400 MHz, DMSO-d₆) δ ppm −133.15 (s, 1F).

### Example 182: 3-Amino-4-(7-fluoro-1H-indazol-4-yl)-5-isopropoxy-8-methyl-1H-1,7-naphthyridin-2-one

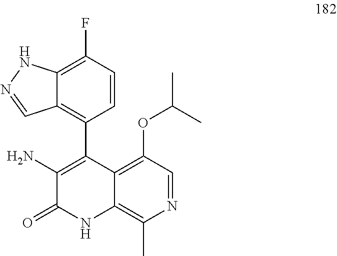 [SMILES: Cc1ncc(OC(C)C)c2c(-c3ccc(F)c4[nH]ncc34)c(N)c(=O)[nH]c12]

### Step 1: tert-Butyl (4-(7-fluoro-1-(tetrahydro-2H-pyran-2-yl)-1H-indazole-4-carbonyl)-5-isopropoxy-2-methylpyridin-3-yl)carbamate

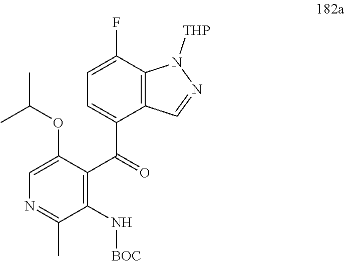

A mixture of compound Int-26 (100 mg, 0.21 mmol), 2-bromopropane (78.42 mg, 0.64 mmol) and K₂CO₃ (88.12 mg, 0.64 mmol) in DMF (1 mL) was stirred at 80° C. for 2 h. After completion, the reaction mixture was filtered and the filtrate was concentrated under reduced pressure to give a residue, which was purified by silica-gel chromatography (elute with EtOAc:PE=0 to 20%) to afford compound 182a (100 mg, 91.8% yield) as a yellow solid. MS (ESI⁺) obsd. [(M+H)⁺]: 513.3.

### Step 2: (3-Amino-5-isopropoxy-2-methylpyridin-4-yl)(7-fluoro-1H-indazol-4-yl)methanone

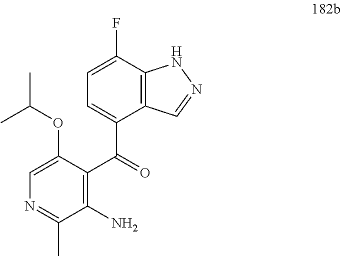 [SMILES: Cc1ncc(OC(C)C)c(C(=O)c2ccc(F)c3[nH]ncc23)c1N]

The title compound was prepared in analogy to the preparation of compound 001b, replacing compound 001a with compound 182a. Compound 182b (60.0 mg) was obtained as a yellow solid. MS (ESI⁺) obsd. [(M+H)⁺]: 329.1.

### Step 3: 2-Chloro-N-(4-(7-fluoro-1H-indazole-4-carbonyl)-5-isopropoxy-2-methylpyridin-3-yl)acetamide

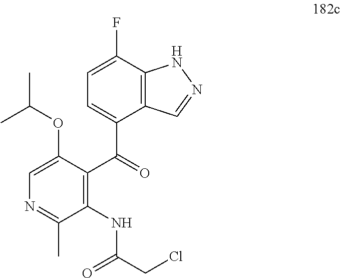 [SMILES: Cc1ncc(OC(C)C)c(C(=O)c2ccc(F)c3[nH]ncc23)c1NC(=O)CCl]

The title compound was prepared in analogy to the preparation of compound 001c, replacing compound 001b with compound 182b. Compound 182c (60.0 mg crude) was obtained as a yellow solid. MS (ESI⁺) obsd. [(M+H)⁺]: 405.1.

### Step 4: 1-(4-(7-Fluoro-1H-indazol-4-yl)-5-isopropoxy-8-methyl-2-oxo-1,2-dihydro-1,7-naphthyridin-3-yl)pyridin-1-ium chloride

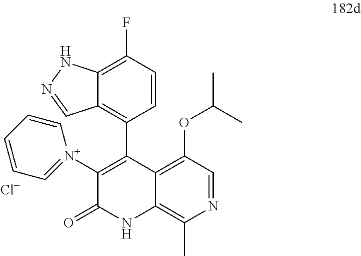 [SMILES: Cc1ncc(OC(C)C)c2c(-c3ccc(F)c4[nH]ncc34)c(-[n+]3ccccc3)c(=O)[nH]c12 ; [Cl-]]

The title compound was prepared in analogy to the preparation of compound 001d, replacing compound 001c with compound 182c. Compound 182d (80.0 mg crude) was obtained as a brown solid. MS (ESI⁺) obsd. [(M-Cl)⁺]: 430.2.

### Step 5: 3-Amino-4-(7-fluoro-1H-indazol-4-yl)-5-isopropoxy-8-methyl-1H-1,7-naphthyridin-2-one

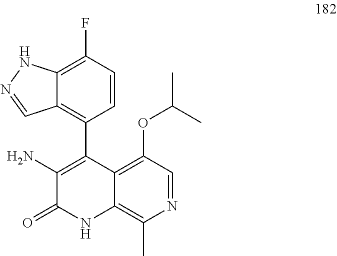 [SMILES: Cc1ncc(OC(C)C)c2c(-c3ccc(F)c4[nH]ncc34)c(N)c(=O)[nH]c12]

The title compound was prepared in analogy to the preparation of Example 001, replacing compound 001d with compound 182d. Example 182 (33.2 mg) was obtained as a yellow solid. MS (ESI⁺) obsd. [(M+H)⁺]: 368.1.

Example 182: ¹H NMR (400 MHz, MeOD-d₄) δ ppm 7.75 (d, J=3.2 Hz, 1H), 7.66 (s, 1H), 7.24 (dd, J=7.6, 10.8 Hz, 1H), 6.98 (dd, J=4.0, 7.6 Hz, 1H), 4.34-4.27 (m, 1H), 2.84 (s, 3H), 0.66 (d, J=6.0 Hz, 3H), 0.42 (d, J=6.0 Hz, 3H). ¹⁹F NMR (376 MHz, MeOD-d₄) δ ppm −135.09 (s, 1F).

The following Examples 183 to 192 were prepared in analogy to the procedure described for the preparation of Example 182, replacing 2-bromopropane with HALOCPD indicated in Table 15 in step 1.

TABLE 15
Compound synthesis and characterization

| | Compound Names and | | |
|---|---|---|---|
| Ex. | Structures | HALOCPD | NMR and (ESI⁺) |
| 183 | 3-Amino-4-(7-fluoro- 1H-indazol-4-yl)-8- methyl-5-(2,2,2- trifuoroethoxy)-1H- 1,7-naphthyridin-2-one 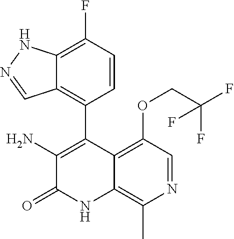 [SMILES: Cc1ncc(OCC(F)(F)F)c2c(-c3ccc(F)c4[nH]ncc34)c(N)c(=O)[nH]c12] | 1,1,1-Trifluoro- 2-iodo-ethane Notes: 25° C. (reaction temperature) | ¹H NMR (400 MHz, DMSO-d₆) δ ppm 13.53 (s, 1H), 12.30 (s, 1H), 7.83 (s, 1H), 7.73 (d, J = 2.8 Hz, 1H), 7.21-7.16 (m, 1H), 6.87-6.84 (m, 1H), 6.44 (s, 2H), 4.45-4.29 (m, 2H), 2.85 (s, 3H). ¹⁹F NMR (377 MHz, DMSO-d₆) δ ppm −73.45 (s, 3F), −133.32 (s, 1F). MS (ESI⁺) obsd. [(M + H)⁺]: 408.1. |
| 184 | 3-Amino-5-ethoxy-4- (7-fluoro-1H-indazol-4- yl)-8-methyl-1H-1,7- naphthyridin-2-one 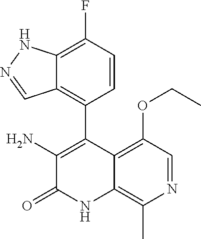 [SMILES: CCOc1cnc(C)c2[nH]c(=O)c(N)c(-c3ccc(F)c4[nH]ncc34)c12] | Iodoethane Notes: 25° C. (reaction temperature) | ¹H NMR (400 MHz, DMSO-d₆) δ = 13.58 (s, 1H), 11.37 (s, 1H), 7.67 (d, J = 2.4 Hz, 1H), 7.61 (s, 1H), 7.21 (dd, J = 7.6, 11.6 Hz, 1H), 6.84 (dd, J = 4.0, 7.6 Hz, 1H), 5.15 (s, 2H), 3.54-3.50 (m, 1H), 3.36-3.33 (m, 1H), 2.59 (s, 3H), 0.17 (t, J = 6.8 Hz, 3H). ¹⁹F NMR (400 MHz, DMSO-d₆) δ = −134.04 (s, 1F). MS (ESI⁺) obsd. [(M + H)⁺]: 354.2. |
| 185 | 3-Amino-5- (difluoromethoxy)-4-(7- fluoro-1H-indazol-4- yl)-8-methyl-1H-1,7- naphthyridin-2-one 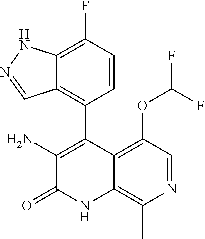 [SMILES: Cc1ncc(OC(F)F)c2c(-c3ccc(F)c4[nH]ncc34)c(N)c(=O)[nH]c12] | Sodium chlorodifluoro- acetate | ¹H NMR (400 MHz, MeOD-d₄) δ ppm 7.82 (s, 1H), 7.72 (d, J = 2.8 Hz, 1H), 7.23 (dd, J = 7.6, 11.2 Hz, 1H), 7.03 (dd, J = 4.0, 7.6 Hz, 1H), 5.99 (t, J = 324 Hz, 1H), 2.76 (s, 3H). ¹⁹F NMR (376 MHz, MeOD-d₄) δ ppm −82.92 (d, J = 165.816 Hz, 1F), −84.29 (d, J = 165.816 Hz, 1F), −134.69 (s, 1F). MS (ESI⁺) obsd. [(M + H)⁺]: 376.2. |
| 186 | 3-Amino-5-(2,2- difluoroethoxy)-4-(7- fluoro-1H-indazol-4- yl)-8-methyl-1H-1,7- naphthyridin-2-one 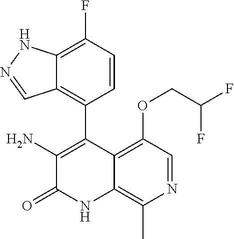 [SMILES: Cc1ncc(OCC(F)F)c2c(-c3ccc(F)c4[nH]ncc34)c(N)c(=O)[nH]c12] | 1,1-Difluoro-2- iodo-ethane Notes: 25° C. (reaction temperature | ¹H NMR (400 MHz, DMSO-d₆) δ ppm 13.62 (br s, 1H), 12.28 (s, 1H), 7.84 (s, 1H), 7.75 (d, J = 2.8 Hz, 1H), 7.22 (dd, J = 8.0, 11.2 Hz, 1H), 6.88 (dd, J = 4.0, 8.0 Hz, 1H), 6.39 (br s, 2H), 5.07- 4.78 (m, 1H), 4.02-3.82 (m, 2H), 2.83 (s, 3H). ¹⁹F NMR (377 MHz, DMSO-d₆) δ ppm −125.45 (s, 2F), −132.99 (s, 1F). MS (ESI⁺) obsd. [(M + H)⁺]: 390.1. |
| 187 | 3-Amino-5- (cyclopropoxy)-4-(7- fluoro-1H-indazol-4- yl)-8-methy-1H-1,7- naphthyridin-2-one 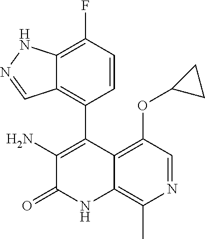 [SMILES: Cc1ncc(OC2CC2)c2c(-c3ccc(F)c4[nH]ncc34)c(N)c(=O)[nH]c12] | Cyclopropyl trifluoromethane- sulfonate Notes: 50° C. (reaction temperature | ¹H NMR (400 MHz, MeOD-d₄) δ ppm 7.90 (s, 1H), 7.70 (d, J = 3.2 Hz, 1H), 7.23 (dd, J = 8.0, 11.2 Hz, 1H), 6.96 (dd, J = 4.0, 8.0 Hz, 1H), 3.54-3.51 (m, 1H), 2.84 (s, 3H), 0.45-0.42 (m, 2H), −0.18-−0.26 (m, 1H), −0.42-−0.44 (m, 1H). ¹⁹F NMR (376 MHz, MeOD-d₄) δ ppm −134.71 (s, 1F). MS (ESI⁺) obsd. [(M + H)⁺]: 366.1. |
| 188 | 3-Amino-5-(3,3- difluoropropoxy)-4-(7- fluoro-1H-indazol-4- yl)-8-methyl-1H-1,7- naphthyridin-2-one 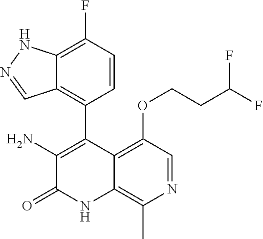 [SMILES: Cc1ncc(OCCC(F)F)c2c(-c3ccc(F)c4[nH]ncc34)c(N)c(=O)[nH]c12] | 1,1-difluoro-3- iodo-propane Notes: 25° C. (reaction temperature | ¹H NMR (400 MHz, MeOD-d₄) δ ppm 7.81 (d, J = 3.2 Hz, 1H), 7.73 (s, 1H), 7.27 (dd, J = 7.6, 10.8 Hz, 1H), 7.03 (dd, J = 4.0, 8.0 Hz, 1H), 4.75-4.46 (m, 1H), 3.84-3.79 (m, 1H), 3.73-3.68 (m, 1H), 2.85 (s, 3H), 1.34-1.12 (m, 2H). ¹⁹F NMR (400 MHz, MeOD-d₄) δ ppm −119.76 (s, 2F), −134.04 (s, 1F). MS (ESI⁺) obsd. [(M + H)⁺]: 404.1. |
| 189 | 3-Amino-5- (cyclobutoxy)-4-(7- fluoro-1H-indazol-4- yl)-8-methyl-1H-1,7- naphthyridin-2-one 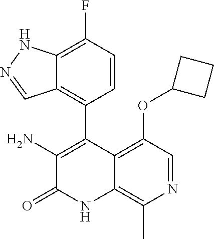 [SMILES: Cc1ncc(OC2CCC2)c2c(-c3ccc(F)c4[nH]ncc34)c(N)c(=O)[nH]c12] | Bromocyclo- butane Notes: 50° C. (reaction temperature | ¹H NMR (400 MHz, MeOD-d₄) δ ppm 7.68 (d, J = 2.8 Hz, 1H), 7.40 (s, 1H), 7.23 (dd, J = 8.0, 11.2 Hz, 1H), 6.99 (dd, J = 3.6, 7.6 Hz, 1H), 4.26-4.19 (m, 1H), 2.65 (s, 3H), 2.03-1.96 (m, 1H), 1.84-1.77 (m, 1H), 1.42-1.34 (m, 2H), 1.09-1.01 (m, 2H). ¹⁹F NMR (376 MHz, MeOD-d₄) δ ppm −135.91 (s, 1F). MS (ESI⁺) obsd. [(M − Cl)⁺]: 380.2. |
| 190 | 3-Amino-4-(7-fluoro- 1H-indazol-4-yl)-8- methyl-5-propoxy-1H- 1,7-naphthyridin-2-one 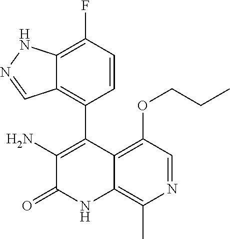 [SMILES: CCCOc1cnc(C)c2[nH]c(=O)c(N)c(-c3ccc(F)c4[nH]ncc34)c12] | 1- Bromopropane | ¹H NMR (400 MHz, MeOD-d₄) δ ppm 7.76 (d, J = 3.2 Hz, 1H), 7.65 (s, 1H), 7.23 (dd, J = 7.6, 10.8 Hz, 1H), 6.99 (dd, J = 4.0, 8.0 Hz, 1H), 3.61-3.58 (m, 1H), 3.49-3.47 (m, 1H), 2.83 (s, 3H), 0.78-0.71 (m, 2H), 0.32 (t, J = 7.6 Hz, 3H). ¹⁹F NMR (376 MHz, MeOD-d₄) δ ppm −134.95 (s, 1F). MS (ESI⁺) obsd. [(M + H)⁺]: 368.1. |
| 191 | 3-Amino-5- (cyclopropylmethoxy)- 4-(7-fluoro-1H-indazol- 4-yl)-8-methyl-1H-1,7- naphthyridin-2-one 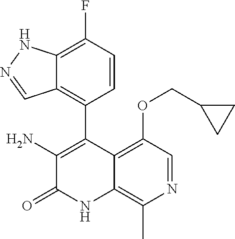 [SMILES: Cc1ncc(OCC2CC2)c2c(-c3ccc(F)c4[nH]ncc34)c(N)c(=O)[nH]c12] | Bromomethyl- cyclopropane | ¹H NMR (400 MHz, MeOD-d₄) δ = 7.76 (d, J = 3.2 Hz, 1H), 7.57 (s, 1H), 7.25-7.21 (m, 1H), 7.02- 6.99 (m, 1H), 3.54-3.50 (m, 1H), 3.26-3.22 (m, 1H), 2.83 (s, 3H), 0.22-0.16 (m, 1H), 0.09- 0.03 (m, 2H), −0.12-−0.16 (m, 1H), −0.37-−0.43 (m, 1H). ¹⁹F NMR (377 MHz, MeOD-d₄) δ = −135.10 (s, 1F). MS (ESI⁺) obsd. [(M + H)⁺]: 380.2. |
| 192 | 3-Amino-4-(7-fluoro- 1H-indazol-4-yl)-8- methyl-5- (trifluoromethoxy)-1H- 1,7-naphthyridin-2-one 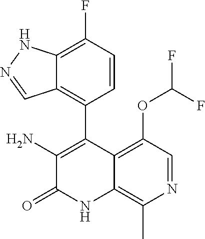 [SMILES: Cc1ncc(OC(F)F)c2c(-c3ccc(F)c4[nH]ncc34)c(N)c(=O)[nH]c12] | Silver trifluoromethane- sulfonate, trifluoromethyl- trimethylsilane, N- fluorobenzene- sulfonamide, selectfluor, 2- fluoropyridine and ScF (instead of K₂CO₃) at 25° C. | ¹H NMR (400 MHz, MeOD-d₄) δ ppm 8.06 (s, 1H), 7.75 (d, J = 3.2 Hz, 1H), 7.26 (dd, J = 7.6, 10.8 Hz, 1H), 7.05 (dd, J = 4.0, 7.6 Hz, 1H), 2.84 (s, 3H). ¹⁹F NMR (376 MHz, MeOD-d₄) δ ppm −59.97 (s, 3F), −134.02 (s, 1F). MS (ESI⁺) obsd. [(M + H)⁺]: 394.1. |

### Example 193: 3-Amino-4-(7-fluoro-1H-indazol-4-yl)-5-(methoxymethyl)-8-methyl-1H-1,7-naphthyridin-2-one

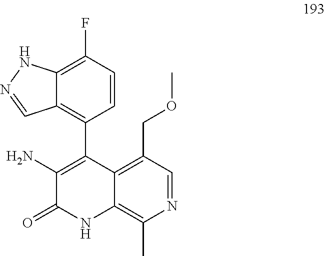 [SMILES: COCc1cnc(C)c2[nH]c(=O)c(N)c(-c3ccc(F)c4[nH]ncc34)c12]

The title compound was prepared in analogy to the preparation of Example 045, replacing Int-2 with Int-27 and compound 045a with 7-fluoro-1-tetrahydropyran-2-yl-indazole-4-carbaldehyde in step 2. Example 193 (11.1 mg) was obtained as a yellow solid. MS (ESI⁺) obsd. [(M+H)⁺]: 354.1.

Example 193: ¹H NMR (400 MHz, MeOD-d₄) δ ppm 8.03 (s, 1H), 7.82 (d, J=3.2 Hz, 1H), 7.33 (dd, J=7.6, 10.8 Hz, 1H), 7.13 (dd, J=4.0, 7.6 Hz, 1H), 3.83 (d, J=12.8 Hz, 1H), 3.43 (d, J=13.2 Hz, 1H), 2.90 (s, 3H), 2.83 (s, 3H). ¹⁹F NMR (376 MHz, MeOD-d₄) δ ppm −132.03 (s, 1F).

### Example 194: 3-Amino-7-bromo-4-(7-fluoro-1H-indazol-4-yl)-6-methyl-1H-1,5-naphthyridin-2-one

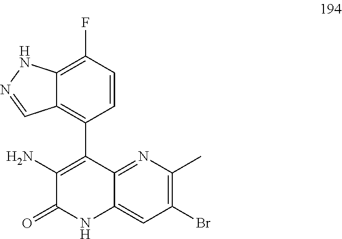 [SMILES: Cc1nc2c(-c3ccc(F)c4[nH]ncc34)c(N)c(=O)[nH]c2cc1Br]

The title compound was prepared in analogy to the preparation of Example 163, replacing 2-chloro-6-cyclopropyl-pyridin-4-amine with 5-bromo-6-methyl-pyridin-3-amine in step 1 of the synthesis of Int-25. Example 194 (10.1 mg) was obtained as a yellow solid. MS (ESI⁺) obsd. [(M+H)⁺]: 388.3.

Example 194: ¹H NMR (400 MHz, DMSO-d₆) δ ppm 13.45 (s, 1H), 12.10 (s, 1H), 7.71 (s, 1H), 7.59 (s, 1H), 7.34 (dd, J=8.0, 11.2 Hz, 1H), 7.11 (dd, J=4.0, 7.6 Hz, 1H), 5.40 (s, 2H), 2.47 (s, 3H).

### Example 195: 3-Amino-7-ethyl-4-(7-fluoro-1H-indazol-4-yl)-6-methyl-1H-1,5-naphthyridin-2-one

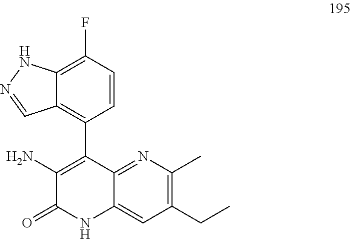 [SMILES: CCc1cc2[nH]c(=O)c(N)c(-c3ccc(F)c4[nH]ncc34)c2nc1C]

### Step 1: 3-Amino-4-(7-fluoro-1H-indazol-4-yl)-6-methyl-7-vinyl-1,5-naphthyridin-2(1H)-one

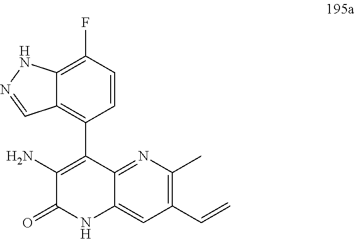 [SMILES: C=Cc1cc2[nH]c(=O)c(N)c(-c3ccc(F)c4[nH]ncc34)c2nc1C]

A mixture of Example 194 (200 mg. 0.52 mmol), vinylboronic acid pinacol ester (159 mg, 1.03 mmol), Pd(dppf)Cl₂ (37.7 mg, 0.05 mmol) and Cs₂CO₃ (336 mg, 1.03 nmol) in 1,4-dioxane (3 mL) and water (0.3 mL) was stirred at 100° C. for 2 b under nitrogen atmosphere. After completion, the reaction was concentrated under vacuum to give a crude residue, which was purified by silica-gel chromatography (elute with EtOAc:PE=0 to 40%) to afford compound 195a (80.0 mg, 46.3% yield) as a yellow solid. MS (ESI⁺) obsd. [(M+H)⁺]: 336.1.

### Step 2: 3-Amino-7-ethyl-4-(7-fluoro-1H-indazol-4-yl)-6-methyl-1H-1,5-naphthyridin-2-one

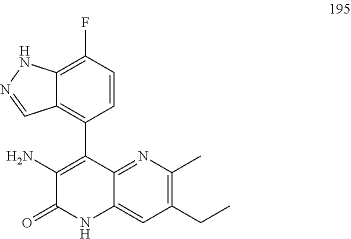 [SMILES: CCc1cc2[nH]c(=O)c(N)c(-c3ccc(F)c4[nH]ncc34)c2nc1C]

A mixture of compound 195a (25.0 mg, 0.07 mmol) and Pd/C (25.0 mg) in Methanol (2 mL)/THE (2 mL) was stirred at 25° C. for 0.2 h under hydrogen atmosphere (15 Psi). After completion, the mixture was filtered and the filtrate was concentrated under vacuum to give a residue, which was purified by prep-HPLC (NH₄HCO₃ as eluent) to afford Example 195 (5.39 mg, 21.4% yield) as a white solid. MS (ESI⁺) obsd. [(M+H)⁺]: 338.1.

Example 195: ¹H NMR (400 MHz, MeOD-d₄) δ ppm 7.62 (d, J=2.8 Hz, 1H), 7.47 (s, 1H), 7.28 (dd, J=8.0, 10.8 Hz, 1H), 7.15 (dd, J=4.0, 8.0 Hz, 1H), 2.71 (q, J=7.6 Hz, 2H), 2.31 (s, 3H), 1.28 (t, J=7.6 Hz, 3H). ¹⁹F NMR (377 MHz, MeOD-d₄) δ ppm −134.89 (s, 1F).

### BIOLOGICAL EXAMPLES

### Example 200: ADP-Glo Assay

Detection of Myt1 kinase activity utilized a recombinant human Myt1 kinase assay measuring the hydrolysis of ATP using a commercially available ADP-Glo Assay (ADP-Glo™ Kinase Assay from Promega, 10000 assays, #V9102). Briefly, 50 nL of compounds (diluted in DMSO) was added to the microplate by ECHO Liquid Handler. After this, 2.5 μL recombinant human Myt1 (full length PKMYT1 recombinant human protein expressed in insect cells) was prepared in reaction buffer (50 mM HEPES (N-2-hydroxyethylpiperazine-N-2-ethane sulfonic acid), 10 mM MgCl₂, 1 mM EGTA (ethylene glycol-bis(β-aminoethyl ether)-N,N,N′,N′-tetraacetic acid), 0.1 mg/mi BSA (bovine serum albumin), 1 mM DTT (Dithiothireitol)) and added to 384 well white microplate (Corning #4512) and incubated at 22° C. for 15 minutes. Ultra-Pure Adenosine Triphosphate (ATP) solution (ADP-Glo kit from Promega) was diluted in reaction buffer and 2.5 μL was added to the microplate, spun down briefly and incubated for 60 minutes at 30° C. The final Myt1 enzyme concentration was 20 nM and the final ATP concentration was 300 μM. After the 60-minute incubation, 5 μL of ADP-Glo reagent was added and the plate was spun briefly and sealed and incubated in the dark for 60 minutes at 22° C. Following this, 10 μL of kinase detection reagent was added per well and the plate was spun briefly, sealed and incubated for 30 minutes at 22° C. in the dark. Luminescence was read using the Envision (250 ms integration). The IC₅₀ and the % max inhibition were calculated for each inhibitor compound tested.

Exemplary prepared compounds and their activities were shown in Table 16 below.

TABLE 16
ADP-Glo assay potency

| Example | PKMYT1 |
|---|---|
| No. | IC₅₀ (nM) |
| 1 | 10.5 |
| 2 | 12.6 |
| 3 | <10.0 |
| 4 | 48.1 |
| 5 | 21.6 |
| 6 | 22.6 |
| 7 | 12.0 |
| 8 | 11.6 |
| 9 | 16.1 |
| 10 | 42.9 |
| 14 | 44.6 |
| 15 | <10.0 |
| 16 | 11.3 |
| 17 | <10.0 |
| 18 | 10.4 |
| 19 | 30.3 |
| 20 | 24.2 |
| 21 | 10.3 |
| 22 | <10.0 |
| 23 | 10.7 |
| 24 | 15.6 |
| 25 | 18.8 |
| 26 | <10.0 |
| 27 | 21.2 |
| 28 | 29.1 |
| 29 | 12.0 |
| 30 | 15.3 |
| 31 | 21.3 |
| 32 | 20.3 |
| 33 | 15.9 |
| 34 | 19.3 |
| 35 | 25.6 |
| 36 | 37.8 |
| 37 | <10.0 |
| 38 | 30.1 |
| 39 | 39.6 |
| 40 | 40.7 |
| 43 | 76.3 |
| 44 | <10.0 |
| 46 | 10.6 |
| 47 | <10.0 |
| 48 | 27.1 |
| 49 | <10.0 |
| 50 | <10.0 |
| 51 | 17.9 |
| 52 | <10.0 |
| 53 | 10.7 |
| 54 | 11.5 |
| 55 | 12.4 |
| 56 | <10.0 |
| 57 | 11.7 |
| 58 | <10.0 |
| 59 | <10.0 |
| 60 | <10.0 |
| 61 | 17.7 |
| 62 | 14.6 |
| 63 | 9.7 |
| 64 | <10.0 |
| 65 | 14.7 |
| 66 | 11.6 |
| 67 | 24.4 |
| 68 | 17.5 |
| 69 | 10.9 |
| 70 | 10.1 |
| 71 | 13.1 |
| 72 | 11.2 |
| 73 | 14.0 |
| 74 | 11.0 |
| 75 | 11.4 |
| 76 | 14.8 |
| 77 | <10.0 |
| 78 | 17.2 |
| 79 | 17.8 |
| 80 | 21.1 |
| 81 | 14.6 |
| 82 | 16.4 |
| 83 | 11.0 |
| 84 | <10.0 |
| 85 | 11.0 |
| 86 | 11.1 |
| 87 | 12.3 |
| 88 | 47.0 |
| 90 | 13.0 |
| 91 | 14.0 |
| 92 | 14.4 |
| 93 | 11.9 |
| 94 | 18.8 |
| 95 | <10.0 |
| 96 | 65.2 |
| 97 | 24.9 |
| 98 | 18.7 |
| 99 | <10.0 |
| 100 | <10.0 |
| 101 | <10.0 |
| 102 | 16.0 |
| 103 | <10.0 |
| 104 | 15.7 |
| 105 | 18.5 |
| 106 | 14.5 |
| 107 | 22.3 |
| 108 | 15.1 |
| 109 | <10.0 |
| 110 | 16.7 |
| 111 | <10.0 |
| 112 | <10.0 |
| 113 | <10.0 |
| 114 | 13.6 |
| 115 | <10.0 |
| 116 | <10.0 |
| 117 | 14.9 |
| 118 | 17.8 |
| 119 | 13.0 |
| 120 | 17.2 |
| 121 | 10.7 |
| 122 | <10.0 |
| 123 | <10.0 |
| 124 | <10.0 |
| 125 | <10.0 |
| 126 | <10.0 |
| 127 | <10.0 |
| 128 | <10.0 |
| 129 | 12.8 |
| 130 | 12.3 |
| 131 | 17.5 |
| 132 | <10.0 |
| 133 | <10.0 |
| 134 | <10.0 |
| 135 | 14.0 |
| 136 | 17.3 |
| 137 | 14.1 |
| 138 | 14.2 |
| 139 | 13.2 |
| 140 | 11.8 |
| 141 | 10.4 |
| 142 | 18.4 |
| 143 | 11.0 |
| 144 | 11.4 |
| 145 | 18.5 |
| 146 | <10.0 |
| 148 | <10.0 |
| 149 | <10.0 |
| 150 | 15.2 |
| 151 | 13.0 |
| 152 | 13.8 |
| 153 | 11.8 |
| 154 | <10.0 |
| 155 | 14.7 |
| 156 | <10.0 |
| 157 | 15.6 |
| 158 | <10.0 |
| 159 | <10.0 |
| 160 | 11.5 |
| 161 | 15.5 |
| 162 | <10.0 |
| 163 | <10.0 |
| 164 | <10.0 |
| 165 | <10.0 |
| 166 | 14.7 |
| 167 | 14.5 |
| 168 | 11.2 |
| 169 | 13.4 |
| 170 | 12.6 |
| 171 | 15.8 |
| 172 | 10.9 |
| 173 | 15.2 |
| 174 | 12.7 |
| 175 | <10.0 |
| 176 | 14.3 |
| 177 | 13.9 |
| 178 | 18.1 |
| 179 | 12.5 |
| 180 | 22.6 |
| 181 | 20.0 |
| 182 | 14.7 |
| 183 | 13.4 |
| 184 | <10.0 |
| 185 | 11.4 |
| 186 | 15.8 |
| 187 | 10.8 |
| 188 | 15.0 |
| 189 | 10.6 |
| 190 | 15.3 |
| 191 | 15.7 |
| 192 | <10.0 |
| 193 | 33.3 |
| 195 | 10.8 |

### Example 201: Human Microsome Stability Assay

The human microsomal stability assay is used for early assessment of metabolic stability of compounds of present invention in human liver microsomes. Human liver microsomes (Cat.NO.: 452117, Corning, USA) were pre-incubated with test compound for 10 minutes at 37° C. in 100 mM potassium phosphate buffer, pH 7.4. The reactions were initiated by adding NADPH regenerating system. The final incubation mixtures contained 1 μM compound, 0.5 mg/mL liver microsomal protein, 1 mM MgCl₂ and 1 mM NADPH in 100 mM potassium phosphate buffer, pH 7.4. After incubation times of 0, 3, 6, 9, 15 and 30 minutes at 37° C., 300 μL of cold acetonitrile (including internal standard) was added to 100 μL incubation mixture to terminate the reaction. Following precipitation and centrifugation, the amount of compound remaining in the samples were determined by LC-MS/MS. Controls of no NADPH regenerating system at zero and 30 minutes were also prepared and analyzed. The compounds of present invention showed good human liver microsome stability determined in the above assay, results are shown in Table 17 below.

$$CL \text{?} ( mL / min / kg ) = \frac{Clint * protein / liver ( mg / g ) * liver / body weight ( g / kg ) / 1000 * liver blood flow}{Liver blood flow + Clint * protien / liver ( mg / g ) * liver / body weight ( g / kg ) / 1000}$$

$$\text{?} \text{indicates text missing or illegible when filed}$$

TABLE 17
Human microsome clearance values of
the compounds of this invention

| Example | Clearance of Human |
|---|---|
| No. | microsome (mL/min/kg) |
| 001 | 6.15 |
| 002 | 6.15 |
| 003 | 8.88 |
| 004 | 6.15 |
| 005 | 6.15 |
| 006 | 6.15 |
| 007 | 9.29 |
| 008 | 6.15 |
| 009 | 6.15 |
| 012 | 6.15 |
| 013 | 6.76 |
| 014 | 6.15 |
| 015 | 7.03 |
| 016 | 8.06 |
| 017 | 8.06 |
| 019 | 6.15 |
| 020 | 6.15 |
| 023 | 8.58 |
| 026 | 8.87 |
| 033 | 6.22 |
| 034 | 6.15 |
| 036 | 6.15 |
| 037 | 8.07 |
| 038 | 6.15 |
| 039 | 6.15 |
| 040 | 6.15 |
| 041 | 6.15 |
| 042 | 6.15 |
| 043 | 6.15 |
| 044 | 6.15 |
| 045 | 6.15 |
| 050 | 6.15 |
| 051 | 6.15 |
| 053 | 8.09 |
| 054 | 6.15 |
| 056 | 7.47 |
| 061 | 6.15 |
| 069 | 7.78 |
| 070 | 6.15 |
| 073 | 6.15 |
| 074 | 6.15 |
| 076 | 8.12 |
| 077 | 8.82 |
| 082 | 6.15 |
| 083 | 6.15 |
| 084 | 6.15 |
| 086 | 6.99 |
| 089 | 6.15 |
| 090 | 6.15 |
| 091 | 6.15 |
| 092 | 6.15 |
| 094 | 6.15 |
| 098 | 7.72 |
| 099 | 6.15 |
| 100 | 6.15 |
| 101 | 6.15 |
| 107 | 7.13 |
| 110 | 6.15 |
| 117 | 7.72 |
| 118 | 6.15 |
| 120 | 6.15 |
| 122 | 6.15 |
| 134 | 6.15 |
| 136 | 6.15 |
| 142 | 6.15 |
| 143 | 6.15 |
| 146 | 6.15 |
| 161 | 6.15 |
| 164 | 6.15 |
| 166 | 6.15 |
| 167 | 6.80 |
| 172 | 6.20 |
| 173 | 6.15 |
| 182 | 6.20 |
| 183 | 6.20 |
| 184 | 6.20 |
| 185 | 6.15 |

### Example 202: Human Hepatocyte Stability Assay

Warm the Thawing Media and incubation Media to 37° C. Thaw Human CryoHepatocytes (Product No. X008001, BioreclamationIVT) and measure cell viability and density, cell suspension is adjusted to 2*10⁶ cells/mL in incubation medium (William's E media containing 10% FBS (Fetal bovine serum), 50 U/mL pen strep, 0.004 mg/mL insulin and 0.4 mM L-Glutamine). Fill incubation plate (Thermo 96 deep well PP plate Cat #260251) with 50 μl of cell suspension. Pre-incubate incubation plate minimum 30 min at 37° C. in CO₂ incubator with appropriate shaking speed (standard 900 rpm). Dilute compound in incubation medium in order to obtain incubation concentration ×2 (1 μM incubation should prepare compound at 2 μM). After preincubation, start kinetics by addition of 50 μl of compound. The 100 μL incubations contained 1*10⁶ cells/mL and 1 μM compound. Samples are in Single (one compound in one well for each time point). Stop reaction by addition of 200 μl of acetonitrile containing an internal standard directly in well at appropriate sampling time. Single copy at t=2, 10, 20, 40, 60 and 120 min. Samples centrifuge at 4000 rpm for 10 min. The samples were assayed by LC/MS/MS.

TABLE 18
Human hepatocyte clearance values
of the compounds of this invention

| | Clearance of Human |
|---|---|
| Example | hepatocyte (μL/min/10⁶ |
| No. | cell) |
| 001 | 2.1 |
| 002 | 1.3 |
| 015 | 2.6 |
| 017 | 1.6 |
| 054 | <1 |
| 070 | 1.4 |
| 099 | <1 |
| 100 | <1 |
| 101 | <1 |
| 133 | 1.7 |
| 136 | 1.4 |
| 146 | 1.2 |
| 163 | <1 |
| 167 | <1 |
| 173 | <1 |
| 183 | <1 |
| 184 | 1.3 |

### Example 203: GSH Adduct Screening Assay

All compounds including positive control (Such as: diclofenac, troglitazone and nefazodone) and solvent control (pure DMSO) are incubated using a 96-deep-well plate (Eppendorf) at 20 μM (addition of 1 μl of 10 mM DMSO stock solution) in 450 μl of 0.1 M sodium phosphate buffer at pH 7.4 containing human liver microsomes (HLM). Microsomal protein concentration is 1 mg/ml. Pipetting is performed using a TECAN pipetting robot. After 5 minutes of preincubation at 37° C. the reaction is started by adding 50 μl of buffer containing GSH (100 mM) and NADPH (20 mM). Fresh stock solutions of GSH and NADPH are prepared before each experiment. The final concentrations are 5 mM for GSH and 1 mM for NADPH. After 60 minutes of incubation at 37° C. (shaking at 800 rpm) the reaction is quenched with 500 μl of cold acetonitrile and centrifuged at 5000×g at 25° C. for 11 minutes. Before analysis the supernatant is split to two fractions, 450 and 400 pl each followed by evaporation using a N₂ stream at 35° C. to a volume of approximately 150 μl.

The analysis was performed on UPLC with Quadrupole Time-of-Flight of mass spectrometer. Data analysis and evaluation is achieved in an automated approach with software (Mass-MetaSite and WebMetaBase) to extract and identify GSH conjugates based on the presence of multiple collision-induced neutral losses and fragment ions specific of glutathione conjugates in the MS spectra. The GSH assay provides a qualitative end point. The assay read-out is binary, i.e., GSH adducts found (Flag) or not (No Flag).

TABLE 19
GSH adducts of this invention

| Example | GSH |
|---|---|
| No. | adduct |
| 001 | No Flag |
| 002 | No Flag |
| 003 | No Flag |
| 015 | No Flag |
| 017 | No Flag |
| 054 | No Flag |
| 124 | No Flag |
| 160 | No Flag |

1. A compound of formula (I), 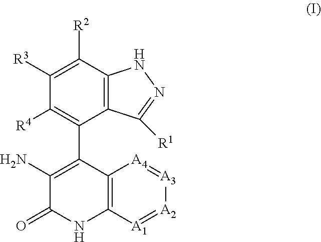
wherein
R¹ is H, halogen or C₁₋₆alkyl;
R² is H, halogen, cyano, C₃₋₇cycloalkyl, C₁₋₆alkyl, C₁₋₆alkoxy, or haloC₁₋₆alkyl;
R³ is H or halogen;
R⁴ is H, halogen, or C₁₋₆alkyl;
A₁ is N or CR⁵;
A₂ is N or CR⁶;
A₃ is N or CR⁷;
A₄ is N or CR⁸;
each of R⁵, R⁶, R⁷ and R⁸ is independently selected from H, halogen, C₁₋₆alkyl, deuterated C₁₋₆alkyl, C₃₋₇cycloalkylC₁₋₆alkyl, (C₁₋₆alkylamino)C₁₋₆alkyl, hydroxyC₁₋₆alkyl, haloC₁₋₆alkyl, C₁₋₆alkoxyC₁₋₆alkyl, C₃₋₇cycloalkyl, haloC₃₋₇cycloalkyl, C₁₋₆alkylC₃₋₇cycloalkyl, C₁₋₆alkoxy, C₃₋₇cycloalkylC₁₋₆alkoxy, (C₁₋₆alkylamino)C₁₋₆alkoxy, (hyroxyl)(C₁₋₆alkyl)C₁₋₆alkoxy, (cyanoC₃₋₇cycloalkyl)C₁₋₆alkoxy, C₁₋₆alkoxy-oxo-C₁₋₆alkoxy, haloC₁₋₆alkoxy, C₃₋₇cycloalkoxy, cyanoC₃₋₇cycloalkoxy, haloC₃₋₇cycloalkoxy, (hyroxyl)(C₁₋₆alkyl)C₃₋₇cycloalkoxy, C₁₋₆alkoxycarbonylC₃₋₇cycloalkoxy, C₂₋₆alkenyl, C₂₋₆alkynyl, hydroxyC₂₋₆alkynyl, C₁₋₆alkoxyC₂₋₆alkynyl, morpholinoC₂₋₆alkynyl, (hyroxyl)(C₁₋₆alkyl)C₂₋₆alkynyl, (C₁₋₆alkoxy)(C₁₋₆alkyl)C₂₋₆alkynyl, (hydroxytetrahydrofuranyl)C₂₋₆alkynyl, (C₁₋₆alkylamino)C₂₋₆alkynyl, (amino)(C₁₋₆alkyl)C₂₋₆alkynyl, C₁₋₆alkoxycarbonyl, C₁₋₆alkylC₂₋₆alkynyl, (hydroxy)(C₁₋₆alkyl)C₂₋₆alkynyl, oxetanylC₂₋₆alkynyl, C₁₋₆alkylamino, acyl, cyano, carboxy, C₁₋₆alkylsulfanyl, C₁₋₆alkylsulfinyl, C₁₋₆alkylsulfonyl, benzyloxy, aryl, 5 to 8 membered heterocyclyl containing one to three heteroatoms selected from N, O, and S, and 5 to 8 membered heteroaryl containing one to three heteroatoms selected from N, O, and S, wherein each of aryl, heterocyclyl, and heteroaryl is independently optionally substituted with halogen, C₁₋₆alkyl, C₃₋₇cycloalkyl, C₁₋₆alkylcarbamoyl, haloC₁₋₆alkyl, or hydroxyl;
or a pharmaceutically acceptable salt thereof.

2. The compound or pharmaceutically acceptable salt thereof according to claim 1, wherein
R¹ is H or C₁₋₆alkyl;
R² is H, halogen, C₁₋₆alkyl, or haloC₁₋₆alkyl;
R³ is H or halogen;
R⁴ is H, halogen, or C₁₋₆alkyl;
A₁ is N or CR⁵;
R⁵ is H, halogen, C₁₋₆alkyl, haloC₁₋₆alkyl, C₁₋₆alkoxy, or C₃₋₇cycloalkyl;
A₂ is N or CR⁶;
R⁶ is H, halogen, C₁₋₆alkyl, hydroxyC₁₋₆alkyl, deuterated C₁₋₆alkyl, haloC₁₋₆alkyl, C₃₋₇cycloalkylC₁₋₆alkyl, C₃₋₇cycloalkyl, haloC₃₋₇cycloalkyl, C₁₋₆alkylC₃₋₇cycloalkyl, C₁₋₆alkoxy, haloC₁₋₆alkoxy, C₃₋₇cycloalkoxy, C₂₋₆alkenyl, C₂₋₆alkynyl, cyano, C₁₋₆alkylsulfanyl, C₁₋₆alkylsulfinyl, C₁₋₆alkylsulfonyl, aryl, 5 to 8 membered heterocyclyl containing one to three heteroatoms selected from N, O, and S, or 5 to 8 membered heteroaryl containing one to three heteroatoms selected from N, O, and S, wherein each of aryl, heterocyclyl, and heteroaryl is independently optionally substituted with halogen, C₁₋₆alkyl, haloC₁₋₆alkyl, or C₁₋₆alkylcarbamoyl;
A₃ is N or CR⁷;
R⁷ is H, halogen, C₁₋₆alkyl, hydroxyC₁₋₆alkyl, haloC₁₋₆alkyl, (C₁₋₆alkylamino)C₁₋₆alkyl, C₃₋₇cycloalkyl, C₁₋₆alkoxy, haloC₁₋₆alkoxy, C₃₋₇cycloalkylC₁₋₆alkoxy, (C₁₋₆alkylamino)C₁₋₆alkoxy, (hyroxyl)(C₁₋₆alkyl)C₁₋₆alkoxy, (cyanoC₃₋₇cycloalkyl)C₁₋₆alkoxy, C₁₋₆alkoxy-oxo-C₁₋₆alkoxy, C₃₋₇cycloalkoxy, cyanoC₃₋₇cycloalkoxy, haloC₃₋₇cycloalkoxy, (hyroxyl)(C₁₋₆alkyl)C₃₋₇cycloalkoxy, C₁₋₆alkoxycarbonylC₃₋₇cycloalkoxy, C₁₋₆alkylamino, C₂₋₆alkenyl, C₂₋₆alkynyl, hydroxyC₂₋₆alkynyl, C₁₋₆alkoxyC₂₋₆alkynyl, morpholinoC₂₋₆alkynyl, (hyroxyl)(C₁₋₆alkyl)C₂₋₆alkynyl, (C₁₋₆alkoxy)(C₁₋₆alkyl)C₂₋₆alkynyl, (hydroxytetrahydrofuranyl)C₂₋₆alkynyl, (C₁₋₆alkylamino)C₂₋₆alkynyl, (amino)(C₁₋₆alkyl)C₂₋₆alkynyl, C₁₋₆alkylC₂₋₆alkynyl, (hydroxy)(C₁₋₆alkyl)C₂₋₆alkynyl, oxetanylC₂₋₆alkynyl, C₁₋₆alkoxycarbonyl, acyl, carboxy, cyano, or 5 to 8 membered heterocyclyl containing one to three heteroatoms selected from N, O, and S, wherein each of heterocyclyl and heteroaryl is independently optionally substituted with C₁₋₆alkyl, C₃₋₇cycloalkyl, haloC₁₋₆alkyl, or hydroxyl;
A₄ is N or CR⁸;
R⁸ is H, halogen, C₁₋₆alkyl, C₁₋₆alkoxy, C₁₋₆alkoxyC₁₋₆alkyl, haloC₁₋₆alkoxy, C₃₋₇cycloalkylC₁₋₆alkoxy, C₃₋₇cycloalkoxy, cyano, or benzyloxy;
or a pharmaceutically acceptable salt thereof.

3. (canceled)

4. The compound or pharmaceutically acceptable salt thereof according to claim 1, wherein R¹ is H.

5. The compound or pharmaceutically acceptable salt thereof according to claim 1, wherein R² is H, fluoro, chloro, methyl, or trifluoromethyl.

6. (canceled)

7. The compound or pharmaceutically acceptable salt thereof according to claim 5, wherein R² is fluoro or chloro.

8. The compound or pharmaceutically acceptable salt thereof according to claim 1, wherein R³ is H or fluoro.

9. (canceled)

10. The compound or pharmaceutically acceptable salt thereof according to claim 1, wherein R⁴ is H, fluoro, chloro, or methyl.

11. (canceled)

12. The compound or pharmaceutically acceptable salt thereof according to claim 1, wherein R⁵ is H, chloro, fluoro, methyl, difluoromethyl, methoxy, or cyclopropyl.

13. (canceled)

14. The compound or pharmaceutically acceptable salt thereof according to claim 12, wherein R⁵ is H, chloro, or methyl.

15. The compound or pharmaceutically acceptable salt thereof according to claim 1, wherein R⁶ is H, fluoro, chloro, bromo, iodo, methyl, trideuteriomethyl, ethyl, isopropyl, trifluoromethyl, trifluoroethyl, difluoroethyl, hydroxyisopropyl, cyclopropylmethyl, cyclopropyl, cyclopentyl, cyclobutyl, fluorocyclopropyl, methylcyclopropyl, dimethylcyclopropyl, methoxy, ethoxy, isopropoxy, trifluoroethoxy, cyclobutoxy, vinyl, isopropenyl, ethynyl, azetidinyl, oxetanyl, morpholino, cyano, methylsulfanyl, methylsulfinyl, methylsulfonyl, phenyl, or a heteroaryl selected from imidazolyl, pyrazolyl, isothiazolyl, pyridyl, pyridazinyl, and thiazolyl, wherein each of phenyl and heteroaryl is independently optionally substituted with fluoro, methyl, trifluoromethyl, or dimethylcarbamoyl.

16. (canceled)

17. (canceled)

18. The compound or pharmaceutically acceptable salt thereof according to claim 15, wherein R⁶ is H, chloro, methyl, trideuteriomethyl, ethyl, isopropyl, difluoroethyl, trifluoroethyl, cyclopropyl, or methylsulfanyl.

19. The compound or pharmaceutically acceptable salt thereof according to claim 1, wherein R⁷ is H, chloro, bromo, methyl, ethyl, hydroxyisopentyl, hydroxyisopropyl, trifluoromethyl, cyclopropyl, 3-(dimethylamino)propyl, methoxy, propoxy, isopropoxy, 1-bicyclo[1.1.1]pentanylmethoxy, difluoromethoxy, trifluoroethoxy, difluoroethoxy, difluoropropoxy, (dimethylamino)ethoxy, methylpropoxy, cyclopropylmethoxy, (1-cyanocyclopropyl)methoxy, 2-ethoxy-2-oxo-ethoxy, cyclobutoxy, cyanocyclobutoxy, fluorocyclobutoxy, 3-(1-methyl-ethyl)cyclobutoxy, 3-methoxycarbonylcyclobutoxy, isopropylamino, vinyl, ethynyl, hydroxybutynyl, methoxypropynyl, morpholinopropynyl, (hydroxyl)(methyl)butynyl, methoxymethylbutynyl, 2-(4-hydroxytetrahydrofuran-3-yl)-1-ethynyl, dimethylaminopropynyl, 3-methyl-1-but-1-ynyl, methylbutynyl, 3-hydroxy-3-methyl-butynyl, 3-methoxy-3-methyl-butynyl, 2-(oxetan-3-yl)-ethynyl, methoxycarbonyl, carboxy, cyano, acetyl, thienyl, thiazolyl, oxazolyl, pyridyl, or a heterocyclyl selected from pyrrolidinyl, morpholino, 2-oxa-6-azaspiro[3.3]heptanyl, 2,6-diazaspiro[3.3]heptanyl, 2,5-diazabicyclo[2.2.1]heptanyl, piperazinyl, 2,5-diazabicyclo[2.2.2]octanyl, piperidyl, oxetanyl, azetidinyl, and 2-oxa-6-azaspiro[3.3]heptanyl, wherein heterocyclyl is optionally substituted with methyl, isopropyl, cyclopropyl, trifluoroethyl, or hydroxyl.

20. (canceled)

21. (canceled)

22. The compound or pharmaceutically acceptable salt thereof according to claim 19, wherein R⁷ is H, chloro, methyl, cyclopropyl, cyclobutoxy, cyclopropylmethoxy, 3-hydroxy-3-methyl-1-but-1-ynyl, or 3-hydroxybut-1-ynyl.

23. The compound or pharmaceutically acceptable salt thereof according to claim 1, wherein R⁸ is H, chloro, fluoro, methyl, ethyl, methoxy, ethoxy, propoxy, isopropoxy, methoxymethyl, difluoromethoxy, trifluoromethoxy, 2,2,2-trifluoroethoxy, 2,2-difluoroethoxy, 3,3-difluoropropoxy, cyclopropylmethoxy, cyclopropoxy, cyclobutoxy, cyano, or benzyloxy.

24. (canceled)

25. The compound or pharmaceutically acceptable salt thereof according to claim 23, wherein R⁸ is ethoxy, difluoromethoxy, trifluoromethoxy, 2,2,2-trifluoroethoxy, cyclopropoxy, cyclobutoxy, or cyano.

26. The compound or pharmaceutically acceptable salt thereof according to claim 1, wherein:
R¹ is H;
R² is halogen;
R³ is H;
R⁴ is H;
A₁ is CR⁵;
R⁵ is H, halogen, or C₁₋₆alkyl;
A₂ is N or CR⁶;
R⁶ is H, halogen, C₁₋₆alkyl, deuterated C₁₋₆alkyl, haloC₁₋₆alkyl, C₃₋₇cycloalkyl, or C₁₋₆alkylsulfanyl;
A₃ is N or CR⁷;
R⁷ is H, halogen, C₁₋₆alkyl, C₃₋₇cycloalkyl, C₃₋₇cycloalkoxy, C₃₋₇cycloalkylC₁₋₆alkoxy, (hyroxyl)(C₁₋₆alkyl)C₂₋₆alkynyl, or hydroxyC₂₋₆alkynyl;
A₄ is N or CR⁸; and
R⁸ is C₁₋₆alkoxy, haloC₁₋₆alkoxy, C₃₋₇cycloalkoxy, or cyano.

27. The compound or pharmaceutically acceptable salt thereof according to claim 26, wherein:
R¹ is H;
R² is fluoro or chloro;
R³ is H;
R⁴ is H;
A₁ is CR⁵;
R⁵ is H, chloro, or methyl;
A₂ is N or CR⁶;
R⁶ is H, chloro, methyl, trideuteriomethyl, ethyl, isopropyl, difluoroethyl, trifluoroethyl, cyclopropyl, or methylsulfanyl;
A₃ is N or CR⁷;
R⁷ is H, chloro, methyl, cyclopropyl, cyclobutoxy, cyclopropylmethoxy, 3-hydroxy-3-methyl-1-but-1-ynyl, or 3-hydroxybut-1-ynyl;
A₄ is N or CR⁸; and
R⁸ is ethoxy, difluoromethoxy, trifluoromethoxy, 2,2,2-trifluoroethoxy, cyclopropoxy, cyclobutoxy, or cyano.

28. A compound selected from:
3-Amino-7-chloro-4-(7-fluoro-1H-indazol-4-yl)-1H-1,5-naphthyridin-2-one,
3-Amino-4-(7-fluoro-1H-indazol-4-yl)-7-methyl-1H-1,5-naphthyridin-2-one,
3-Amino-6-chloro-4-(7-fluoro-1H-indazol-4-yl)-7-methyl-1H-1,5-naphthyridin-2-one,
3-Amino-7-fluoro-4-(7-fluoro-1H-indazol-4-yl)-1H-1,5-naphthyridin-2-one,
3-Amino-4-(7-fluoro-1H-indazol-4-yl)-7-(trifluoromethyl)-1H-1,5-naphthyridin-2-one,
3-Amino-4-(7-fluoro-1H-indazol-4-yl)-7-methoxy-1H-1,5-naphthyridin-2-one,
4-[7-Amino-8-(7-fluoro-1H-indazol-4-yl)-6-oxo-5H-1,5-naphthyridin-3-yl]-N,N-dimethyl-benzamide,
3-Amino-4-(7-fluoro-1H-indazol-4-yl)-7-(3-methylimidazol-4-yl)-1H-1,5-naphthyridin-2-one,
3-Amino-4-(7-fluoro-1H-indazol-4-yl)-7-(1H-pyrazol-5-yl)-1H-1,5-naphthyridin-2-one,
3-Amino-4-(7-fluoro-1H-indazol-4-yl)-7-isothiazol-5-yl-1H-1,5-naphthyridin-2-one,
3-Amino-4-(7-fluoro-1H-indazol-4-yl)-7-[2-(trifluoromethyl)-4-pyridyl]-1H-1,5-naphthyridin-2-one,
3-Amino-4-(7-fluoro-1H-indazol-4-yl)-7-pyridazin-4-yl-1H-1,5-naphthyridin-2-one,
3-Amino-4-(7-fluoro-1H-indazol-4-yl)-7-(2-pyridyl)-1H-1,5-naphthyridin-2-one,
3-Amino-4-(7-fluoro-1H-indazol-4-yl)-7-(5-fluoro-2-pyridyl)-1H-1,5-naphthyridin-2-one,
3-Amino-7-cyclopropyl-4-(7-fluoro-1H-indazol-4-yl)-1H-1,5-naphthyridin-2-one,
3-Amino-4-(7-fluoro-1H-indazol-4-yl)-7-thiazol-2-yl-1H-1,5-naphthyridin-2-one,
3-Amino-4-(7-fluoro-1H-indazol-4-yl)-6,7-dimethyl-1H-1,5-naphthyridin-2-one,
3-Amino-6-cyclopropyl-4-(7-fluoro-1H-indazol-4-yl)-7-methyl-1H-1,5-naphthyridin-2-one,
3-Amino-4-(7-fluoro-1H-indazol-4-yl)-7-morpholino-1H-1,5-naphthyridin-2-one,
3-Amino-4-(7-fluoro-1H-indazol-4-yl)-7-(1-hydroxy-1-methyl-ethyl)-1H-1,5-naphthyridin-2-one,
3-Amino-6-bromo-4-(7-fluoro-1H-indazol-4-yl)-7-methyl-1H-1,5-naphthyridin-2-one,
3-Amino-4-(7-fluoro-1H-indazol-4-yl)-7-methyl-6-(4-methylpiperazin-1-yl)-1H-1,5-naphthyridin-2-one,
3-Amino-4-(7-fluoro-1H-indazol-4-yl)-6-(isopropylamino)-7-methyl-1H-1,5-naphthyridin-2-one,
3-Amino-4-(7-fluoro-1H-indazol-4-yl)-6-(3-hydroxypyrrolidin-1-yl)-7-methyl-1H-1,5-naphthyridin-2-one,
3-Amino-4-(7-fluoro-1H-indazol-4-yl)-7-methyl-6-morpholino-1H-1,5-naphthyridin-2-one,
3-Amino-4-(7-fluoro-1H-indazol-4-yl)-7-methyl-6-(2-oxa-6-azaspiro[3.3]heptan-6-yl)-1H-1,5-naphthyridin-2-one,
3-Amino-4-(7-fluoro-1H-indazol-4-yl)-7-methyl-6-(6-methyl-2,6-diazaspiro[3.3]heptan-2-yl)-1H-1,5-naphthyridin-2-one,
3-Amino-4-(7-fluoro-1H-indazol-4-yl)-6-(4-isopropylpiperazin-1-yl)-7-methyl-1H-1,5-naphthyridin-2-one,
3-Amino-6-(4-cyclopropylpiperazin-1-yl)-4-(7-fluoro-1H-indazol-4-yl)-7-methyl-1H-1,5-naphthyridin-2-one,
3-Amino-4-(7-fluoro-1H-indazol-4-yl)-7-methyl-6-(5-methyl-2,5-diazabicyclo[2.2.1]heptan-2-yl)-1H-1,5-naphthyridin-2-one,
3-Amino-4-(7-fluoro-1H-indazol-4-yl)-7-methyl-6-(5-methyl-2,5-diazabicyclo[2.2.2]octan-2-yl)-1H-1,5-naphthyridin-2-one,
3-Amino-4-(7-fluoro-1H-indazol-4-yl)-6-(3-hydroxy-3-methyl-butyl)-7-methyl-1H-1,5-naphthyridin-2-one,
3-Amino-4-(1H-indazol-4-yl)-7-methyl-1H-1,5-naphthyridin-2-one,
3-Amino-7-cyclopropyl-4-(1H-indazol-4-yl)-1H-1,5-naphthyridin-2-one,
3-Amino-4-(1H-indazol-4-yl)-7-thiazol-2-yl-1H-1,5-naphthyridin-2-one,
3-Amino-7-chloro-4-(6-fluoro-1H-indazol-4-yl)-1H-1,5-naphthyridin-2-one,
3-Amino-7-chloro-4-(7-chloro-1H-indazol-4-yl)-1H-1,5-naphthyridin-2-one,
3-Amino-7-chloro-4-(7-methyl-1H-indazol-4-yl)-1H-1,5-naphthyridin-2-one,
3-Amino-7-chloro-4-(6,7-difluoro-1H-indazol-4-yl)-1H-1,5-naphthyridin-2-one,
3-Amino-7-chloro-4-(5-fluoro-1H-indazol-4-yl)-1H-1,5-naphthyridin-2-one,
3-Amino-7-chloro-4-[7-(trifluoromethyl)-1H-indazol-4-yl]-1H-1,5-naphthyridin-2-one,
3-Amino-7-chloro-4-(7-fluoro-5-methyl-1H-indazol-4-yl)-1H-1,5-naphthyridin-2-one,
3-Amino-7-chloro-4-(5-chloro-7-fluoro-1H-indazol-4-yl)-1H-1,5-naphthyridin-2-one,
3-Amino-4-(7-fluoro-1H-indazol-4-yl)-7-methyl-6-piperazin-1-yl-1H-1,5-naphthyridin-2-one,
3-Amino-7-chloro-4-(3-methyl-1H-indazol-4-yl)-1H-1,5-naphthyridin-2-one,
3-Amino-6-(cyclobutoxy)-4-(7-fluoro-1H-indazol-4-yl)-7-methyl-1H-1,5-naphthyridin-2-one,
3-Amino-6-(cyclopropylmethoxy)-4-(7-fluoro-1H-indazol-4-yl)-7-methyl-1H-1,5-naphthyridin-2-one,
3-Amino-4-(7-fluoro-1H-indazol-4-yl)-6,7-dimethyl-1H-quinolin-2-one,
3-Amino-6-chloro-4-(7-fluoro-1H-indazol-4-yl)-7,8-dimethyl-1H-1,5-naphthyridin-2-one,
3-Amino-8-chloro-4-(7-fluoro-1H-indazol-4-yl)-1H-1,5-naphthyridin-2-one,
3-Amino-4-(7-fluoro-1H-indazol-4-yl)-8-methoxy-7-methyl-1H-1,5-naphthyridin-2-one,
3-Amino-7-chloro-4-(7-fluoro-1H-indazol-4-yl)-6-methyl-1H-1,5-naphthyridin-2-one,
3-Amino-8-chloro-4-(7-fluoro-1H-indazol-4-yl)-7-methyl-1H-1,5-naphthyridin-2-one,
3-Amino-4-(7-fluoro-1H-indazol-4-yl)-7,8-dimethyl-1H-1,5-naphthyridin-2-one,
3-Amino-7-chloro-6-cyclopropyl-4-(7-fluoro-1H-indazol-4-yl)-1H-1,5-naphthyridin-2-one,
3-Amino-7-chloro-4-(7-fluoro-1H-indazol-4-yl)-6,8-dimethyl-1H-1,5-naphthyridin-2-one,
3-Amino-8-chloro-4-(7-fluoro-1H-indazol-4-yl)-6,7-dimethyl-1H-1,5-naphthyridin-2-one,
1-[[7-Amino-8-(7-fluoro-1H-indazol-4-yl)-3-methyl-6-oxo-5H-1,5-naphthyridin-2-yl]oxymethyl]cyclopropanecarbonitrile,
3-Amino-6-(2,2-difluoroethoxy)-4-(7-fluoro-1H-indazol-4-yl)-7-methyl-1H-1,5-naphthyridin-2-one,
3-Amino-4-(7-fluoro-1H-indazol-4-yl)-6-methoxy-7-methyl-1H-1,5-naphthyridin-2-one,
Ethyl 2-[[7-amino-8-(7-fluoro-1H-indazol-4-yl)-3-methyl-6-oxo-5H-1,5-naphthyridin-2-yl]oxy]acetate,
3-Amino-6-(difluoromethoxy)-4-(7-fluoro-1H-indazol-4-yl)-7-methyl-1H-1,5-naphthyridin-2-one,
3-Amino-6-(1-bicyclo[1.1.1]pentanylmethoxy)-4-(7-fluoro-1H-indazol-4-yl)-7-methyl-1H-1,5-naphthyridin-2-one,
3-Amino-4-(7-fluoro-1H-indazol-4-yl)-6-isopropoxy-7-methyl-1H-1,5-naphthyridin-2-one,
3-Amino-4-(7-fluoro-1H-indazol-4-yl)-7-methyl-6-propoxy-1H-1,5-naphthyridin-2-one,
3-Amino-4-(7-fluoro-1H-indazol-4-yl)-7-methyl-6-(2,2,2-trifluoroethoxy)-1H-1,5-naphthyridin-2-one,
Methyl 3-[[7-amino-8-(7-fluoro-1H-indazol-4-yl)-3-methyl-6-oxo-5H-1,5-naphthyridin-2-yl]oxy]cyclobutanecarboxylate,
3-Amino-6-[2-(dimethylamino)ethoxy]-4-(7-fluoro-1H-indazol-4-yl)-7-methyl-1H-1,5-naphthyridin-2-one,
3-Amino-4-(7-fluoro-1H-indazol-4-yl)-7-methyl-6-(4-piperidyl)-1H-1,5-naphthyridin-2-one,
3-Amino-4-(7-fluoro-1H-indazol-4-yl)-6-(3-hydroxy-3-methyl-but-1-ynyl)-7-methyl-1H-1,5-naphthyridin-2-one,
3-Amino-4-(7-fluoro-1H-indazol-4-yl)-6-(3-methoxyprop-1-ynyl)-7-methyl-1H-1,5-naphthyridin-2-one,
3-Amino-4-(7-fluoro-1H-indazol-4-yl)-6-(3-methoxy-3-methyl-but-1-ynyl)-7-methyl-1H-1,5-naphthyridin-2-one,
3-Amino-4-(7-fluoro-1H-indazol-4-yl)-6-[2-(4-hydroxytetrahydrofuran-3-yl)ethynyl]-7-methyl-1H-1,5-naphthyridin-2-one,
3-Amino-4-(7-fluoro-1H-indazol-4-yl)-6-(3-hydroxybut-1-ynyl)-7-methyl-1H-1,5-naphthyridin-2-one,
3-Amino-6-[3-(dimethylamino)prop-1-ynyl]-4-(7-fluoro-1H-indazol-4-yl)-7-methyl-1H-1,5-naphthyridin-2-one,
3-Amino-4-(7-fluoro-1H-indazol-4-yl)-7-methyl-6-(3-morpholinoprop-1-ynyl)-1H-1,5-naphthyridin-2-one,
3-Amino-6-(3-amino-3-methyl-but-1-ynyl)-4-(7-fluoro-1H-indazol-4-yl)-7-methyl-1H-1,5-naphthyridin-2-one,
3-Amino-4-(7-fluoro-1H-indazol-4-yl)-6-(2-hydroxy-2-methyl-propoxy)-7-methyl-1H-1,5-naphthyridin-2-one,
3-[[7-Amino-8-(7-fluoro-1H-indazol-4-yl)-3-methyl-6-oxo-5H-1,5-naphthyridin-2-yl]oxy]cyclobutanecarbonitrile,
trans-3-Amino-6-(3-fluorocyclobutoxy)-4-(7-fluoro-1H-indazol-4-yl)-7-methyl-1H-1,5-naphthyridin-2-one,
3-Amino-6-(3,3-difluoropropoxy)-4-(7-fluoro-1H-indazol-4-yl)-7-methyl-1H-1,5-naphthyridin-2-one,
3-Amino-4-(7-fluoro-1H-indazol-4-yl)-7-methyl-6-(1-methyl-4-piperidyl)-1H-1,5-naphthyridin-2-one,
7-Amino-8-(7-fluoro-1H-indazol-4-yl)-2,3-dimethyl-5H-pyrido[2,3-b]pyrazin-6-one,
7-Amino-8-(7-fluoro-1H-indazol-4-yl)-2,4-dimethyl-5H-pyrido[3,2-d]pyrimidin-6-one,
3-Amino-7-cyclopropyl-4-(7-fluoro-1H-indazol-4-yl)-6-methyl-1H-1,5-naphthyridin-2-one,
3-Amino-6-[3-(dimethylamino)propyl]-4-(7-fluoro-1H-indazol-4-yl)-7-methyl-1H-1,5-naphthyridin-2-one,
Methyl 7-amino-8-(7-fluoro-1H-indazol-4-yl)-3-methyl-6-oxo-5H-1,5-naphthyridine-2-carboxylate,
3-Amino-4-(7-fluoro-1H-indazol-4-yl)-6-(1-hydroxy-1-methyl-ethyl)-7-methyl-1H-1,5-naphthyridin-2-one,
7-Amino-8-(7-fluoro-1H-indazol-4-yl)-3-methyl-6-oxo-5H-1,5-naphthyridine-2-carboxylic acid,
3-Amino-8-chloro-4-(7-fluoro-1H-indazol-4-yl)-6-methyl-1H-1,5-naphthyridin-2-one,
3-Amino-6-chloro-4-(7-fluoro-1H-indazol-4-yl)-8-methyl-1H-1,5-naphthyridin-2-one,
7-Amino-8-(7-fluoro-1H-indazol-4-yl)-3-methyl-6-oxo-5H-1,5-naphthyridine-2-carbonitrile,
3-Amino-4-(7-fluoro-1H-indazol-4-yl)-7-methyl-6-(oxetan-3-yl)-1H-1,5-naphthyridin-2-one,
3-Amino-4-(7-fluoro-1H-indazol-4-yl)-6,8-dimethyl-1H-1,5-naphthyridin-2-one,
3-Amino-6-chloro-7-cyclopropyl-4-(7-fluoro-1H-indazol-4-yl)-1H-1,5-naphthyridin-2-one,
3-Amino-4-(7-fluoro-1H-indazol-4-yl)-6-[3-(1-hydroxy-1-methyl-ethyl)cyclobutoxy]-7-methyl-1H-1,5-naphthyridin-2-one,
3-Amino-4-(7-fluoro-1H-indazol-4-yl)-7-methyl-6-(trifluoromethyl)-1H-1,5-naphthyridin-2-one,
3-Amino-4-(7-fluoro-1H-indazol-4-yl)-7-methyl-6-(1-methylazetidin-3-yl)-1H-1,5-naphthyridin-2-one,
3-Amino-7-chloro-4-(7-fluoro-1H-indazol-4-yl)-8-methyl-1H-1,5-naphthyridin-2-one,
3-Amino-7-cyclopropyl-4-(7-fluoro-1H-indazol-4-yl)-8-methyl-1H-1,5-naphthyridin-2-one, or
3-Amino-4-(7-fluoro-1H-indazol-4-yl)-8-methyl-7-(trideuteriomethyl)-1H-1,5-naphthyridin-2-one,
3-Amino-4-(7-fluoro-1H-indazol-4-yl)-5-methoxy-7-methyl-1H-quinolin-2-one,
3-Amino-5-chloro-4-(7-fluoro-1H-indazol-4-yl)-7-methyl-1H-quinolin-2-one,
3-Amino-6-bromo-4-(7-fluoro-1H-indazol-4-yl)-7-methyl-1H-quinolin-2-one,
3-Amino-4-(7-fluoro-1H-indazol-4-yl)-7-methyl-6-(2-oxa-6-azaspiro[3.3]heptan-6-yl)-1H-quinolin-2-one,
3-Amino-4-(7-fluoro-1H-indazol-4-yl)-7-methyl-6-[1-(2,2,2-trifluoroethyl)-4-piperidyl]-1H-quinolin-2-one,
3-Amino-4-(7-fluoro-1H-indazol-4-yl)-6,7-dimethyl-1H-1,8-naphthyridin-2-one,
3-Amino-6-bromo-8-chloro-4-(7-fluoro-1H-indazol-4-yl)-7-methyl-1H-1,5-naphthyridin-2-one,
3-Amino-8-chloro-6-cyclopropyl-4-(7-fluoro-1H-indazol-4-yl)-7-methyl-1H-1,5-naphthyridin-2-one,
3-Amino-4-(7-fluoro-1H-indazol-4-yl)-6,7,8-trimethyl-1H-1,5-naphthyridin-2-one,
3-Amino-8-chloro-6-ethynyl-4-(7-fluoro-1H-indazol-4-yl)-7-methyl-1H-1,5-naphthyridin-2-one,
6-Acetyl-3-amino-8-chloro-4-(7-fluoro-1H-indazol-4-yl)-7-methyl-1H-1,5-naphthyridin-2-one,
3-Amino-8-chloro-4-(7-fluoro-1H-indazol-4-yl)-7-methyl-6-vinyl-1H-1,5-naphthyridin-2-one,
3-Amino-6,8-dicyclopropyl-4-(7-fluoro-1H-indazol-4-yl)-7-methyl-1H-1,5-naphthyridin-2-one,
3-Amino-7-bromo-4-(7-fluoro-1H-indazol-4-yl)-8-methyl-1H-1,5-naphthyridin-2-one,
3-Amino-4-(7-fluoro-1H-indazol-4-yl)-7-iodo-8-methyl-1H-1,5-naphthyridin-2-one,
3-Amino-7-(azetidin-1-yl)-4-(7-fluoro-1H-indazol-4-yl)-8-methyl-1H-1,5-naphthyridin-2-one,
7-Amino-8-(7-fluoro-1H-indazol-4-yl)-4-methyl-6-oxo-5H-1,5-naphthyridine-3-carbonitrile,
3-Amino-7-cyclobutyl-4-(7-fluoro-1H-indazol-4-yl)-8-methyl-1H-1,5-naphthyridin-2-one,
3-Amino-4-(7-fluoro-1H-indazol-4-yl)-8-methyl-7-pyridazin-4-yl-1H-1,5-naphthyridin-2-one,
3-Amino-4-(7-fluoro-1H-indazol-4-yl)-8-methyl-7-thiazol-2-yl-1H-1,5-naphthyridin-2-one,
3-Amino-4-(7-fluoro-1H-indazol-4-yl)-8-methyl-7-(oxetan-3-yl)-1H-1,5-naphthyridin-2-one,
3-Amino-4-(7-fluoro-1H-indazol-4-yl)-8-methyl-7-vinyl-1H-1,5-naphthyridin-2-one,
3-Amino-7-ethynyl-4-(7-fluoro-1H-indazol-4-yl)-8-methyl-1H-1,5-naphthyridin-2-one,
3-Amino-4-(7-fluoro-1H-indazol-4-yl)-7-isopropenyl-8-methyl-1H-1,5-naphthyridin-2-one,
3-Amino-7-cyclopentyl-4-(7-fluoro-1H-indazol-4-yl)-8-methyl-1H-1,5-naphthyridin-2-one,
3-Amino-4-(7-fluoro-1H-indazol-4-yl)-7-isopropyl-8-methyl-1H-1,5-naphthyridin-2-one,
3-Amino-7-ethyl-4-(7-fluoro-1H-indazol-4-yl)-8-methyl-1H-1,5-naphthyridin-2-one,
3-Amino-4-(7-fluoro-1H-indazol-4-yl)-8-methyl-7-(1-methylcyclopropyl)-1H-1,5-naphthyridin-2-one,
3-Amino-7-(2,2-dimethylcyclopropyl)-4-(7-fluoro-1H-indazol-4-yl)-8-methyl-1H-1,5-naphthyridin-2-one,
3-Amino-7-(cyclopropylmethyl)-4-(7-fluoro-1H-indazol-4-yl)-8-methyl-1H-1,5-naphthyridin-2-one,
3-Amino-7-(2-fluorocyclopropyl)-4-(7-fluoro-1H-indazol-4-yl)-8-methyl-1H-1,5-naphthyridin-2-one,
3-Amino-4-(7-fluoro-1H-indazol-4-yl)-8-methyl-7-(2,2,2-trifluoroethyl)-1H-1,5-naphthyridin-2-one,
3-Amino-7-(1,1-difluoroethyl)-4-(7-fluoro-1H-indazol-4-yl)-8-methyl-1H-1,5-naphthyridin-2-one,
3-Amino-7-fluoro-4-(7-fluoro-1H-indazol-4-yl)-8-methyl-1H-1,5-naphthyridin-2-one,
3-Amino-4-(7-fluoro-1H-indazol-4-yl)-7-methoxy-8-methyl-1H-1,5-naphthyridin-2-one,
3-Amino-4-(7-fluoro-1H-indazol-4-yl)-8-methyl-7-(2,2,2-trifluoroethoxy)-1H-1,5-naphthyridin-2-one,
3-Amino-4-(7-fluoro-1H-indazol-4-yl)-7-isopropoxy-8-methyl-1H-1,5-naphthyridin-2-one,
3-Amino-7-(cyclobutoxy)-4-(7-fluoro-1H-indazol-4-yl)-8-methyl-1H-1,5-naphthyridin-2-one,
3-Amino-7-ethoxy-4-(7-fluoro-1H-indazol-4-yl)-8-methyl-1H-1,5-naphthyridin-2-one,
3-Amino-4-(7-fluoro-1H-indazol-4-yl)-8-methyl-7-methylsulfanyl-1H-1,5-naphthyridin-2-one,
3-Amino-4-(7-fluoro-1H-indazol-4-yl)-8-methyl-7-methylsulfinyl-1H-1,5-naphthyridin-2-one,
3-Amino-4-(7-fluoro-1H-indazol-4-yl)-8-methyl-7-methylsulfonyl-1H-1,5-naphthyridin-2-one,
3-Amino-4-(7-fluoro-1H-indazol-4-yl)-8-methyl-7-[rac-(1R,2R)-2-methylcyclopropyl]-1H-1,5-naphthyridin-2-one,
3-Amino-8-(difluoromethyl)-4-(7-fluoro-1H-indazol-4-yl)-7-methyl-1H-1,5-naphthyridin-2-one,
3-Amino-4-(7-chloro-1H-indazol-4-yl)-7,8-dimethyl-1H-1,5-naphthyridin-2-one,
3-Amino-6-bromo-4-(7-fluoro-1H-indazol-4-yl)-8-methyl-1H-1,5-naphthyridin-2-one,
3-Amino-4-(7-fluoro-1H-indazol-4-yl)-6-(3-hydroxy-3-methyl-but-1-ynyl)-8-methyl-1H-1,5-naphthyridin-2-one,
3-Amino-4-(7-fluoro-1H-indazol-4-yl)-6-(3-methoxy-3-methyl-but-1-ynyl)-8-methyl-1H-1,5-naphthyridin-2-one,
3-Amino-4-(7-fluoro-1H-indazol-4-yl)-8-methyl-6-[2-(oxetan-3-yl)ethynyl]-1H-1,5-naphthyridin-2-one,
3-Amino-4-(7-fluoro-1H-indazol-4-yl)-7-methyl-6-(2-thienyl)-1H-1,5-naphthyridin-2-one,
3-Amino-4-(7-fluoro-1H-indazol-4-yl)-7-methyl-6-thiazol-4-yl-1H-1,5-naphthyridin-2-one,
3-Amino-4-(7-fluoro-1H-indazol-4-yl)-7-methyl-6-thiazol-2-yl-1H-1,5-naphthyridin-2-one,
3-Amino-4-(7-fluoro-1H-indazol-4-yl)-7-methyl-6-oxazol-2-yl-1H-1,5-naphthyridin-2-one,
3-Amino-4-(7-fluoro-1H-indazol-4-yl)-7-methyl-6-(2-pyridyl)-1H-1,5-naphthyridin-2-one,
3-Amino-4-(7-fluoro-1H-indazol-4-yl)-7-methyl-6-(3-methylbut-1-ynyl)-1H-1,5-naphthyridin-2-one,
3-Amino-6-ethyl-4-(7-fluoro-1H-indazol-4-yl)-7-methyl-1H-1,5-naphthyridin-2-one,
3-Amino-7-ethyl-4-(7-fluoro-1H-indazol-4-yl)-1H-1,5-naphthyridin-2-one,
3-Amino-4-(7-chloro-1H-indazol-4-yl)-6,7-dimethyl-1H-1,5-naphthyridin-2-one,
3-Amino-5-chloro-4-(7-fluoro-1H-indazol-4-yl)-7,8-dimethyl-1H-1,6-naphthyridin-2-one,
3-Amino-4-(7-fluoro-1H-indazol-4-yl)-7,8-dimethyl-2-oxo-1H-1,6-naphthyridine-5-carbonitrile,
3-Amino-5-chloro-7-cyclopropyl-4-(7-fluoro-1H-indazol-4-yl)-8-methyl-1H-1,6-naphthyridin-2-one,
3-Amino-5-chloro-7-cyclopropyl-4-(7-fluoro-1H-indazol-4-yl)-1H-1,6-naphthyridin-2-one,
3-Amino-5-chloro-4-(7-fluoro-1H-indazol-4-yl)-8-methyl-1H-1,6-naphthyridin-2-one, 3-amino-7-cyclopropyl-4-(7-fluoro-1H-indazol-4-yl)-8-methyl-1H-1,6-naphthyridin-2-one,
3-Amino-7-cyclopropyl-4-(7-fluoro-1H-indazol-4-yl)-5-methyl-1H-1,6-naphthyridin-2-one,
3-Amino-7-cyclopropyl-4-(7-fluoro-1H-indazol-4-yl)-2-oxo-1H-1,6-naphthyridine-5-carbonitrile,
3-Amino-7-cyclopropyl-5-(difluoromethoxy)-4-(7-fluoro-1H-indazol-4-yl)-1H-1,6-naphthyridin-2-one,
3-Amino-7-cyclopropyl-4-(7-fluoro-1H-indazol-4-yl)-5-methoxy-1H-1,6-naphthyridin-2-one,
3-amino-8-fluoro-4-(7-fluoro-1H-indazol-4-yl)-5-methoxy-1H-1,6-naphthyridin-2-one,
3-Amino-8-chloro-4-(7-fluoro-1H-indazol-4-yl)-5-methoxy-1H-1,6-naphthyridin-2-one,
3-Amino-4-(7-fluoro-1H-indazol-4-yl)-5-methoxy-8-methyl-1H-1,6-naphthyridin-2-one,
3-Amino-4-(7-fluoro-1H-indazol-4-yl)-5-methoxy-8-methyl-1H-1,7-naphthyridin-2-one,
3-Amino-4-(7-fluoro-1H-indazol-4-yl)-5-methoxy-6,8-dimethyl-1H-1,7-naphthyridin-2-one,
3-Amino-6-chloro-4-(7-fluoro-1H-indazol-4-yl)-8-methyl-1H-1,7-naphthyridin-2-one,
3-Amino-5-chloro-4-(7-fluoro-1H-indazol-4-yl)-8-methyl-1H-1,7-naphthyridin-2-one,
3-Amino-4-(7-fluoro-1H-indazol-4-yl)-5,8-dimethyl-1H-1,7-naphthyridin-2-one,
3-Amino-5-ethyl-4-(7-fluoro-1H-indazol-4-yl)-8-methyl-1H-1,7-naphthyridin-2-one,
3-Amino-5-fluoro-4-(7-fluoro-1H-indazol-4-yl)-8-methyl-1H-1,7-naphthyridin-2-one,
3-Amino-5-benzyloxy-8-chloro-4-(7-fluoro-1H-indazol-4-yl)-1H-1,7-naphthyridin-2-one,
3-Amino-5-benzyloxy-4-(7-fluoro-1H-indazol-4-yl)-8-methyl-1H-1,7-naphthyridin-2-one,
3-Amino-4-(7-fluoro-1H-indazol-4-yl)-5-isopropoxy-8-methyl-1H-1,7-naphthyridin-2-one,
3-Amino-4-(7-fluoro-1H-indazol-4-yl)-8-methyl-5-(2,2,2-trifluoroethoxy)-1H-1,7-naphthyridin-2-one,
3-Amino-5-ethoxy-4-(7-fluoro-1H-indazol-4-yl)-8-methyl-1H-1,7-naphthyridin-2-one,
3-Amino-5-(difluoromethoxy)-4-(7-fluoro-1H-indazol-4-yl)-8-methyl-1H-1,7-naphthyridin-2-one,
3-Amino-5-(2,2-difluoroethoxy)-4-(7-fluoro-1H-indazol-4-yl)-8-methyl-1H-1,7-naphthyridin-2-one,
3-Amino-5-(cyclopropoxy)-4-(7-fluoro-1H-indazol-4-yl)-8-methyl-1H-1,7-naphthyridin-2-one,
3-Amino-5-(3,3-difluoropropoxy)-4-(7-fluoro-1H-indazol-4-yl)-8-methyl-1H-1,7-naphthyridin-2-one,
3-Amino-5-(cyclobutoxy)-4-(7-fluoro-1H-indazol-4-yl)-8-methyl-1H-1,7-naphthyridin-2-one,
3-Amino-4-(7-fluoro-1H-indazol-4-yl)-8-methyl-5-propoxy-1H-1,7-naphthyridin-2-one,
3-Amino-5-(cyclopropylmethoxy)-4-(7-fluoro-1H-indazol-4-yl)-8-methyl-1H-1,7-naphthyridin-2-one,
3-Amino-4-(7-fluoro-1H-indazol-4-yl)-8-methyl-5-(trifluoromethoxy)-1H-1,7-naphthyridin-2-one,
3-Amino-4-(7-fluoro-1H-indazol-4-yl)-5-(methoxymethyl)-8-methyl-1H-1,7-naphthyridin-2-one,
3-Amino-7-bromo-4-(7-fluoro-1H-indazol-4-yl)-6-methyl-1H-1,5-naphthyridin-2-one, and
3-Amino-7-ethyl-4-(7-fluoro-1H-indazol-4-yl)-6-methyl-1H-1,5-naphthyridin-2-one,
or a pharmaceutically acceptable salt thereof.

29. A process for the preparation of a compound having the structure of formula (I), 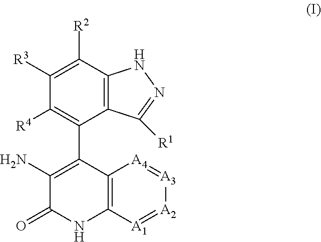
comprising one of the following steps:
(a) decomposition of pyridinium salt of formula (IX) 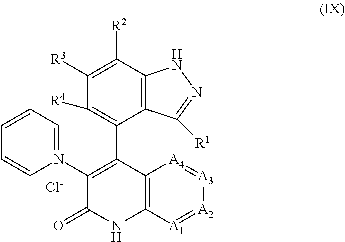
with hydrazine hydrate;
(b) cross coupling of compound of formula (XVII), 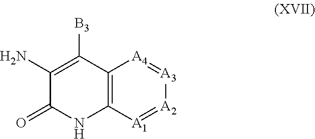
and compound of formula (XVIII), 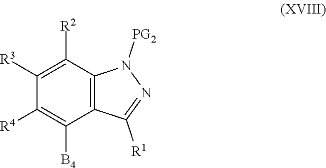
in the presence of a catalyst to provide compound of formula (XIX) 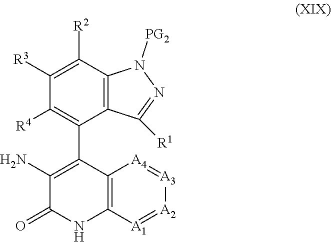
followed by deprotecting of the compound of formula (XIX) in the presence an acid,
(c) decomposition of pyridinium salts of formula (XXIV) 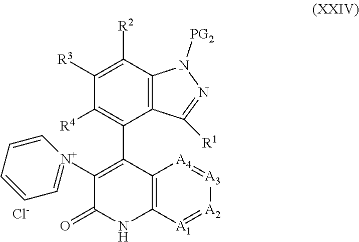
with hydrazine hydrate to afford compound of formula (XIX), 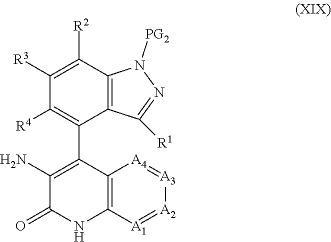
followed by deprotection of compound of formula (XIX) in the presence of an acid;
wherein B³ is halogen, preferably bromo or iodide; B⁴ is boronic acid, boronic ester or tributyltin, PG₂ is hydrogen or a protecting group, preferably tetrahydropyranyl group, the acid is preferably trifluoroacetic acid or HCl, the catalyst is preferably Pd(dtbpf)Cl₂, Pd(dppf)Cl₂ or Pd(PPh₃)₄;
wherein:
R¹ is H, halogen or C₁₋₆alkyl;
R² is H, halogen, cyano, C₃₋₇cycloalkyl, C₁₋₆alkyl, C₁₋₆alkoxy, or haloC₁₋₆alkyl;
R³ is H or halogen;
R⁴ is H, halogen, or C₁₋₆alkyl;
A₁ is N or CR⁵;
A₂ is N or CR⁶;
A₃ is N or CR⁷;
A₄ is N or CR⁸; and
each of R⁵, R⁶, R⁷ and R⁸ is independently selected from H, halogen, C₁₋₆alkyl, deuterated C₁₋₆alkyl, C₃₋₇cycloalkylC₁₋₆alkyl, (C₁₋₆alkylamino)C₁₋₆alkyl, hydroxyC₁₋₆alkyl, haloC₁₋₆alkyl, C₁₋₆alkoxyC₁₋₆alkyl, C₃₋₇cycloalkyl, haloC₃₋₇cycloalkyl, C₁₋₆alkylC₃₋₇cycloalkyl, C₁₋₆alkoxy, C₃₋₇cycloalkylC₁₋₆alkoxy, (C₁₋₆alkylamino)C₁₋₆alkoxy, (hyroxyl)(C₁₋₆alkyl)C₁₋₆alkoxy, (cyanoC₃₋₇cycloalkyl)C₁₋₆alkoxy, C₁₋₆alkoxy-oxo-C₁₋₆alkoxy, haloC₁₋₆alkoxy, C₃₋₇cycloalkoxy, cyanoC₃₋₇cycloalkoxy, haloC₃₋₇cycloalkoxy, (hyroxyl)(C₁₋₆alkyl)C₃₋₇cycloalkoxy, C₁₋₆alkoxycarbonylC₃₋₇cycloalkoxy, C₂₋₆alkenyl, C₂₋₆alkynyl, hydroxyC₂₋₆alkynyl, C₁₋₆alkoxyC₂₋₆alkynyl, morpholinoC₂₋₆alkynyl, (hyroxyl)(C₁₋₆alkyl)C₂₋₆alkynyl, (C₁₋₆alkoxy)(C₁₋₆alkyl)C₂₋₆alkynyl, (hydroxytetrahydrofuranyl)C₂₋₆alkynyl, (C₁₋₆alkylamino)C₂₋₆alkynyl, (amino)(C₁₋₆alkyl)C₂₋₆alkynyl, C₁₋₆alkoxycarbonyl, C₁₋₆alkylC₂₋₆alkynyl, (hydroxy)(C₁₋₆alkyl)C₂₋₆alkynyl, oxetanylC₂₋₆alkynyl, C₁₋₆alkylamino, acyl, cyano, carboxy, C₁₋₆alkylsulfanyl, C₁₋₆alkylsulfinyl, C₁₋₆alkylsulfonyl, benzyloxy, aryl, 5 to 8 membered heterocyclyl containing one to three heteroatoms selected from N, O, and S, and 5 to 8 membered heteroaryl containing one to three heteroatoms selected from N, O, and S, wherein each of aryl, heterocyclyl, and heteroaryl is independently optionally substituted with halogen, C₁₋₆alkyl, C₃₋₇cycloalkyl, C₁₋₆alkylcarbamoyl, haloC₁₋₆alkyl, or hydroxyl.

30. (canceled)

31. A pharmaceutical composition comprising a compound or pharmaceutically acceptable salt thereof according to claim 1, and a pharmaceutically acceptable excipient.

32-37. (canceled)

38. A method for the treatment of cancer, which method comprises administering an effective amount of the compound or pharmaceutically acceptable salt thereof according to claim 1.

39. (canceled)
